(19) United States
(12) Patent Application Publication
(10) Pub. No.: US 2022/0412998 A1
(43) Pub. Date: Dec. 29, 2022

(54) METHODS AND SYSTEMS FOR ASSAY REFINEMENT

(71) Applicant: Nautilus Biotechnology, Inc., Seattle, WA (US)

(72) Inventors: Vadim LOBANOV, Seattle, WA (US); Jarrett EGERTSON, Rancho Palos Verdes, CA (US); Shunqiang WANG, Foster City, CA (US); Pierre INDERMUHLE, Berkeley, CA (US); Gregory KAPP, San Carlos, CA (US); Ryan SEGHERS, Kirkland, WA (US); Siavash YOUSEFI, San Jose, CA (US)

(21) Appl. No.: 17/848,169

(22) Filed: Jun. 23, 2022

Related U.S. Application Data
(60) Provisional application No. 63/214,297, filed on Jun. 24, 2021.

(51) Int. Cl. G01N 35/00; G16B 30/00; G01N 33/68; C12N 15/10; G01N 21/64

(52) U.S. Cl. CPC G01N 35/00613; G16B 30/00; G01N 33/6842; C12N 15/1065; G01N 21/643; G01N 21/6428; G01N 2021/6439

(57) ABSTRACT

Methods for performing procedures on single analytes at single-analyte resolution are disclosed. The methods utilize an iterative approach to performing a sequence of steps during a single-analyte process. Control of the single-analyte process is achieved by implementing actions during each iteration based upon one or more determined process metrics. Systems are also detailed for implementing the disclosed methods at single-analyte resolution.

### CROSS-REFERENCE TO RELATED APPLICATION

This application claims priority to U.S. Provisional Patent Application No. 63/214,297, filed on Jun. 24, 2021, entitled “Methods and Systems for Assay Refinement,” which is hereby incorporated by reference in its entirety for all purposes.

### BACKGROUND OF THE INVENTION

The present invention is particularly useful to the field of single-molecule assays. More particularly, the present invention is useful for the determination of sequences of processes when configuring single-molecule assays.

Conventional single-molecule assays include systems and methods that permit the study of molecular properties or characteristics for molecules on an individual basis. Such single-molecule assays also include systems and methods that permit the study of interactions between an individual molecule and one or more other molecules. Single-molecule assays are of wide interest in the genomic, transcriptomic, proteomic, and metabolomic fields due to their potential to identify and quantify various markers for intra- and/or intercellular composition and variability. Some such single-molecule assays are configured variously to achieve different types of measurements depending upon variables such as sample type and measurement sensitivity.

Given the above background, what is needed in the art are improved systems and methods for detecting, characterizing, or manipulating molecules in bulk or for detecting, characterizing, or manipulating analytes other than molecules such as biological cells, organelles, tissues, or the like.

### SUMMARY OF THE INVENTION

The present disclosure addresses the shortcomings disclosed above by providing systems and methods for assay refinement.

One aspect of the present disclosure is directed to providing a method for controlling a single-analyte process. The method includes performing an iterative process until a determinant criterium has been achieved. The iterative process includes at least two cycles. Each cycle includes determining an uncertainty metric for a single analyte based upon a single-analyte data set. Each cycle includes implementing an action on a single-analyte system based upon the uncertainty metric, in which single-analyte system includes a detection system that is configured to obtain a physical measurement of the single analyte at single-analyte resolution. Moreover, each cycle further includes updating the single-analyte data set after implementing the action on the single-analyte system.

Another aspect of the present disclosure is directed to providing a method for controlling a single-analyte process. The method includes performing an iterative process until a determinant criterium has been achieved. The iterative process includes at least two cycles. Each cycle in the at least two cycles includes combining data from a single-analyte data set including data from more than one data source to determine a process metric for a single analyte. Each cycle further includes implementing an action on a single-analyte system based upon the process metric. The single-analyte system includes a detection system that is configured to obtain a physical measurement of the single analyte at single-analyte resolution. Each cycle includes updating the single-analyte data set after implementing the action on the single-analyte system.

Yet another aspect of the present disclosure is directed to providing a method for controlling the processes of a single-analyte process. The method includes performing an iterative process until a determinant criterium has been achieved. The iterative process includes at least two cycles. Each cycle includes determining a process metric for a single analyte based upon a single-analyte data set. Moreover, each cycle includes implementing an action on a single-analyte system that alters a source of uncertainty based upon the process metric. The single-analyte system includes a detection system that is configured to obtain a physical measurement of the single analyte at single-analyte resolution. Furthermore, each cycle includes updating the single-analyte data set after implementing the action on the single-analyte system.

Yet another aspect of the present disclosure is directed to providing a method for controlling the processes of a single-analyte process. The method includes performing an iterative process until a completion criterium has been achieved. The iterative process includes at least two cycles. Each cycle in the at least two cycles includes determining a curated uncertainty metric a plurality of single analytes based upon a single-analyte data set. Moreover, each cycle includes implementing an action on a single-analyte system based upon the curated uncertainty metric. The single-analyte system includes a detection system that is configured to obtain a physical measurement at single-analyte resolution of each single analyte of the plurality of single analytes. Further, each cycle includes updating the single-analyte data set after implementing the action on the single-analyte system.

### BRIEF DESCRIPTION OF THE DRAWINGS

FIGS. 1A-1B illustrate bulk resolution and single-analyte resolution observations of single-analyte systems, in accordance with some embodiments of the present disclosure, in which FIG. 1A depicts the system under normal conditions and FIG. 1B depicts the system in the presence of a contaminated buffer.

FIGS. 2A-2D depict determination of single-analyte resolution, in accordance with some embodiments of the present disclosure. FIG. 2A depicts 2-dimensional physical measurements and FIG. 2B depicts a 1-dimensional histogram for two single analytes that is considered resolved at single-analyte resolution, in accordance with some embodiments of the present disclosure. FIG. 2C depicts 2-dimensional physical measurements and FIG. 2D depicts a 1-dimensional histogram for two single analytes that is considered not resolved at single-analyte resolution, in accordance with some embodiments of the present disclosure.

FIG. 3 shows a block diagram for a single-analyte process that includes an iterative process, in accordance with some embodiments of the present disclosure.

FIG. 4 illustrates data exemplary data trends for an uncertainty metric during a single-analyte process, in accordance with some embodiments of the present disclosure.

FIGS. 5A-5B depicts block diagrams for configurations of iterative processes, in accordance with some embodiments of the present disclosure, which FIG. 5A depicts a regimented iterative approach and FIG. 5B depicts a step-wise iterative approach.

FIG. 6 shows a hierarchical structure for cycles, procedures, and sub-procedures of a single-analyte process, in accordance with some embodiments of the present disclosure.

FIG. 7 illustrates a block diagram for a method of configuring actions in a single-analyte process, in accordance with some embodiments of the present disclosure.

FIG. 8 depicts a sample preparation scheme from the collection of a sample including single analytes through the preparation of an array of single analytes for an analysis, in accordance with some embodiments of the present disclosure.

FIG. 9 shows an exemplary fluidics system schematic for a single-analyte system, in accordance with some embodiments of the present disclosure.

FIGS. 10A-10B illustrate a single-analyte detection system for a single-analyte system, in accordance with some embodiments of the present disclosure, which FIG. 10A illustrates the use of an excitation source to stimulate a fluorescent label on a single analyte and FIG. 10B illustrates the emission of fluorescence from a labeled single analyte to a detector in the detection system.

FIG. 11 depicts a method for configuring actions for an iterative process based upon selected outcomes, in accordance with some embodiments of the present disclosure.

FIG. 12 shows a block diagram for a single-analyte process, in accordance with some embodiments of the present disclosure.

FIG. 13 illustrates a single-analyte system comprising multiple processors, in accordance with some embodiments of the present disclosure.

FIG. 14 depicts a block diagram for a single-analyte process, in accordance with some embodiments of the present disclosure.

FIG. 15A-15I shows various alterations and/or manipulations that could occur to a single analyte during a single-analyte process, in accordance with some embodiments of the present disclosure.

FIG. 16 illustrates data flow and/or information flow between various components of a single-analyte system, in accordance with some embodiments of the present disclosure.

FIG. 17 depicts a method for determining process metrics and rules for process metrics prior to, during, or after a single-analyte process, in accordance with some embodiments of the present disclosure.

FIG. 18 shows the computational time scale for various algorithms that is implemented during a single-analyte process, in accordance with some embodiments of the present disclosure.

FIG. 19 illustrates a method of configuring a single-analyte process then implementing the single-analyte process with an iterative process, in accordance with some embodiments of the present disclosure.

FIG. 20 depicts a fluorescence-based affinity reagent binding assay, in accordance with some embodiments of the present disclosure.

FIG. 21 shows a barcode-based affinity reagent binding assay, in accordance with some embodiments of the present disclosure.

FIG. 22 illustrates an Edman-type degradation fluorosequencing assay, in accordance with some embodiments of the present disclosure.

FIG. 23 depicts an Edman-type affinity binding sequencing assay, in accordance with some embodiments of the present disclosure.

FIG. 24 shows a computer system, in accordance with some embodiments of the present disclosure.

FIGS. 25A-25B illustrate a single-analyte synthesis process, in accordance with some embodiments of the present disclosure, which FIG. 25A illustrates an ideal single-analyte synthesis process and FIG. 25B illustrates a single-analyte process with random errors that is addressable by an iterative single-analyte process.

FIG. 26 depicts a single-analyte fabrication process, in accordance with some embodiments of the present disclosure.

FIG. 27 shows a fluidic cartridge with a fluidic stagnation region, in accordance with some embodiments of the present disclosure.

FIGS. 28A, 28B, and 28C illustrate information and/or data flow in centralized, distributed, and decentralized systems, respectively, in accordance with some embodiments of the present disclosure.

FIG. 29 depicts an Edman-type degradation method, in accordance with some embodiments of the present disclosure.

FIGS. 30A-30E show an Edman-type degradation sequence for a polypeptide comprising post-translational modifications at specific amino acid residues, in accordance with some embodiments of the present disclosure.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first array could be termed a second array, and, similarly, a second array could be termed a first array, without departing from the scope of the present disclosure. The first array and the second array are both arrays, but they are not the same array.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description includes example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

Furthermore, as used herein, the term “dynamically” means an ability to update a program while the program is currently running.

Additionally, the terms “client,” “subject,” and “user” are used interchangeably herein unless expressly stated otherwise.

Moreover, as used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments, n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

The present disclosure provides methods and systems that are used to detect, characterize, or manipulate analytes. For purposes of illustration the systems and methods will be exemplified in the context of detecting, characterizing, or manipulating analytes at single-analyte resolution. In some embodiments, single-analyte systems include any system in which single analytes (such as single molecules), or complexes thereof, are observable and/or capable of being manipulated in a spatially- and/or temporally-separated fashion. For example, in some embodiments, a single-analyte detection system spatially and/or temporally resolves an individual analyte from all other analytes in a sample from which the analyte was obtained or in which the analyte is observed. Achieving high-confidence observations in single-analyte systems varies significantly from bulk characterization systems with regard to minimizing observation uncertainty. Any form of observation, such as physical measurements, will include some uncertainty, arising in part from both the system used to perform the measurement and the intrinsic uncertainty of observing a physical system. In some embodiments, bulk observations reduce the complexity of observation uncertainty in a bulk system by averaging over an ensemble of molecules or interactions, thereby offsetting or averaging out many of the false observations that give rise to uncertainty; the bulk observation is often a close approximation of the mean behavior of the system. By contrast, in a system comprising single analytes, any given observation of a single analyte is typically treated independently of other single analytes in the system. For example, in some embodiments, offsetting or averaging out of false observations is be possible; the observation is either representative of the single analyte, or not representative of the single analyte. Moreover, in some embodiments, stochastic behavior of a single analyte under observation, or gaps in the continuity of the observation, results in apparent absence of detection or otherwise lead to erroneous conclusions about the presence, absence, or characteristics of the single analyte. Methods and systems set forth herein provide advantages in improving detection of single analytes and improving confidence in conclusions about the presence, absence, or characteristics of the single analyte. It will be understood that various aspects or embodiments of the methods and systems set forth herein need not be limited to detecting, characterizing, or manipulating analytes at single-analyte resolution. For example, in some embodiments, aspects and embodiments of the present disclosure are extended to detection, characterization or manipulation of analytes in bulk.

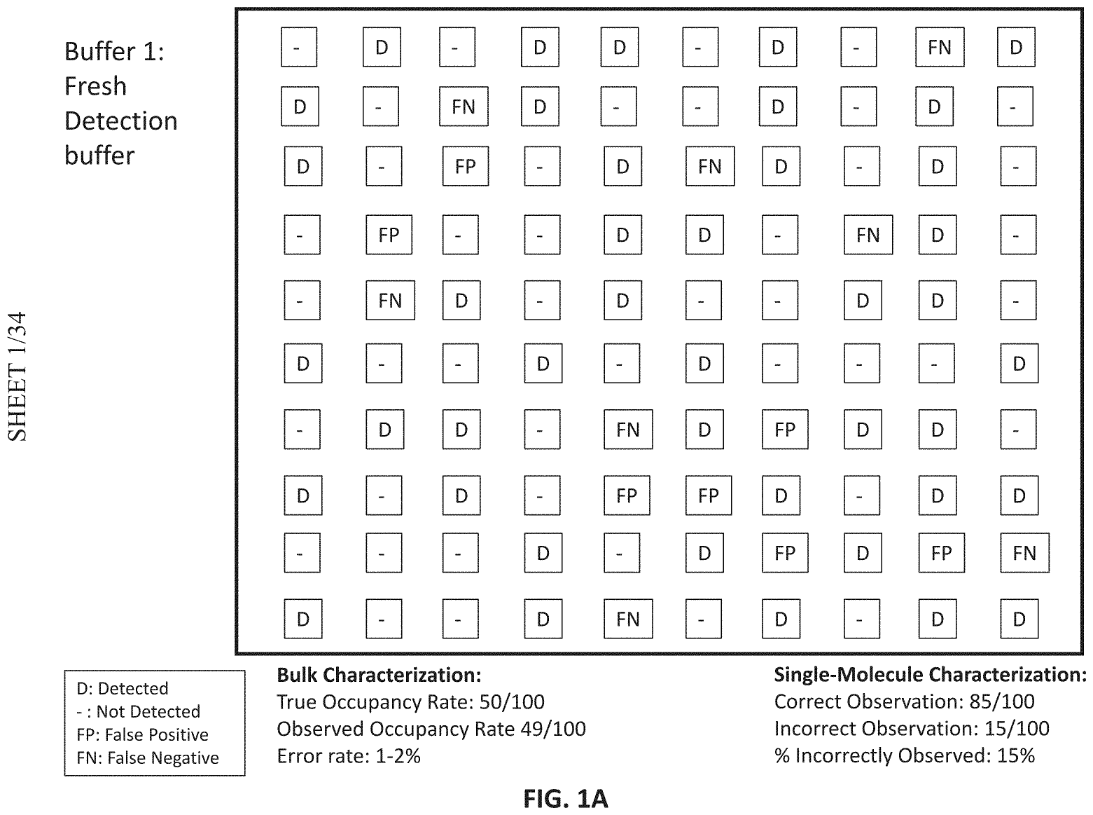

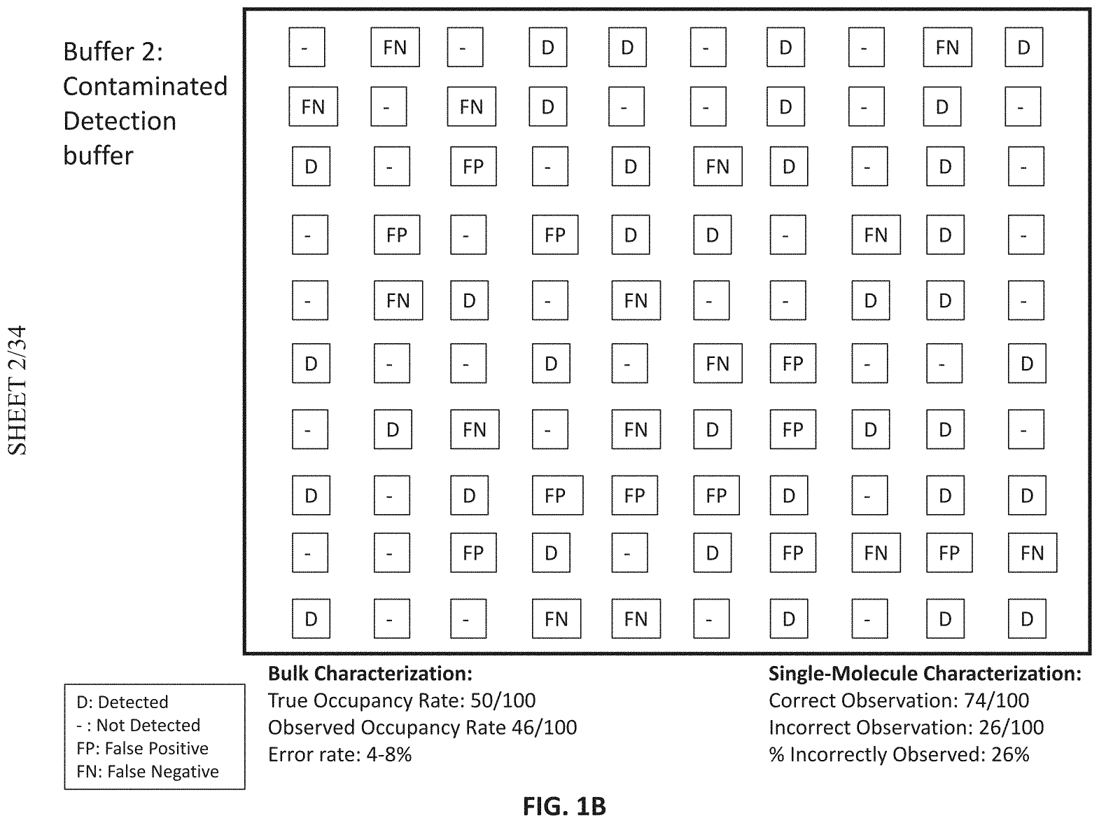

An example of the difference in uncertainty between bulk and single-analyte systems is illustrated in FIGS. 1A and 1B. FIG. 1A depicts an array comprising 100 possible binding sites. In some embodiments, an observation is made to determine the presence of molecules on the array in the presence of a fresh detection buffer. In some embodiments, such as in the case of a bulk system, the total quantity of molecules is determined by a bulk measurement that combines signals over all 100 sites of the array, such as total fluorescence intensity collected by a single pixel observing all 100 of the sites simultaneously. In some embodiments, such as in the case of a single-molecule characterization, the determination of total quantity of molecules is made by individually detecting a presence of a molecule at each of the 100 array sites, such as fluorescence intensity detected at each site by a discrete pixel or cluster of pixels that does not receive substantial signal from any other site in the array (e.g., each of the sites is resolved from the other sites). The array of FIG. 1A is shown from an omniscient perspective with the ground truth of each site shown, where “D” is a true detection, “-” is a true absence, “FP” is a false positive detection, and “FN” is a false negative detection. In some embodiments, it is assumed that any observation uncertainty arises from the method of observation for FIG. 1A. For a bulk characterization, the total number of molecules on the array is observed to be 49 out of the 100 possible due to the total number of true detections and false positive detections, whereas the actual number of molecules on the array is 50 out of the 100 possible. This would suggest an ˜2% uncertainty in the bulk observation. In some embodiments, for the single-molecule system, the determination of the presence of molecules on the array is performed on a site-by-site basis. In this case, 85 out of the 100 sites would be observed correctly, suggesting an ˜15% uncertainty in the single-molecule observation.

FIG. 1B shows an identical system to the system depicted in FIG. 1A, only differing in the presence of a contaminated detection medium. In some embodiments, the contaminated detection medium increases the rate of false detections, with false negatives more likely than false positives. In some embodiments, such as in the case of FIG. 1B, uncertainty arises from both the method of detection as well as the components of the system itself (e.g., the contaminated medium). For the bulk characterization, the total number of molecules on the array is observed to be 46 out of the 100 possible, suggesting an ˜8% bulk observation uncertainty in the presence of the contaminated buffer. For the single-molecule characterization, 74 out of the 100 sites would be observed correctly, suggesting a ˜26% single-molecule observation uncertainty in the presence of the contaminated buffer. FIGS. 1A-1B demonstrate how, in some embodiments, increased sources of uncertainty substantially increase the relative difference in observation uncertainty between a bulk system and a single-analyte system.

Accordingly, in any physical system including some source of observation uncertainty, a single analyte might not be described with high confidence through a single observation. Rather, in some embodiments, a collection of observations is obtained in a single-analyte system through performing a series of observations of each single analyte within the single-analyte system. In some embodiments, the collection of observations is combined to achieve benefits that derive from bulk characterizations. For example, in some embodiments, an observation, such as a detection of the presence of a single analyte at a location on a surface, is duplicated or replicated one or more times to build a collection of observation for the single analyte that collectively increases the confidence in the observation. Likewise, in some embodiments, a series of physically unique observations of a single analyte is made, such as a series of affinity binding observations by affinity reagents with differing binding characteristics, that collectively form a collection of observations for the single analyte.

In some embodiments, observation uncertainty in a single-analyte system arises from the physical mode of observation, as well as external factors such as reagent quality, user error, and system error. While certain sources of uncertainty are intrinsic and unavoidable due to physical phenomena such as entropy and chemical degradation, other sources of uncertainty are identifiable and, in some embodiments, correctable during operation of a single-analyte system. In some embodiments, although sources of uncertainty are identifiable, the impact of the sources of uncertainty vary on an analyte-by-analyte basis. Consequently, in some embodiments, in a multi-step single-molecule process (as is necessary to build an observation ensemble for each single molecule), any given step in the process fails for any given single analyte being observed. A primary challenge of building a robust single-analyte system is determining how to carry out a multi-step process efficiently given this often stochastic analyte-by-analyte variability. The methods and systems set forth herein are useful for overcoming such challenges.

Recognized herein are methods and systems for controlling single-analyte systems including one or more sources of uncertainty. In some embodiments, an iterative approach is utilized to assess observation uncertainty before, during, or after a step in a single-analyte process and, based upon the uncertainty or a change therein, adapt the process to another configuration such as an optimal configuration. In some embodiments, the iterative approach provided advantages of permitting flexible process methods that allow a single-analyte system to be applied to a broad range of problems, and/or permitting sources of observation uncertainty to be identified and, if possible, corrected or mitigated as the process is running, thereby increasing the overall confidence level of the process.

In some embodiments, the iterative approach described herein includes the steps: of determining a process metric from a single-analyte data set; implementing an action on a single-analyte system based upon the process metric, where the single-analyte system comprises a detection system that is configured to obtain a physical measurement of the single analyte at single-analyte resolution; and updating the set of single-analyte system data after implementing the action on the single-analyte system. In some embodiments, the set of system data includes data from multiple data sources, including the physical measurements, instrument metadata, sample metadata, and cumulative or prior-collected data. In some embodiments, the action that is implemented on the single-analyte system alters a source of uncertainty that affects the single-analyte process. In some embodiments, an iterative approach to a single-analyte process occurs in a system with a plurality of single analytes, in which a process metric is determined independently for each single analyte of the plurality of single analytes.

In some embodiments, a single-analyte process utilizes an iterative approach for various purposes, including maintaining system function (analogously referred to as ‘hygiene’) for a single-analyte system, or improving the outcome of a single-analyte process performed on a single-analyte system. In some embodiments, an iterative approach is utilized to maintain system function or hygiene and improve the outcome of a single-analyte process performed on a single-analyte system. In some embodiments, maintaining system function or hygiene of a single-analyte system includes implementing one or more actions that correct, alter, or repair the system to improve the system performance and/or decrease sources of uncertainty in single-analyte characterizations performed by the single-analyte system. For example, in some embodiments, an iterative process is configured to identify and/or address sources of decreased confidence in physical measurements performed on a single-analyte system (e.g., contaminated reagents, malfunctioning sensors, malfunctioning hardware, etc.), thereby increasing the confidence of physical measurements that are utilized to characterize a single analyte in the single-analyte system. In some embodiments, improving the outcome of the single-analyte process includes any optimization, refinement, or economization of the single-analyte process with respect to the desired process outcome. For example, in some embodiments, an iterative approach is utilized for a single-analyte assay process to increase the speed of the assay, decrease the material or reagent cost of the assay, or increase the confidence of the assay results. In some embodiments, an iterative process of the present disclosure is manual, automated, or partially automated. Accordingly, in some embodiments, one or more steps in an interactive process set forth herein is manual or automated.

### Definitions

As used herein, the term “site” refers to a location in an array where a particular analyte (e.g., protein, peptide or unique identifier label) is present. In some embodiments, a site includes a single analyte or a population of several analytes of the same species (e.g., an ensemble of the analytes). In some embodiments, a site includes a population of different analytes. Sites are typically discrete. In some embodiments, the discrete sites are contiguous or separated by interstitial spaces. In some embodiments, an array useful herein includes, for example, sites that are separated by less than 100 microns, 10 microns, 1 micron, 100 nm, 10 nm or less. In some embodiments, an array includes sites that are separated by at least 10 nm, 100 nm, 1 micron, 10 microns, or 100 microns. In some embodiments, the sites each have an area of less than 1 square millimeter, 500 square microns, 100 square microns, 10 square microns, 1 square micron, 100 square nm or less. In some embodiments, an array includes sat least about 1×10⁴, 1×10⁵, 1×10⁶, 1×10⁷, 1×10⁸, 1×10⁹, 1×10¹⁰, 1×10¹¹, 1×10¹², or more sites. The term “address,” when used in the context of an array, is intended to be synonymous with the term “site.”

As used herein, in some embodiments, the term “array” refers to a population of analytes (e.g., proteins) that are associated with unique identifiers such that the analytes is distinguished from each other. In some embodiments, a unique identifier is, for example, a solid support (e.g., particle or bead), site on a solid support, tag, label (e.g., luminophore), or barcode (e.g., nucleic acid barcode) that is associated with an analyte and that is distinct from other identifiers in the array. In some embodiments, analytes re associated with unique identifiers by attachment, for example, via covalent bonds or non-covalent bonds (e.g., ionic bond, hydrogen bond, van der Waals forces, electrostatics etc.). In some embodiments, an array includes different analytes that are each attached to different unique identifiers. In some embodiments, an array includes different unique identifiers that are attached to the same or similar analytes. In some embodiments, an array includes separate solid supports or separate sites that each bear a different analyte. In some embodiments, the different analytes are identified according to the locations of the solid supports or sites.

As used herein, the term “single analyte” refers to a chemical entity that is individually manipulated or distinguished from other chemical entities. In some embodiments, a single analyte possesses a distinguishing property such as volume, surface area, diameter, electrical charge, electrical field, magnetic field, electronic structure, electromagnetic absorbance, electromagnetic transmittance, electromagnetic emission, radioactivity, atomic structure, molecular structure, crystalline structure, or a combination thereof. In some embodiments, the distinguishing property of a single analyte is a property of the single analyte that is detectable by a detection method that possesses sufficient spatial resolution to detect the individual single analyte from any adjacent single analytes. In some embodiments, a single analyte includes a single molecule, a single complex of molecules, a single particle, or a single chemical entity comprising multiple conjugated molecules or particles. In some embodiments, a single analyte is distinguished based on spatial or temporal separation from other analytes, for example, in a system or method set forth herein. Moreover, in some embodiments, reference herein to a ‘single analyte’ in the context of a composition, system or method does not exclude application of the composition, system or method to multiple single analytes that are manipulated or distinguished individually, unless indicated contextually or explicitly to the contrary.

As used herein, the term “single-analyte system” refers to an interconnected series of components configured to manipulate or distinguish an analyte individually. In some embodiments, a single-analyte system is a closed or open system with respect to energy transfer and/or mass transfer. In some embodiments, a single-analyte system further comprises a component that is configured to detect and/or manipulate one or more single analytes at a resolution that distinguishes each of the analytes individually. In some embodiments, a single-analyte system includes one or more surfaces, boundaries, interfaces, supports or media that includes or are in contact with a single analyte. In some embodiments, a single-analyte system manipulates or distinguishes more than one analyte, so long as at least one of the analytes is manipulated or distinguished individually.

As used herein, the term “single-analyte process” refers to detection or manipulation of one or more analytes at a resolution that distinguishes the one or more analytes individually. In some embodiments, a single-analyte process detects, synthesizes, or manipulates a single analyte at a resolution that distinguishes the analyte individually. In some embodiments, a single-analyte process detects, synthesizes, or manipulates multiple single analytes at a resolution that distinguishes at least one of the analytes from the others.

As used herein, the term “single-analyte data set” refers to information that is obtained from, or characterizes, at least one analyte on an individual basis. In some embodiments, a single-analyte data set includes information that is obtained with respect to a single-analyte system. In some embodiments, a single-analyte data set includes data that is collected, obtained, or compiled from one or more than one data source, such as an analog device, a digital device, a user input, or a combination thereof. In some embodiments, a single-analyte data set includes observed information, measured information, calculated information, derived information, predicted information, reference information, stored information, user-defined information, process information, or a combination thereof. In some embodiments, a single-analyte data set includes a fixed record or is alterable by the removal of information, addition of information, rearrangement of information, reassignment of information, updating of information, revision of information, or a combination thereof. In some embodiments, a single-analyte data set includes a digital record, a non-digital record, or a combination thereof. In some embodiments, a single-analyte data set includes generated, stored, or manipulated by a user or an electronic device, such as a computer, processor, server, tablet, or mobile phone. In some embodiments, a single-analyte data set includes stored, transmitted, or manipulated in a non-transitory computer readable medium. In some embodiments, a single-analyte data set includes one or more data types, such as integer data, floating-point number data, text data, string data, Boolean data, or a combination thereof.

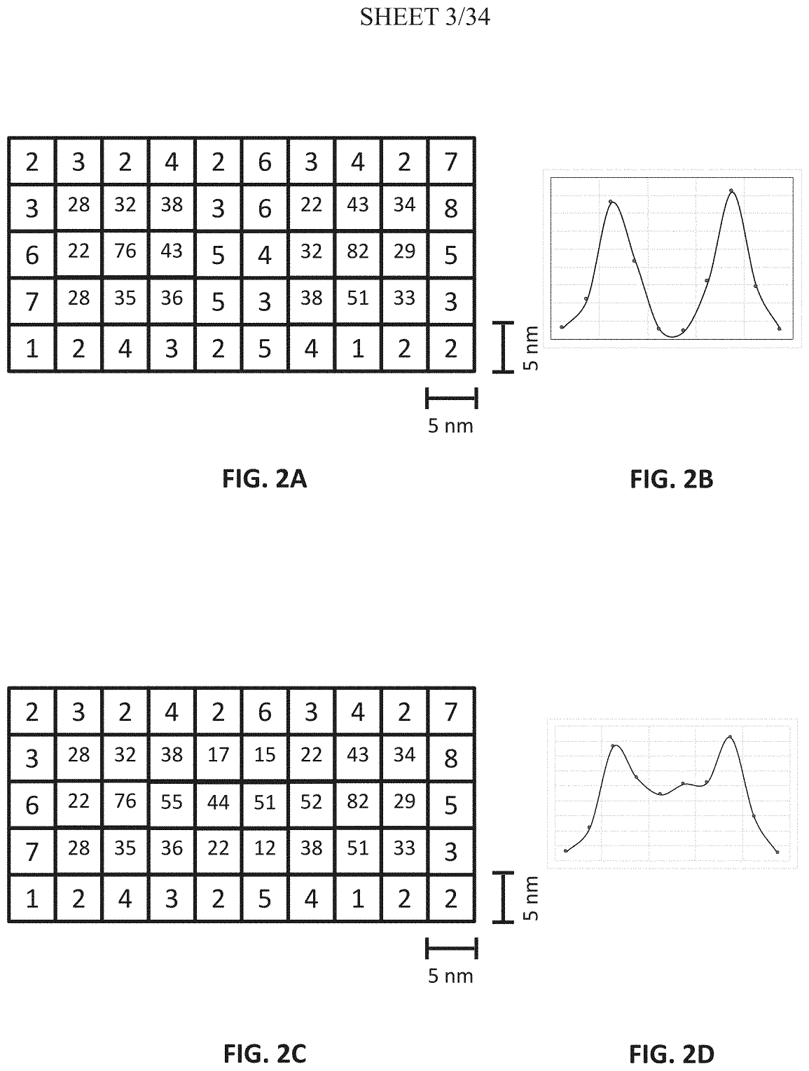

As used herein, the term “single-analyte resolution” refers to the detection of, or ability to detect, an analyte on an individual basis, for example, as distinguished from its nearest neighbor. In some embodiments, the nearest neighbor of a single analyte includes a support, surface, interface, or medium with which the single analyte associates, or an adjacent analyte (whether the adjacent analyte is a single analyte or member of an ensemble of analytes). In some embodiments, single-analyte resolution is defined by a spatial and/or temporal length scale with respect to one or more individual analytes. In some embodiments, single-analyte resolution is achieved when a detection mode is configured to observe a single analyte at the spatial and/or temporal scale of the single analyte. For example, in some embodiments, an optical fluorescence detector is capable of resolving an analyte of at least 10 nanometers (nm) in size if a fluorescent signal from the analyte is present for at least 1 second (s). In some embodiments, the optical fluorescence detector is capable of resolving two analytes from each other when the two analytes are spatially separated by at least 10 nanometers (nm). In some embodiments, single-analyte resolution is associated with a spatial distribution, peak signal intensity, average signal intensity, or signal distribution obtained by a detecting device (e.g., a sensor) at a discrete spatial location. For example, in some embodiments, a pixel-based optical detector detects a single analyte at single-analyte resolution if an optical signal is detected at a plurality of pixels with a particular signal intensity profile, and the pixels are surrounded by a region with a signal intensity that matches an expected background intensity. FIGS. 2A-2D depict examples of a pixel-based detector results with differing signal profiles. FIG. 2A depicts exemplary signal intensity data from a pixel-based detector with each pixel representing an approximately 5 nm by 5 nm spatial region. The pixel-based detector collects physical data for an array of single analytes with a predicted size of 10-20 nm. FIG. 2B depicts a cross-sectional plot of the pixel-based signal-intensity data shown in FIG. 2A. The intensity data suggests two distinct single analytes that are distinct from the surrounding background medium and spatially separated from each other, with a size of approximately 10 to 15 nm for each single analyte. In some embodiments, the data from FIGS. 2A-2B is considered to have single-analyte resolution. FIGS. 2C-2D depict data collected in an identical fashion to the data shown in FIGS. 2A-2B, but with a differing intensity profile. Based upon the data in FIGS. 2C-2D, the pixel-based detector might be considered to individually detect two single analytes or to detect an ensemble of two analyte. In some embodiments, this depends, for example, upon parameters applied to identify peaks when analyzing the data. Accordingly, the data from FIGS. 2C-2D might not be considered single-analyte resolution.

As used herein, the term “bulk,” when used in reference to manipulating or detecting a plurality of analytes, means manipulating or detecting the analytes as an ensemble, whereby individual analytes in the ensemble are not necessarily resolved from each other. In some embodiments, the term is used in reference to a system, process, or data set that includes or derives from an ensemble or plurality of analytes. In some embodiments, the properties, characteristics, behaviors, and other features of a bulk system, process, or data set derives in whole or in part from a collection, combination or average of the properties, characteristics, behavior, or other features of the ensemble or plurality of analytes. In some embodiments, a bulk property, characteristic, or behavior is determined or measured by a system that is also configured to determine a single-analyte property, characteristic, or behavior. In some embodiments, a, a bulk property, characteristic, or behavior is determined or measured on a system that is configured to determine or measure bulk properties, characteristics, or behaviors.

As used herein, the term “process metric” refers to a representation of a characteristic, property, effect, behavior, performance, or variability within a method or system. In some embodiments, the representation is quantitative (e.g., a numerical value or measure) or qualitative (e.g., a score or non-numeric identifier). In some embodiments, the method is a single-analyte method. In some embodiments, the system is a single-analyte system. In some embodiments, a process metric is a representation of a characteristic, property, effect, behavior, performance, or variability of a component of a single-analyte method or system other than the single analyte used in the method or system. In some embodiments, a process metric is composed in numeric or non-numeric forms, including single values, sets, matrices, tensors, or a combination thereof. In some embodiments, a process metric includes categorized or enumerated metrics, including binary, trinary, and polynary groups (e.g., pass/fail, type 1/type 2/type 3, etc.). In some embodiments, a process metric is a direct measure of uncertainty in a single-analyte method or system, i.e., an uncertainty metric. In some embodiments, a process metric is an indirect measure of uncertainty in a single-analyte method or system, such as an uncertainty proxy, a correlative, a leading indicator, a lagging indicator, a counter-indicator, an analogue, or a combination thereof. In some embodiments, a process metric is determined from a single-analyte data set. In some embodiments, a process metric is derived from a single-analyte data set including information and/or data collected from or pertaining to a single-analyte system. In some embodiments, information and/or data collected from a single-analyte method or system includes physical measurements, instrument metadata, sensor data, algorithm data, algorithm metadata, or a combination thereof. In some embodiments, information and/or data pertaining to a single-analyte method or system include user-supplied single-analyte information (e.g., sample source), externally-supplied single-analyte information (e.g., supplier reagent or analyte data), cumulative information (e.g., prior-collected data), reference information (e.g., a database), identification information (e.g., barcodes, serial numbers, QR codes, etc.), or a combination thereof. In some embodiments, a process metric is determined from a single-analyte data set by any of a variety of data analysis methods, including for example, extracting a process metric, calculating a process metric, inferring a process metric, decoding a process metric, deciphering a process metric, deconvoluting a process metric, compiling a process metric, receiving a process metric, or a combination thereof. In some embodiments, a process metric is determined by a user input, a processor-implemented algorithm, or a combination thereof.

As used herein, a “qualitative process metric” refers to a process metric that is manipulable or manipulated by a non-mathematical operation. In some embodiments, qualitative process metrics include enumerated and categorized metrics (e.g., binary, trinary, and polynary groupings), classifiers, user-defined metrics, or a combination thereof. In some embodiments, a qualitative process metric includes mathematical values that are manipulated in a non-mathematical operation.

As used herein, a “quantitative process metric” refers to a process metric that is manipulable or manipulated by one or more mathematical operations. In some embodiments, a quantitative process metric includes one or more numeric values. In some embodiments, a quantitative process metric includes a variable, a function, or an equation. For example, in some embodiments, a quantitative process metric is expressed as a function of one or more variables, such as a function of one or more other process metrics.

As used herein, the term “curated process metric” refers to a process metric that is determined from one or more other process metrics. In some embodiments, a curated process metric is determined from one or more process metrics for a single analyte. In some embodiments, a curated process metric is determined from one or more process metrics from each single analyte of a plurality of single analytes. In some embodiments, a curated process metric includes a qualitative process metric or a quantitative process metric. In some embodiments, a curated process metric includes a value that is determined from statistically or mathematically manipulating a set of process metrics, such as a mean value, a median value, a mode, a range, a consensus value, a maximum value, a minimum value, a moment, a center, a centroid, an expansion, a contraction, an integral, a derivative, or a combination thereof.

As used herein, the term “uncertainty metric” refers to a representation of variability with respect to a characteristic, property or effect that is observed in a method or system. In some embodiments, the representation is quantitative (e.g., a numerical value or measure) or qualitative (e.g., a score or non-numeric identifier). In some embodiments, the method is a single-analyte method. In some embodiments, the system is a single-analyte system. In some embodiments, the characteristic, property, or effect pertains to a single analyte measured at single-analyte resolution within a single-analyte method or system. In some embodiments, the characteristic, property, or effect pertains to a plurality of single analytes that are measured at single-analyte resolution within a single-analyte method or system. In some embodiments, an uncertainty metric pertains to a measure of error and/or bias in a single-analyte method or system. In some embodiments, an uncertainty metric includes various sources of uncertainty, such as parameter uncertainty, parametric uncertainty, structural uncertainty, algorithmic uncertainty, experimental uncertainty, inference uncertainty, and interpolation uncertainty. In some embodiments, an uncertainty metric pertaining to a measure of error and/or bias in the single-analyte method or system is characterized as stochastic, random, systematic, variable, and/or fixed. In some embodiments, an uncertainty metric is described with respect to a temporal or spatial scale of a single-analyte method or system. In some embodiments, an uncertainty metric is derived with regard to a set of data derived from a single-analyte method or system, including measured or observed data, as well as data determined from measured or observed data. In some embodiments, an uncertainty metric is determined for any continuum or grouping of data regarding a single analyte or a single-analyte method or system, such as point data, time-series data, panel data, cross-sectional data, aggregate data, multivariate data, data distributions, data populations, or continuous data sets. In some embodiments, an uncertainty metric is determined for any type of behavior of a single-analyte method or system, including for example, stochastic, probabilistic, or deterministic systems. In some embodiments, an uncertainty metric includes a qualitative and/or a quantitative measure of uncertainty within or related to the single-analyte method or system. In some embodiments, a qualitative uncertainty metric includes non-numeric or subjective measures of uncertainty (e.g., high, medium, or low background signal). In some embodiments, a quantitative uncertainty metric includes, but is not limited to, metrics such as confidence interval, confidence level, prediction interval, tolerance interval, Bayesian interval, sensitivity coefficient, confidence region, confidence band, error propagation, uncertainty propagation, correlation coefficient, coefficient of determination, mean, median, mode, variance, standard deviation, coefficient of variation, percentile, range, skewness, kurtosis, L-moment, or index of dispersion. In some embodiments, an uncertainty metric includes an enumerated or categorized metric. In some embodiments, an enumerated or categorized uncertainty metric includes any metric for which the metric is classified into distinct groupings or categories (e.g., type 1/type 2/type 3; increase/neutral/decrease, etc.). In some embodiments, an enumerated or categorized uncertainty metric includes a binary metric (e.g., within detection range/outside of detection range, etc.). In some embodiments, an uncertainty metric is determined by any suitable method, including statistical models, stochastic models, correlation models, weighted models, and inference. In some embodiments, an uncertainty metric is determined by a user or by an algorithm configured to determine the uncertainty metric.

As used herein, the term “iterative process” refers to a cyclical procedure in which each cycle (e.g., iteration) of the procedure includes one or more shared sub-procedures or steps. In some embodiments, a single-analyte process includes one or more iterative processes. In some embodiments, an iterative process includes a defined sub-procedure, step, series of steps, or series of sub-procedures that is common to some or all the cycles of the iterative process. In some embodiments, an iterative process includes a variable sub-procedure, step, series of sub-procedures, or series of steps that is common to some or all the cycles of the iterative process. In some embodiments, an iterative process includes a sub-procedure, step, series of sub-procedures, or series of steps that is performed for at least one cycle of the iterative process, but not performed for at least one other cycle of the iterative process. In some embodiments, an iterative process includes one or more nested iterative processes. For example, in some embodiments, one iterative process is nested in a cycle of another iterative process. In some embodiments, an iterative process includes one or more iterative processes that are carried out serially. For example, in some embodiments, one iterative process follows another iterative process. In some embodiments, an iterative process includes a defined or undefined number of cycles or repetitions. In some embodiments, an iterative process terminates when a criterium is achieved. In some embodiments, an iterative process terminates at a defined, automatic, or pre-determined point, such as a time, a time interval, a number of cycles, a number of sub-procedures, or a combination thereof. In some embodiments, a defined, automatic, or pre-determined point for terminating an iterative process is user-defined, or calculated, predicted, or estimated by a computer process. In some embodiments, steps or sub-procedures of an iterative process include physical operations, computational operations, algorithmic operations, logical operations, or a combination thereof.

As used herein, the term “action,” when used in reference to an iterative process, refers to a step, sub-procedure, series of steps, or series of sub-procedures of the iterative process. In some embodiments, the action is implemented within a single-analyte system in response to the determination of a process metric (e.g., an uncertainty metric). In some embodiments, an action is implemented in response to a value of a process metric, or a change or trend in a process metric. In some embodiments, an action is implemented within a single-analyte system to alter a process metric. In some embodiments, an action is implemented in response to a single process metric. In some embodiments, an action is implemented in response to more than one process metric. In some embodiments, an action is implemented only if particular values are simultaneously determined for two or more process metrics. In some embodiments, an action includes a physical operation, mechanical operation, signal transmission operation, energy transduction operation, computational operation, algorithmic operation, logical operation, or a combination thereof. In some embodiments, an action is defined or self-limited (e.g., rinsing for 1 minute). In some embodiments, an action is recursive, iterative, or otherwise defined by one or more performance criteria (e.g., rinsing until an effluent pH is measured to be greater than pH 7.0). In some embodiments, an action initiates, terminates, pauses, resumes, gates, attenuates, activates or inhibits an operation such as a physical operation, mechanical operation, signal transmission operation, energy transduction operation, computational operation, algorithmic operation or logical operation. In some embodiments, an action is performed one or more times per iteration of an iterative process, such as about 1, 2, 3, 4, 5, 6, 7, 8, 9, 10, or more than 10 times. In some embodiments, an action is performed a minimum number of times per iteration of an iterative process, such as at least about 1, 2, 3, 4, 5, 6, 7, 8, 9, 10, or more time(s). In some embodiments, an action is performed a maximum number of times per iteration of an iterative process, such as no more than about 10, 9, 8, 7, 6, 5, 4, 3, 2, or 1 time(s). In some embodiments, an action is interrupted, pre-empted, altered, or cancelled during an iteration of an iterative process.

As used herein, the term “step,” when used in reference to a single-analyte process, refers to a procedure that is a component of the single-analyte process. In some embodiments, an action implemented within a single-analyte system includes one or more steps. In some embodiments, a step is a procedure that occurs during an iterative process. For example, in some embodiments, a step is performed during one or more cycle of an iterative process. In some embodiments, a step is a procedure that occurs during a single-analyte process but does not occur during an iterative process. In some embodiments, a step includes a physical operation, computational operation, algorithmic operation, logical operation, or a combination thereof. In some embodiments, a step is a mandatory or an optional procedure for a single-analyte process. In some embodiments, a step is a mandatory or an optional procedure for an iterative process. In some embodiments, a step is repeated one or more times during a single-analyte process. In some embodiments, a step includes one or more sub-procedures that constitute the step. For example, in some embodiments, a rinsing step on a single-analyte system includes sub-procedures such as fluid injection, fluid sensing, and fluid extraction. As used herein, the term “sub-procedure” refers to a specific or isolated action that occurs within a single-analyte system. In some embodiments, a sub-procedure includes a physical operation, computational operation, algorithmic operation, logical operation, or a combination thereof. In some embodiments, an action or step includes one or more sub-procedures. In some embodiments, an action or step includes a sequence or series of sub-procedures. In some embodiments, a sequence or series of sub-procedures is a fixed sequence or series of sub-procedures. In some embodiments, a sequence or series of sub-procedures is a variable sequence or series of sub-procedures. In some embodiments, an action or a step includes a fixed or variable number of sub-procedures, such as about 1, 2, 3, 4, 5, 6, 7, 8, 9, 10, or more than 10 sub-procedures. In some embodiments, an action or a step includes a minimum number of sub-procedures, such as at least about 1, 2, 3, 4, 5, 6, 7, 8, 9, 10, or more than 10 sub-procedures. In some embodiments, an action or a step includes a minimum number of sub-procedures, such as no more than about 10, 9, 8, 7, 6, 5, 4, 3, 2, or less than 2 sub-procedures.

As used herein, the term “user,” when used in reference to a system or method, refers to a subject who interacts with the system or method, for example, by providing an input to the system or method or by receiving an output from the system or method. In some embodiments, the system is a single-analyte system. In some embodiments, the method is a single-analyte method. Exemplary inputs/outputs include, but are not limited to, an analyte, a reagent, a product, a material, a substance, a fluid, a solid, a datum, an instruction, an algorithm, a decision, or a combination thereof. In some embodiments, a user initiates, monitors, alters, maintains, or terminates a method or system. In some embodiments, a user initiates or implements an action, step, or sub-procedure on a system or in a method. In some embodiments, a user initiates or implements an action, step, or sub-procedure on a system, or in a method, due to information or a prompt delivered from the system or method. In some embodiments, a user initiates or implements an action, step, or sub-procedure on a system, or in a method, without information or a prompt delivered from the system or method. In some embodiments, a user initiates or implements an action, step, or sub-procedure on a system, or in a method, that intervenes in an automated process. In some embodiments, a user interacts with a system or method in any capacity, including providing reagents and/or analytes, preparing reagents and/or analytes, providing information, operating a single-analyte system, providing inputs or instructions to a single-analyte system and/or a single-analyte process, and receiving information from a single-analyte system and/or a single-analyte process. In some embodiments, a user is a human subject, such as a human operator of the system or method or a third-party human who is permitted to provide an input to the system or method. In some embodiments, a user is a non-human subject such as an external computer system that is configured to provide an input to the system or method.

As used herein, the term “characterization” refers to the determination of a property, characteristic, behavior, interaction, identity, or a combination thereof, for example, within a single-analyte or bulk system. In some embodiments, a system or method is configured to provide a single-analyte characterization, a bulk characterization, or a combination thereof. In some embodiments, a system or method is configured for the purposes of providing a characterization. In some embodiments, a system or method provides a characterization as a portion of a process involving a single analyte or a bulk analyte.

As used herein, the term “physical measurement,” when used in reference to an analyte, refers to an empirical observation of the analyte. In some embodiments, the physical measurement is performed at a resolution that distinguishes a single analyte or at a lower resolution that observes a plurality of analytes in bulk. In some embodiments, a physical measurement provides a measure of a property, characteristic, behavior, interaction, identity, or a combination thereof for a single analyte or a plurality of analytes in bulk. In some embodiments, a physical measurement is a qualitative measurement (e.g., hydrophobic/hydrophilic) or a quantitative measurement (e.g., a measured pKₐ or isoelectric point). In some embodiments, a physical measurement is performed by a detection system or detection device that is configured to perform the physical measurement. In some embodiments, a physical measurement is based upon a passive observation of an analyte behavior (e.g., scintillation counting of radioactive decay). In some embodiments, a physical measurement is based upon an active observation of a chemical or physical interaction with a single analyte or a plurality of analytes in bulk (e.g., light scattering, light absorption, deflection in an electric field, etc.). In some embodiments, physical measurements include, but are not limited to, optical measurements (e.g., UV absorption, VIS absorption, IR absorption, luminescence, polarity, luminescence lifetime, resonance Raman or surface plasmon resonance), electrical measurements (e.g., field effect perturbation, potentiometry, coulometry, amperometry or voltammetry), magnetic measurements (magnetic moment, magnetic spin or nuclear magnetic resonance), mass measurements (e.g., mass spectroscopy), thermal measurements (e.g., calorimetry), or analytical separation measurements (e.g., chromatography or electrophoresis).

As used herein, the term “detection system,” when used in reference to an analyte, refers to a system that is configured to determine the presence or absence of the analyte. In some embodiments, the system is configured to resolve a single analyte or to observe a plurality of analytes in bulk. In some embodiments, a detection system is configured to determine the presence or absence of a single analyte or bulk analyte through a characterization or a physical measurement. In some embodiments, a detection system includes a sensing system that is configured to determine the presence or absence of an analyte, for example, at single-analyte resolution or at bulk analyte resolution. In some embodiments, a sensing system includes one or more sensors that detect a presence or absence of a signal from an analyte, for example, at single-analyte resolution or at bulk analyte resolution. In some embodiments, a sensing system includes a passive sensing system if it measures a presence or absence of a single analyte or a bulk analyte without creating a physical interaction with the single analyte or the bulk analyte. In some embodiments, a sensing system includes an active sensing system if it measures a presence or absence of a single analyte or a bulk analyte by creating a physical interaction with the single analyte or the bulk analyte. In some embodiments, an active sensing system includes one or more interaction components that create a physical interaction with a single analyte or a bulk analyte. In some embodiments, an interaction component provides a material, reagent, energy, stress, or field to a single analyte or bulk system.

As used herein, the term “solid support” refers to a substrate that is insoluble in aqueous liquid. In some embodiments, the substrate is rigid. In some embodiments, the substrate is nonporous or porous. In some embodiments, the substrate is capable of taking up a liquid (e.g., due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon, cyclic olefins, polyimides etc.), nylon, ceramics, resins, Zeonor, silica or silica-based materials including silicon and modified silicon, carbon, metals, inorganic glasses, optical fiber bundles, gels, and polymers.

As used herein, the term “cumulative data,” when used in reference to one or more analytes, refers to information from prior-collected detection of the one or more analytes. In some embodiments, cumulative data includes data concerning a single-analyte, a single-analyte system, and/or a single-analyte process. In some embodiments, cumulative data includes data concerning bulk analytes, systems for detecting bulk analytes or methods for detecting bulk analytes. In some embodiments, cumulative data includes a compilation of prior-collected data sets. In some embodiments, cumulative data includes distillation and/or mining of prior-collected data sets. In some embodiments, cumulative data includes data collected from prior runs of a detection process, such as a process identical to a current single-analyte process, or a process differing from a current single-analyte process. In some embodiments, cumulative data includes single-analyte data sets collected on instruments other than a single-analyte system. In some embodiments, cumulative data includes proprietary and/or internal knowledge that has been collected with respect to a single-analyte, a single-analyte system, and/or single-analyte process. In some embodiments, cumulative data is utilized as a reference source for configuring actions, steps, procedures, and/or sub-procedures before, during, or after a single-analyte process or other process set forth herein.

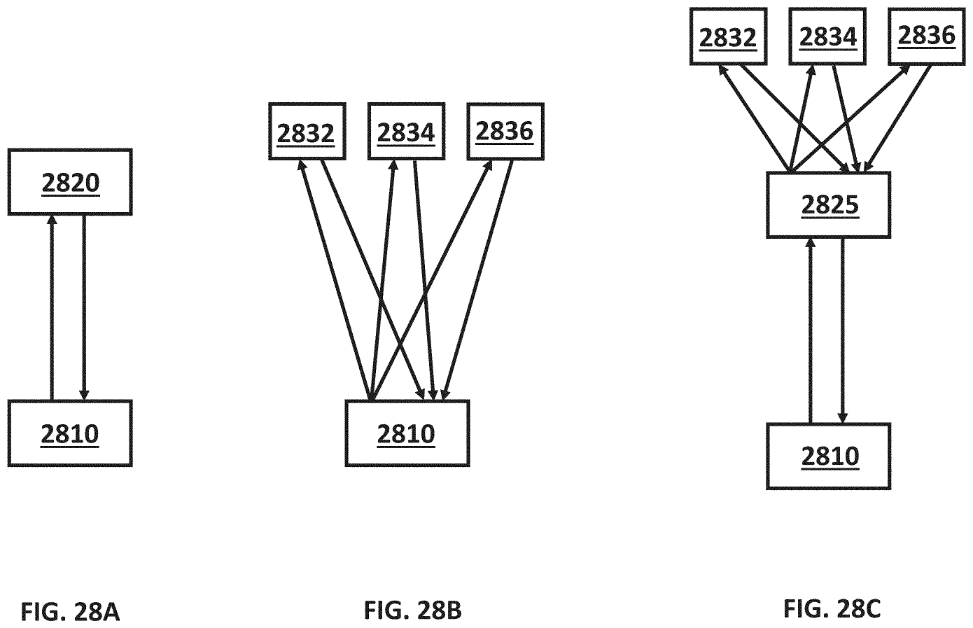

As used herein, the term “centralized,” when used in reference to a data source or algorithm, refers to a singular or consolidated node that controls information flow in a single-analyte system. FIG. 28A illustrates a centralized system in which a single-analyte system 2810 sends or receives information from a centralized node 2820. For example, in some embodiments, a “centralized data source,” refers to a single sensor (e.g., a CMOS sensor) that provides one or a plurality of measurements to a single-analyte system. In some embodiments, a “centralized algorithm,” refers to an algorithm that performs all tasks of the algorithm on a single processor or network of processors. As used herein, the term “decentralized,” when used in reference to a data source or algorithm, refers to a series of nodes that control information flow in a single-analyte system, in which each node is configured to control information flow independently of another node of the series of nodes. FIG. 28B illustrates a decentralized system, in which a single-analyte system 2810 sends or receives information from a series of independent nodes 2832, 2834, and 2836 without an intermediate node to control the information flow to the single-analyte system. For example, in some embodiments, a “decentralized data source,” refers to a network of sensors in which a sensor pushes data or has data pulled independently of other sensors in the network. In some embodiments, a “decentralized algorithm,” refers to an algorithm in which various tasks of the algorithm are distributed across a network of independently-functioning processors. As used herein, the term “distributed,” when used in reference to a data source or algorithm, refers to a series of nodes that control information flow in a single-analyte system under the control of a central node. FIG. 28C illustrates a distributed system, in which a single-analyte system 2810 sends or receives information from a series of independent nodes 2832, 2834, and 2836 via an intermediate node 2825 that controls the information flow to the single-analyte system. For example, in some embodiments, a “distributed data source,” refers to a network of sensors that collectively push data or have data pulled by a control algorithm. In some embodiments, a “distributed algorithm,” refers to an algorithm that distributes algorithm tasks to a network of processors under the control of a central processor.

### Single-Analyte Processes

Described herein are single-analyte systems and processes that utilize one or more iterative processes. In some embodiments, the present disclosure provides a method for controlling a single-analyte process, the method comprising performing an iterative process until a determinant criterium has been met, in which the iterative process comprises the steps of: determining a process metric (e.g., an uncertainty metric) for a single analyte based upon a single-analyte data set; implementing an action on a single-analyte system based upon the process metric, in which the single-analyte system comprises a detection system that is configured to obtain a physical measurement of the single analyte at single-analyte resolution; and updating the single-analyte data set after implementing the action on the single-analyte system.

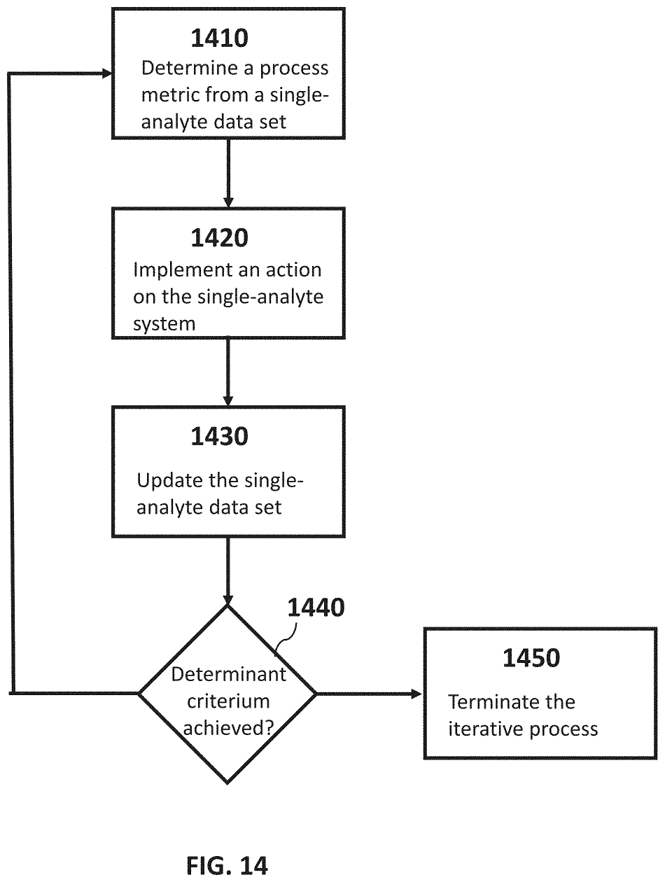

FIG. 14 depicts an iterative process in accordance with some embodiments disclosed herein. In some embodiments, a cycle of an iterative process includes the step of determining a process metric (e.g., an uncertainty metric) from a single-analyte data set 1410. In some embodiments, an action is implemented on a single-analyte system 1420 based upon the process metric obtained in step 1410. In some embodiments, subsequent to implementing the action on the single-analyte system 1420, the single-analyte data set is updated 1430. In some embodiments, after updating the single-analyte data set 1430, a decision 1440 is made regarding whether a determinant criterium for terminating the iterative process has been achieved. In some embodiments, if a determinant criterium has been achieved, the iterative process is terminated 1450. In some embodiments, if a determinant criterium has not been achieved, the iterative process is continued, for example, by performing another cycle of the iterative process. The skilled person will readily recognize that the iterative process is modified in some such embodiments. For example, in some embodiments, the decision 1440 regarding a determinant criterium is performed at any point during a cycle of the iterative process. In some embodiments, the decision 1440 regarding a determinant criterium is performed more than once during a cycle of the iterative process. In some embodiments, one or more additional undescribed steps, procedures, or sub-procedures is included within one or more cycles of the iterative process.

Also described herein is a method for controlling a single-analyte process, the method comprising performing an iterative process until a determinant criterium has been met, in which the iterative process comprises the steps of: combining data from a single-analyte data set comprising data from more than one data source to determine a process metric for a single analyte; implementing an action on a single-analyte system based upon the process metric, in which the single-analyte system comprises a detection system that is configured to obtain a physical measurement of the single analyte at single-analyte resolution; and updating the single-analyte data set after implementing the action on the single-analyte system.

Also described herein is a method for controlling the processes of a single-analyte process, the method comprising performing an iterative process until a determinant criterium has been met, in which the iterative process comprises the steps of: determining a process metric for a single analyte based upon a single-analyte data set; implementing an action on a single-analyte system that alters a source of uncertainty based upon the process metric, in which the single-analyte system comprises a detection system that is configured to obtain a physical measurement of the single analyte at single-analyte resolution; and updating the single-analyte data set after implementing the action on the single-analyte system.

Also described herein is a method for controlling the processes of a single-analyte process, the method comprising performing an iterative process until a completion criterium has been met, in which the iterative process comprises the steps of: determining a curated uncertainty metric for a plurality of single analytes based upon a single-analyte data set; implementing an action on a single-analyte system based upon the curated uncertainty metric, in which the single-analyte system comprises a detection system that is configured to obtain a physical measurement at single-analyte resolution of each single analyte of the plurality of single analytes; and updating the single-analyte data set after implementing the action on the single-analyte system.

In some embodiments, the methods and systems described herein are advantageously applied to single-analyte systems that are configured to provide single-molecule characterization of a single analyte, or a plurality of single analytes, at single-analyte resolution (e.g., an array of sites that are each attached to a single analyte). In some embodiments, the methods and system are used for an application of a single-analyte system, including the synthesis, fabrication, manipulation, and/or degradation of single analytes, as well as the assaying of single analytes. In some embodiments, a single-analyte process includes a synthesis, fabrication, manipulation, and/or degradation process that is coupled with an assay process, for example an assay to characterize a single analyte during the synthesis, fabrication, manipulation, or degradation process. In some embodiments, a single-analyte system includes one or more biological single analytes (e.g., polypeptides, polynucleotides, polysaccharides, metabolites, cofactors, etc.), one or more non-biological single analytes (e.g., organic or inorganic nanoparticles), or a combination thereof.

In some embodiments, synthesis of biological single analytes includes a single-analyte process that modifies the chemical structure of a biological single analyte, including, for example, growth, catalyzed growth, addition of a moiety, removal of a moiety, rearrangement of chemical bonds in a moiety, polymerization, concatenation, extrusion, conjugation, reaction, deposition, post translational modification of protein, or a combination thereof. In some embodiments, fabrication of biological single analytes includes a single-analyte process that forms a useful structure or device from a biological single analyte, including nano-device formation, nanofluidics, and self-assembling devices. In some embodiments, non-covalent manipulation of biological single analytes includes a process that does not alter the primary chemical structure or composition of a biological single analyte, including, for example, crystallization, folding, nucleation, recrystallization, re-folding, denaturation, non-covalent complex formation, repositioning, re-orientation, extraction from a fluid sample, separation from at least one other analyte, purification from a sample, delivery to a vessel or solid support, removal from a vessel or solid support, transfer via a fluidic apparatus or process, transfer via charge attraction or repulsion, transfer via magnetic attraction or repulsion, absorption of energy (e.g., radiation), or confinement. In some embodiments, degradation of a biological single analyte includes a process that decreases or reduces the primary structure of a biological single analyte, including, for example, cleavage, elimination, decomposition, digestion, sloughing, dissociation, lysis, oxidative decomposition, reductive decomposition, enzymatic degradation (e.g., proteolysis of proteins or nucleolysis of nucleic acids), photodegradation or photolysis, or thermal decomposition.

In some embodiments, synthesis of non-biological single analytes includes a single-analyte process that modifies the chemical structure of a non-biological single analyte, including, for example, growth, catalyzed growth, addition of a moiety, removal of a moiety, rearrangement of chemical bonds in a moiety, polymerization, concatenation, extrusion, conjugation, reaction, deposition, crystallization, nucleation, or a combination thereof. In some embodiments, fabrication of non-biological single analytes includes a single-analyte process that forms a useful structure or device from a non-biological single analyte, including, for example, nano-device formation (e.g., nano-circuits), nanofluidics (e.g., nano-pumps), and self-assembling devices. In some embodiments, non-covalent manipulation of non-biological single analytes includes a process that does not alter the primary chemical structure or composition of a non-biological single analyte, including for example, crystallization, nucleation, recrystallization, disassembly, non-covalent complex formation, repositioning, re-orientation, extraction from a fluid sample, separation from at least one other analyte, purification from a sample, delivery to a vessel or solid support, removal from a vessel or solid support, transfer via a fluidic apparatus or process, transfer via charge attraction or repulsion, transfer via magnetic attraction or repulsion, absorption of energy (e.g., radiation), or confinement. In some embodiments, degradation of a non-biological single analyte includes a process that decreases or reduces the primary structure of a non-biological single analyte, including, for example, cleavage, elimination, decomposition, dissociation, oxidative decomposition, reductive decomposition, enzymatic degradation, non-enzymatic degradation, catalytic degradation, photodegradation or photolysis, or thermal decomposition.

In some embodiments, an assay of a single analyte includes any process that is intended to determine presence, absence, a location, an identity, a property, a characteristic, a behavior, or an interaction of the single analyte (e.g., a biological single analyte or a non-biological single analyte), including, for example, single analyte chemical property determination, single analyte identification, single analyte characterization, single analyte categorization, single analyte quantification, single analyte sequencing, and single analyte binding assays. In some embodiments, a single-analyte process incorporates an assaying process to provide a physical characterization of a single analyte during a non-assay single-analyte process.

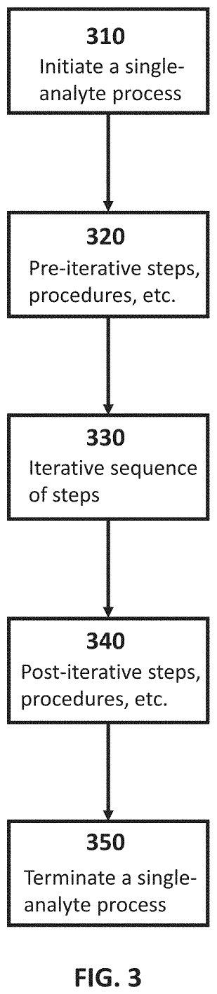

In some embodiments, a single-analyte process includes a plurality of steps, actions, procedures, or sub-procedures that are performed during the course of the single-analyte process. In some embodiments, the plurality of steps, actions, procedures, or sub-procedures includes physical operations (e.g., operation of a hardware component), computational operations, algorithmic operations, logical operations, or a combination thereof. In some embodiments, a single-analyte process of the present disclosure includes an iterative sequence of steps, in which the iterative sequence of steps includes one or more repeated steps, actions, procedures, or sub-procedures. FIG. 3 presents a flowchart depicting a simplified single-analyte process comprising an iterative sequence of steps. Block 310 depicts the initiation of single-analyte process. In some embodiments, initiation includes any step, procedure, or sub-procedure that begins a single-analyte process, such as providing an analyte, a reagent, or an initiation instruction. In some embodiments after initiation 310, a single-analyte process includes a sequence of one or more pre-iteration steps, procedures, or sub-procedures 320. In some embodiments, following any pre-iterations steps, procedures, or sub-procedures 320, a single-analyte process includes an iterative sequence of steps 330. In some embodiments, after completion of the iterative sequence of steps 330, the single-analyte process optionally include any post-iteration steps, procedures, or sub-procedures 340. In some embodiments, the single-analyte process then proceeds to a termination step, procedure, or sub-procedure 350. In some embodiments, it will be recognized that the single-analyte process described in FIG. 3 is modified to include, for example, additional iterative sequences of steps 330 and additional post-iteration steps, procedures, or sub-procedures 340 between the additional sequences of steps 330.

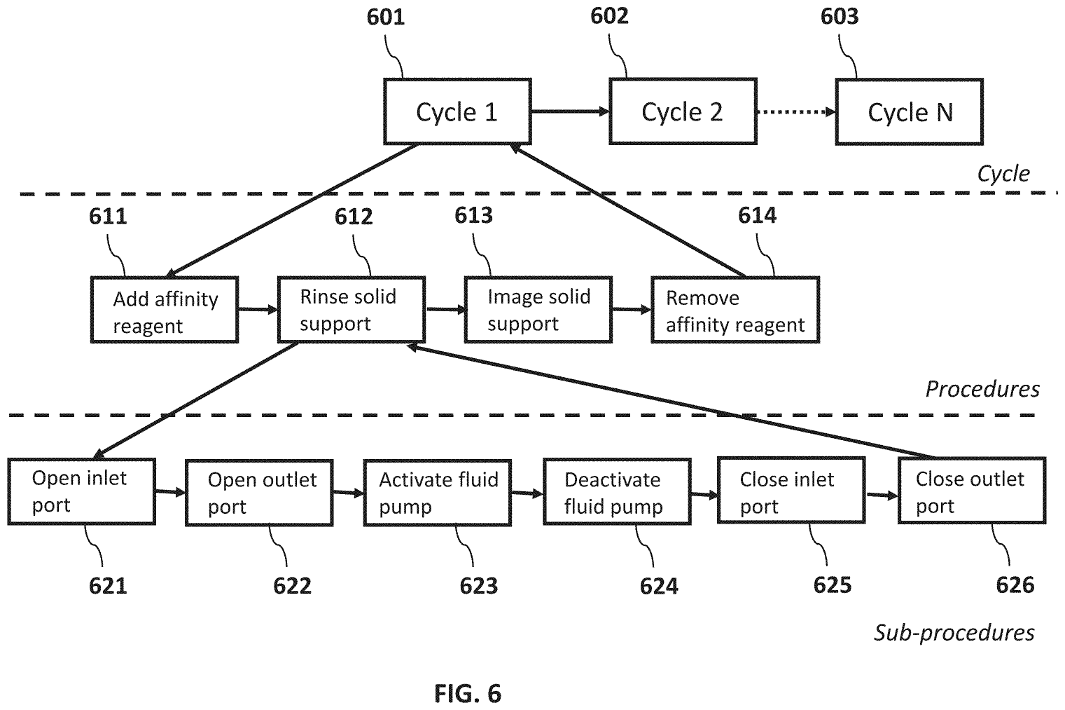

In some embodiments, a single-analyte process includes a sequence of steps, procedures, or sub-procedures that collectively form the single-analyte process. In some embodiments, a sequence of steps includes a nested structure of procedures and sub-procedures. For example, in some embodiments, a step of a sequence of steps includes a sequence of procedures, and/or the sequence of procedures includes a sequence of sub-procedures. FIG. 6 illustrates the structure of a sequence of steps for a single-analyte assay process comprising affinity reagent binding measurements. In some embodiments, the single-analyte process includes a sequence of N successive cycles 601, 602, . . . , 603, in which each cycle includes multiple procedures. Cycle 1 is shown to comprise an affinity reagent binding procedure 611, a solid support rinsing procedure 612, a solid support imaging procedure 613, and an affinity reagent binding removal procedure 614. In some embodiments, each successive cycle (e.g., 602, 603) includes an identical or similar set of procedures. For example, in some embodiments, cycle 602 includes procedures 611, 612, 613 and 614, as cycle 603. It will be understood that all cycles performed in an iterative process set forth herein need not be identical nor even similar to each other. For example, in some embodiments, cycle 602 includes a differing sequence of procedures in comparison to cycle 601, cycle 602 omits at least one procedure included in cycle 601, or cycle 602 adds at least one procedure that was not performed in cycle 601.

In some embodiments, as exemplified in FIG. 6, one or more of the procedures include multiple sub-procedures. The solid support rinsing procedure is shown to comprise an inlet port opening sub-procedure 621, an outlet port opening sub-procedure 622, a fluid pump activation sub-procedure 623, a fluid pump deactivation sub-procedure 624, an inlet port closing sub-procedure 625, and an outlet port closing sub-procedure 626. In some embodiments, each procedure of the single-analyte process depicted in FIG. 6 includes an identical, similar, or differing sequence of sub-procedures.

In some embodiments, a sequence of steps (e.g., cycles, procedures, or sub-procedures) is determined before a single-analyte process has been initiated. In some embodiments, a sequence of steps is determined or modified after a single-analyte process has been initiated. For example, in some embodiments, a sequence of steps is modified in response to information obtained from a previous step, for example, in accordance with systems and methods set forth herein for controlling single-analyte processes. In some embodiments, a sequence of steps is determined before an iterative process within a single-analyte process has been initiated. In some embodiments, a sequence of steps is determined or modified after an iterative process within a single-analyte process has been initiated. For example, in some embodiments, a sequence of steps in an iterative process is modified in response to information obtained from some or all previous step in the iterative process, for example, in accordance with systems and methods set forth herein for controlling single-analyte processes. In some embodiments, a sequence of steps is determined before a single-analyte process or before an iterative process, and then is altered during the iterative process. In some embodiments, a sequence of steps is determined during an iterative process. In some embodiments, a single step of the sequence of steps is determined or modified during an iteration of the iterative process. In other embodiments, two or more steps of a sequence of steps are determined during an iteration of the iterative process.

In some embodiments, a sequence of steps (e.g., cycles, procedures, or sub-procedures) is classified depending upon when it is configured and/or how it is applied in a single-analyte process. In some embodiments, a sequence of steps, procedures, or sub-procedures is classified as a preliminary, partial, full, or altered sequence of steps, procedures, or sub-procedures. In some embodiments, a preliminary sequence of steps includes a sequence of steps that is determined before a single-analyte process is initiated or a sequence of steps that is determined before an iterative process is initiated. In some embodiments, a partial sequence of steps includes a sequence of steps that does not include a complete prescription for a single-analyte process. For example, in some embodiments, a partial sequence of steps includes instructions (e.g., sequences of cycles, procedures, or sub-procedures) for a set number of cycles (e.g., 10, 20, 30, 40, or 50 cycles) of a single-analyte process that requires or otherwise includes more than 50 cycles. In some embodiments, a partial sequence of steps includes a discontinuous sequence of steps with inter-sequence gaps intended to be controlled by an iterative process. In some embodiments, a full sequence of steps includes a sequence of steps that includes a complete prescription for the completion of a single-analyte process. For example, in some embodiments, a full sequence of steps includes a complete set of instructions for a single-analyte process (e.g., synthesis, fabrication, manipulation, degradation or assay), including all cycles, procedures, and/or sub-procedures to perform the process. In some embodiments, a full sequence of steps includes a “standard” prescription for a single-analyte process, in which an iterative process is to be implemented to customize control of the process. In some embodiments, a preliminary sequence of steps is a partial or full sequence of steps. For example, in some embodiments, a partial sequence of steps is provided to a single-analyte process for a purpose such as establishing a baseline measure of one or more process metrics before initiating an iterative process. In some embodiments, a full sequence of steps is provided to a single-analyte process as a consensus sequence of steps for a single-analyte process, in which an iterative process is initiated if one or more process metrics suggest that the performance of the process is not achieving an expected outcome.

In some embodiments, an altered sequence of steps includes a sequence of steps that has been altered from a prior prescription of a single-analyte process. In a first example, a full sequence of steps is revised after an iterative process, thereby providing an altered sequence of steps. In a further example, the altered sequence of steps of the first example is provided to a second single-analyte process and subsequently altered by another iterative process, thereby providing a second altered sequence of steps. In some embodiments, an altered sequence of steps is a partial or full sequence of steps. For example, in some embodiments, an altered sequence of steps is provided as a partial sequence of steps if a prior single-analyte process has previously demonstrated unreliable behavior after a particular number of steps of a full sequence of steps. In some embodiments, an altered sequence of steps is provided as a partial sequence of steps if particular steps have been found to be optional, in which an iterative process is implemented to decide whether or not to perform the optional steps. In some embodiments, an altered sequence of steps is provided as a full sequence of steps if the full sequence of steps is parameterized by information derived from a preliminary single-analyte data set (i.e., information on single-analyte type, reagent types, or final product alters the parameterization of a full sequence of steps for the same basic process).

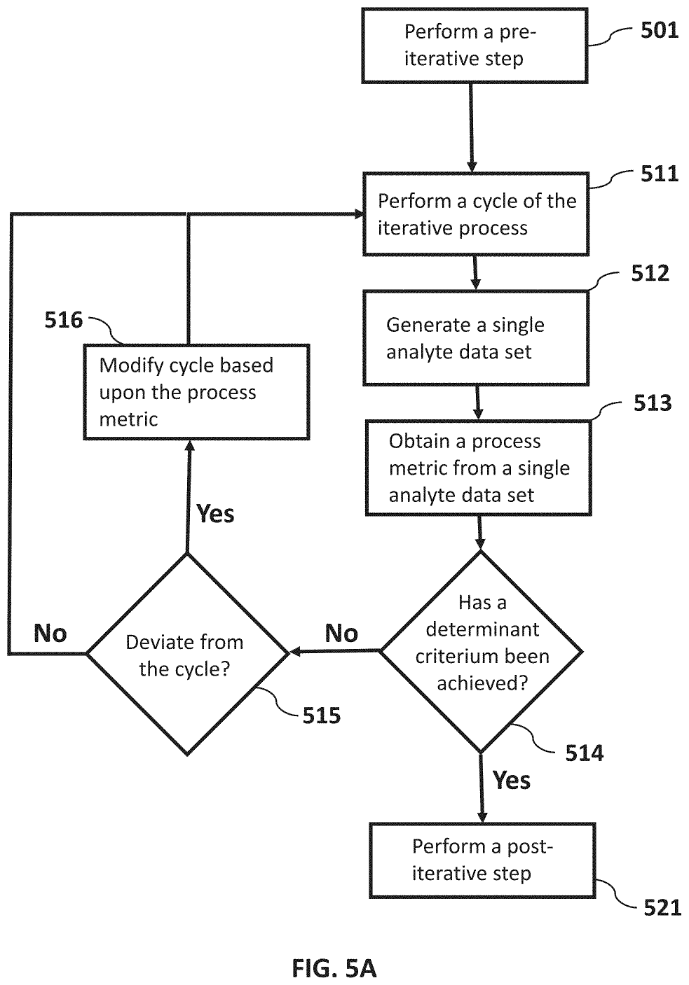

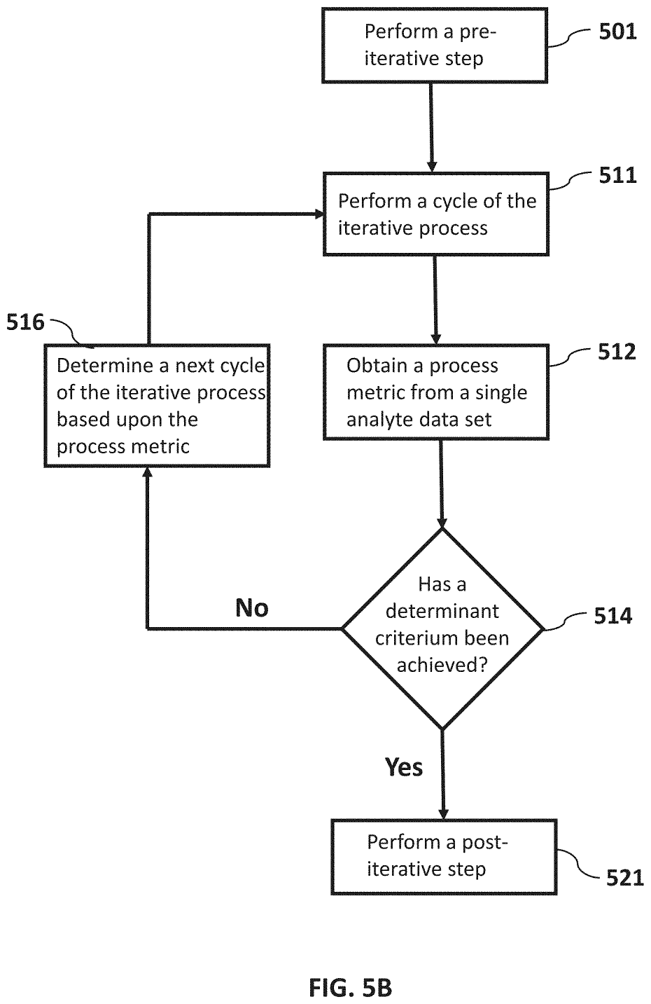

In some embodiments, a single-analyte process, as described herein, includes an iterative process that is configured to formulate, alter, or improve a sequence of steps for the single-analyte process. In some embodiments, formulating a sequence of steps for the single-analyte process includes generating and/or configuring a sequence of one or more steps that collectively form the single-analyte process. In some embodiments, altering a sequence of steps for the single-analyte process includes adding steps, removing steps, repeating steps, rearranging steps, or a combination thereof. In some embodiments, improving a sequence of steps includes reducing the number of steps, reducing an input to the single-analyte process (e.g., reagents, energy, time), improving the quality of an outcome of the single-analyte process, improving the likelihood that an outcome of the single-analyte process will be achieved, or a combination thereof. FIGS. 5A-5B provide flowcharts depicting approaches for determining a sequence of steps for an iterative single-analyte process. FIG. 5A depicts a regimented approach to determining a sequence of steps for an iterative single-analyte process. In some embodiments, a regimented approach begins with determining a preliminary cycle, in which each cycle includes a sequence of procedures. In some embodiments, the preliminary cycle includes one or more pre-iterative steps 501 that are performed before initiating the iterative process. In some embodiments, the iterative process is initiated by performing a cycle of the iterative process 511 and generating a single-analyte data set 512. In some embodiments, the iterative process continues by obtaining a process metric from the single-analyte data set 513. In some embodiments, if the process metric does prompt altering one or more procedures of the cycle, a decision 514 is made regarding whether the process metric indicates the achieving of a determinant criterium. In some embodiments, if a determinant criterium has been achieved, the single analyte proceeds to an optional post-iterative step 521. In some embodiments, the optional post iterative step 521 includes terminating the single-analyte process, for example, after a predetermined threshold has been achieved (e.g., completion of a predetermined number of cycles) or based on the process metric obtained from a previous cycle (e.g., acquiring sufficient data to satisfy an objective such as identifying an analyte of interest). In some embodiments, if the decision 514 is made that a determinant criterium has not been achieved, a second decision 515 whether to deviate from the sequence of steps is made based upon the obtained process metric 513. In some embodiments, if the decision to deviate 515 is made, then one or more steps, procedures, or sub-procedures of the cycle is then be modified or altered 516 based upon the process metric (e.g., by an algorithm, by a user input). In some embodiments, a subsequent cycle is modified or altered 516 based upon the determined process metric or another process metric, for example, by adding a process to the cycle, removing a process from the cycle, or changing the sequence of processes in the cycle. In some embodiments, if the process metric does not indicate the need to deviate 515 from the sequence of steps, the single-analyte process is continued by proceeding to the next cycle of the iterative process 511. In some embodiments, the iterative process then proceeds to the next cycle of the iterative process 511 without modification based upon the process metric. Aspects of the regimented iterative process shown in FIG. 5A are demonstrated in Examples 1, 2, 4, 7, and 12 below.

FIG. 5B depicts a step-wise approach to determining a sequence of steps for a single-analyte process. In some embodiments, a step-wise approach is implemented in the absence of a preliminary sequence of steps, or at the completion of a partial sequence of steps. In some embodiments, a single-analyte process includes one or more pre-iterative steps 501 that are performed before initiating an iterative process. In some embodiments, the iterative process is initiated by performing a step from the preliminary sequence of steps 511 and determining a single-analyte data set. In some embodiments, the iterative process continues by obtaining a process metric from the single-analyte data set 512. In some embodiments, a decision 514 is made regarding whether the process metric indicates the achieving of a determinant criterium. In some embodiments, if a determinant criterium has been achieved, the single analyte proceeds to an optional post-iterative step 521. In some embodiments, if a determinant criterium has not been achieved, a next step or a partial sequence of steps is determined based upon the determined process metric 516. In some embodiments, the iterative process then proceeds to the next step of the sequence of steps 511 based upon the determined next step or partial sequence of steps. Aspects of the iterative process shown in FIG. 5B are demonstrated in Examples 3, 7, 10, and 11 below.

In some embodiments, an iterative process within a single-analyte process proceeds until a determinant criterium has been achieved. In some embodiments, a determinant criterium includes a fixed criterium which is not altered prior to the completion of an iterative process. For example, in some embodiments, the determinant criterium that is used to determine whether or not to proceed with an iterative process is defined by a manufacturer as a system preset or by a user based on a priori information. In some embodiments, a determinant criterium includes a variable criterium which is altered before the completion of an iterative process. For example, in some embodiments, the determinant criterium that is used to determine whether or not to proceed with an iterative process is a variable criterium that is modified, at least in part, based on a process metric (or other information) obtained during the course of performing the iterative process. In some embodiments, a determinant criterium excludes all fixed criteria or any particular fixed criterium set forth herein. In some embodiments, a determinant criterium excludes all variable criteria or any particular variable criterium set forth herein.

In some embodiments, as exemplified above, a determinant criterium that is based on a fixed criterium is a manually-defined criterium (e.g., specified by a user) or is an automatically-defined criterium (e.g., programmed into an algorithm). In some embodiments, a manually-defined criterium or automatically-defined criterium provides an initiation criterium for a variable criterium. In some embodiments, a variable criterium is modified, at least in part, based on a manually defined criterium or automatically defined criterium. In some embodiments, manually defined determinant criterium or automatically defined determinant criterium, is specific to a particular single-analyte or to a particular single-analyte process. For example, a single-molecule proteomic assay includes a first suite of determinant criteria that differ from a second suite of determinant criteria for a single-molecule transcriptomic assay. However, in some embodiments, within this example, certain members of the first suite of determinant criteria overlap or be identical to certain members of the second suite of determinant criteria. Moreover, determinant criteria need not be specific to a particular single-analyte or single-analyte process, for example, instead being general to a class of single analytes or a class of single-analyte processes.

In some embodiments, a determinant criterium is provided to a system or method set forth herein before, during, or after the initiation of an iterative process. In some embodiments, determinant criterium is based, at least in part, upon data provided to an algorithm before, during, or after the initiation of an iterative process. For example, in some embodiments, the information indicates the type of single-analyte process to be performed, an expected initial state of the single analyte, an expected final state of the single analyte, or any other known information. In some embodiments, user provides the information to an algorithm that subsequently defines a determinant criterium prior to initiating the iterative process. In another example, a single-analyte system collects an initial data set at the initiation of a single-analyte process and subsequently define a determinant criterium.

In some embodiments, an iterative process is completed when an unforced determinant criterium has been achieved. In some embodiments, an unforced determinant criterium includes any determinant criterium that is achieved due to the intended performance of the iterative process. In some embodiments, an unforced determinant criterium is user-defined, or automatically defined (e.g., algorithmically-defined). In some embodiments, an unforced determinant criterium includes a determinant criterium that is calculated, compiled, derived, or inferred from data collected during a single-analyte process. In some embodiments, an unforced determinant criterium is selected from the group consisting of: a fixed number of cycles of the iterative process, for example, each of the cycles comprising one or more processes of an iterative process exemplified forth herein; a maximum number of cycles of the iterative process, for example, each of the cycles comprising one or more processes of an iterative process exemplified forth herein; a minimum number of cycles of the iterative process, for example, each of the cycles comprising one or more processes of an iterative process exemplified forth herein; the process metric (e.g., uncertainty metric) traversing a threshold value; a categorized value of the process metric (e.g., uncertainty metric) changing from a first categorized value to a second categorized value; a trend in the process metric (e.g., uncertainty metric); a pattern in the process metric (e.g., uncertainty metric); and obtaining a final characterization of the single analyte.

In some embodiments, a single-analyte process includes an iterative process that iterates for a particular number of cycles, in which, for example, each of the cycles comprises one or more processes of an iterative process exemplified forth herein. In some embodiments, an iterative process iterates for a minimum number of cycles of at least about 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 3000, 4000, 5000, 6000, 7000, 8000, 9000, 10000, 25000, 50000, 100000, or more cycles. In some embodiments, an iterative process iterates for a maximum number of cycles of no more than about 100000, 50000, 25000, 10000, 9000, 8000, 7000, 6000, 5000, 4000, 3000, 2000, 1900, 1800, 1700, 1600, 1500, 1400, 1300, 1200, 1100, 1000, 950, 900, 850, 800, 750, 700, 650, 600, 550, 500, 450, 400, 350, 300, 250, 200, 190, 180, 170, 160, 150, 140, 130, 120, 110, 100, 90, 80, 70, 60, 50, 45, 40, 35, 30, 25, 20, 15, 10, 9, 8, 7, 6, 5, 4, 3, or fewer cycles.

In some embodiments, the fixed number of cycles, the maximum number of cycles, or the minimum number of cycles of an iterative process is determined based upon a preliminary single-analyte data set. In some embodiments, a preliminary single-analyte data set includes one or more pieces of information that are used to determine a number of cycles for the iterative process. In some embodiments, the one or more pieces of information includes user-provided information (e.g., type of single analyte, type of single-analyte process, etc.), stored or reference information (e.g., prior process configurations, prior process results, cumulative data etc.), preliminary single-analyte physical data, and a preliminary single-analyte process metric (e.g., uncertainty metric). For example, in some embodiments, a preliminary single-analyte data set includes user-provided data on sample type, analyte type and/or properties, user-provided and/or supplier-provided reagent information, and/or preliminary or baseline physical measurements of a single analyte or other system component. In some embodiments, a preliminary single-analyte data set includes cumulative data that has been stored from previous runs of a similar sample and/or single analyte. In some embodiments, preliminary single-analyte physical data or a preliminary single-analyte process metric (e.g., uncertainty metric) is determined before a single-analyte process or before an iterative process. For example, in some embodiments, a background or baseline value for a physical measurement (e.g., an autofluorescence value for an optical measurement) is collected before a single-analyte process has been initiated. In some embodiments, a preliminary process metric is calculated after a preliminary sequence of steps, and the preliminary process metric is utilized during an initial cycle of an iterative process of the single-analyte process.

In some embodiments, a determinant criterium indicates, for example, a prescribed quantity of cycles of an iterative process, such as a fixed number of cycles, a maximum number of cycles, or a minimum number of cycles. In some embodiments, a determinant criterium is provided to a method or system of the present disclosure at any time before, during, or after the initiation of a single-analyte process or an iterative process. In some embodiments, the determinant criterium is provided or altered before a first cycle of an iterative process comprising one or more processes of an iterative process exemplified herein (e.g., the process described in FIG. 14). In some embodiments, the determinant criterium is provided or altered after a first cycle of an iterative process comprising one or more processes of an iterative process exemplified forth herein (e.g., the process described in FIG. 14).

In some embodiments, a determinant criterium is provided or altered based, at least in part, upon a default value or a user-defined value, for example, a value that functions as a threshold. In some embodiments, a default value is a specified value for a quantity of cycles that has been pre-determined, for example, based upon an instrumental configuration, an analyte type, or a process type. In some embodiments, a user-defined value is a specified value for a quantity of cycles that is provided by a user to a single-analyte system before, during, or after the initiation of a single-analyte process or an iterative process. For example, in some embodiments, a user is prompted to provide a quantity of iterations for a single-analyte process before initiating the process. In some embodiments, the determinant criterium is based, at least in part, upon a default value or a user-defined value before a first cycle of the iterative process comprising one or more processes of an iterative process exemplified herein (e.g., the process described in FIG. 14). In some embodiments, the fixed number of cycles, the maximum number of cycles, or the minimum number of cycles is determined based, at least in part, upon a default value or a user-defined value after a first cycle of the iterative process comprising one or more processes of an iterative process exemplified herein (e.g., the process described in FIG. 14).

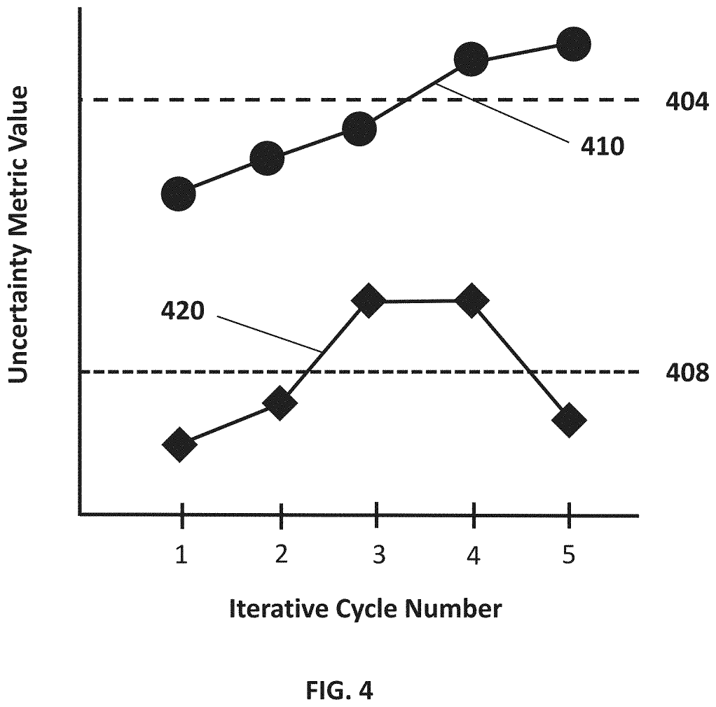

In some embodiments, an unforced determinant criterium for completing an iterative process includes a process metric (e.g., uncertainty metric) determined relative to a threshold value for the process metric (e.g., uncertainty metric). In some embodiments, a threshold value includes a standard value, a benchmark value, a targeted value, a failsafe value, a maximum value, or a minimum value for a process metric (e.g., uncertainty metric). In some embodiments, a process metric (e.g., uncertainty metric) traverses a threshold value when the numerical difference between the process metric (e.g., uncertainty metric) and the threshold value reverses its sign (i.e., turns from negative to positive, or vice versa). In some embodiments, a process metric (e.g., uncertainty metric) traverses a threshold value when an enumerated or categorized value changes (e.g., changes from “unidentified” to “identified”). In some embodiments, the process metric (e.g., uncertainty metric) traversing a threshold value includes the process metric (e.g., uncertainty metric) increasing above a threshold value. In some embodiments, the process metric (e.g., uncertainty metric) traversing a threshold value includes the process metric (e.g., uncertainty metric) decreasing below a threshold value. FIG. 4 depicts a graph plotting the values of a first uncertainty metric (shown as circles) and the values of a second uncertainty metric (shown as diamonds) as measured for each cycle of a hypothetical iterative process. The values of the first uncertainty metric are plotted with respect to a first threshold value 404 for the first uncertainty metric. The values of the second uncertainty metric are plotted with respect to a second threshold value 408 for the second uncertainty metric. An increasing trendline 410 is observed for the first uncertainty metric. In some embodiments, the first uncertainty metric is determined to have traversed a threshold value at cycle 4 when the value of the uncertainty metric rises above the threshold value 404. In some embodiments, a variable trendline 408 is observed for the second uncertainty metric. In some embodiments, the second uncertainty metric is determined to traverse a threshold at cycle 3 when it rises above the second threshold value 408, or at cycle 5 when it falls back below the uncertainty threshold 408. In some embodiments, the threshold value is determined based upon a preliminary single-analyte data set. In some embodiments, the threshold value is a default value or a user-defined value.

In some embodiments, an unforced determinant criterium for completing an iterative process includes a change in an enumerated or categorized value determined for a process metric (e.g., uncertainty metric). In some embodiments, an enumerated or categorized value for a process metric (e.g., uncertainty metric) include a binary, a trinary, or a polynary group. In some embodiments, enumerated or categorized values of a process metric (e.g., uncertainty metric) are classified by a qualitative or quantitative definition. In some embodiments, enumerated or categorized values of a process metric (e.g., uncertainty metric) are manually determined or determined by a non-manual method (e.g., a computer-implemented algorithm). In some embodiments, a determinant criterium for an iterative process includes determining a change in an enumerated or a categorized value from a first value to a second value. For example, in some embodiments, the first value and/or the second value is a member of a binary group, for example a binary group selected from ON/OFF, NORMAL/NOT NORMAL, NORMAL/ERROR, OBSERVED/NOT OBSERVED, POSITIVE/NEGATIVE, OPEN/CLOSED, STOP/GO, PAUSE/RESUME, READY/NOT READY, FAIL/PASS, and MATCH/NO MATCH. In some embodiments, the first value and/or the second value is a member of a trinary or polynary pair group in which the determinant criterium is achieved when the first value changes to a second value. For example, in some embodiments, the determinant criterium is achieved when the first value changes to any other value of the trinary or polynary group (e.g., type 1 to type 2, type 3, or type 4). In some embodiments, the determinant criterium is achieved when the first value changes to a particular other value of the trinary or polynary group (type 1 to type 3, but not type 2 or type 4).

In some embodiments, an unforced determinant criterium for completing an iterative process includes a trend of a process metric (e.g., uncertainty metric). In some embodiments, a trend of a process metric (e.g., uncertainty metric) includes a consistent direction of change in the process metric (e.g., uncertainty metric) over a plurality of steps or cycles. In some embodiments, a trend of a process metric (e.g., uncertainty metric) is an increasing trend, a neutral trend, or a decreasing trend. In some embodiments, a trend of a process metric (e.g., uncertainty metric) is characterized as having a mathematical relationship as a function of process time, step or cycle number, or other process parameter. For example, in some embodiments, a trend of a process metric (e.g., uncertainty metric) is characterized as linear, polynomial, geometric, exponential, logarithmic, sigmoidal, sinusoidal, or a combination thereof. In some embodiments, a trend is determined over a minimum number of steps or cycles, for example, at least about 2, 3, 4, 5, 6, 7, 8, 9, 10, 15, 20, 25, 30, 35, 40, 45, 50, 60, 70, 80, 90, 100, 150, 200, 250, 300, 400, 500, 600, 700, 800, 900, 1000, or more steps or cycles. In some embodiments, a trend is determined over a maximum number of steps or cycles, for example, no more than about 1000, 900, 800, 700, 600, 500, 400, 300, 250, 200, 150, 100, 90, 80, 70, 60, 50, 45, 40, 35, 30, 25, 20, 15, 10, 9, 8, 7, 6, 5, 4, 3, or fewer steps or cycles.

In some embodiments, an unforced determinant criterium for completing an iterative process is based on a change in trend of a process metric (e.g., uncertainty metric). For example, in some embodiments, an unforced determinant criterium is satisfied when the slope of a trend crosses a threshold. In some embodiments, an unforced determinant criterium is satisfied when the derivative of a trend crosses a threshold. In some embodiments, the threshold in these examples is a minimum value, a maximum value, a banded range delineated by a maximum and minimum value, a deviation from a specified trend (e.g., a correlation coefficient), or the like.

In some embodiments, a n unforced determinant criterium for completing an iterative process includes a pattern of a process metric (e.g., uncertainty metric). In some embodiments, a pattern of a process metric (e.g., uncertainty metric) includes a repeated behavior in the process metric (e.g., uncertainty metric) over a plurality of steps or cycles. In some embodiments, a pattern of a process metric (e.g., uncertainty metric) is characterized, for example, as an arithmetic pattern, a geometric pattern, a diverging pattern, a converging pattern, an oscillatory pattern, an alternating pattern, a static pattern, a repeating pattern, an expanding pattern, a contracting pattern, or a combination thereof. In some embodiments, a pattern is determined for a quantitative process metric (e.g., a quantitative uncertainty metric). In some embodiments, a pattern is determined for a qualitative process metric (e.g., a qualitative uncertainty metric) (e.g., present, present, absent, present, present, absent, etc.).

In some embodiments, an unforced determinant criterium for completing an iterative process includes one or more threshold characteristics of an analyte. For example, in some embodiments, an iterative process for characterizing a single analyte is terminated based upon obtaining a characterization of the single analyte that correlates with one or more threshold characteristics. In some embodiments, a characteristic of a single analyte that is determined from an iterative process to correlate with a threshold characteristic is considered a ‘final characterization.’ In some embodiments, this is determined whether the characteristic is observed before, after or during the final cycle of the iterative process. In some embodiments, a final characterization of a single analyte is utilized to confirm the completion of a single-analyte process. For example, in some embodiments, a final characterization of a single analyte is utilized to obtain an identity for the single analyte, obtain a physical property of the single analyte (e.g., size, polarity, electrical charge, absorption spectrum, emission spectrum, etc.), confirm a complete synthesis of the single analyte, confirm a fabrication of the single analyte, confirm a manipulation of the single analyte, determine a state for the single analyte (e.g., a post-translational modification state, an activation state, an oxidation state, etc.), determine an interaction of the single analyte (e.g., analyte-ligand binding, analyte-catalyzed reaction, etc.), determining a structure of the single analyte (e.g., atomic structure, molecular structure, crystal structure, etc.), or a combination thereof.

In some embodiments, an iterative process is completed when a forced determinant criterium has been achieved. In some embodiments, a forced determinant criterium includes any determinant criterium that is achieved due to a premature, unexpected, unscheduled, or unplanned deviation in the performance of the iterative process. In some embodiments, an unplanned deviation includes a technical deviation, an algorithmic deviation, or a combination thereof. In some embodiments, a technical deviation includes unexpected or unwanted departure from normal or intended operation of a component of a single-analyte system. For example, in some embodiments, technical deviations include erroneous operations of system hardware, hardware damage, and user-driven hardware errors. In some embodiments, an algorithmic deviation includes unexpected or unwanted departure from normal or intended operation of an algorithm of a single-analyte system. For example, in some embodiments, algorithmic deviations include conflicting algorithmic calculations and non-converging algorithmic calculations. In some embodiments, a forced determinant criterium includes a user input or a system feedback.

In some embodiments, forced determinant criterium comprising a user input includes any premature, unexpected, unscheduled, or unplanned user-initiated interventions in the performance of an iterative process during a single-analyte process. In some embodiments, a user input includes one or more user-specified, user-defined, or user-selected instructions that cause a deviation in the performance of a single-analyte process or an iterative process. For example, in some embodiments, a single-analyte process includes one or more prompts to a user to provide information or an instruction that includes the termination of an iterative process. In some embodiments, a user input is prompted by a single-analyte system, or is unprompted by the system. In some embodiments, a user input includes an input selected from the group consisting of: an instruction to discontinue the single-analyte process; an instruction to discontinue the iterative process; an instruction to alter a sequence of steps of the single-analyte process; an instruction to alter a sequence of steps of the iterative process; a manual identification of a trend in the process metric (e.g., uncertainty metric); a manual identification of a pattern in the process metric (e.g., uncertainty metric); a manual identification of a categorized value of the process metric (e.g., uncertainty metric); and a manual confirmation of a characterization of the single analyte.

In some embodiments, forced determinant criterium comprising system feedback includes any unexpected, unscheduled, or unplanned system-initiated interventions in the performance of an iterative process during a single-analyte process. In some embodiments, system feedback includes one or more system-specified, system-defined, or system-selected instructions that cause a change in the performance of a single-analyte process or an iterative process. In some embodiments, system feedback includes an automated system feedback to the single-analyte process. In some embodiments, system feedback includes a request for a user input. In some embodiments, system feedback is caused by a temporary system failure mode (e.g., low reagent levels) or permanent system failure mode (e.g., a failed circuit board). In some embodiments, system feedback, for example, comprises a feedback selected from the groups consisting of: a critical reagent level; an addressable hardware failure mode; a non-addressable hardware failure mode; a software failure mode; a critical environmental condition; and an unexpected external condition.

In some embodiments, a critical environmental condition includes any change in a physical environment adjacent to a single-analyte system that impacts the function of the system. For example, in some embodiments, critical environmental conditions include changes in temperature, gas pressure, gas composition (e.g., humidity), liquid pressure, liquid composition, orientation, velocity, acceleration, force, momentum, vibration, irradiation, electric field, magnetic field, or a combination thereof. In some embodiments, an unexpected external condition includes any disruptive event external to a single-analyte system that impacts the function of the system. In some embodiments, an unexpected external event is anthropogenic or naturally-occurring. For example, in some embodiments, an unexpected external condition includes a natural disaster such as an earthquake, a tsunami, an avalanche, a tornado, a hurricane, a thunderstorm, a flood, a blizzard, a windstorm, a sinkhole, a volcanic eruption, a wildfire, a solar flare, or a combination thereof. In another example, an unexpected external condition includes an anthropogenic event, such as an explosion, an impact, a gas leak, a water leak, a power failure, a power surge, a cyberattack, an improper system installation, an improper process setup, or a combination thereof.

In some embodiments, an iterative loop is completed when two or more determinant criteria have been achieved. For example, in some embodiments, an iterative loop is completed when a final characterization of a single analyte has been obtained and a process metric (e.g., uncertainty metric) for the characterization has traversed (e.g., exceeded or regressed below) a threshold value. In some embodiments, an iterative loop is completed when a first determinant criterium has been achieved and a second determinant criterium has not been achieved. For example, in some embodiments, an iterative loop is completed when a process metric (e.g., uncertainty metric) has exceeded a threshold value and the value of the process metric (e.g., uncertainty metric) does not have an oscillatory pattern over a defined number of cycles. In some embodiments, the determinant criterium includes the enumerated or categorized value of a first process metric (e.g., uncertainty metric) changing and the enumerated or categorized value of a second process metric (e.g., uncertainty metric) changing. In some embodiments, the determinant criterium includes the enumerated or categorized value of a first process metric (e.g., uncertainty metric) changing and the enumerated or categorized value of a second process metric (e.g., uncertainty metric) not changing.

In some embodiments, an iterative process of a single-analyte process includes a step of implementing an action on a single-analyte system based upon a process metric. In some embodiments, each iteration of a single-analyte process includes a step of implementing an action on the single-analyte system based upon the process metric. In some embodiments, a first action is implemented during a first iteration or cycle of an iterative process, and/or a second action is implemented during a second iteration or cycle of the iterative process. In some embodiments, the second action is selected and/or implemented independently of the first action. In some embodiments, the second action is different from the first action, for example, with respect to the reagent(s) used, duration of a chemistry or detection step, detection parameters (e.g., detector gain, luminescence excitation intensity or wavelength, luminescence emission intensity or wavelength etc.), number or duration of wash steps, temperature, an analysis or other algorithm utilized, or the like. In some embodiments, a first action is implemented during a first iteration of an iterative process, and a second action is implemented during a second iteration of the iterative process, in which the second action is selected and/or implemented based upon the first action. For example, in some embodiments, a first cycle of an iterative process includes the action of pausing a single-analyte process and altering the configuration of a hardware component, and a second cycle of the iterative includes implementing a new sequence of steps based upon the altered configuration of the hardware component.

In some embodiments, an action is implemented in a single-analyte system or method by performing the steps of: determining the action based upon a process metric (e.g., a process metric obtained from the single-analyte system or method); and implementing the action in the single-analyte system or method. In some embodiments, the determining the action based upon the process metric includes receiving a user input, performing an automated selection, performing a semi-automated selection, or a combination thereof. In some embodiments, receiving a user input includes providing a process metric to a user, and receiving a selection of an action from a list of possible actions, thereby receiving the user input. For example, in some embodiments, a single-analyte system provides a prompt to a user through a graphic user interface that permits the user to select an action from a list of possible actions. In some embodiments, performing an automated selection includes selecting an action from a list of possible actions utilizing one or more pre-configured rules for selecting the action based upon the determined process metric. In some embodiments, an automated selection is performed by a computer-implemented algorithm such as a remote server or a processor associated with a hardware component. In some embodiments, performing a semi-automated selection includes an automated selection process that includes an outside input or intervention during the selection process. For example, in some embodiments, a semi-automated process includes a process that includes a first computer-implemented reduction of a list of possible actions, followed by final selection of an action by a user from the reduced list of possible actions. In another example, a semi-automated selection includes an automated selection of an action from a list of possible actions, followed by the prompting of a user to approve the selected action before the action is implemented. In some embodiments, an action is selected from a list of possible actions. In some embodiments, an action s selected from a list of actions based upon a pre-determined logical structure (e.g., if process metric A has a value of B, then implement action C). In some embodiments, a set of possible actions is determined based upon a process metric (e.g., an uncertainty metric) and an action from the set of possible actions is selected based upon an additional input (e.g., a user input, the same process metric, a second process metric, etc.). In some embodiments, the action is selected from the group consisting of: pausing the single-analyte process; altering a sequence of steps for the single-analyte process; identifying a next step of a sequence of steps for the single-analyte process; performing a related process on the single analyte; performing the related process on a second single analyte; and continuing a sequence of steps for the single-analyte process.

In some embodiments, an action that is implemented during an iterative process includes pausing the process. In some embodiments, a pausing of the single-analyte process includes a duration that is defined prior to initiating the iterative process, or prior to a step in which the pause is implemented. In some embodiments, a pause includes a duration that is determined from a process metric or other information obtained during the iterative process, for example, during a step that precedes the step in which the pause is implemented. In some embodiments, a pause has an indefinite duration. In some embodiments, a pausing of the single-analyte process includes a temporary pausing of the single-analyte system. In some embodiments, a pausing of the single-analyte process includes a permanent pausing of the single-analyte process. In some embodiments, a pausing of the single-analyte process includes one or more additional actions that occur during the pausing. In some embodiments, the one or more additional actions is determined based upon a process metric (e.g., an uncertainty metric). In some embodiments, the one or more additional actions is implemented in order to alter a process metric (e.g., an uncertainty metric), alter a single-analyte system, provide an additional characterization of a single analyte, or a combination thereof. In some embodiments, pausing the single-analyte process includes an action selected from the group consisting of reconfiguring the detection system, recalibrating the detection system, repairing the detection system, calling to a second detection system, adding a second single analyte to the detection system, stabilizing the single analyte in the detection system, refreshing a computer-implemented algorithm, updating the computer-implemented algorithm, receiving a user input, and a combination thereof. In some embodiments, reconfiguring the detection system includes any changes to hardware and other components of a single-analyte system, such as replacement of a component, rearrangement of a component, adjustment of a component (e.g., changes in position or orientation), removal of a component, addition of a components, or a combination thereof. In some embodiments, recalibrating the detection system includes a reassessment of the output of a component of the single-analyte system against a known standard. For example, in some embodiments, an optical sensor is recalibrated against a characterized light source to confirm the sensor output, such as total sensed light intensity or signal-to-noise ratio. In some embodiments, repairing the detection system includes replacing or fixing damaged or defective components of a single-analyte detection system. For example, in some embodiments, an invariant signal from a sensor (e.g., no detected signal, constant detected signal when no signal should be present, etc.) includes a process metric pattern that prompts repair of a potentially damaged sensor. In some embodiments, calling to a second detection system includes performing a related process or action on a second detection system. In some embodiments, the second detection system is a component of the single-analyte system or is a component of a separate system. For example, in some embodiments, a single-analyte process calls to a second detection system to perform an identical step or sequence of steps on a replicate or control single analyte. In some embodiments, a single-analyte process calls to a second detection system (e.g., a higher-resolution physical measuring device or a different type of physical measuring device) to perform a step or a sequence of steps on the same single analyte. In some embodiments, a single-analyte process calls to a second detection system on a separate instrument to perform a bulk characterization of a plurality of single analytes. In some embodiments, adding a second single analyte to the detection system includes adding any additional single analyte to the detection system, such as a replicate single analyte, a duplicate single analyte, a control single analyte, an inert single analyte, or a combination thereof. For example, in some embodiments, a second single analyte is introduced into the detection system to provide a complementary, confirmatory, or contrasting source of comparison to the first single analyte when both single analytes are subjected to the same physical characterizations. In some embodiments, stabilizing the single analyte in the detection system includes any procedure that attempts to preserve or reduce the likelihood of damage or degradation to the single analyte during the pausing of the single-analyte process. For example, in some embodiments, a single analyte is stored at a reduced temperature or in an environment with reduced amounts of irradiation. In some embodiments, a single analyte is stored in the presence of a buffer that reduces the likelihood of degradative chemistries occurring. In some embodiments, refreshing a computer-implemented algorithm includes restarting or re-initializing a computer-implemented algorithm during a single-analyte process. For example, in some embodiments, a computer-implemented algorithm is restarted due to a non-converging or erroneous calculation. In some embodiments, updating a computer-implemented algorithm includes updating a source code or an input to the algorithm during the single-analyte process. For example, in some embodiments, a computer-implemented algorithm is updated to provide an enhanced version of an algorithm (e.g., a more accurate version, a more computationally-efficient version, etc.). In some embodiments, an iterative process is paused to receive a user input. For example, in some embodiments, an iterative process is configured to automatically pause and await a user input when a particular value of a process metric is determined. In in some embodiments, an iterative process is configured to automatically pause until a user performs a physical action on the single-analyte system (e.g., refilling a reagent, replacing, or repairing a hardware component, etc.). In some embodiments, pausing the single-analyte process includes receiving a user input and performing an action selected from the group consisting of reconfiguring the detection system, recalibrating the detection system, repairing the detection system, calling to a second detection system, adding a second single analyte to the detection system, stabilizing the single analyte in the detection system, refreshing a computer-implemented algorithm, updating the computer-implemented algorithm.

In some embodiments, an iterative process includes resuming (e.g., unpausing) a previously paused single-analyte process. In some embodiments, an iterative process includes, after implementing an action and before updating a single-analyte data set, unpausing the single-analyte process. For example, in some embodiments, a single-analyte process is paused during an iterative process to re-calibrate a component (e.g., a sensor), and then is subsequently resumed once the re-calibration is complete but before a single-analyte data set has been updated. In some embodiments, an iterative process includes, after implementing an action and after updating a single-analyte data set, unpausing the single-analyte process. For example, in some embodiments, a single-analyte process is stopped to adjust the orientation of a single analyte relative to a detection system based upon a process metric (e.g., an image quality metric). In some embodiments, the orientation of the single analyte is adjusted one or more times and the process metric updated until the process metric is determined to meet a target value or range. In some embodiments, once the target value or range for the process metric has been met, the single-analyte process is resumed. In some embodiments, an iterative process includes, after implementing one or more actions and after updating a single-analyte data set one or more times, unpausing the single-analyte process. For example, in some embodiments, an iterative process includes the actions of implementing a pause and performing a related process on a second single analyte before unpausing the single-analyte process. In some embodiments, an iterative includes implementing one or more actions and/or updating a single-analyte data set after implementing an action before unpausing the single-analyte process. In some embodiments, an iterative process that has been paused includes an embedded iterative process comprising one or more steps: of implementing an action; updating a single-analyte data set; determining a process metric based upon the updated single-analyte data set; and unpausing the single-analyte process if a determinant criterium for ending the embedded iterative process (e.g., an uncertainty metric decreasing, etc.) is achieved.

In some embodiments, an iterative process includes altering or updating a sequence of steps (e.g., cycles, procedures, or sub-procedures) for the single-analyte process. In some embodiments, the alteration includes adding steps, removing steps, repeating steps, rearranging steps during the single-analyte process or the iterative process; or a combination thereof. In some embodiments, the iterative process includes, before altering a sequence of steps, providing the sequence of steps for the single-analyte process. For example, in some embodiments, a preliminary sequence of steps (e.g., a standard protocol, a baseline protocol) is performed in the single-analyte process. In some embodiments, a preliminary sequence of steps is configured based upon an initial process metric that is determined from a preliminary single-analyte data set. In some embodiments, a sequence of steps is provided before the iterative process, such as before the initiation of the single-analyte process or before the initiation of the iterative process. In some embodiments, a sequence of steps is provided after initiating the iterative process. In some embodiments, a regimented approach to a single-analyte process (e.g., the process depicted in FIG. 5A) includes the altering or updating of a sequence of steps that is provided to the iterative process.

In some embodiments, an iterative process includes identifying a next step, procedure, or sub-procedure of a sequence of steps, procedures, or sub-procedures. In some embodiments, identifying a next step, procedure, or sub-procedure of a sequence of steps, procedures, or sub-procedures includes identifying a next single step, procedure, or sub-procedure of the sequence of steps, procedures, or sub-procedures. For example, in some embodiments, an iterative process is configured to only select a single step per cycle or iteration of the iterative process to increase the likelihood of obtaining a desired or informative result after each step of the iterative process. In some embodiments, identifying a next step, procedure, or sub-procedure of a sequence of steps, procedures, or sub-procedures includes identifying a next two or more steps, procedures, or sub-procedures of the sequence of steps, procedures, or sub-procedures. For example, in some embodiments, an iterative process is configured to select a new or updated sequence of steps for the single-analyte process, then continue to update or alter the new or updated sequence of steps during successive cycles or iterations of the single-analyte process. In some embodiments, a step-wise approach to a single-analyte process (e.g., the process depicted in FIG. 5B) includes identifying a next step, procedure, or sub-procedure of a sequence of steps, procedures, or sub-procedures.

In some embodiments, a single-analyte process or an iterative process includes a step of performing a related process on the single analyte. For example, in some embodiments, a single protein analyte is detected or characterized using a first protein detection assay (e.g., a multistep probe binding assay) and/or the single protein analyte is detected or characterized using a second protein detection assay (e.g., an Edman-type protein sequencing assay). In some embodiments, the related process includes a single-analyte process performed at single-analyte resolution, or a bulk analyte process. In some embodiments, the related process includes a synthesis, fabrication, manipulation, degradation, or assaying process. In some embodiments, the related process is selected to increase the utility of the single-analyte process. In some embodiments, the related process is necessary. In some embodiments, the related process facilitates achieving a targeted final outcome for the single-analyte process. For example, in some embodiments, a single-analyte synthesis process includes one or more intermediate steps that involve a manipulation or degradation of the single analyte (e.g., cleaving an unwanted fragment from the single analyte, etc.). In some embodiments, a single-analyte fabrication process includes one or more intermediate steps that involve a synthesis, manipulation, or degradation of the single analyte. In some embodiments, a single-analyte assay process includes a manipulation or degradation of the single analyte that permits the assaying process to occur with a modified single analyte.

In some embodiments, performing a related process on a single-analyte includes performing the same single-analyte process on the single analyte. In some embodiments, a single-analyte process comprising the action of performing the same single-analyte process on the single analyte occurs on the original detection system or a second detection system. For example, in some embodiments, performing the same single-analyte process on the single analyte occurs on the original detection system under different detection conditions or parameters. In some embodiments, performing the same single-analyte process on the single analyte occurs on a second detection system that is configured to perform a physical measurement of the single analyte under differing conditions (e.g., utilizing a higher resolution sensor).

In some embodiments, performing a related process on a single analyte includes performing a differing process on the single analyte. In some embodiments, the differing process includes a differing single-analyte process or a bulk analyte process. For example, in some embodiments, performing a related process on the single analyte includes performing a second single-analyte process that differs with respect to a physical measurement performed on the single analyte during the single-analyte process. In some embodiments, performing a related process on the single analyte includes performing a bulk analyte process on the single analyte or a plurality of analytes comprising the single analyte to obtain a bulk characterization of an analyte property (e.g., an average value of an analyte property measured by the single-analyte process). In some embodiments, a differing process is performed on the same detection system as the single-analyte process. In some embodiments, a differing process is performed on a second detection system. In some embodiments, the second detection system differs from the original detection system with respect to one or more components, for example for performing a differing process or performing a similar process that differs with respect to accuracy, precision, or resolution. In some embodiments, the second detection system is identical to the original detection system, for example for performing a replicate process on the single analyte.

In some embodiments, performing a related process on a single analyte includes performing a reconfigured single-analyte process on the single analyte, for example, the reconfigured single-analyte process including obtaining a second physical measurement on the single analyte at single-analyte resolution. In some embodiments, the reconfigured single-analyte process is reconfigured with respect to one or more process parameter of the single-analyte process. In some embodiments, the one or more process parameter is, for example, selected from the group consisting of process length, process environment, process orientation, process sensitivity, process data collection rate, process data collection amount, process instrumentation, fluid flow rate, total fluid volume, fluid charging time, fluid incubation time, fluid discharging time, fluid composition, light irradiation time length, light irradiation intensity, detectable label composition, detectable label quantity, algorithm configuration, algorithm type, algorithm initialization parameters, algorithm convergence criterium, and a combination thereof.

In some embodiments, a single-analyte process (e.g., an iterative single-analyte process) includes a step of performing a related process on a second single analyte. In some embodiments, a second single analyte includes a single analyte such as a second single analyte that is identical to the first single analyte (e.g., a duplicate single analyte, a replicate single analyte, etc.), a second single analyte that is obtained from the same sample as the first single analyte (e.g., a duplicate aliquot from the sample), a control single analyte (e.g., a positive or negative control analyte), a standard single analyte (i.e., a single analyte that provides a measurable reference property), or an inert single analyte. In some embodiments, a second single analyte includes a measurable similarity or difference to the first single analyte with respect to a property of the single analyte, such as a chemical structure (e.g., folded vs. unfolded polypeptides; crystalline vs. amorphous crystal structure; linear vs. branched structure, etc.), a chemical composition (e.g., differing polypeptide isoforms; truncated or degraded polypeptides; functionalized vs. non-functionalized nanoparticles, etc.), a chemical state (e.g., electrically-charged vs. -uncharged; folded vs. denatured, etc.), or a combination thereof.

In some embodiments, performing a related process on a second single analyte includes a single-analyte process performed at single-analyte resolution, or a bulk analyte process. In some embodiments, the related process includes a synthesis, fabrication, manipulation, degradation, or assaying process. In some embodiments, a related process is selected to provide a comparison between the first single analyte undergoing the first single-analyte process and the second single analyte undergoing the related process. For example, in some embodiments, a single-analyte process is performed on a first single analyte under a first set of conditions and is performed on a second single analyte under a second set of conditions to determine a more efficient technique for performing the process. In some embodiments, a first single analyte and a second single analyte undergo identical single-analyte processes to provide a comparison between the outcomes of the single-analyte processes (e.g., a statistical comparison of outcomes). In some embodiments, a first single analyte and a second single analyte undergoes identical single-analyte processes but only one of the two single analytes is assayed or physically characterized to reduce process time or cost. In some embodiments, a related process is selected to provide a differing outcome or product for the second single analyte. For example, in some embodiments, the related process includes or omit processes (e.g., synthesis, fabrication, manipulation, degradation) for the second single analyte relative to the single-analyte process for the first single analyte. For example, in some embodiments, a second polypeptide single analyte undergoes a related process that includes an enzymatic treatment to produce an untreated first single analyte and a treated second single analyte. In some embodiments, performing a related process on the second single-analyte comprises performing the same single-analyte process on the second single analyte. For example, in some embodiments, a first single protein analyte is detected or characterized using a first protein detection assay set forth herein and/or a second single protein analyte is detected or characterized using the first protein detection assay. In some embodiments, the same single-analyte process occurs on the original detection system or a second detection system. In some embodiments, performing a related process on a second single analyte includes performing a differing process on the second single analyte. For example, in some embodiments, a first single protein analyte is detected or characterized using a first protein detection assay (e.g., a multistep probe binding assay set forth herein) and/or a second single protein analyte is detected or characterized using a second protein detection assay (e.g., an Edman-type protein sequencing assay). In some embodiments, a differing process that is applied to a second single-analyte includes a single-analyte process or a bulk analyte process that differs from a single-analyte process or a bulk analyte process that was applied to a first single-analyte. In some embodiments, a differing process is performed on the original detection system as the single-analyte process. In some embodiments, a differing process is performed on a second detection system. In some embodiments, a second detection system differs from the original detection system with respect to one or more components, for example for performing a differing process or performing a similar process that differs with respect to accuracy, precision, or resolution. In some embodiments, a second detection system is identical to the original detection system, for example for performing a replicate process on the second single analyte.

In some embodiments, performing a related process on the second single analyte includes performing a reconfigured single-analyte process on the second single analyte, in which the reconfigured single-analyte process comprises obtaining the physical measurement on the second single analyte at single-analyte resolution. In some embodiments, the reconfigured single-analyte process is reconfigured with respect to one or more process parameter of the single-analyte process. In some embodiments, the one or more process parameter is selected from the group consisting of process length, process environment, process orientation, process sensitivity, process data collection rate, process data collection amount, process instrumentation, fluid flow rate, total fluid volume, fluid charging time, fluid incubation time, fluid discharging time, fluid composition, light irradiation time length, light irradiation intensity, detectable label composition, detectable label quantity, algorithm configuration, algorithm type, algorithm initialization parameters, algorithm convergence criterium, and a combination thereof. In some embodiments, the second single analyte is selected from the group consisting of a replicate single analyte, a duplicate single analyte, a control single analyte, a standard single analyte, an inert single analyte, and a combination thereof. In some embodiments, a control single analyte includes any single analyte with a known or characterized behavior or lack thereof when undergoing the same process or physical measurement as the single analyte. In some embodiments, a standard single analyte includes any single analyte with a known or characterized behavior that predictably corresponds to the behavior of the single analyte. In some embodiments, an inert single analyte includes any single analyte that is known to not participate in a single-analyte process or is known not to provide a signal during a physical measurement.

In some embodiments, a process metric is determined before, during, or after a single-analyte process or an iterative process thereof. In some embodiments, a process metric is determined from a preliminary single-analyte data set that is collected before a single-analyte process is initiated, after a single-analyte process is initiated, before an iterative process is initiated, or after an iterative process is initiated. In some embodiments, determining a process metric (e.g., an uncertainty metric) includes one or more of the steps of deriving a value from the single-analyte data set, and deriving the process metric (e.g., an uncertainty metric) based upon the value derived from the single-analyte data set. In some embodiments, the deriving the value from the single-analyte data set includes extracting the value from the single-analyte data set. In some embodiments, extracting the value from a single-analyte data set includes identifying and/or transferring a value from the single-analyte data set to an algorithm configured to perform an iterative process without altering the value. For example, in some embodiments, an extracted value includes a value from a physical measurement (e.g., voltage, light intensity, signal lifetime, etc.) or a selected value from a set of instrument metadata or sample metadata. In some embodiments, the deriving the value from the single-analyte data set comprises calculating the value from the single-analyte data set. In some embodiments, calculating the value from a single-analyte data set includes one or more of extracting a value from the single-analyte data set, and converting the value to a new value through one or more mathematical (e.g., equations, etc.) or logical operations (e.g., for an extracted value between X and Y, the process metric has a value of Z, etc.). For example, in some embodiments, a single-analyte process includes calculating image quality metrics utilizing pixel identification and classification techniques. In another example, a single-analyte process includes calculating a single analyte property (e.g., a kinetic binding constant) from a value of instrument metadata (e.g., a temperature). In some embodiments, deriving a process metric includes deriving the process metric from a reference source based upon the value derived from the single-analyte data set. For example, in some embodiments, a derived value is utilized to look up a process metric in a reference source (e.g., a database, a reference table, an internet or intranet source, a user-defined source, etc.) or a cumulative data source. In some embodiments, deriving a process metric from a reference source includes extracting the uncertainty metric from the reference source (e.g., transferring a value from a tabulated set of reference values). In some embodiments, deriving a process metric from a reference source includes calculating the process metric based upon a value derived from the reference source.

In some embodiments, a single-analyte process (e.g., an iterative single-analyte process) includes determining a process metric in which the process metric is an uncertainty metric. In some embodiments, the uncertainty metric includes a measure of an error or a bias in the single-analyte system. In some embodiments, the error and/or the bias is characterized as a stochastic, systematic, random, variable, or fixed error or bias, or a combination thereof. In some embodiments, an uncertainty metric is determined for a characterization of a single analyte that is generated by a single-analyte system, such as an uncertainty metric for a property, characteristic, behavior, interaction, or effect of the single analyte, or an uncertainty metric for a physical measurement used to determine a property, characteristic, behavior, interaction, or effect of the single analyte. For example, in some embodiments, an uncertainty metric for a sequence or structure determination of a biomolecular single analyte (e.g., polypeptide, polynucleotide, etc.) includes a confidence level for the sequence or structure determination. In some embodiments, a physical property (e.g., pair-wise binding dissociation constant) of a single analyte is determined, with an associated uncertainty metric comprising a confidence interval for the property measurement. In some embodiments, an uncertainty metric for a physical measurement of a single analyte includes a statistical measure of the physical measurement data for the single analyte, or a sampling thereof (e.g., a mean, median, variance, standard deviation, p-value, t-test metric, etc.). In some embodiments, an uncertainty metric is determined for a system parameter or system component, other than the single analyte, that is utilized in a single-analyte process. For example, in some embodiments, an uncertainty metric comprising a statistical metric (e.g., mean, variance, p-value, etc.) is calculated for data provided by an instrument sensor (e.g., a thermocouple, a mass flow sensor) to assess the uncertainty of a physical measurement performed on the single-analyte system. In some embodiments, an uncertainty metric for a system parameter or system component provides a measure of uncertainty for the single analyte, for example by proxy, by correlation, or by a causal relationship. For example, in some embodiments, a system parameter (e.g., temperature) is a proxy or be correlated to a rate of false positive or false negative physical measurements, thereby providing a measure of uncertainty based on the observed system parameter. In some embodiments, the uncertainty metric includes an uncertainty metric for an observation, a measurement, or a detection for a property, characteristic, or effect of the single analyte. In some embodiments, an uncertainty metric includes a statistical metric selected from the group consisting of a confidence interval, a confidence level, a prediction interval, a tolerance interval, a Bayesian interval, a sensitivity coefficient, a confidence region, a confidence band, an error propagation, an uncertainty propagation, a correlation coefficient, a coefficient of determination, a mean, a median, a mode, a variance, a standard deviation, a coefficient of variation, a percentile, a range, a skewness, a kurtosis, an L-moment, and an index of dispersion.

In some embodiments, an uncertainty metric, such as a statistical metric, s utilized to determine an action that is to be implemented on a single-analyte system during an iterative process. In some embodiments, an uncertainty metric includes any measure of variability in a single-analyte system, including variability with respect to any one of instrument data, instrument metadata, sample data, sample metadata, and single-analyte characterizations. In some embodiments, an uncertainty metric is determined by calculating a metric from data that is included within a single-analyte data set. In some embodiments, an uncertainty metric is determined by calculating a metric from a subset or sample of data within a single-analyte data set. For example, in some embodiments, an uncertainty metric for the temperature within a fluidic cell is calculated by sampling a subset of a time-temperature series for a thermocouple within the fluidic cell over a fixed period of time and deriving a standard deviation from the subset of time-temperature data. In some embodiments, an uncertainty metric is determined by applying a statistical model, such as a deterministic model, a stochastic model, a probabilistic model, an inferential model, or a combination thereof.

In some embodiments, a single-analyte process utilizes an inferential method to determine a characterization of a single-analyte or an outcome for a single-analyte, as set forth herein. In some embodiments, an inferential method apply any suitable inferential technique, such as frequentist inference, Bayesian inference, likelihood-based inference, Akaike information criterion inference, or a combination thereof. In some embodiments, an inference approach is utilized to form and/or test a hypothesis for a characterization of a single analyte during a single-analyte process. For example, in some embodiments, during a single-analyte assay process, a hypothesis for the characterization of a single analyte is continually or periodically updated based upon the input of new data obtained from a single-analyte system into an inferential model. In a specific embodiment of this example, a single-polypeptide identification assay is utilized an inferential model (e.g., a Bayesian inference) to individually update a set of identity hypotheses based upon the sequential collection of affinity reagent binding measurements. In some embodiments, an identity for a single polypeptide is determined by calculating an uncertainty metric (e.g., a Bayesian likelihood score) for each identity hypothesis in the set of identity hypotheses until a single hypothesis rises above a threshold value for the likelihood score. In some embodiments, an inference approach is utilized to form and/or test a hypothesis for an instrument hygiene-related problem. For example, in some embodiments, an instrument-related error (e.g., poor data signal-to-noise ratio) that increases the uncertainty of a single-analyte characterization is attributable to multiple possible problems (i.e., error hypotheses), including hardware- and software-related errors. In some embodiments, an inferential approach is utilized to collect information on the system status and/or performance and apply the information to each error hypothesis via an inference method. In some embodiments, based upon the most likely error hypothesis, an action is implemented on the single-analyte system to correct the source of the error. Exemplary inferential approaches used in a method set forth herein are set forth in U.S. Pat. Nos. 10,473,654 and 11,282,585, and U.S. Pat App. Ser. No. 63/254,420, each of which is incorporated herein by reference in its entirety for all purposes.

In some embodiments, a process metric utilized to select and/or implement an action in a single-analyte system includes a curated process metric. In some embodiments, a curated process metric includes any process metric that is determined from one or more other process metrics. In some embodiments, a curated process metric is used similarly to other process metrics set forth herein. For example, in some embodiments, a curated process metric includes a quantitative process metric that is calculated utilizing one or more other process metrics. In some embodiments, a curated process metric includes a qualitative process metric, such as a sorted or ranked metric (e.g., an image is assigned a curated process metric of “fail” if 6 of 10 image-quality process metrics fail to meet threshold values). In some embodiments, determining a process metric for a single analyte based upon a single-analyte data set includes the steps of: determining one or more process metrics based upon the single-analyte data set; and determining a process metric that is selected from the one or more process metrics

In some embodiments, a curated process metric (e.g., a curated uncertainty metric) includes a user input, such as a weighting or ranking by a user, or a confirmation of a processor-determined metric value. In some embodiments, the determining one or more curated process metrics (e.g., curated uncertainty metrics) comprises one or more of the steps of: providing a value derived from the single-analyte data set to a user; obtaining an input from the user based upon the providing the value; and determining a curated process metric (e.g., a curated uncertainty metric) based upon the input from the user.

In some embodiments, a user is provided a value from a single-analyte data set that comprises a process metric. In some embodiments, a curated process metric (e.g., a curated uncertainty metric) includes a weighted metric, a correlated metric, a ranked metric, or an enumerated or categorized metric. In some embodiments, a curated process metric includes a qualitative process metric (e.g., a qualitative uncertainty metric). For example, in some embodiments, a curated process metric includes a pass/fail metric for a single-analyte data set based upon a count of how many process metrics (e.g., data quality metrics) fall within a threshold range. In some embodiments, a curated process metric includes a quantitative process metric (e.g., a quantitative uncertainty metric). For example, in some embodiments, a curated process metric includes a score calculated by combining one or more process metrics by mathematical operations (e.g., addition, subtraction, etc.). In some embodiments, determining a curated process metric for a single analyte based upon the single-analyte data set comprises determining two or more process metrics (e.g., uncertainty metrics) for the single analyte and determining the curated process metric from the two or more process metrics. In some embodiments, implementing an action on the single-analyte system is based upon a first process metric of the two or more process metrics for the single analyte. For example, in some embodiments, a curated process metric includes a ranked list of process metrics based upon a deviation from an expected range, and an action to be implemented is chosen based upon the top-ranked process metric. In some embodiments, implementing an action on the single-analyte system is based upon at least two process metrics of the two or more process metrics for the single analyte. For example, in some embodiments, an action to be implemented is chosen by calculating a curated process metric comprising a score of process metrics whose values lie outside a defined threshold range for each process metric.

In some embodiments, an iterative approach to determining or modifying a sequence of steps of a single-analyte process utilizes a single-analyte data set. In some embodiments, the single-analyte data set includes information that is utilized to determine one or more process metrics. In turn, in some embodiments, the one or more process metrics is utilized to determine a subsequent action of the single-analyte system. In some embodiments, a single-analyte data set includes data from one or more data sources, including sources within the system and sources external to the system. In some embodiments, the single-analyte data set includes instrument data, sample data, measurement data, cumulative data, reference data, user-supplied data, externally-supplied data, or a combination thereof. In some embodiments, the instrument data includes instrument metadata, instrument sensor data, instrument environmental data, instrument user-defined data, or a combination thereof. For example, in some embodiments, a single-analyte data set includes a time-series of measurements from an instrument sensor suite and accompanying metadata (e.g., notation of actions, procedures, etc. being implemented on the system). In some embodiments, a single-analyte data set includes a time-series of measurements from an instrument sensor suite and accompanying instrument environmental data (e.g., external temperature, external humidity, internal temperature, etc.). In some embodiments, the sample data includes user-defined sample data, instrument-defined sample data, sample tracking data, or a combination thereof. For example, in some embodiments, a single-analyte data set includes user-input data concerning the source and collection method of a sample. In some embodiments, a single-analyte data set includes vendor-supplied information on reagent composition for reagents utilized during a single-analyte synthesis or fabrication. In some embodiments, a single-analyte data set includes a time-series of sample handling information (e.g., time-temperature history). In some embodiments, the measurement data includes a physical measurement of the single analyte. For example, in some embodiments, measurement data includes data such as imaging data, spectral emission data, spectral absorption data, and any other appropriate physical measurement that the single-analyte system is configured to obtain from a single analyte. In some embodiments, the physical measurement includes a plurality of physical measurements of the single analyte. In some embodiments, the physical measurement includes a set or compilation of physical measurements of the single analyte. For example, in some embodiments, a single-analyte data set includes a video of a single analyte, in which each frame of the video includes image data of the single analyte. In some embodiments, the cumulative data includes data from a previous performance of the iterative process or the single-analyte process. For example, in some embodiments, cumulative data includes all prior data related to a single analyte involved in a current single-analyte process, or a subset thereof. In some embodiments, the cumulative data includes data from an earlier step or cycle of a current performance of the iterative process. In some embodiments, the single-analyte data set includes a set of cumulative data that is extracted or derived from a larger set of cumulative data. For example, in some embodiments, a single-analyte data set includes data that is selectively extracted from a larger set of cumulative data based upon the type of single analyte and the specific action to be implemented on the single-analyte system.

In some embodiments, determining a process metric includes calculating the process metric (e.g., uncertainty metric) from the single-analyte data set. In some embodiments, a single-analyte data set includes data from two or more data sources. In some embodiments, two or more data sources are independently selected from the group consisting of a measurement device, a sensor, a user input, a reference source, and an external source. In some embodiments, a measurement device provides physical characterization data with regard to the single analyte. For example, in some embodiments, a measurement device provides a characterizing measurement of a single analyte, including but not limited to a measure of light absorbance (e.g., an IR or UV spectrum), a measure of light emission (e.g., a fluorescence measurement), a measure of mass (e.g., a mass spectrum), a measure of size, a measure of position, a measure of velocity, or a response to an electric field or a magnetic field. In some embodiments, a measurement device provides additional instrument metadata concerning a state, configuration, or function of the measurement device during a single-analyte process. In some embodiments, a sensor produces additional physical measurements of system components other than the single analyte during a single-analyte process. For example, in some embodiments, a sensor provides a measurable parameter of a system component, including but not limited to temperature, pressure, fluid flow rate, light intensity, force, strain, length, width, height, volume, velocity, a measure of deformation, a measure of contraction, a measure of compression, a measure of rotation, or a measure of displacement. In some embodiments, a sensor provides additional instrument metadata concerning a state, configuration, or function of the measurement device during a single-analyte process. In some embodiments, a user input includes data related to known information (e.g., sample types, protocol type, etc.) and process instructions (e.g., process length, targeted outcomes, etc.). In some embodiments, a user input includes manual data observations during a single-analyte process. For example, in some embodiments, a user input includes manual identification of data features (e.g., image features, spectral features, etc.). In some embodiments, reference source data includes tabulated values, empirical correlated data, theoretical data, and any described or observed patterns or trends of such data types. For example, in some embodiments, a reference source includes, but is not limited to, a tabulated chart (e.g., a steam table), a reference database (GenBank, UniProt, PubMed, NCBI, etc.), a textbook, a journal article, or a patent publication. In some embodiments, an external data source includes any data supplied by a third party, such as reagent characterization data, external single-analyte measurements, and proprietary or secret information (e.g., sharing of unpublished data), and vendor-supplied reference materials. In some embodiments, a datum from any possible data source is stored within a set of cumulative data.

In some embodiments, a process metric is determined from one or more data sources. In some embodiments, a process metric is extracted, derived, or otherwise calculated from data obtained from the one or more data sources. In some embodiments, a process metric is extracted, derived, or otherwise calculated from data obtained from at least two data sources. In some embodiments, a process metric is extracted, derived, or otherwise calculated by combining a first datum from a first data source with a second datum from a second data source. For example, in some embodiments, a process metric is determined by calculating a difference between a first datum from a physical measurement data set and a second datum from a cumulative data set. In some embodiments, a process metric is extracted, derived, or otherwise calculated based upon a datum from a first data source if a datum from a second data source meets a criterium. For example, in some embodiments, a first process metric is calculated from physical measurement data if a datum from an instrument metadata source is within a specified range. In some embodiments, a process metric is extracted, derived, or otherwise calculated based upon a datum from a first data source based upon a datum from a second data source. For example, in some embodiments, a process metric for a physical measurement data set is determined by a first empirical correlation if a datum from an instrument metadata set is within a first range or is determined by a second empirical correlation if a datum from the instrument metadata set is outside of the first range. In some embodiments, the process metric is calculated using data from a single data source of the two or more data sources. In some embodiments, the process metric is calculated using data from more than one data source of the two or more data sources.

In some embodiments, a single-analyte process, or an iterative process thereof, utilizes a processor-implemented or computer-implemented algorithm. In some embodiments, a processor-implemented or computer-implemented algorithm is configured to perform a task within a single-analyte system, including collecting a datum for a single-analyte data set, compiling a single-analyte data set, analyzing a single-analyte set, determining a process metric based upon a single-analyte data set, determining an action for a single-analyte process, configuring an action for the single-analyte process, configuring a sequence of steps for a single-analyte process, updating or modifying a sequence of steps for a single-analyte process, controlling a component of a single-analyte system, requesting user input to a single-analyte process, receiving user input to a single-analyte process, requesting external input to a single-analyte process, receiving external input to a single-analyte process, or a combination thereof. In some embodiments, a single-analyte system includes one or more computer-implemented algorithms selected from the group consisting of a data collection algorithm, a data analysis algorithm, a decision algorithm, a control algorithm, a communications algorithm, and a combination thereof. In some embodiments, the single-analyte system comprises a computer-implemented algorithm. In some embodiments, the single-analyte system comprises two or more data analysis algorithms. In some embodiments, the two or more data analysis algorithms comprise a partial data analysis algorithm, a full data analysis algorithm, or a combination thereof. In some embodiments, a partial data analysis algorithm is configured to provide a preliminary analysis or provide an analysis of a partial set of single-analyte data. For example, in some embodiments, a partial data analysis algorithm is utilized to determine if a set of physical measurement data for a single analyte achieves a threshold value for a data quality metric before moving on to a subsequent physical measurement of the single analyte. In some embodiments, an output from a partial data analysis algorithm includes a process metric (e.g., an uncertainty metric). In some embodiments, a partial data analysis algorithm utilizes a subset of data included in a single-analyte data set or a complete set of data included in a single-analyte data set. In some embodiments, a full data analysis algorithm is utilized based upon the output of a partial data analysis algorithm (e.g., a partial data analysis algorithm is unable to resolve a process metric sufficiently, thereby invoking use of the full data analysis algorithm). In some embodiments, a full data analysis algorithm is invoked independently of a partial data analysis algorithm. In some embodiments, a full data analysis algorithm is configured to provide a complete analysis of a single-analyte data set. In some embodiments, a full data analysis algorithm includes a higher degree of computational complexity and/or a longer computational time scale than a partial data analysis algorithm. For example, in some embodiments, a full data analysis algorithm is configured to provide a complete characterization of a single analyte (e.g., a structural identification or an identity) during a single-analyte process. In some embodiments, a full data analysis algorithm utilizes a subset of data included in a single-analyte data set or a complete set of data included in a single-analyte data set. In some embodiments, determining a process metric for a single analyte comprises one or more steps of: providing a single-analyte data set to one or more computer-implemented algorithms; and determining the process metric using the one or more computer-implemented algorithms.

In some embodiments, implementing an action on a single-analyte system based upon a process metric includes: providing the process metric to a decision algorithm of the single-analyte process system; determining an action based upon the providing the process metric to the decision algorithm; and providing an instruction comprising the action from the decision algorithm to a control algorithm of the single-analyte system.

In some embodiments, a single-analyte process incorporates one or more iterative processes. In some embodiments, an iterative process is utilized to identify and/or address one or more sources of uncertainty during a single-analyte process. In some embodiments, an iterative process is initiated as a first step of the single-analyte process. In some embodiments, an iterative process is initiated after a preliminary sequence of steps is completed. In some embodiments, an iterative process is initiated after a preliminary sequence of steps has been configured, but before the preliminary sequence of steps has been completed. In some embodiments, a preliminary sequence of steps includes one or more processes that prepare a single-analyte system for a single-analyte process. For example, in some embodiments, a preliminary sequence of steps includes preparing a single-molecule array for a single-molecule assaying process (e.g., polypeptide or polynucleotide identification, polypeptide, or polynucleotide sequencing, etc.). In some embodiments, a preliminary sequence of steps for preparing a single-molecule array includes one or more of the steps of providing a solid support that is configured to generate a single-molecule array, rinsing the solid support to remove unbound materials, rinsing the solid support to remove unwanted materials, depositing single-molecule attachment groups (e.g., functional groups, DNA concatemers, DNA origami) in an array on the solid support surface, detecting the presence of an array of single-molecule attachment groups on the solid support (e.g., via fluorescence microscopy, atomic force microscopy, surface plasmon resonance, etc.), attaching individual molecules (e.g., polypeptides, polynucleotides, etc.) to each single-molecule attachment group, providing control groups (e.g., fluorescence markers) or standard groups (e.g., known polypeptide standards) to the single-molecule array, detecting the presence of an array of single-molecule control groups or standard groups on the solid support detecting the presence of an array of single molecules attached to single-molecule attachment groups on the solid support, registering the position of each detected single molecule and/or single-molecule attachment group relative to a fixed position or location on the solid support, and obtaining a preliminary physical measurement of each single-molecule site on the solid support to provide a preliminary or background measurement of the single-molecule array.

In some embodiments, a single-analyte process is discontinued after the completion of an iterative loop. For example, in some embodiments, a determinant criterium for discontinuing an iterative loop of a single-analyte process includes obtaining a final characterization of a single analyte, thereby confirming the completion of a single-analyte synthesis, fabrication, manipulation, degradation, or assay. In some embodiments, a single-analyte process is continued after the completion of an iterative loop. For example, in some embodiments, an iterative process is initiated due to the determination of a value of a process metric outside of a normal range of values, and is terminated when the value of the process metric is determined to have returned to within the normal range of values.

In some embodiments, an iterative process is initiated if an initiation criterium is achieved. In some embodiments, an initiation criterium includes an event, situation, or system state that provokes the use of an iterative process. In some embodiments, an initiation criterium includes: a process metric traversing a threshold value (e.g., an uncertainty metric exceeding the threshold value); a user-specified input (e.g., an instruction to increase data precision); an unexpected property, characteristic, behavior, or interaction of the single analyte (e.g., a previously-unobserved single-analyte behavior); a time constraint (e.g., a need to complete a process by a fixed time); a logistical constraint (e.g., a need to complete a process before using all of a reagent); an unexpected single-analyte system behavior (e.g., a fluctuating internal temperature); or a combination thereof.

In some embodiments, a single-analyte process includes the step of, after performing an iterative process, performing an additional process for the single analyte. In some embodiments, the additional process includes an additional physical measurement of the single analyte. In some embodiments, the additional physical measurement is the same as a physical measurement that was performed earlier in the single-analyte process. In some embodiments, the additional physical measurement is a differing physical measurement from a physical measurement that was performed earlier in the single-analyte process. In some embodiments, the differing physical measurement includes a complementary characterization of the single analyte (e.g., confirming an initial characterization of the single analyte). In some embodiments, the performing of an additional process using the single analyte comprises altering the single analyte. In some embodiments, altering the single analyte includes one or more processes selected from the group consisting of: altering the single analyte structurally; altering the single analyte chemically; altering the single analyte physically; altering an orientation of the single analyte; altering a position of the single analyte; and a combination thereof.

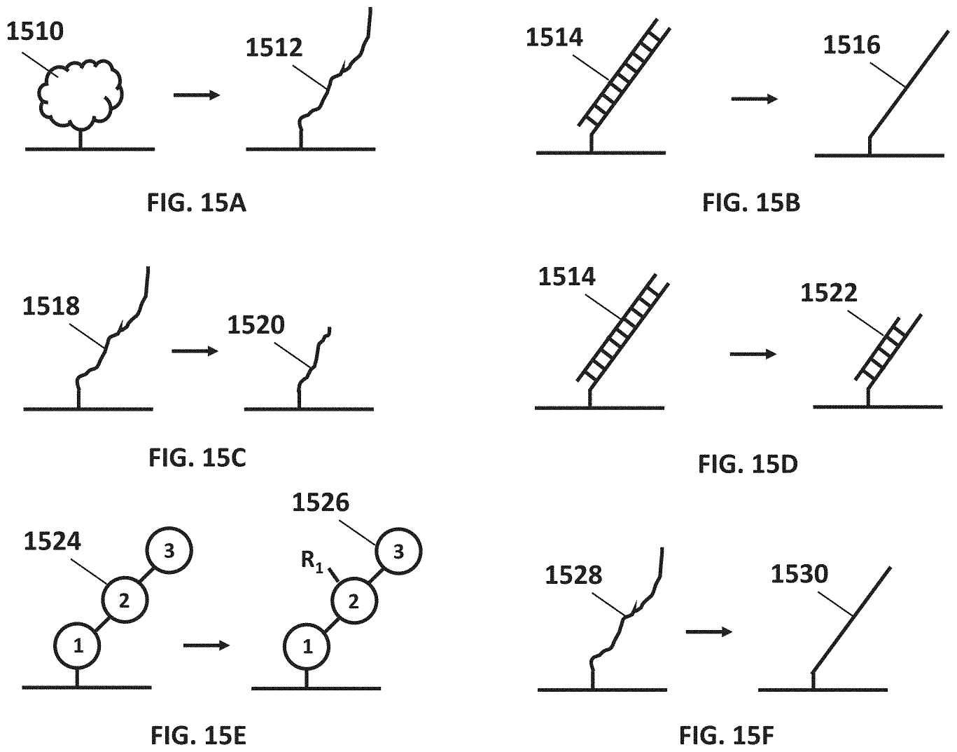

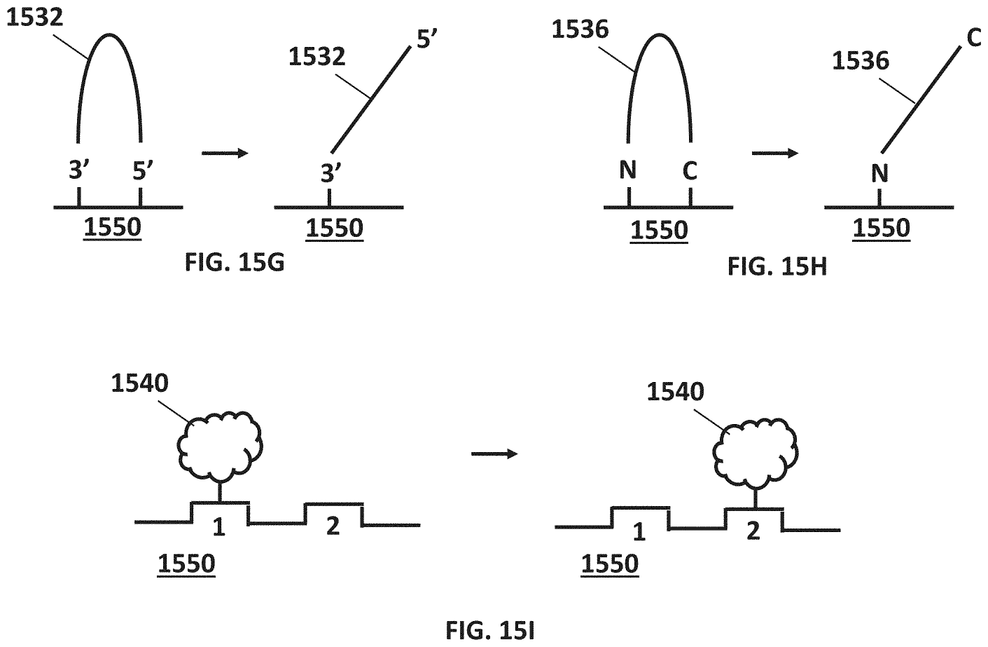

FIGS. 15A-15I illustrate various alterations of a single analyte. FIGS. 15A-15D depict altering a single analyte structurally. In some embodiments, a structural alteration of a single analyte includes a reversible or irreversible change in the shape or connectivity of the single analyte. FIG. 15A illustrates a structural alteration by the denaturation of a polypeptide 1510 into a denatured polypeptide 1512. FIG. 15B illustrates a structural alteration by the denaturation of a double-stranded polynucleotide 1514 into a denatured (single-stranded) polynucleotide 1516. FIG. 15C illustrates a structural alteration by the proteolytic cleavage of a polypeptide 1518 into a polypeptide fragment 1520. FIG. 15D illustrates a structural alteration by the restriction cleavage of a polynucleotide 1514 into a polynucleotide fragment 1522. In some embodiments, a chemical alteration of a single analyte includes any change in the chemical composition and/or behavior of the single analyte. FIG. 15E illustrates a chemical alteration of a single analyte 1524 (e.g., polypeptide, polynucleotide) by the addition of a functional group (R1) to form a functionalized single analyte 1526. In some embodiments, a physical alteration of a single analyte includes any change in the single analyte that is induced by an applied force (e.g., a shear stress) or an applied field (e.g., an electrical or magnetic field). FIG. 15F depicts a physical alteration of a single analyte (e.g., a polypeptide, a polynucleotide, etc.) 1528 by an external force or an external field to create an extended single analyte 1530. In some embodiments, an alteration of the orientation of a single analyte includes any change in a portion of the single analyte relative to a second portion of the single analyte. FIG. 15G illustrates a polynucleotide 1532 coupled to a solid support 1550 at the 3′ terminus and 5′ terminus of the polynucleotide 1532. Uncoupling the 5′ terminus from the solid support 1550 alters the orientation of the 5′ terminus relative to the 3′ terminus. FIG. 15H illustrates a polypeptide 1536 coupled to a solid support 1550 at the C terminus and N terminus of the polypeptide 1536. Uncoupling the C terminus from the solid support 1550 alters the orientation of the C terminus relative to the N terminus. In some embodiments, altering a position of a single analyte includes altering the physical location where a single analyte is located and/or observed. FIG. 15I depicts a single analyte 1540 (e.g., a polypeptide, a nanoparticle, etc.) coupled to a solid support 1550 at address 1 at a first time point. At a second time point, the location of single analyte 1540 has been altered to address 2 on the solid support 1550.

In some embodiments, the performing of an additional process using the single analyte includes altering an environment of the single analyte. In some embodiments, altering the environment includes one or more of: altering a temperature; altering a pressure; altering an electrical field; altering a magnetic field; altering a fluid; altering an entity other than the single analyte; and a combination thereof.

In some embodiments, performing an additional process using the single analyte includes stabilizing the single analyte. In some embodiments, stabilizing the single analyte includes a process to preserve or protect the structure and/or function of the single analyte. In some embodiments, stabilizing methods include adding stabilizing reagents, removing de-stabilizing reagents, altering a temperature or pressure, storing the single analyte in a preserving environment, or a combination thereof.

In some embodiments, a single-analyte process includes the step of, after performing an iterative process, discontinuing the single-analyte process. In some embodiments, discontinuing the single-analyte process includes an action such as stabilizing the single-analyte, removing the single analyte from the detection system, replacing the single-analyte with a second single analyte, adding the second single analyte to the detection system, reconfiguring the detection system, recalibrating the detection system, calling to a second detection system, refreshing a computer-implemented algorithm, updating the computer-implemented algorithm, and a combination thereof.

In some embodiments, a single-analyte process includes one or more subsidiary steps. In some embodiments, a subsidiary step includes any function of the single-analyte system that maintains the function of the system independent of the single-analyte process. In some embodiments, a subsidiary step includes maintenance functions and error handling functions. For example, in some embodiments, during a single-analyte process, a single-analyte system recognizes a maintenance function such as a depleted reagent, a dirty filtration element, or an expiring component per a manufacturer's specification. In some embodiments, the single-analyte system implements an action to maintain system function based upon the maintenance function. In some embodiments, a single-analyte system recognizes a damaged or malfunctioning component and prompt a technician to address the error. In some embodiments, a subsidiary step is automated or prompts a user input. For example, in some embodiments, a single-analyte system is configured to automatically replace a depleted reagent, or a depleted reagent is replaced by a user of the single-analyte system. In some embodiments, a subsidiary step occurs in parallel with a single-analyte process (i.e., a background system function) or is sequenced with a single-analyte process or an iterative process thereof (e.g., a process is paused to replace a depleted reagent).

In some embodiments, a subsidiary step is indicated and/or implemented based upon a single-analyte data set. In some embodiments, a subsidiary step is indicated and/or implemented based upon a process metric derived from a single-analyte data set. In some embodiments, a single-analyte process includes the steps of: determining a process metric for a process component based upon the set of single-analyte system data; and implementing a subsidiary action on a single-analyte system based upon the process metric.

In some embodiments, a process metric that determines a subsidiary action is calculated from the single-analyte data set. In some embodiments, a process metric that determines a subsidiary action is used or determined similarly to other process metrics set forth herein. In some embodiments, a subsidiary action is determined based upon a process metric similarly to other single-analyte process actions set forth herein. In some embodiments, the process metric includes a value from the single-analyte data set (e.g., instrument metadata such as fluid level or fluid composition). In some embodiments, determining a process metric includes the steps of deriving a value from the single-analyte data set, and deriving the process metric from a reference source based upon the value derived from the single-analyte data set. In some embodiments, a process metric for a subsidiary step includes an environmental metric for the detection system (e.g., external temperature, external pressure, external humidity, etc.). In some embodiments, a process metric for a subsidiary step includes a system-state metric. In some embodiments, the system-state metric indicates, for example, a normal state, an error state, an idle state, an operating state, or a combination thereof. For example, in some embodiments, a system-state metric manifests as a warning or an alarm due to a low reagent level or due to movement of a system component beyond its designed boundaries. In some embodiments, a system-state metric includes an ON/OFF state for a pump or valve, thereby possibly indicating fluid flow within the single-analyte system. In some embodiments, a system-state metric includes two or more states. For example, in some embodiments, an ON or OFF state for a valve includes an operating state and an error state if the valve is not set in its intended position.

In some embodiments, a single-analyte process includes, before performing an iterative process, providing a sequence of steps for the single-analyte process. In some embodiments, a sequence of steps includes a plurality of steps for the single-analyte process. In some embodiments, a plurality of steps includes a step of performing a physical measurement on the single analyte. In some embodiments, two or more steps of the plurality of steps includes performing the physical measurement on the single analyte. In some embodiments, a step of the sequence of steps is performed before the iterative process. In some embodiments, a plurality of steps of the sequence of steps is performed before the iterative process. In some embodiments, a single-analyte process includes, before the iterative process, obtaining a preliminary single-analyte data set. In some embodiments, a sequence of steps for single-analyte process is based upon the preliminary single-analyte data set. In some embodiments, a sequence of steps is determined similarly to other methods set forth herein.

In some embodiments, a single-analyte process includes, after an iterative process, providing a subsequent sequence of steps for the single-analyte process. In some embodiments, a subsequent sequence of steps includes a subsequent plurality of steps for the single-analyte process. In some embodiments, a subsequent plurality of steps includes a step of performing a physical measurement on the single analyte. In some embodiments, two or more steps of a subsequent plurality of steps includes performing the physical measurement on the single analyte. In some embodiments, a single-analyte process includes, after an iterative process, obtaining a single-analyte data set. In some embodiments, a subsequent sequence of steps is determined similarly to other methods set forth herein.

In some embodiments, an iterative approach to a single-analyte process is advantageous for any one of several reasons, including: altering a total number of performed steps during a single-analyte process; altering a total amount of time for the single-analyte process; altering a total amount of reagent or material consumed by the single-analyte process; increasing the likelihood of obtaining a successful result from the single-analyte process; altering the efficiency of a single-analyte process; increasing the confidence level of the characterization of a single-analyte process; decreasing an uncertainty level for the successful completion of a step within a single-analyte process; or a combination thereof.

In some embodiments, altering a total number of performed steps during a single-analyte process includes increasing or decreasing the total number of performed steps. For example, in some embodiments, it is advantageous to eliminate unnecessary steps from a standard or baseline protocol by implementing an iterative process. In some embodiments, it is advantageous to add steps that increase the likelihood of obtaining a successful result in comparison to a baseline or standard protocol for a single-analyte process. In some embodiments, altering a total amount of time for a single-analyte process includes increasing or decreasing the total amount of time. For example, in some embodiments, it is advantageous to obtain a single-analyte identity from a single-analyte assay with fewer assaying steps relative to a baseline or standard assaying protocol, or relative to an equivalent bulk assaying protocol. In some embodiments, it is advantageous to obtain a single-analyte identity from a single-analyte assay with more assaying steps to increase the confidence of the identity relative to a baseline or standard assaying protocol, or relative to an equivalent bulk assaying protocol. In some embodiments, altering a total amount of a reagent or material consumed by a single-analyte process includes increasing or decreasing the amount of reagent or material consumed. For e some embodiments, it is advantageous to decrease the quantity of a rare, limited, or expensive material or reagent by implementing an iterative process that facilitates reduced reagent or material usage relative to a baseline or standard protocol, or relative to an equivalent bulk protocol. In some embodiments, it is advantageous to increase the usage of a reagent or material relative to a baseline or standard protocol, or relative to an equivalent bulk protocol, such as increased use of a rinsing reagent to improve removal of a reagent or material during a single-analyte process. In some embodiments, altering the efficiency of a single-analyte process includes increasing or decreasing the efficiency. some embodiments, it is advantageous to increase the efficiency of a single-analyte process relative to a baseline or standard protocol, or relative to an equivalent bulk protocol, such as by implementing an iterative process that attempts to optimize process performance. In some embodiments, a user specifies a decreased efficiency to save time or cost relative to a baseline or standard protocol, or relative to an equivalent bulk protocol, and an iterative process is implemented to facilitate obtaining a satisfactory result within the user-imposed limitation.

In some embodiments, an iterative process alters a total number of performed steps, procedures, or sub-procedures in a single-analyte process, for example by removing unnecessary steps, procedures, or sub-procedures, or by adding necessary steps, procedures, or sub-procedures. In some embodiments, a completed single-analyte process includes a total number of performed steps. In some embodiments, a total number of performed steps of a single-analyte process after the determinant criterium is achieved is greater than or less than a total number of steps of a preliminary sequence of steps for the single-analyte process. In some embodiments, a total number of performed steps of a single-analyte process after the determinant criterium is achieved is greater than or less than a total number of steps of a comparative process such as a baseline or standard process, or a bulk process. In some embodiments, an iterative process reduces the total number of performed steps relative to a preliminary sequence of steps or a comparative process by at least about 1%, 5%, 10%, 15%, 20%, 25%, 30%, 35%, 40%, 45%, 50%, 55%, 60%, 65%, 70%, 75%, 80%, 85%, 90%, 95%, 99%, or more. In some embodiments, an iterative process reduces the total number of performed steps relative to a preliminary sequence of steps or a comparative process by no more than about 99%, 95%, 90%, 85%, 80%, 75%, 70%, 65%, 60%, 55%, 50%, 45%, 40%, 35%, 30%, 25%, 20%, 15%, 10%, 5%, 1%, or less. In some embodiments, an iterative process increases the total number of performed steps relative to a preliminary sequence of steps or a comparative process by at least about 1%, 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, or more. In some embodiments, an iterative process increases the total number of performed steps relative to a preliminary sequence of steps or a comparative process by no more than about 1000%, 500%, 400%, 300%, 200%, 100%, 95%, 90%, 85%, 80%, 75%, 70%, 65%, 60%, 55%, 50%, 45%, 40%, 35%, 30%, 25%, 20%, 15%, 10%, 5%, 1%, or less.

In some embodiments, a single analyte process is characterized by a total elapsed process time. In some embodiments, the total elapsed process time refers to the length of time from the initiation of the single-analyte process to the completion of the single-analyte process. In some embodiments, the total elapsed process time excludes delays due to system malfunctions, external interruptions, or other sources of delay. In some embodiments, an iterative process in a single-analyte process alters the total elapsed process time, for example by increasing or reducing the total number of performed steps, procedures, or sub-procedures. In some embodiments, a total elapsed time of a single-analyte process after the determinant criterium is achieved is greater than or less than a predicted elapsed time based upon a preliminary sequence of steps for the single-analyte process. In some embodiments, a total elapsed time of a single-analyte process after the determinant criterium is achieved is greater than or less than a total elapsed time of a comparative process such as a baseline or standard process, or a bulk process. In some embodiments, an iterative process reduces the total elapsed time of a single-analyte process relative to a predicted elapsed time-based upon a preliminary sequence of steps or a comparative process by at least about 1%, 5%, 10%, 15%, 20%, 25%, 30%, 35%, 40%, 45%, 50%, 55%, 60%, 65%, 70%, 75%, 80%, 85%, 90%, 95%, 99%, or more. In some embodiments, an iterative process reduces the total elapsed time of a single-analyte process relative to a predicted elapsed time-based upon a preliminary sequence of steps or a comparative process by no more than about 99%, 95%, 90%, 85%, 80%, 75%, 70%, 65%, 60%, 55%, 50%, 45%, 40%, 35%, 30%, 25%, 20%, 15%, 10%, 5%, 1%, or less. In some embodiments, an iterative process increases the total elapsed time relative to a preliminary sequence of steps or a comparative process by at least about 1%, 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, or more. In some embodiments, an iterative process increases the total elapsed time relative to a preliminary sequence of steps or a comparative process by no more than about 1000%, 500%, 400%, 300%, 200%, 100%, 95%, 90%, 85%, 80%, 75%, 70%, 65%, 60%, 55%, 50%, 45%, 40%, 35%, 30%, 25%, 20%, 15%, 10%, 5%, 1%, or less.

In some embodiments, an iterative process during a single-analyte process decreases one or more measures of uncertainty with respect to the single-analyte system and/or the single-analyte process. In some embodiments, an iterative process reduces an uncertainty metric with respect to a characterization of a single analyte. For example, in some embodiments, an iterative process is utilized to increase the confidence level of a characterization that a single analyte has been properly synthesized at the completion of a single-analyte synthesis process. In some embodiments, an iterative process is utilized to increase the confidence level of a single-analyte identification at the completion of a single-analyte identification assay. In some embodiments, an iterative process reduces an uncertainty metric with respect to a datum collected during a single-analyte process. For example, in some embodiments, a measurement of a single-analyte property is repeated during an iterative process to decrease the likelihood of a false positive or a false negative measurement. In some embodiments, the uncertainty metric for the single analyte after the iterative process shows a decreased level of uncertainty relative to the uncertainty metric for the single analyte before the iterative process.

In some embodiments, an iterative process includes a step of updating the single-analyte data set before implementing the action on the single-analyte system. In some embodiments, a single-analyte data set is updated for a purpose such as configuring the action before implementing the action on the single-analyte system, or confirming the need to perform the action (e.g., checking the accuracy of a process metric upon which the action is based, confirming that a source of uncertainty has not resolved before implementing an action to address the uncertainty).

In some embodiments, the methods for configuring a single-analyte process set forth herein are readily extended to single-analyte systems comprising a plurality of single analytes. For example, in some embodiments, a plurality of single analytes is detected, characterized, or manipulated using an array of sites, each of the sites attached to a single analyte, or using other multiplex formats. In some embodiments, a plurality of single analytes is detected, characterized, or manipulated in parallel using a multiplex format, such as an array of single analytes. In some embodiments, a plurality of single analytes is detected, characterized, or manipulated serially (e.g., one single analyte after another) using a multiplex format. In some embodiments, a multiplex single-analyte system includes conceivably tens, hundreds, thousands, millions, billions, trillions, or higher numbers of single-analytes. In some embodiments, the iterative process methods detailed herein are extended to single-analyte systems comprising a plurality of single analytes if the single-analyte system is configured to obtain physical measurements and/or characterizations of each single analyte at single-analyte resolution.

In some embodiments, a single-analyte process for a single-analyte system comprising a plurality of single analytes includes an iterative process. In some embodiments, an iterative process for a single-analyte system comprising a plurality of single analytes includes a step of determining a curated process metric (e.g., a curated uncertainty metric) for the plurality of single analytes. In some embodiments, the determining of a curated process metric includes the steps of: determining a plurality of process metrics comprising a process metric for each single analyte of the plurality of single analytes; and determining a curated process metric based upon the plurality of process metrics.

In some embodiments, the determining of a curated process metric based upon the plurality of process metrics includes calculating a curated process metric from the plurality of process metrics (e.g., determining a mean or a median value). In some embodiments, the determining of a curated process metric based upon the plurality of process metrics includes a data reduction or data analysis method such as: extracting one or more process metrics from a plurality of process metrics; removing one or more process metrics from a plurality of process metrics; ranking each process metric of a plurality of process metrics; categorizing each process metric of a plurality of process metrics; or a combination thereof.

In some embodiments, a data reduction or data analysis method produces a reduced, sorted, categorized, or ordered plurality of process metrics. In some embodiments, a curated process metric is determined from a reduced, sorted, categorized, or ordered plurality of process metrics by calculating the curated process metric from the reduced, sorted, categorized, or ordered plurality of process metrics. In some embodiments, a curated process metric is determined from a reduced, sorted, categorized, or ordered plurality of process metrics by determining a consensus process metric. In some embodiments, a consensus process metric includes a process metric value that applies to a representative subset of the plurality of single analytes, such as a simple majority, a relative majority, a simple minority, a relative minority, or a median. For example, in some embodiments, a single-analyte assay includes a determination of a source for a plurality of single analytes from an unknown source. In some embodiments, based upon a preliminary single-analyte data set, a consensus process metric for the plurality of single analytes is determined during an iterative process, and a consensus action is implemented based upon the consensus process metric that represents the next most informative measurement for characterizing the source of the single analytes. In some embodiments, a plurality of single analytes is measured during a step of a single-analyte fabrication process. In some embodiments, based upon the measurements of the plurality of single analytes, a consensus process metric is estimated that represents the likelihood that the fabrication step succeeded for a specified set of single analytes. In some embodiments, if the consensus process metric is found to fall below a threshold value, the step, a procedure thereof, or a sub-procedure thereof, is repeated to increase the likelihood that the fabrication step succeeded for a specified set of the single analytes. In some embodiments, an iterative process includes the steps of: determining a consensus process metric (e.g., a consensus uncertainty metric) for a plurality of single analytes; and implementing an action on the single-analyte system based upon the consensus process metric.

### Single-Analyte Data Sources

In some embodiments, data is collected, compiled, manipulated, and/or applied before, during or after a single-analyte process to form a single-analyte data set. In some embodiments, data is collected, compiled, manipulated, and/or applied before, during or after an iterative process of a single-analyte process to form, manipulate, or otherwise utilize a single-analyte data set. In some embodiments, a single-analyte data set is applied before, during, or after a single-analyte process and/or an iterative process thereof for one or more purposes, including: facilitating the control of a single-analyte process and/or an iterative process thereof; confirming the outcome of a single-analyte process and/or an iterative process thereof; optimizing or refining a single-analyte process and/or an iterative process thereof; providing a repository of data for the performing of subsequent single-analyte processes and/or iterative processes thereof; or a combination thereof. In some embodiments, a single-analyte process and/or an iterative process thereof utilizes one or more single-analyte data sets during a single-analyte process and/or an iterative process thereof. For example, in some embodiments, a single-analyte process or an iterative process thereof utilizes a first single-analyte data set that comprises invariant information (e.g., vendor-supplied reagent information; process start time; user-supplied process parameters, etc.), and a second single-analyte data set that comprises variable information (e.g., single-analyte characterization measurements; system sensor readings; ambient environmental data, etc.).

In some embodiments, a single-analyte process utilizes one or more single-analyte data sets. In some embodiments, an iterative process of a single-analyte process utilizes one or more single-analyte data sets. In some embodiments, a single-analyte process and/or an iterative process utilizes at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 600, 700, 800, 900, 1000, or more single-analyte data sets. In some embodiments, a single-analyte process and/or an iterative process utilizes no more than about 1000, 900, 800, 700, 600, 500, 450, 400, 350, 300, 250, 200, 150, 100, 95, 90, 85, 80, 75, 70, 65, 60, 55, 50, 45, 40, 35, 30, 25, 20, 19, 18, 17, 16, 15, 14, 13, 12, 11, 10, 9, 8, 7, 6, 5, 4, 3, 2 or fewer single-analyte data sets.

In some embodiments, data is collected from one or more data sources before, during or after a single-analyte process. In some embodiments, data is collected from one or more data sources before, during or after an iterative process of a single-analyte process. In some embodiments, data sources include any source of information that is included in a single-analyte data set. In some embodiments, a single-analyte data set includes a datum from a single data source. In some embodiments, a single-analyte data source includes data from at least about 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, 1000, or more data sources. In some embodiments, a single-analyte data set includes data from no more than about 1000, 900, 800, 700, 600, 500, 400, 300, 200, 100, 95, 90, 85, 80, 75, 70, 65, 60, 55, 50, 45, 40, 35, 30, 25, 20, 15, 10, 9, 8, 7, 6, 5, 4, 3, 2, or fewer data sources. In some embodiments, a single-analyte data set includes data that is derived or calculated from one or more data sources. For example, in some embodiments, a single-analyte data set consists exclusively of data that is calculated from one or more single-analyte data sets, in which each single-analyte data set of the one or more single-analyte data sets comprise data collected from at least one data source.

In some embodiments, a single-analyte process as set forth herein utilizes one or more single-analyte data sets. In some embodiments, an iterative process of a single-analyte process as set forth herein utilizes one or more single-analyte data sets. In some embodiments, an algorithm of a single-analyte process utilizes one or more single-analyte data sets. In some embodiments, utilization of a single-analyte data set includes a data processing activity, including obtaining a value of a datum from a single-analyte data set, adding a value of a datum to a single-analyte data set, removing a value of a datum from a single-analyte data set, altering a value of a datum within a single-analyte data set, determining a value (e.g., a process metric) from a datum of a single-analyte data set, compiling a plurality of data into a single-analyte data set, concatenating a plurality of data into a single-analyte data set, and generating a second single-analyte data set utilizing a datum from a first single-analyte data set by any of the data processing activities set forth herein. In some embodiments, utilization of one or more single-analyte data sets includes the use of one or more algorithms (e.g., computer-implemented algorithms, etc.), as set forth herein. In some embodiments, a single-analyte process, an iterative process thereof, and/or an algorithm thereof utilizes two or more single-analyte data sets simultaneously. In some embodiments, simultaneous utilization of two or more single-analyte data sets includes manipulating data from a first single-analyte data set utilizing data from a second single-analyte data set. For example, in some embodiments, one or more data from a first single-analyte data set is altered (e.g., corrected or updated) based upon one or more data of instrument metadata (e.g., temperature, pressure, etc.) obtained from a second single-analyte data set. In some embodiments, a third single-analyte data set comprising one or more process metrics is generated by deriving the process metrics from one or more data of a first single-analyte data set and optionally, utilizing one or more data from a second single-analyte data set while deriving the process metrics. In some embodiments, simultaneous utilization of two or more single-analyte data sets includes simultaneous manipulation of data from both of a first single-analyte data set and a second single-analyte data set. For example, in some embodiments, data from a first single-analyte data set comprising physical measurements of a single analyte and data from a second single-analyte data set comprising cumulative data of physical measurements is simultaneously sorted and/or categorized for the purpose of comparing the physical measurements of the single analyte to the cumulative data.

In some embodiments, a single-analyte process, an iterative process thereof, and/or an algorithm thereof utilizes two or more single-analyte data sets sequentially. In some embodiments, the sequential utilization of two or more single-analyte data sets includes processing one or more data from a first single-analyte data set, and then processing data one or more data from a second single-analyte data set. For example, in some embodiments, a first single-analyte data set comprising instrumental metadata is altered by a data noise reduction process before the data from the first single-analyte data set is utilized to perform a data correction process on measurement data from a second single-analyte process. In some embodiments, sequential utilization of two or more single-analyte data sets further comprises a Boolean or logical operation. In some embodiments, a Boolean operation includes determining if a second single-analyte data set should be processed based upon information determined from a first single-analyte data set. For example, in some embodiments, a first single-analyte data set is processed to determine a first process metric and, if the first process metric meets a specified condition, a second single-analyte data set is processed to determine a second process metric. In some embodiments, a logical operation includes determining which second single-analyte data set should be processed based upon information determined from a first single-analyte data set. For example, in some embodiments, a first single-analyte data set is processed to determine a first process metric and, based upon a value of the first process metric, a second single-analyte data set is selected from two or more single-analyte data sets and processed to determine a second process metric.

In some embodiments, a single-analyte process, an iterative process thereof, and/or an algorithm thereof is configured to utilize differing single-analyte data sets at differing times, under differing circumstances, and/or during differing conditions. In some embodiments, a first single-analyte data set is used once during a single-analyte process and/or an iterative process thereof, and a second single-analyte data set is used more than once during the single-analyte process and/or iterative process thereof. For example, in some embodiments, an invariant single-analyte data set comprising sample data is utilized at the initiation of a single-analyte process to configure an initial sequence of steps for the single-analyte process, and a variable single-analyte data set comprising physical measurement data is used thereafter to implement the single-analyte process and/or iterative processes thereof. In some embodiments, a first single-analyte data set is utilized to record all process-related information during an iterative process, and a second single-analyte data set is utilized only at the termination of the iterative process to record a subset of the process-related information during an iterative process. In some embodiments, a first single-analyte data set and a second single-analyte data set are used in a patterned or conditioned sequence. For example, in some embodiments, a datum from a first single-analyte data set is utilized to initiate an iterative process and a datum from a second single-analyte data set is utilized to terminate the iterative process. In some embodiments, an iterative process utilizes data from a first single-analyte data set until a condition is achieved, then utilize data from a second single-analyte data set.

In some embodiments, an action implemented during a single-analyte process and/or an iterative process thereof utilizes one or more data from one or more single-analyte data sets. In some embodiments, utilization of one or more single-analyte data sets while implementing an action includes utilizing one or more single-analyte data sets to select the action, utilizing one or more single-analyte data sets to configure the action (e.g., configuring steps, procedures, and/or sub-procedures comprising the action), and/or utilizing one or more single-analyte data sets while performing the action (e.g., determining a process metric that controls when the action is terminated). In some embodiments, an action implemented during a single-analyte process and/or an iterative process thereof is configured based upon one or more data from one or more single-analyte data sets. In some embodiments, a parameter of an action implemented during a single-analyte process and/or an iterative process thereof is configured based upon one or more data from one or more single-analyte data sets. For example, in some embodiments, a length of a pause during a single-analyte process is configured based upon one or more data from one or more single-analyte data sets.

In some embodiments, a single-analyte data set includes data that is externally collected, internally collected, or derived before, during, or after a single-analyte process. In some embodiments, a single-analyte data set includes data that is a combination of externally-collected data, internally-collected data, and/or derived data. For example, in some embodiments, a single-analyte data set includes user-input data regarding a single analyte and physical measurements obtained by the single-analyte system. In some embodiments, externally-collected data includes any data that originates external to a single-analyte system, such as third-party information, reference information, user-supplied information collected on a differing system, and the like. For example, in some embodiments, externally-collected data includes reagent composition data provided by vendors, or tabular data from a reference source (e.g., a textbook). In some embodiments, internally-collected data includes any data that originates within a single-analyte system, such as single-analyte physical measurements, instrument data, user-supplied information collected within the single-analyte system, cumulative data, and the like. For example, in some embodiments, internally-collected data includes a set of single-analyte image data collected by an optical device, or includes a set of cumulative single-analyte image data collected during prior single-analyte processes. In some embodiments, derived data includes data that is determined by data manipulation of other data (e.g., calculating, sorting, categorizing, decoding, etc.). In some embodiments, a derived datum is determined based upon one or more data, including externally-collected data, internally-collected data, or a combination thereof. For example, in some embodiments, derived data includes one or more process metrics that are calculated or otherwise determined from externally-collected data or internally-collected data.

In some embodiments, a single-analyte data set includes data that is invariant, variable, or cumulative. In some embodiments, invariant data includes any datum that has a temporally-fixed value after being incorporated into a single-analyte data set. For example, in some embodiments, a single-analyte data set includes an invariant list of composition information for all reagents utilized during a single-analyte process. In some embodiments, a single-analyte data set includes an invariant compilation of all physical measurement data obtained during a single-analyte process. In some embodiments, variable data includes any datum that is expected to have a temporally-changing value after being incorporated into a single-analyte data set. For example, in some embodiments, a single-analyte data set includes one or more process metrics whose values are updated at various times, such as during each cycle of an iterative process. In some embodiments, cumulative data includes any datum retained or stored from previous single-analyte processes. For example, in some embodiments, a cumulative single-analyte data set comprises a compilation of process metrics from all known prior runs of a single-analyte process involving the same single-analyte as a current process. In some embodiments, a single-analyte data set comprising cumulative data includes data such as prior analyte information, prior physical measurements, prior instrument data, prior process results, prior process configurations (e.g., sequences of steps, procedures, and/or sub-procedures), or a combination thereof. In some embodiments, cumulative data is compiled, aggregated, or curated. In some embodiments, cumulative data is altered or updated before, during, or after the performing of a single-analyte process and/or an iterative process thereof.

In some embodiments, a single-analyte data set includes reference data. In some embodiments, reference data includes any datum that is obtained from a publicly available source. In some embodiments, reference data includes tabular data, theoretical equations and/or values derived therefrom, empirical correlations and/or values derived therefrom, published data from sources such as textbooks, journal articles, manufacturer-provided materials, websites, and databases (e.g., the U.S. NIST Chemistry Webbook). In some embodiments, reference data includes a datum that is mined, calculated, extrapolated, or otherwise derived from a reference source. For example, in some embodiments, a single-analyte data set includes information regarding a physical property of a single analyte, in which the information is data-mined by an algorithm from a database of peer-reviewed publications. In some embodiments, reference data is compiled, aggregated, or curated. In some embodiments, reference data is altered or updated before, during, or after the performing of a single-analyte process and/or an iterative process thereof.

In some embodiments, a single-analyte data set includes cumulative data. In some embodiments, cumulative data includes a plurality of internally-collected data that has been collected with regard to a single-analyte system, a single-analyte process, a single-analyte, or a combination thereof. In some embodiments, cumulative data includes both internally-collected data and reference data. In some embodiments, cumulative data includes internally collected data while excluding reference data, or vice versa. In some embodiments, cumulative data includes relationships (e.g., correlations, mechanistic effects, etc.) between process metrics (e.g., uncertainty metrics) and system performance and/or single-analyte behaviors and/or properties. In some embodiments, cumulative data is utilized to configure an action during a single-analyte process and/or an iterative process thereof as set forth herein. In some embodiments, cumulative data is used to configure a sequence of steps, procedures, or sub-procedures during a single-analyte process and/or an iterative process thereof as set forth herein. In some embodiments, cumulative data is utilized s a predictive reference for an outcome of an implemented action during a single-analyte process and/or an iterative process thereof. For example, in some embodiments, an action in a single-analyte system is selected and/or implemented based upon a determined process metric (e.g., an uncertainty metric) with reference to a prior action and/or outcome in a single-analyte data set comprising cumulative data, in which the cumulative data was obtained from a single-analyte process where a similar or identical process metric existed. In some embodiments, cumulative data is utilized as a bounding reference for choosing and/or implementing an action during a single-analyte process and/or an iterative process thereof. For example, in some embodiments, an action from a list of possible actions in a single-analyte system is eliminated from consideration as a possible choice based upon a single-analyte data set comprising cumulative data when a determined process metric of the single-analyte system is determined to be similar or identical to a process metric of the cumulative data. In some embodiments, cumulative data is updated during a single-analyte process and/or an iterative process thereof to include a datum collected, determined, or derived during the single-analyte process. In some embodiments, an action is determined and/or implemented during a single-analyte process and/or an iterative process thereof utilizing cumulative data that includes a datum collected, determined, or derived during the same single-analyte process. For example, in some embodiments, a single-analyte synthesis process includes a repeated step (e.g., a rinsing step) in which the step is configured during each repetition of the step utilizing cumulative data comprising process parameters (e.g., rinse time length, rinse reagent volume, etc.) and associated process metrics that facilitate the configuration of the step. In some embodiments, a single-analyte process includes performing an iterative process until a determinant criterium has been met, in which the iterative process comprises the steps of: determining a process metric for a single analyte based upon a single-analyte data set comprising cumulative data; implementing an action on a single-analyte system based upon the process metric, the cumulative data, or a combination thereof, in which the single-analyte system comprises a detection system that is configured to obtain a physical measurement of the single analyte at single-analyte resolution; updating the cumulative data of the single-analyte data set after implementing the action on the single-analyte system; and determining the process metric for the single analyte based upon the single-analyte data set comprising the updated cumulative data. For example, in some embodiments, a physical characterization of a single analyte occurs via an iterative process that generates one or more physical measurements of the single analyte or the single-analyte system. In some embodiments, the one or more physical measurements is added to the cumulative data of a single-analyte data set during the iterative process. In some embodiments, at the completion of the iterative process, the physical characterization of the single analyte is performed again utilizing the most updated cumulative data to generate an updated physical characterization of the single analyte.

In some embodiments, a datum from a single-analyte data set is utilized during a single-analyte process and/or an iterative process thereof. In some embodiments, all data from a single-analyte data set is utilized during a single-analyte process and/or an iterative process thereof. In some embodiments, a subset of data from a single-analyte data set is utilized during a single-analyte process and/or an iterative process thereof. In some embodiments, data or subsets of data is utilized in any order or sequence, such as simultaneously, consecutively, non-consecutively, sequentially, non-sequentially, randomly, or a combination thereof.

In some embodiments, a single-analyte data set includes a reduced single-analyte data set. In some embodiments, a reduced single-analyte data set includes data that is collected, compiled, or derived from one or more larger single-analyte data sets. In some embodiments, a reduced single-analyte data set is formed by any suitable data reduction method, such as removing data from a single-analyte data set (e.g., unwanted data, unneeded data, statistically-invalid data, etc.), extracting a subset of data from a larger first single-analyte data set into a smaller second single-analyte data set, averaging data from one or more single-analyte data sets into a smaller averaged single-analyte data set, and/or sorting or categorizing a larger single-analyte data set by one or more data measures, then dividing the larger single-analyte data set into two or more smaller single-analyte data sets. For example, in some embodiments, a step of a single-analyte process includes repeatedly measuring a single analyte (e.g., by imaging, by spectroscopic analysis, etc.) and compiling the measurements into a first single-analyte data set. Thereafter, in some embodiments, a reduced single-analyte data set is formed by averaging the individual measurements from the first single-analyte data set and storing them as in a reduced second single-analyte data set. In some embodiments, a step of a single-analyte process includes optically observing an array of addresses on a solid support to determine which array addresses produce an optical signal (e.g., fluorescence, luminescence) indicating that an address is occupied by a single analyte. In some embodiments, a first single-analyte data set comprising array addresses and observed presence or absence of an optical signal is sorted according to addresses with a signal and addresses absent a signal, and the first single-analyte data set is divided into two reduced single-analyte data sets (e.g., a set of addresses with observed signal and a set of addresses with an absence of signal).

In some embodiments, a single-analyte data set is structured in any of a variety of forms. In some embodiments, exemplary data forms include single values, arrays, lists, trees, hash tables, and derived data structures. In some embodiments, arrays include unsorted and sorted arrays. In some embodiments, lists include unsorted, sorted, and circular lists. In some embodiments, trees include binary trees, binary search trees, AVL trees, Red-black trees, splay trees, treaps, and B-trees. In some embodiments, derived data structures include data stacks, data heaps, and data queues.

In some embodiments, a single-analyte data set is formed, manipulated, and/or applied by one or more algorithms as set forth herein. In some embodiments, a single-analyte data comprising information from two or more data sources is formed, manipulated, and/or applied by one or more algorithms as set forth herein. In some embodiments, an algorithm that forms, manipulates, or applies a datum from a single-analyte data set is a computer-implemented algorithm, as set forth herein. In some embodiments, a single-analyte data set is stored in a digital or non-digital form. For example, in some embodiments, a single-analyte data set is stored on a non-transitory computer-readable medium. In some embodiments, a single-analyte data set is stored for a defined duration of time, such as for the length of a single-analyte process or an iterative process thereof, or permanently (e.g., stored within a cumulative data set). In some embodiments, a single-analyte data set is stored temporarily. For example, in some embodiments, a single-analyte data set is stored temporarily during the performing of a calculation during a cycle of an iterative process. In some embodiments, a single-analyte data set is stored temporarily on a transitory computer-accessible medium (e.g., random access memory) or is stored temporarily on a non-transitory computer-accessible medium (e.g., a hard drive).

In some embodiments, a single-analyte data set includes data from one or more decentralized, distributed, or centralized data sources. In some embodiments, a decentralized or distributed data source includes a network of sensors that supply data and/or process metrics to a single-analyte data set. In some embodiments, a decentralized or distributed data source includes a set of algorithms that independently or cooperatively process data to calculate values (e.g., process metrics) for a single-analyte data set. In some embodiments, a single-analyte data set includes data that is pulled from a decentralized, distributed, or centralized data source. For example, in some embodiments, a single-analyte data set includes various calculated process metrics in which each process metric is pulled from a different node of a decentralized or distributed data source. In some embodiments, a single-analyte data set includes data pulled from a centralized data source such as a reference source. In some embodiments, a single-analyte data set includes data that is pushed from a decentralized, distributed, or centralized data source. For example, in some embodiments, a decentralized or distributed data source pushes values for calculated process metrics to the single-analyte data set from various nodes of the data source at varying times based upon the time when calculations are completed.

### Process Metrics and Uncertainty Metrics in Single-Analyte Systems

In some embodiments, a single-analyte process and/or an iterative process thereof utilizes one or more process metrics to determine and/or implement an action on a single-analyte system. In some embodiments, a process metric includes any measure of characteristic, property, effect, behavior, performance, or variability within a single-analyte system. In some embodiments, the one or more process metrics includes an uncertainty metric. In some embodiments, an uncertainty metric includes any measure of variability with respect to a characteristic, property or effect that is observed in a single-analyte system. In some embodiments, process metrics include quantitative process metrics and qualitative process metrics. In some embodiments, a quantitative process metric includes any process metric with a measured or sensed numeric value. In some embodiments, a qualitative process metric includes any process metric with a non-numeric value and/or a classified value. For example, in some embodiments, a process metric is considered a qualitative process metric if the metric is determined by a sorting of data into a category “1” or category “2.” In some embodiments, despite the numeric values of categories “1” and “2,” the broad and/or non-objective categorization of the metric causes the metric to be defined as a qualitative process metric.

In some embodiments, a process metric includes or is derived from information in a single-analyte system. In some embodiments, a process metric includes information concerning a single analyte or a component thereof (e.g., a reagent utilized to synthesize the single analyte). In some embodiments, information concerning a single analyte, or a component thereof, includes physical measurements of the single analyte or component thereof, physical characterizations of the single analyte or component thereof, externally-supplied information regarding the single analyte or component thereof, and measurements of variability for any physical measurements and/or physical characterizations of the single analyte or a component thereof. In some embodiments, a process metric includes information concerning a component of a single analyte system other than a single analyte. In some embodiments, information concerning a component of a single analyte system other than a single analyte includes physical measurements of the component other than the single analyte, physical characterizations of the component other than the single analyte, externally-supplied information regarding the component other than the single analyte, and measurements of variability for any physical measurements and/or physical characterizations of the component other than the single analyte.

In some embodiments, a process metric includes a sensed parameter. In some embodiments, a sensed parameter includes any metric within or related to a single-analyte system that is directly measured by a sensor or a measurement device. In some embodiments, sensors are electronically-actuated devices that convert a voltage or amperage signal into a measurement (e.g., thermocouples, photosensors, pressure transducers, etc.). In some embodiments, a sensed parameter includes a direct measurement of voltage or amperage, or a property derived therefrom (e.g., temperature, pressure, flow rate, velocity, etc.). In some embodiments, a sensed parameter includes a manual measurement of a metric within or related to a single-analyte system. For example, in some embodiments lengths, weights, and other properties are measured manually or by a separate instrument then logged in a single-analyte system before, during, or after a single-analyte process.

In some embodiments, a process metric includes an indirect parameter. In some embodiments, an indirect parameter includes any metric within or related to a single-analyte system that is not directly sensed by a sensor or a measurement device. In some embodiments, an indirect parameter includes parameters that are inferred, calculated, or otherwise derived from other metrics. In some embodiments, indirect parameters are determined via known relationships (e.g., correlations, empirical equations, tabular data, etc.) or is determined through the operation of a single-analyte system or a related system. In some embodiments, indirect parameters include bulk, overall, or global parameters. In some embodiments, an indirect parameter is calculated or otherwise determined from one or more sensed parameters (e.g., a temperature-dependent correlation, temperature- and pressure-dependent gas laws, etc.). In some embodiments, indirect parameters include physical property measurements (e.g., strain rate, heat transfer coefficient, viscosity, density, rate of reaction, etc.) that are calculated from one or more sensed parameters. In some embodiments, indirect parameters include dimensionless properties (e.g., Reynolds number, Nusselt number, Schmidt number, etc.) that correlate to the physical function of a single-analyte system or a component thereof.

In some embodiments, a process metric includes an enumerated or categorized metric. In some embodiments, an enumerated or categorized metric includes any metric whose value is classified into two or more values. In some embodiments, enumerated or categorized metrics include binary, trinary, or polynary metrics. In some embodiments, enumerated or categorized metrics are determined by the sorting and/or categorization of sensed parameters or indirect parameters. For example, in some embodiments, a group of pixel sensors corresponding to a single analyte is assigned values of “Detected” or “Not Detected” based upon measured voltages of each pixel sensor of the group of pixel sensors. In some embodiments, if a sufficient number of pixel sensors achieve a threshold sensed voltage or the cumulative sensed voltage of the group of pixel sensors exceeds a threshold value, an enumerated or categorized value of “Detected,” is input for the group of pixel sensors. In some embodiments, an enumerated or categorized metric is determined by the sorting and/or categorization of one or more process metrics, such as about 2, 3, 4, 5, 6, 7, 8, 9, 10, 15, 20, 25, 30, 40, 50, 60, 70, 80, 90, 100, or more than 100 process metrics. In some embodiments, an enumerated or categorized metric is determined for each single analyte of a plurality of single analytes. In some embodiments, an enumerated or categorized metric is determined from a plurality of process metrics, for example based upon an average, median, or count of the plurality of process metrics. For example, in some embodiments, a step of a single-analyte synthesis or fabrication process is enumerated or categorized as “Pass” or “Fail” based upon a total quantity of expected products that are detected amongst a plurality of single analytes. In some embodiments, the step is assigned the metric of “Pass” if the total quantity of expected products exceeds a threshold value that has been specified for the step.

In some embodiments, a process metric is a spatially-variable or temporally-variable. In some embodiments, a process metric is spatially-invariant or temporally-invariant. In some embodiments, a spatially-variable process metric is any process metric whose value is determined to be non-uniform within a defined measurement region. In some embodiments, a temporally-variable process metric is any process metric whose value is determined to be non-uniform over a defined time period. In some embodiments, a spatially-invariant process metric is any process metric whose value is determined to be uniform within a defined measurement region. In some embodiments, a temporally-invariant process metric is any process metric whose value is determined to be uniform over a defined time period. In some embodiments, a spatially-variable process metric is temporally-variable or temporally-invariant. For example, in some embodiments, the magnitude of a fluorescent signal at a fixed location is temporally-variable due to photobleaching of a fluorophore giving rise to the signal. In some embodiments, the magnitude of autofluorescence at a fixed location on a solid support is spatially-varied but temporally invariant due to the material composition (e.g., intrinsic fluorescence). In some embodiments, a temporally-variable process metric is spatially-variable or spatially-invariant. For example, in some embodiments, a standard deviation of a physical measurement is temporally-variable (e.g., changing with successive measurements) but is spatially-variable or spatially-invariant for each single analyte of an array of single analytes. In some embodiments, the spatial variability of a process metric is determined based upon a given length, area, or volume of a spatial region. For example, in some embodiments, a small region is spatially invariant but a group comprising a plurality of small regions is spatially variable. In some embodiments, the temporal variability of a process metric is determined based upon a given period of time. For example, in some embodiments, a process metric is invariant over a short time interval but is observed to vary over a longer time interval. In some embodiments, the variability of spatial or temporal process metrics is assessed based upon comparison of two or more point or instantaneous values, or by comparison of an average or weighted value, such as an integration or a moving average.

In some embodiments, a process metric is measured or determined at a designated time interval. In some embodiments, a time interval is a fixed time interval (e.g., a measurement every 10 seconds). In some embodiments, a time interval is a variable time interval. In some embodiments, a variable time interval is linked to one or more steps, procedures, or sub-procedures during a single-analyte process and/or an iterative process thereof (e.g., a measurement after each rinsing procedure). In some embodiments, two or more process metrics are determined at the same designated time interval. In some embodiments, two or more process metrics are determined at differing time intervals. In some embodiments, a time interval is determined based upon the length of time of an action, a step, a procedure, a sub-procedure, or a sequence of steps, procedures, and/or sub-procedures. For example, in some embodiments, a rinsing process is controlled utilizing a process metric comprising a concentration of a reagent. In some embodiments, a time interval for determining the concentration process metric is based upon the total configured time length of the rinsing sub-procedures. In some embodiments, a process metric is determined at a time interval based upon the time-related function of a component of a single-analyte system. For example, in some embodiments, a stepper motor for a translation stage that positions a single-analyte beneath a measurement device is configured to receive electrical impulses that initiate a step of the motor at milli-second intervals. In some embodiments, a position algorithm calculates a position-based process metric (e.g., distance to a registration target) on a sub-millisecond time interval and relay start/stop instructions to the stepper motor to achieve precise positional control. In some embodiments, a computer-implemented algorithm is configured to determine a process metric within a time interval that cannot be achieved by a user (e.g., a human subject).

In some embodiments, a process metric is stored within a single-analyte data set. In some embodiments, a process metric is stored outside of a single-analyte data set. In some embodiments, a current value of a process metric within a single-analyte data set is updated each time the process metric is updated. In some embodiments, a current value of a process metric within a single-analyte data set is updated due to an action, step, procedure, or sub-procedure occurring during a single-analyte process and/or an iterative step thereof. In some embodiments, a single-analyte data set includes a plurality of values of a process metric, such as a time series or a history. In some embodiments, a process metric within a single-analyte data set is utilized by one or more algorithms as set forth herein. For example, in some embodiments, a process metric is utilized by a hardware driver or other hardware control algorithm to configure the performance of a hardware component, and is further utilized by a process control algorithm that implements an iterative process during a single-analyte process. In some embodiments, a process metric is utilized by only one algorithm. For example, in some embodiments, a process metric is determined only for a process control algorithm that implements an iterative process during a single-analyte process. In some embodiments, a process metric is stored on a non-transitory computer-readable medium (e.g., a hard drive). In some embodiments, a process metric is stored on a transitory, computer-readable medium (e.g., random access memory). In some embodiments, a process metric is stored temporarily, such as for the time length of a single-analyte process, an iterative process thereof, an action, or a step, procedure, or sub-procedure thereof. In some embodiments, a process metric is stored permanently, for example within a cumulative single-analyte data set.

In some embodiments, a process metric includes a measure of variability within a single-analyte system. In some embodiments, a process metric includes a proxy measure of variability if the metric has a known relationship to a source of variability within a single-analyte system. For example, in some embodiments, a temperature is correlated to a false detection rate for a physical measurement such that the temperature is utilized as a proxy value for an uncertainty level of the physical measurement. In some embodiments, a sequence of steps of a single-analyte process is determined, in whole or in part, by a relationship between a proxy measure of variability and a property, effect, behavior, identity, or characterization of a single analyte. For example, in some embodiments, a single-analyte process and/or an iterative process thereof proceeds so long as a proxy measure of variability (e.g., temperature, pressure, fluid Reynolds number, etc.) is normal with respect to a threshold value (e.g., a maximum and/or minimum value of the proxy measure). In some embodiments, a single-analyte process and/or an iterative process thereof pauses or be altered if a proxy measure of variability (e.g., temperature, pressure, fluid Reynolds number, etc.) is abnormal with respect to a threshold value (e.g., traversing a maximum and/or minimum value of the proxy measure). In some embodiments, a process metric includes an uncertainty metric. In some embodiments, an uncertainty metric includes any measure of variability with respect to a characteristic, property or effect that is observed in a single-analyte system. In some embodiments, an uncertainty metric is determined from one or more data, such as process metrics. In some embodiments, an uncertainty metric is determined by a method such as a statistical calculation or an empirical correlation.

In some embodiments, an uncertainty metric includes a measure of variability with respect to a process metric. In some embodiments, an uncertainty metric includes a statistical measure of variability of a process metric such as confidence interval, confidence level, or standard deviation. In some embodiments, an uncertainty metric comprising a measure of variability with respect to a process metric is utilized to determine if and/or how the process metric is applied during a single-analyte process and/or an iterative process thereof. For example, in some embodiments, is be utilized to determine if a rinsing process has been satisfactorily completed. In some embodiments, an uncertainty metric with respect to a process metric is utilized to select an action during a single-analyte process and/or an iterative process thereof as set forth herein. In some embodiments, an uncertainty metric with respect to a process metric is utilized to select, configure, and/or implement a step, procedure, or sub-procedure during a single-analyte process and/or an iterative process thereof.

In some embodiments, an uncertainty metric includes a measure of variability with respect to a physical characterization of a single analyte. In some embodiments, an uncertainty metric includes a statistical measure of variability of a physical characterization of a single analyte such as confidence interval, confidence level, or standard deviation. In some embodiments, an uncertainty metric comprising a measure of variability with respect to a physical characterization of a single-analyte is applied during a single-analyte process and/or an iterative process thereof. For example, in some embodiments, a confidence level for a physical characterization of a single analyte is utilized to determine if additional physical measurements of the single analyte should be obtained. In some embodiments, an uncertainty metric with respect to a physical characterization of a single analyte is utilized to select an action during a single-analyte process and/or an iterative process thereof as set forth herein. In some embodiments, an uncertainty metric with respect to a physical characterization of a single analyte is utilized to select, configure, and/or implement a step, procedure, or sub-procedure during a single-analyte process and/or an iterative process thereof as set forth herein.

In some embodiments, an action performed on a single-analyte system is selected, configured, and/or implemented based upon a process metric. In some embodiments, an action performed on a single-analyte system is selected, configured, and/or implemented based upon an uncertainty metric. For example, in some embodiments, a single-analyte system performs an iterative process that repeats a physical measurement of a single analyte until an uncertainty metric for the physical measurement (e.g., a data quality metric for the physical measurement data) increases above a threshold level. In some embodiments, two or more actions are selected, configured, and/or implemented based upon a process metric. For example, in some embodiments, if a temperature stability metric suggests a system temperature instability has occurred during a physical measurement, an iterative process is altered to repeat the physical measurement and pause the single-analyte process until the temperature stability metric has achieved a value that suggests the system temperature has been stabilized. In some embodiments, two or more actions are selected, configured, and/or implemented based upon an uncertainty metric. For example, in some embodiments, an iterative process is paused and one or more steps of the single-analyte process altered based upon an uncertainty metric suggesting that a most recent step of a single-analyte process decreased the confidence of single-analyte characterization.

In some embodiments, an action in a single-analyte system is selected and/or implemented based upon two or more process metrics (e.g., uncertainty metrics) by utilizing a decision hierarchy. In some embodiments, a decision hierarchy includes one or more rules, standards, or practices for determining an action during a single-analyte process and/or an iterative process thereof. In some embodiments, an action is selected from a decision hierarchy if a rule is met based upon the determined conditions for the two or more process metrics. Table I depicts a decision hierarchy for an exemplary system based upon two process metrics. In some embodiments, each process metric of the two process metrics (e.g., metric 1 and metric 2) is evaluated with respect to a rule for the metric (e.g., process metric>threshold value). In some embodiments, rules, standards, or practices for establishing a decision hierarchy are determined by methods as set forth herein. In some embodiments, each process metric of the two process metrics is assigned a value of “true” or “false” in the decision hierarchy based upon a respective rule. Table I shows how different combinations of meeting or not meeting the rule for each of the two or more process metrics cause a different action to be chosen for a single-analyte process. In some embodiments, a decision hierarchy is automatically implemented within a single-analyte process or an iterative process thereof. In some embodiments, a decision hierarchy includes decisions that require a user input.

TABLE I

| | | Metric 1 | |
|---|---|---|---|
| | | TRUE | FALSE |
| Metric 2 | TRUE | Action 1 | Action 1 |
| | TRUE | FALSE | Action 3 |

### Determining Actions During Single-Analyte Processes

Described herein are methods and system for control of single-analyte processes that are implemented on single-analyte systems. The single-analyte processes utilize an iterative process to control the steps, procedures, or sub-procedures that comprise the single-analyte process. In some embodiments, an iterative process utilizes one or more process metrics (e.g., uncertainty metrics) to select and implement an action on the single-analyte system. In some embodiments, an action that is selected and/or implemented on a single-analyte system during a single-analyte process is determined based upon a targeted or defined outcome for the single-analyte process. In some embodiments, an outcome of a single-analyte process includes a qualitative outcome (e.g., determining a single-analyte identity), a quantitative outcome (e.g., determining a single-analyte kinetic rate constant), or a combination thereof.

In some embodiments, the control of a single-analyte process is based, in whole or in part, upon a targeted or defined outcome for the single-analyte process. In some embodiments, a targeted outcome includes an outcome for a single-analyte process that is ideal or preferred. For example, in some embodiments, a targeted outcome includes a desired process efficiency, or minimized usage of a reagent during the single-analyte process. In some embodiments, a defined outcome includes an outcome for a single-analyte process that must occur to have the single-analyte process be considered completed. For example, in some embodiments, a defined outcome includes the completion of a synthesis process, or the measurement of a single-analyte property during a single-analyte assay. In some embodiments, a single-analyte process includes more than one targeted and/or defined outcome. In some embodiments, a single-analyte process includes more than one targeted and/or defined outcome with a hierarchy, ranking, or ordering of importance for achieving the outcome before the completion of the single-analyte process. For example, in some embodiments, a single-analyte assay includes a targeted outcome of characterizing a plurality of single analytes with 95% efficiency, unless achieving that level of efficiency requires utilizing more than a threshold quantity of a rare and/or expensive reagent.

In some embodiments, determining if an outcome has been achieved is based, in whole or in part, upon one or more characterizations of a single analyte. For example, in some embodiments, a single-analyte synthesis process with a desired outcome of producing a particular product includes one or more physical measurements to provide a characterization that confirms the proper synthesis of the particular product. In some embodiments, a single-analyte assay process with a targeted outcome of identifying 90% of a plurality of single analytes include one or more physical measurements of each single analyte of the plurality of single analytes that facilitate determining identity characterizations for each single analyte of the plurality of single analytes. In some embodiments, a characterization of a single analyte includes determining a property, behavior, effect, interaction, or identity of the single analyte. In some embodiments, a characterization of a single analyte includes a qualitative characterization (e.g., a polypeptide identity), a quantitative characterization (e.g., a polypeptide isoelectric point), or a combination thereof (e.g., a polypeptide identity and an associated confidence level for the identification). In some embodiments, characterizing a single analyte includes confirming a known property, behavior, effect, interaction, or identity for the single analyte. For example, in some embodiments, a synthesized or fabricated single analyte (e.g., a polynucleotide) is characterized as possessing an expected and/or known property for the single analyte (e.g., a polynucleotide sequence). In some embodiments, characterizing a single analyte includes determining an unknown property, behavior, effect, interaction, or identity for the single analyte. For example, in some embodiments, a random polypeptide from a polypeptide sample of unknown composition is characterized to determine an identity of the unknown polypeptide.

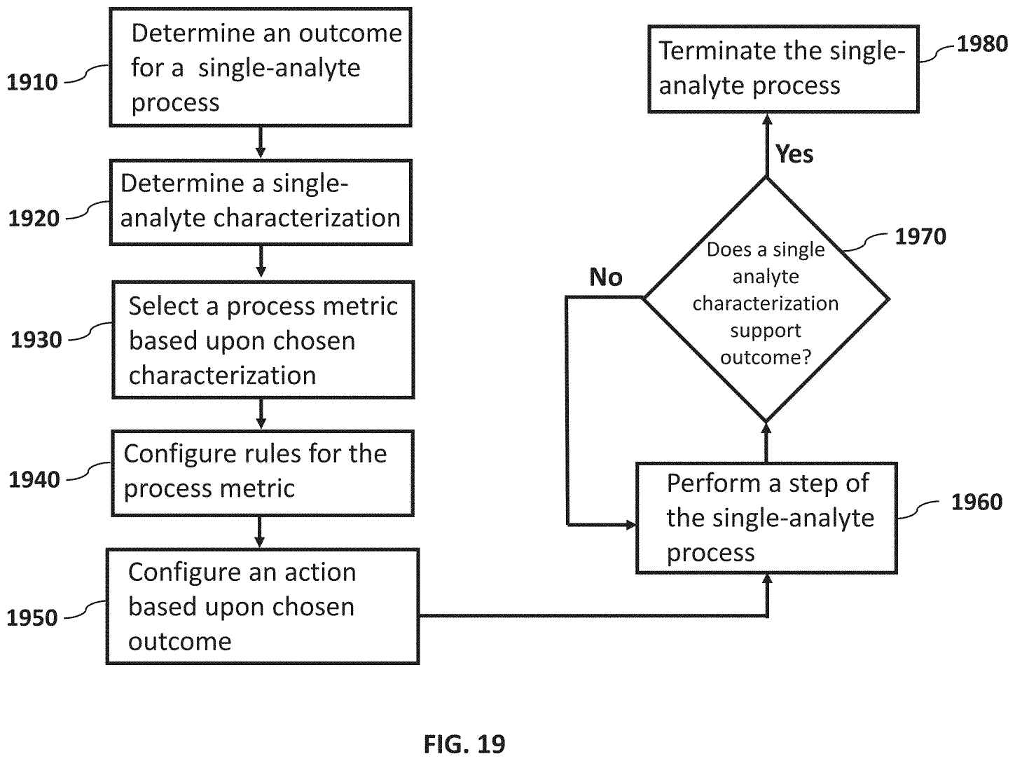

FIG. 19 depicts a method for performing a single-analyte process scheme including the determination of one or more outcomes for the process, in accordance with some embodiments. In some embodiments, an outcome, or a plurality of outcomes, is determined 1910 for a single-analyte process. In some embodiments, based upon the one or more determined outcomes 1910, a single-analyte characterization that confirms the one or more outcomes 1910 is determined 1920. In some embodiments, subsequently or simultaneously to determining a relevant single-analyte characterization, a process metric or a plurality of process metrics is selected 1930 based upon their relevance to determining if one or more of the determined outcomes 1910 are being achieved when the single-analyte process is performed. In some embodiments, after selecting the one or more process metrics 1930, rules for the one or more process metrics are configured 1940 to provide guidance on how the one or more process metrics should be interpreted or handled during the single-analyte process. In some embodiments, subsequently or simultaneously, an action or a plurality of actions is configured 1950 to permit an iterative process to be implemented during a single-analyte process. In some embodiments, the configured rules 1940 and configured actions 1950 are provided to a single-analyte system (e.g., provided to one or more algorithms implemented by one or more processors of the single-analyte system) and one or more steps of a single-analyte process is performed 1960. In some embodiments, the one or more iterative processes utilizing the configured rules 1940 and configured actions 1950 is performed during the performing of the one or more steps of the single-analyte process 1960. In some embodiments, during the performing of the single-analyte process, a single-analyte characterization is performed, and the single-analyte characterization is compared to the one or more outcomes to determine if the one or more outcomes have been achieved 1970. In some embodiments, if a single-analyte characterization does not support an outcome having been achieved, the single-analyte process is continued 1950 by performing one or more additional steps. In some embodiments, if a single-analyte characterization does support an outcome having been achieved, the single-analyte process is terminated 1980.

In some embodiments, one or more outcomes of a single-analyte process is defined before, or during a single-analyte process. In some embodiments, an outcome of a single-analyte process is supplied by a user. In some embodiments, an outcome of a single-analyte process is automatic or pre-defined. For example, in some embodiments, a single-analyte system is configured to automatically perform a single-analyte process with a pre-defined set of one or more outcomes. In some embodiments, a single-analyte system automatically determines one or more outcomes for a single-analyte process based upon one or more data within a single-analyte data set. For example, in some embodiments, a single-analyte system configures a single-analyte process based upon preliminary single-analyte data supplied by a user. In some embodiments, a single-analyte system automatically determines one or more outcomes for a single-analyte process based upon an input provided by a user, such as a user-defined outcome. In some embodiments, an outcome is changed, switched, reordered, eliminated, or otherwise altered during a single-analyte process. In some embodiments, an outcome is changed, switched, reordered, eliminated, or otherwise altered automatically or based upon a user input during a single-analyte process. For example, in some embodiments, a single-analyte synthesis process with a defined outcome of a final product includes an outcome adjusted if facing a shortage of a reagent. In some such embodiments, a user is prompted to choose between attempting to complete the synthesis despite the lack of reagent, or stabilizing the intermediary product until more reagent is supplied.

In some embodiments, the present disclosure provides a method for controlling a single-analyte process, the method comprising: determining an outcome for the single-analyte process; and performing an iterative process until a determinant criterium has been met, in which the iterative process comprises the steps of: determining a process metric for a single analyte based upon a single-analyte data set; implementing an action on a single-analyte system based upon the process metric and/or the outcome for the single-analyte process, in which the single-analyte system comprises a detection system that is configured to obtain a physical measurement of the single analyte at single-analyte resolution; and updating the single-analyte data set after implementing the action on the single-analyte system. In some embodiments, the iterative process includes the step of after updating the single-analyte data set, updating the outcome for the single-analyte process.

In some embodiments, the present disclosure provides a method for controlling a single-analyte process, the method comprising: performing an iterative process until a determinant criterium has been met, in which the iterative process comprises the steps of: determining an outcome for the single-analyte process based upon a single-analyte data set; determining a process metric for a single analyte based upon the single-analyte data set; implementing an action on a single-analyte system based upon the process metric and/or the outcome for the single-analyte process, in which the single-analyte system comprises a detection system that is configured to obtain a physical measurement of the single analyte at single-analyte resolution; and updating the single-analyte data set after implementing the action on the single-analyte system. In some embodiments, determining an outcome occurs after the initiation of a single-analyte process or an iterative process thereof. For example, in some embodiments, for a single-analyte identification assay (e.g., a single-molecule polypeptide identification assay), an algorithm configured to analyze single-analyte characterization data, thereby identifying the single analyte, determines that characterization data collected during the process does not conform to any previously-observed single analytes, and subsequently defines an outcome to more thoroughly characterize the unknown single analyte (e.g., additional cycles of characterization) to provide more information on the new single analyte for future single-analyte identification assays.

In some embodiments, a targeted or defined outcome for a single-analyte process is utilized to configure and/or control the single-analyte process. In some embodiments, a method of performing a single-analyte process includes one or more of the steps of: determining one or more outcomes for a single-analyte process; determining one or more process metrics that correspond to each outcome of the one or more outcomes; determining a rule for each process metric of the one or more process metrics that correspond to each outcome of the one or more outcomes; configuring one or more actions based upon each rule, standard or practice; implementing a single-analyte process including an action of the one or more actions; updating a single-analyte data set after implementing the single-analyte process; re-determining one or more process metrics that correspond to each outcome of the one or more outcomes based upon the updated single-analyte data set; and re-determining the rule for each process metric based upon the updated single-analyte data set. In some embodiments, a method of performing a single-analyte process includes the step of providing a single-analyte system that is configured to perform the single-analyte process as set forth herein. In some embodiments, one or more of the steps exemplified forth herein occurs before the providing of the single-analyte system. For example, in some embodiments, manufacturer-established outcomes or rules for process metrics is determined before a single-analyte system is provided to a user. In some embodiments, one or more of the steps exemplified forth herein occurs after the providing of the single-analyte system. For example, in some embodiments, user-established outcomes or rules for process metrics are determined after a single-analyte system is provided to a user. In some embodiments, some steps exemplified forth herein is omitted. For example, in some embodiments, one or more process metrics that correspond to each outcome of the one or more outcomes is not re-determined based upon the updated single-analyte data set. In some embodiments, the rule for each process metric is not re-determined based upon the updated single-analyte data set.

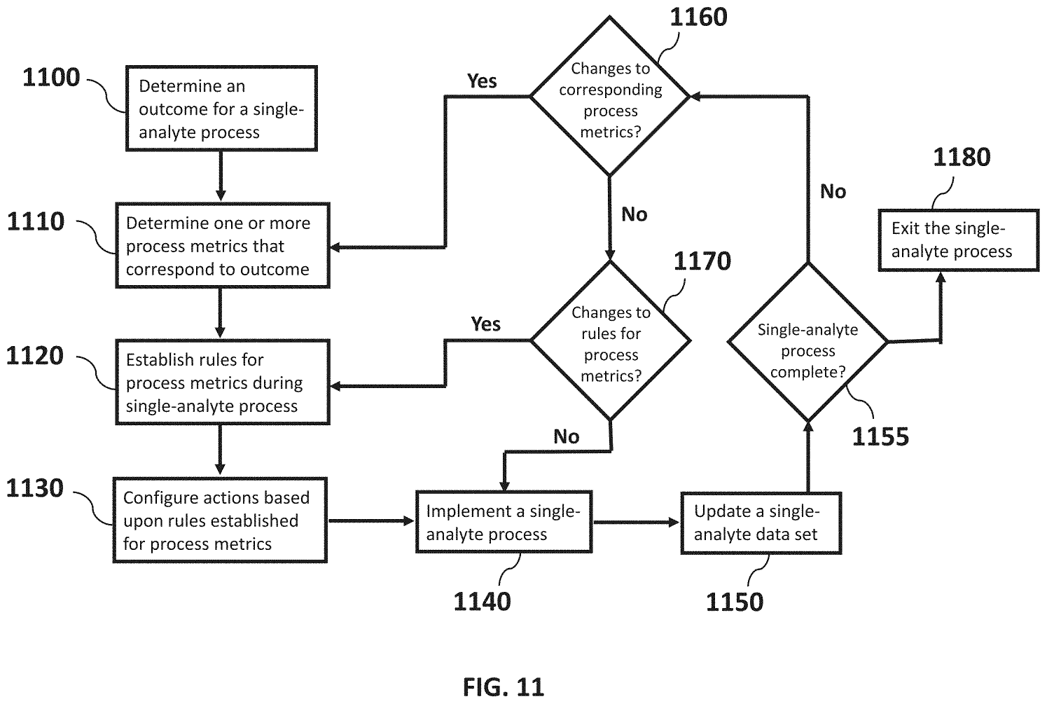

FIG. 11 depicts an exemplary embodiment of a single-analyte process. In some embodiments, one or more outcomes is determined 1100 for the single-analyte process. In some embodiments, based upon the determining one or more outcomes 1100, one or more is determined 1110 that correspond to the determined outcomes. In some embodiments, the one or more metrics that correspond to the determined outcomes is determined independently of and/or before the one or more outcomes have been determined. In some embodiments, the process metrics that correspond to the one or more outcomes is determined by any of a variety of methods, such as prior system characterization, known relationships, correlations, analysis of prior single-analyte processes, etc. In some embodiments, after determining one or more metrics 1110 that correspond to determined outcomes, a rule for each process metric of the one or more process metrics is determined 1120. In some embodiments, a rule for a process metric includes an appropriate criterium, threshold value, range, or state that is related to a likelihood for achieving a targeted or desired outcome. For example, in some embodiments, a rule of a maximum amount of reagent utilized per process cycle is established for a particular reagent based upon a targeted outcome of minimizing the amount of reagent consumed during a single-analyte process. In some embodiments, after determining a rule 1120 for each process metric of the one or more process metric, one or more actions is configured 1130 for each rule. For example, in some embodiments, given a process metric with an expected normal range, a first action is configured for the situation in which the process metric is determined to be within the normal range, and a second action is configured for the situation in which the process metric is determined to be outside the normal range. In some embodiments, given a first process metric, a first action is configured for the first process metric for the situation in which a second process metric is determined to have a certain value, and a second action is configured for the first process metric for the situation in which the second process metric is determined to not have a certain value. In some embodiments, after configuring the actions 1130 for each process metric, a single-analyte process is implemented 1140 according to the established outcomes, rules, standards, practices, and/or actions. In some embodiments, a single-analyte process includes an iterative process as described herein. In some embodiments, while implementing a single-analyte process 1140, a single-analyte data set is updated 1150. In some embodiments, one or more process metrics is updated when the single-analyte data set is updated 1150. In some embodiments, after the updating of a single-analyte data set, it is determined if the single-analyte process has been completed 1155. In some embodiments, if the process is determined to be complete 1155, the single-analyte process is exited 1180. Otherwise, in some embodiments, the single-analyte data set is evaluated 1160 to determine if any correspondences between process metrics and outcomes need to be adjusted. In some embodiments, if an altered correspondence between a process metric and an outcome is expected based upon a single-analyte data set, the correspondence between process metrics and outcomes is re-determined 1110. In some embodiments, the single-analyte data set is evaluated 1170 to determine if a rule for a process metric needs to be adjusted. For example, in some embodiments, a configured step, procedure, or sub-procedure of an action is found to be ineffective to alter a process metric, thereby requiring adjustment. In some embodiments, if a rule for a process metric needs to be adjusted, the rule is re-determined 1120.

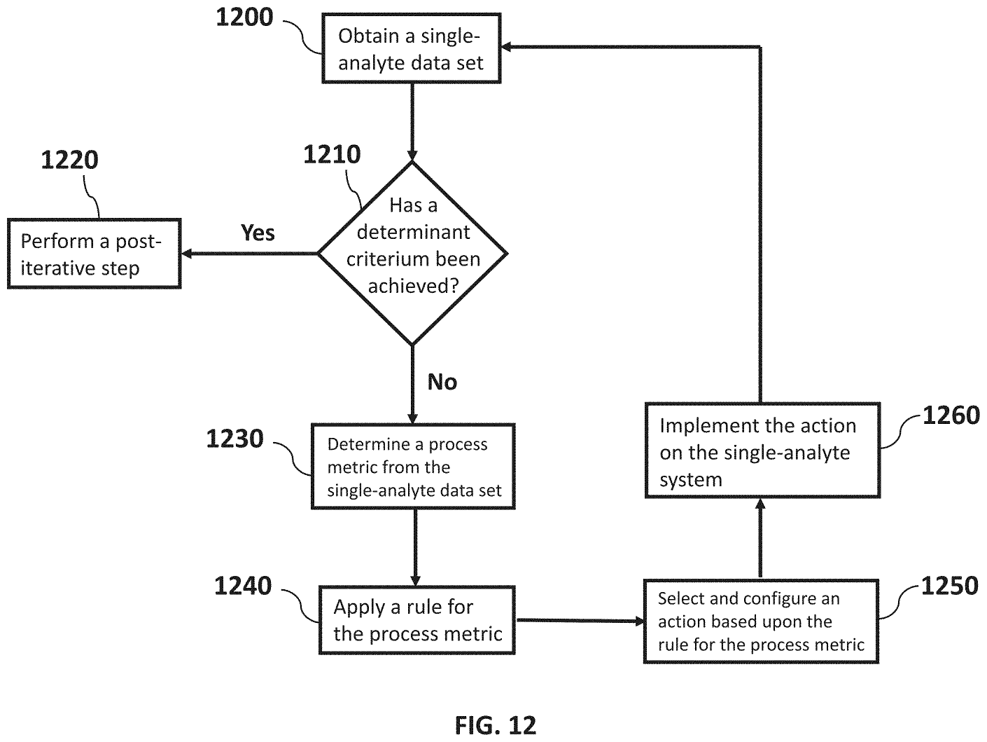

FIG. 12 depicts an exemplary embodiment of the utilization of outcome-based rules, standards, or practices for a process metric during a single-analyte process comprising an iterative process. In some embodiments, an iterative process includes a step of obtaining 1200 a single-analyte data set. In some embodiments, the single-analyte data set is analyzed to determine 1210 if a determinant criterium for ending the iterative process has been met. In some embodiments, if a determinant criterium has been met, the iterative process is exited and, optionally, one or more post-iterative steps are performed 1220. In some embodiments, if a determinant criterium has not been met, one or more process metrics is determined 1230 from a single-analyte data set. In some embodiments, based upon the determined process metrics and an existing set of rules, practices, or standards for the one or more process metrics, a rule is applied 1240 to at least one process metric of the one or more process metrics. In some embodiments, after applying 1240 a rule to at least one process metric of the one or more process metrics, an action is selected and/or configured 1250 based upon the rule. In some embodiments, subsequently, the action is implemented 1260 on the single-analyte system and an updated single-analyte data set is obtained 1200. In some embodiments, the iterative process continues in this fashion until a determinant criterium 1210 has been met.

In some embodiments, a single-analyte process includes a step of determining an outcome for the single-analyte process. In some embodiments, an outcome is selected from: an efficiency with respect to a single-analyte above a threshold value; an efficiency with respect to a single-analyte system component above a threshold value; a maximized likelihood of obtaining a specified outcome; a minimized likelihood of obtaining a failed outcome; a minimized likelihood of a negative impact on a single analyte; an absolute or relative time length for the single-analyte process; a minimized time length for the single-analyte process; a processivity rate for a single-analyte process; a minimized uncertainty level for a physical characterization of a single analyte; a minimized uncertainty level for an outcome of a single-analyte process; or a combination thereof. In some embodiments, an efficiency with respect to a single analyte includes outcome metrics with respect to the single analyte, such as percentage of single analytes characterized, percentage of single analytes synthesized, etc. In some embodiments, an efficiency with respect to a single-analyte system component includes an outcome metric with respect to a process or system parameter, such as a minimized amount of reagent used, a minimized use time for an instrument, a minimized cost per process run, etc. In some embodiments, a processivity rate includes a rate of process performance, such as a per analyte rate of synthesis, a per analyte rate of assay, a number of processes performed per unit time, etc.

In some embodiments, an outcome of a single-analyte process is determined to correspond to one or more process metrics. In some embodiments, a correspondence between a process metric and an outcome of a single-analyte process is a direct correspondence if the outcome is based upon the process metric. For example, in some embodiments, a process metric of total elapsed process time directly corresponds to a targeted outcome of not exceeding a maximum elapsed process time. In some embodiments, a correspondence between a process metric and an outcome of a single-analyte process is an indirect correspondence if the outcome is not based upon the process metric. In some embodiments, indirectly corresponding process metrics include proxy values, correlated values, or predictive relationships. For example, in some embodiments, a pattern of ambient temperature instability is predictive of an increased likelihood of a single-analyte process failing. In some embodiments, an outcome is determined by determining a process metric comprising a single-analyte characterization. In some embodiments, a single-analyte characterization includes a characteristic with regard to the single analyte that is determined from a plurality of physical measurements of the single analyte during a single-analyte process. For example, in some embodiments, an outcome of a proteomic assay is determined by determining an identity of a polypeptide via a plurality of physical measurements of the polypeptide.

In some embodiments, correspondence between outcomes of single-analyte processes and process metrics measured or determined therein are determined from any of a variety of sources. In some embodiments, a correspondence between an outcome of a single-analyte process and a process metric is determined by a user of a single-analyte system, a supplier of a single-analyte system, a reference source (e.g., a published article), an algorithm (e.g., a machine-learning algorithm), or a combination thereof. In some embodiments, a correspondence between an outcome of a single-analyte process and a process metric is determined at any time before, during, or after the initiation of a single-analyte process. In some embodiments, a correspondence between an outcome of a single-analyte process and a process metric is determined prior to the providing of a single-analyte system to a user. In some embodiments, a correspondence between an outcome of a single-analyte process and a process metric is determined by a user before initiating the single-analyte process. In some embodiments, a correspondence between an outcome of a single-analyte process and a process metric is determined at the initiation of a single-analyte process (e.g., by prompting a user input). In some embodiments, a previously-undescribed correspondence between an outcome of a single-analyte process and a process metric is determined after the initiation of a single-analyte process (e.g., by the analysis of a single-analyte data set). In some embodiments, a correspondence between an outcome of a single-analyte process and a process metric is removed before, during, or after the initiation of a single-analyte process (e.g., automatically or via a user input).

In some embodiments, a rule for a process metric is established before, during, or after the initiation of a single-analyte process. In some embodiments, a rule refers to any criterium, threshold value, range, or state of a process metric that predicts, suggests, infers, or otherwise forecasts a likelihood of achieving an outcome during a single-analyte process as set forth herein. In some embodiments, a rule for a process metric is formulated as a normal value, a minimum value, a maximum value, a critical value, a normal or standard range or ranges, a list, a ranked list, a hierarchy, a sequence, a pattern, or other form for a given type of process metric. For example, in some embodiments, a binary process metric includes a rule indicating that one of the binary states is a “normal” state and the other state is an “abnormal” state. In some embodiments, a rule for a first process metric is determined, in whole or in part, by a second process metric. For example, in some embodiments, an image in an imaging data set is only utilized for analysis if the image meets a rule for an overall image quality metric. In turn, in some embodiments, the overall image quality metric is based upon a weighted or ranked combination of other individual image quality metrics.

In some embodiments, a rule delineates values of process metrics into two or more categories or classifiers (e.g., low, normal, high, etc.). In some embodiments, each category or classifier of a rule for a process metric corresponds to performing a particular action during a single-analyte process. In some embodiments, a first category or classifier of a rule for a process metric corresponds to a performing a first action during a single-analyte process, and a second category or classifier of a rule for a process metric corresponds to a performing a second action during a single-analyte process. In some embodiments, two categories or classifiers for a rule for a process metric correspond to the same action being performed during a single-analyte process. In some embodiments, two categories or classifiers for a rule for a process metric correspond to differing configurations of the same action being performed during a single-analyte process. For example, in some embodiments, differing categories of a rule correspond to a process step with differing configurations of procedures or sub-procedures. In some embodiments, a rule for a process metric is determined by a user of a single-analyte system, a supplier of a single-analyte system, a reference source (e.g., a published article), an algorithm (e.g., a machine-learning algorithm), or a combination thereof.

In some embodiments, an action performed during a single-analyte process is configured based upon a rule for a process metric as set forth herein. In some embodiments, an action for a single-analyte process is configured by a user of a single-analyte system, a supplier of a single-analyte system, a reference source (e.g., a published article), an algorithm (e.g., a machine-learning algorithm), or a combination thereof. In some embodiments, a configured action for a rule of a process metric is selected from the group consisting of: pausing the single-analyte process; altering a sequence of steps for the single-analyte process; identifying a next step of a sequence of steps for the single-analyte process; performing a related process on the single analyte; performing the related process on a second single analyte; and continuing a sequence of steps for the single-analyte process. In some embodiments, configuring an action that corresponds to a rule of a process metric includes configuring a step, procedure, or sub-procedure for the single-analyte process. For example, in some embodiments, a single-analyte process is paused if an uncertainty metric for a physical measurement exceeds a threshold value. In some embodiments, the pausing action is configured with one or more steps or procedures that seek to determine, mitigate, ameliorate, or otherwise reduce a source of uncertainty for the physical measurement. In some embodiments, an action corresponding to a rule of a process metric is configured before, during, or after the initiation of a single-analyte process. In some embodiments, an action is configured or re-configured after one or more single-analyte data sets have been collected during a single-analyte process.

### Configuring Actions During Single-Analyte Processes

Described herein are methods for performing and controlling a single-analyte process performed on a single-analyte system. In some embodiments, a single-analyte process utilizes an iterative process to determine a sequence of actions, steps, procedures, or sub-procedures during the single-analyte process. In some embodiments, a single-analyte process utilizes an iterative process to alter a pre-determined sequence of actions, steps, procedures, or sub-procedures during the single-analyte process. In some embodiments, the methods and system described herein are applied to any of a variety of single-analyte processes, including single-analyte synthesis, single-analyte fabrication, single-analyte manipulation, and single-analyte assay, on a single-analyte system. It shall be understood that the systems and methods described herein are exemplary and any of a variety of methods or systems can be similarly deployed.

In some embodiments, a single-analyte process includes a sequence of steps that, collectively, achieve or substantially achieve a targeted or defined outcome. In some embodiments, a single-analyte process includes an iterative process that determines, in whole or in part, the sequence of steps for the single-analyte process. In some embodiments, a single-analyte process proceeds by the iterative methods set forth herein. In some embodiments, an iterative process includes a cycle of determining one or more process metrics from a single-analyte data set, implementing an action on a single-analyte system based upon the one or more process metrics, and updating the single-analyte data set after implementing the action on the single-analyte system. In some embodiments, actions that are implemented on a single-analyte system are selected and configured based upon an established set of rules, standards, or practices for the one or more process metrics determined from a single-analyte data set. In some embodiments, rules, standards, or practices are determined from a single-analyte data set by the methods set forth herein. In some embodiments, each action that is configured to be implemented on a single-analyte system includes one or more steps that are to be performed on a single-analyte system. In some embodiments, each step of the configured one or more steps includes one or more procedures and/or sub-procedures that are implemented on the single-analyte system. Accordingly, in some embodiments, an action configured to be implemented on a single-analyte system, or a step, procedure, and/or sub-procedure thereof, is linked to one or more process metrics determined from a single-analyte data set.

In some embodiments, a process metric utilized for selecting, configuring, and/or implementing an action on a single-analyte system during a single-analyte process includes an uncertainty metric. In some embodiments, an uncertainty metric includes a measure of variability for any component, aspect, or parameter of a single-analyte system, such as a variability of system measurements, variability of system performance, and variability of physical observations of single analytes and any properties, effects, behaviors, or interactions derived therefrom. In some embodiments, an uncertainty metric describes variability in a single-analyte system that arise due to one or more sources of bias, one or more sources of error, or a combination thereof. In some embodiments, an uncertainty metric is derived from a single-analyte data set by a method set forth herein. In some embodiments, one or more actions that are configured to be implemented on a single-analyte system is based upon a value of an uncertainty metric.

Accordingly, in some embodiments, a single-analyte system is configured to generate data that is utilized for determining one or more process metrics (e.g., uncertainty metrics) that are determined to relate to the outcome of a single-analyte process. For example, in some embodiments, a single-analyte system is configured to incorporate one or more sensors that provide instrumental metadata that is utilized for determining the variability of physical measurements collected on a single analyte. In some embodiments, a single-analyte system and processes performed thereupon is analyzed to determine one or more process metrics, including uncertainty metrics, that relates to the outcome of a single-analyte process. In some embodiments, all information available to a single-analyte system is combined and applied during a single-analyte process to achieve control of the process in a manner that increases the likelihood of attaining the targeted or defined outcome.

In some embodiments, an action that is implemented during a single-analyte process is configured based upon one or more process metrics that are determined during the single-analyte process. In some embodiments, an action is implemented during a single-analyte process to increase the likelihood of attaining a targeted or defined outcome for the process. Specifically, in some embodiments, an action is implemented during a single-analyte process that increases the likelihood of attaining a targeted or defined outcome, including correcting process inefficiencies, addressing system errors, applying prior knowledge to improve a single-analyte process, acquiring knowledge for future runs of a process, increasing confidence in attaining an outcome, economizing a single-analyte process (e.g., with respect to time, cost, etc.), or a combination thereof.

In some embodiments, an objective for the action is determined with respect to a purpose for the action. In some embodiments, an objective for an action includes a state, a value, or any other criterium that indicates that the purpose of the action was achieved. For example, in some embodiments, an action includes pausing a single-analyte process for the purpose of addressing an error in detected fluid flow rates. In some embodiments, an objective for the action includes detecting a fluid flow rate within a normal range. In some embodiments, an action is configured to be complete when an objective is attained. In some embodiments, an action is configured to continue until an objective is attained. In some embodiments, an action is determined without a specified objective. For example, in some embodiments, an action includes altering a sequence of steps to include a duplicate physical measurement of a single analyte. In some embodiments, the action is completed without any objective for the performing of the duplicate physical measurement (e.g., no requirement for the physical measurement to satisfy a data quality metric). In some embodiments, objectives for an action are determined before, during, or after the initiation of a single-analyte process. In some embodiments, an objective for an action is re-determined during a single-analyte process.

In some embodiments, an action implemented during a single-analyte process is configured before, during, or after the initiation of the single-analyte process. In some embodiments, an action implemented during a single-analyte process is re-configured during a single-analyte process. For example, in some embodiments, an iterative process is controlled, in whole or in part, by an image quality process metric that varies due to sources of vibration in the system. In some embodiments, an action to pause the iterative process and dampen a vibrational source is re-configured if the image quality process metric is not observed to sufficiently improve upon dampening the vibrational source. In some embodiments, an action is configured before a single-analyte system is provided to a user. For example, in some embodiments, a single-analyte system includes a manufacturer-supplied algorithm that is configured to perform one or more actions. In some embodiments, an action is configured by a user after a single-analyte system has been provided to the user. For example, in some embodiments, a user provides a threshold value of a process metric to configure the initiation or termination of an action during an iterative process. In some embodiments, an action is automatically configured, for example by an algorithm.

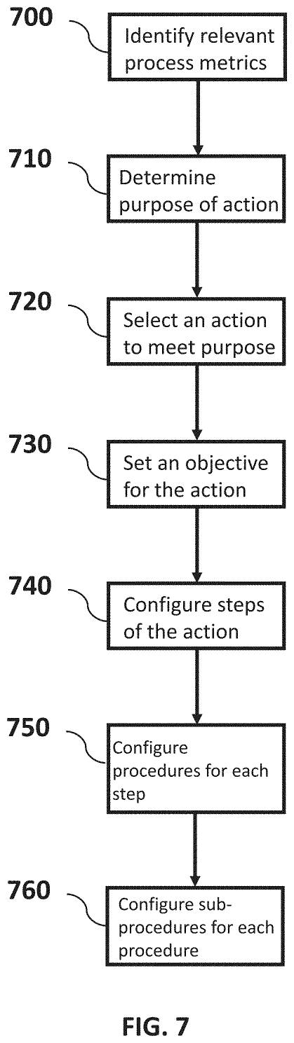

FIG. 7 depicts a method for configuring an action for a single-analyte process. In some embodiments, a first step for configuring an action includes identifying 700 one or more process metrics that is available during a single-analyte process. In some embodiments, a process metric is identified at any time prior to the configuration of the action and includes process metric relationships identified for other processes (e.g., single-analyte or bulk processes). In some embodiments, after the identifying 700 of the one or more process metrics, a purpose for an action is determined 710. In some embodiments, a purpose for an action is determined before process metrics are identified 700. In some embodiments, after a purpose has been determined 710, an action is selected 720 to meet the determined purpose. In some embodiments, after selecting an action 720 to meet the determined purpose, an objective for the action is set 730. In some embodiments, after selecting an action 720, and optionally setting an objective 730, one or more steps is configured 740 to carry out the action on a single-analyte system. In some embodiments, one or more procedures is configured 750 for at least a step of the one or more steps. In some embodiments, one or more sub-procedures is configured 760 for at least one procedure of the one or more procedures. In some embodiments, an action is configured from one or more pre-determined steps, procedures, or sub-procedures. For example, in some embodiments, a single-analyte system is provided with pre-defined procedures or sub-procedures that is implemented within a single-analyte process.

In some embodiments, an action implemented during a single-analyte process includes one or more steps that, in turn, includes one or more procedures or sub-procedures. In some embodiments, the procedures or sub-procedures includes specific activities that are implemented on the single-analyte system to complete a specified step while performing an action. In some embodiments, configuring a procedure or sub-procedures includes specifying one or more parameters that govern the implementation of the procedure and/or sub-procedure on the single-analyte system. In some embodiments, parameters include time durations, spatial lengths, areas, volumes, flow rates, heating rates, mass quantities, concentrations, etc. In some embodiments, a parameter for a procedure and/or sub-procedure is determined based upon a process metric. For example, in some embodiments, an exposure length for an image during an optical measurement is increased based upon an image-related process metric such as an image quality metric. In some embodiments, a parameter for a procedure and/or sub-procedure includes a known or characterized relationship with a process metric. For example, in some embodiments, a parameter is determined utilizing an equation that is a function of the process metric. In some embodiments, a parameter is looked up from a reference based upon the process metric. In some embodiments, a parameter is related to the same process metric upon which the action is based. For example, in some embodiments, a parameter includes a known correlation with a process metric. In some embodiments, a parameter is a process metric (e.g., a system temperature is utilized as a proxy value for an uncertainty metric). In some embodiments, a parameter is related to a differing process metric than the process metric upon which the action is based. For example, in some embodiments, altering a parameter (e.g., a volume, a flow rate) during an action causes more than one process metric to change.

In some embodiments, a single-analyte system produces one or more single-analyte data sets that are utilized when implementing a single-analyte process of the present disclosure. In some embodiments, the single-analyte data set includes information and/or data from one or more data sources as set forth herein. In some embodiments, data derived from any of a variety of data sources includes information from which a process metric is derived. In some embodiments, a data source of a single-analyte process includes any system, subsystem, component, process, or input that is available before or during a single-analyte process. In some embodiments, a system, subsystem, component, process, or input is analyzed to determine process metrics and/or relationships between process metrics and process outcomes. In some embodiments, an analysis of a system, subsystem, component, process, or input includes determining a source of uncertainty, an uncertainty metric, and/or an action that addresses the source of uncertainty for the system, subsystem, component, process, or input.

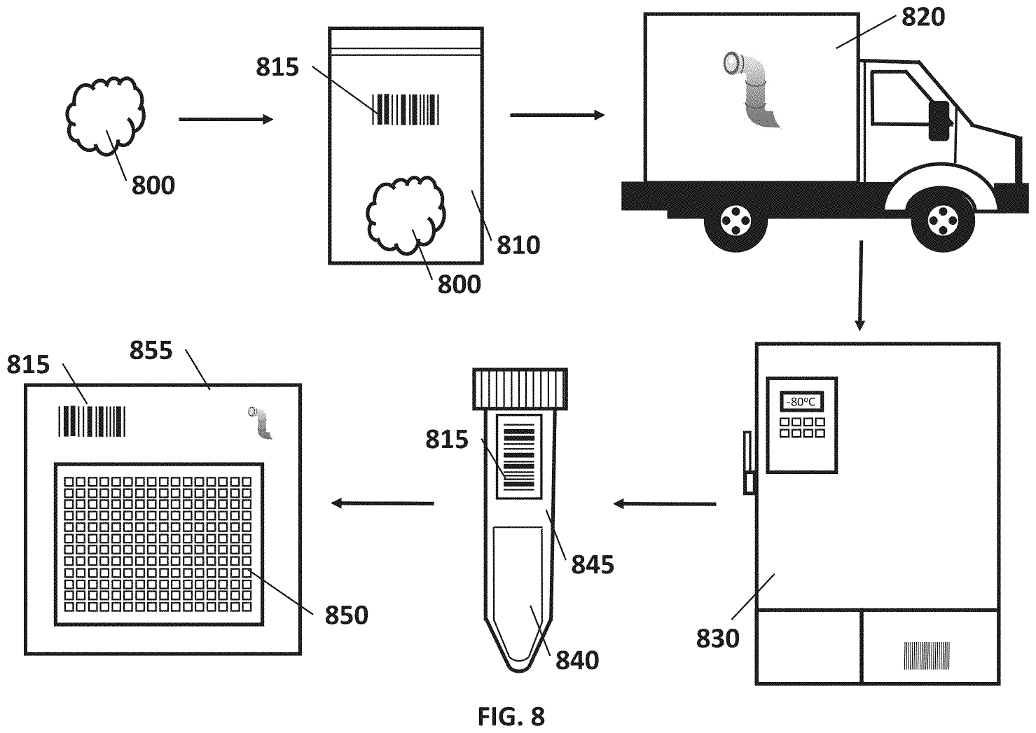

FIGS. 8-10B and 13 illustrate various exemplary aspects of single-analyte system and processes. In some embodiments, each system and/or process is analyzed to determine measurable process metrics and sources of uncertainty.

FIG. 8 illustrates an exemplary sample preparation process that is a source of process metrics for a single-analyte data set. In some embodiments, a sample 800 comprising one or more single analytes is collected into a sample collection container 810. In some embodiments, the sample 800 or container 810 is assigned a tracking code 815 (e.g., barcode, QR code, etc.) that allows the sample to be tied to other events and conditions before, during, and after a single-analyte process. In some embodiments, the collected sample 800 is subsequently transported 820 to a site where a single-analyte process occurs. In some embodiments, during transport 820 or otherwise before a single-analyte process, the sample 800 is stored 830 under one or more environmental conditions. In some embodiments, the storage 830 conditions (e.g., times, temperatures, etc.) that the sample 800 experiences are associated with a sample tracking code 815 to generate a sample handling history for the sample 800. In some embodiments, prior to a single-analyte process, the sample 800 further undergoes one or more single-analyte preparation processes. In some embodiments, the single-analyte preparation processes include transferring the sample 800 from the first sample collection container 810 to one or more single-analyte preparation containers 845, and undergoing various processes (e.g., separation, concentration, dilution, purification, etc.) to generate one or more medium 840 comprising single-analytes derived from the sample 800. In some embodiments, each single-analyte preparation process is tracked by a tracking code 815, thereby adding information to the sample handling history for the sample 800. In some embodiments, after any single-analyte preparation processes, single-analytes is finalized for analysis and characterization by adding the single-analyte medium 840 to a single-analyte retaining device 855 that is utilized in a single-analyte system during a single-analyte process. In some embodiments, the single-analyte retaining device 855 includes an array 850 that separates each single-analyte to a unique, resolvable position on the array 850 for analysis. In some embodiments, the single-analyte retaining device 855 includes the tracking code 815, thereby carrying any single-analyte sample handling history to be utilized as a part of a single-analyte data set during a single-analyte process. In some embodiments, one or more of the steps exemplified in the context of FIG. 8 is omitted.

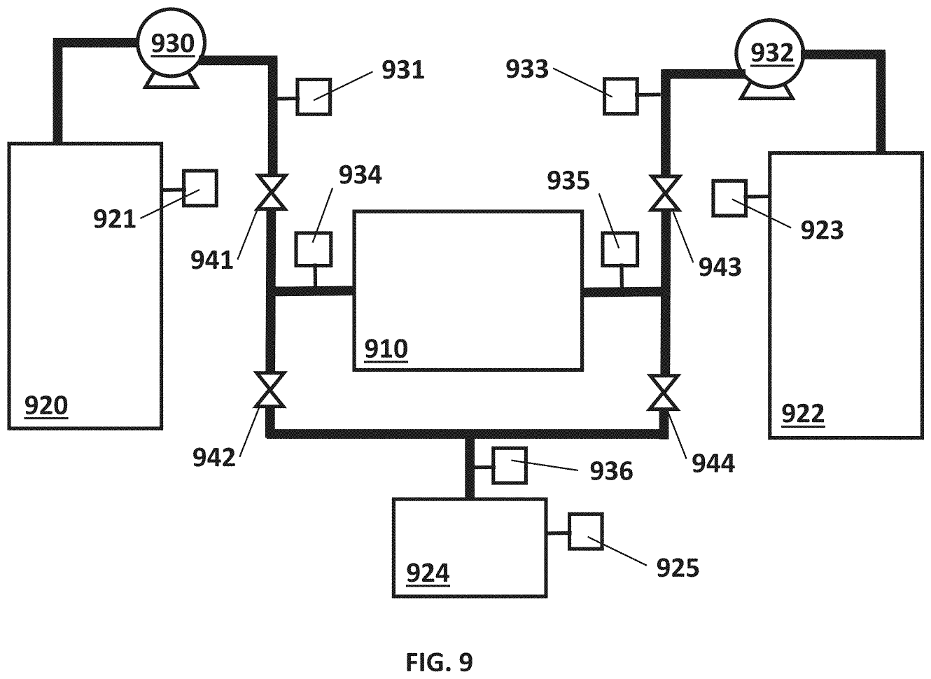

FIG. 9 illustrates an exemplary fluidics system for a single-analyte process. In some embodiments, the fluidics system is configured to provide one or more fluids to a single-analyte retaining device 910, such as the one described in FIG. 8. In some embodiments, the single-analyte retaining device 910 includes a flow cell, chip or cartridge. In some embodiments, the single-analyte retaining device 910 is fluidically connected to a first fluidic reservoir 920 comprising one or more reservoir sensors 921 (e.g., level sensors, composition sensors, pH sensors, etc.), and a second fluidic reservoir 922 comprising one or more reservoir sensors 923. In some embodiments, a first fluid is transferred from the first fluidic reservoir 920 by a first pump 930 that is associated with one or more pump sensors 931 (e.g., flow sensors, pressure sensors, power sensors, etc.). In some embodiments, a second fluid is transferred from the second fluidic reservoir 922 by a second pump 932 that is associated with one or more pump sensors 933. In some embodiments, the directionality and/or rate of transfer of fluids into the single-analyte retaining device 910 is further controlled by valves 941, 942, 943, and 944. In some embodiments, the second pump 932 is omitted, for example, in configurations in which first and second fluids are actuated via valves in fluid communication with a single pump. In some embodiments, fluid transfer into and out of the single-analyte retaining device 910 is monitored by one or more sensors 934 and 935 (e.g., flow sensors, pressure sensors, composition sensors, etc.). In some embodiments, fluid is transferred to an additional reservoir or manifold 924 before or after transfer to the single-analyte retaining device 910. In some embodiments, the additional reservoir or manifold 924 includes one or more sensors 925 (e.g., level sensors, composition sensors, pH sensors, etc.). In some embodiments, fluid transfer into and out of the additional reservoir or manifold 924 is monitored by one or more sensors 936.

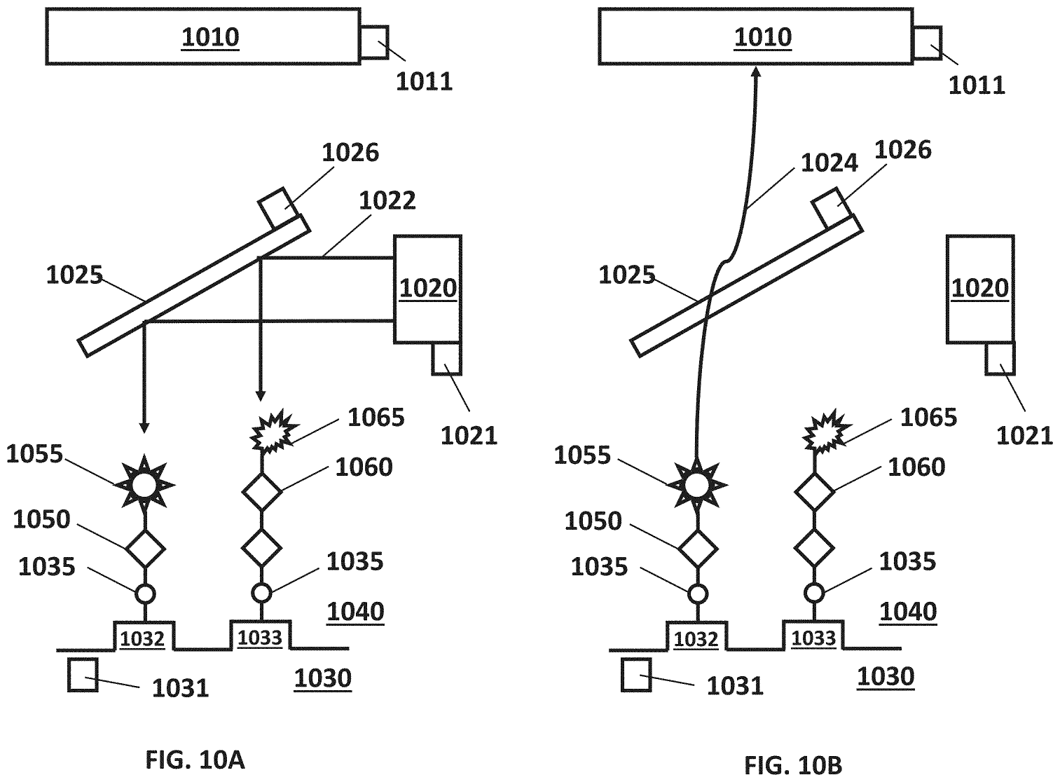

FIGS. 10A-10B illustrate an exemplary system and method for performing a physical measurement on one or more single analytes at single-analyte resolution. FIG. 10A depicts an excitation step of a single-analyte characterization method comprising a solid support 1030 comprising resolvable binding sites 1032 and 1033. In some embodiments, the solid support 1030 is coupled to one or more sensors (e.g., position sensors, pitch sensors, etc.). The solid support 1030 is coupled to a first single analyte 1050 by a linking group 1035 between the first single analyte 1050 and the first binding site 1032. The first single analyte is further coupled to a first detectable label 1055 (e.g., a fluorophore). The solid support 1030 is coupled to a second single analyte 1060 by a linking group 1035 between the second single analyte 1060 and the second binding site 1033. The second single analyte is further coupled to a second detectable label 1065 (e.g., a fluorophore). In some embodiments, an excitation source 1020 provides an exciting signal 1022 (e.g., UV, VIS or IR irradiation) that is received by one or more of the detectable labels 1055 and 1065. In some embodiments, the excitation source includes one or more sensors (e.g., power sensors, etc.). In some embodiments, the excitation source is paired with one or more signal-shaping components 1025 (e.g., mirrors, apertures, filters, etc.) that facilitate the transmission of the exciting signal 1022 to the detectable labels 1055 and 1065. In some embodiments, the signal-shaping components 1025 includes one or more sensors 1026 (e.g., position sensors, orientation sensors, etc.). In some embodiments, the system includes a detection sensor 1010 (e.g., camera) that is configured to receive a detection signal from a single analyte 1050 and 1060, or a detectable label 1055 and 1065 thereof. In some embodiments, the detection sensor 1010 includes additional sensors 1011 (e.g., position sensors, orientation sensors, etc.). FIG. 10B depicts a detection step of a single-analyte characterization method. In some embodiments, after receiving an exciting signal 1022 from the excitation source 1020, a detectable label 1055 of the first single analyte 1050 emits a detection signal 1024 that is received by the detection sensor 1010. In some embodiments, the signal-shaping components 1025 is configured to facilitate the transmission of the detection signal 1024 to the detection sensor 1010. In some embodiments, the components of the system of FIGS. 10A and 10B are exemplary and one or more of the components is omitted or replaced to achieve results desired for a particular single-analyte process.

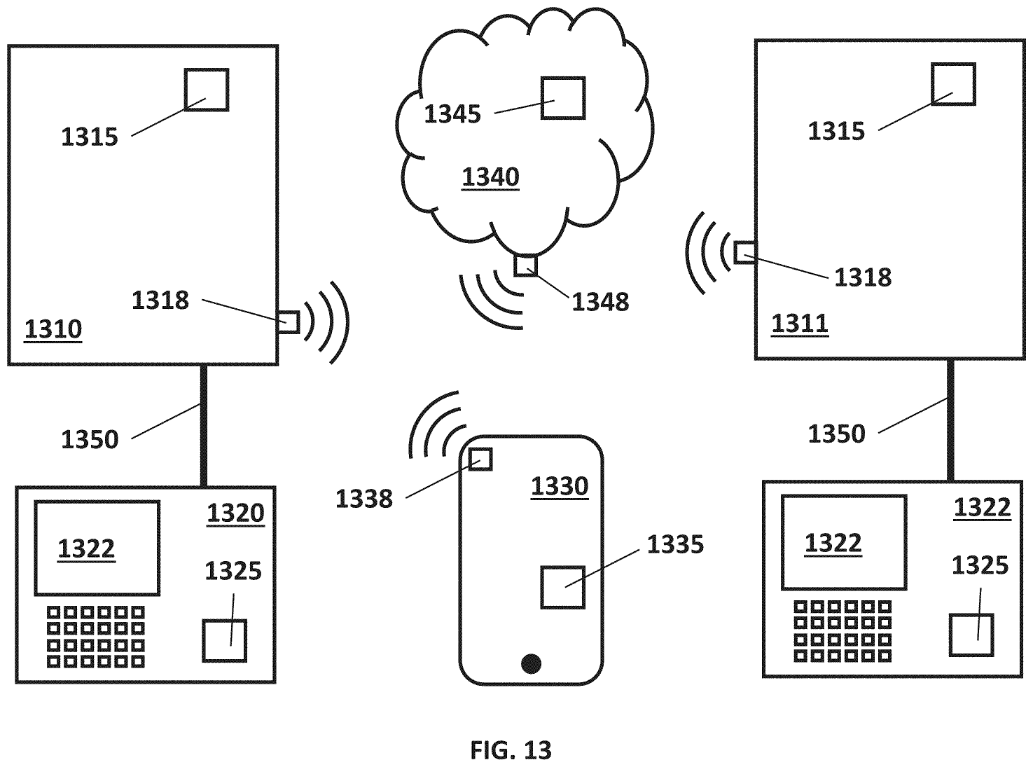

FIG. 13 illustrates a processor network that implements a single-analyte process of the present disclosure. In some embodiments, A single-analyte system includes a first single-analyte device 1310, and optionally a second single-analyte device 1311. In some embodiments, the first single-analyte device 1310 and the second single-analyte device 1311 include one or more processors 1315 that are configured to perform one or more processor-implemented algorithms during a single-analyte process. In some embodiments, the first single-analyte device 1310 and/or the second single-analyte device 1311 includes a data transmission device 1318 (e.g., a wireless device) that is configured to transmit information from a single-analyte device processor 1315 to one or more other processors (e.g., a wireless device). In some embodiments, the first single-analyte device 1310 and/or the second single-analyte device 1311 is connected with 1350 or includes a user interface 1320. In some embodiments, the user interface includes a graphical user interface 1322 and one or more processors 1325 that are configured to perform one or more processor-implemented algorithms during a single-analyte process. In some embodiments, the first single-analyte device 1310 and/or the second single-analyte device 1311 transmits information to and/or receive information from a data transmission device 1348 of an external network 1340 (e.g., a server, a cloud-based server) comprising one or more processors 1345 that are configured to perform one or more processor-implemented algorithms during a single-analyte process. In some embodiments, the first single-analyte device 1310 and/or the second single-analyte device 1311 transmits information to and/or receive information from a data transmission device 1338 of a user-controlled handheld device 1330 (e.g., a cellular phone, a tablet computer, etc.) that comprises one or more processors 1335 that are configured to perform one or more processor-implemented algorithms during a single-analyte process. In some embodiments, the components of the system of FIG. 13 are exemplary and one or more of the components is omitted or replaced to achieve results desired for a particular single-analyte process.

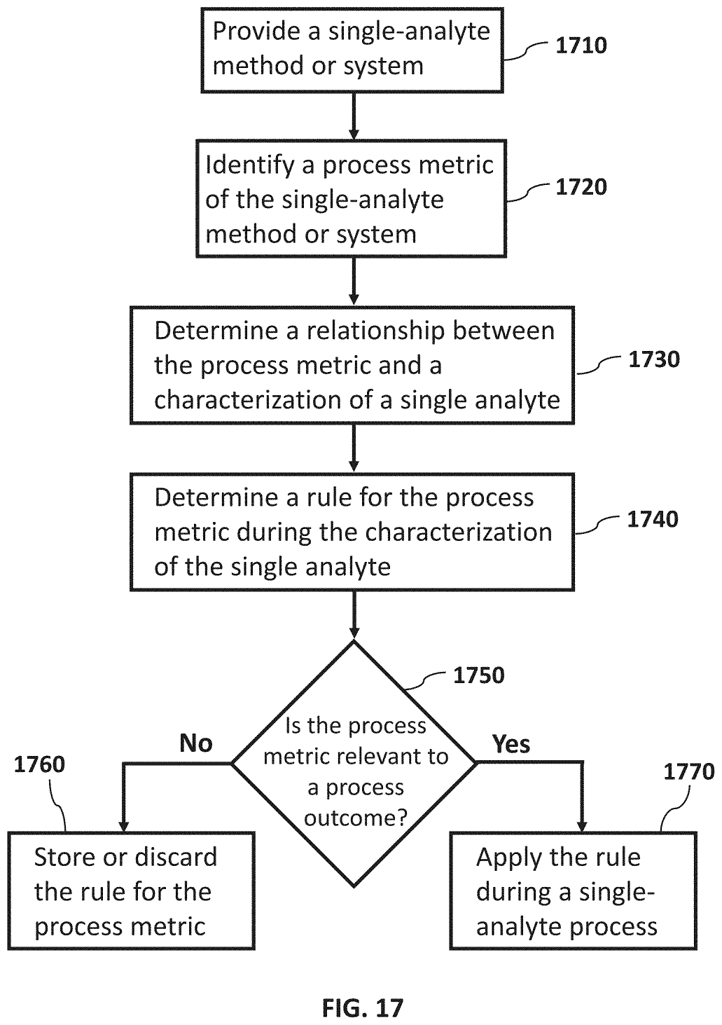

FIG. 17 provides a scheme analyzing a process, method, or system to identify relevant process metrics for a single-analyte process. In some embodiments, a single-analyte method or system is provided for analysis 1710. In some embodiments, a process metric or a plurality of process metrics is identified 1720 from the provided system or method 1710. In some embodiments, after determining one or more process metrics 1720, a subset of process metrics that are relevant to a single-analyte characterization (i.e., have a relationship with the single-analyte characterization) is determined 1730 from the one or more process metrics. In some embodiments, after determining one or more relevant process metrics 1730 for the single-analyte characterization, one or more rules is determined 1740 for the subset of process metrics. In some embodiments, after determining one or more rules 1740 for the subset of process metrics, a decision is made 1750 whether a process metric is relevant to a chosen outcome for a single-analyte process. In some embodiments, if a process metric is relevant to the chosen outcome, rules for the process metric is applied 1770 by a single-analyte system for use during a single-analyte process. In some embodiments, if the process metric is not relevant to the chosen outcome, rules for the process metric are discarded or stored 1760 for use in a subsequent single-analyte process.

In some embodiments, a process metric is analyzed to determine if a relationship exists between the process metric and a single-analyte characterization. In some embodiments, a process metric includes a relationship with a single-analyte characterization if the process metric affects the determination of the single-analyte characterization. For example, in some embodiments, a process metric is utilized when determining a single-analyte characterization (e.g., used for a calculation). In some embodiments, a process metric includes a measure of variability or uncertainty that is utilized when determining an uncertainty level for a single-analyte characterization (e.g., used to calculate a confidence level). In some embodiments, a process metric is correlated to a measure of variability or uncertainty of a single-analyte characterization (e.g., a physical measurement is excluded from a single-analyte characterization calculation if a process metric during the physical measurement suggests an increased likelihood that the physical measurement was invalid). In some embodiments, one or more process metrics is determined to have a relationship with a single-analyte characterization. In some embodiments, a process metric of one or more process metrics that have a relationship to a single-analyte characterization is used to determine if an outcome has been achieved before the termination of a single-analyte process.

Table II provides possible process metrics that could be derived from components of a single-analyte system, such as those shown in FIGS. 8-10 and 13. Table II includes the type of metric (e.g., fixed or variable), exemplary method(s) of measurement, and time when measurement occurs (e.g., the times are exemplary and depending upon the needs of the user measurement occurs at other times alternatively or additionally to those shown). For example, in some embodiments, the average spacing of analyte binding sites on a solid support includes a fixed value throughout a single-analyte process. In some embodiments, the average spacing of analyte binding sites is measured by sampling random solid supports after a batch has been produced but before the solid support is used in a single-analyte process. In some embodiments, the average spacing of analyte binding sites is measured by a surface metrology method. In some embodiments, the data used to calculate the average spacing of analyte binding sites is be used to calculate a standard deviation of the data to provide an uncertainty metric for the solid support.

TABLE II

| | | | | Time of Measurement | | |
|---|---|---|---|---|---|---|
| | | | | | Before | During |
| | | | | During | Single- | Single- |
| Process | Type | | Method of | Component | Analyte | Analyte |
| Metric | Fixed | Variable | Measurement | Fabrication | Process | Process |

Sample Handling and Storage

| | | | | |
|---|---|---|---|---|
| Sample state | | X | User | X |
| | | | Observation | |
| Sample | X | | User | X |
| weight | | | Observation | |
| Sample | | X | Thermocouple | X |
| temperature | | | | |
| Sample | | X | Calculated | X |
| temperature | | | | |
| variance | | | | |
| Sample | X | | User | X |
| storage | | | Observation | |
| material | | | | |
| Sample | | X | Hygrometer | X |
| humidity | | | | |
| Sample | | X | Calculated | X |
| humidity | | | | |
| variance | | | | |
| Storage gas | X | | Gas analyzer | X |
| composition | | | | |
| Storage gas | X | | Flow meter | X |
| quantity | | | | |
| Storage | X | | Chromatography | X |
| liquid | | | | |
| composition | | | | |
| Storage | X | | Flow meter | X |
| liquid | | | | |
| quantity | | | | |
| Single | X | | Weighing | X |
| analyte yield | | | | |
| Single | X | | Chromatography | X |
| analyte | | | | |
| purity | | | | |
| Single | | X | Light | X |
| analyte | | | absorbance | |
| concentration | | | | |
| Single | | X | Chromatography | X |
| analyte | | | | |
| buffer | | | | |
| composition | | | | |
| Single | | X | Thermocouple | X |
| analyte | | | | |
| temperature | | | | |

Algorithms and Processors

| | | | | | | |
|---|---|---|---|---|---|---|
| Processor | X | | Clock speed | X | | |
| Speed | | | | | | |
| Processor | | X | Calculated | | X | X |
| bandwidth | | | | | | |
| Processing | | X | Processor clock | | X | X |
| time | | | | | | |
| Processor | | X | Thermocouple | | X | X |
| temperature | | | | | | |
| Data upload | | X | Calculated | | | X |
| rate | | | | | | |
| Data upload | | X | Calculated | | | X |
| rate variance | | | | | | |
| Data | | X | Calculated | | | X |
| download | | | | | | |
| rate | | | | | | |
| Data | | X | Calculated | | | X |
| download | | | | | | |
| rate variance | | | | | | |
| Calculation | | X | Calculated | | X | X |
| precision | | | | | | |
| Calculation | | X | Calculated | | X | X |
| accuracy | | | | | | |

Fluidic Systems

| | | | | |
|---|---|---|---|---|
| Fluid | X | Capacitance | X | X |
| reservoir | | sensor | | |
| level | | | | |
| Fluid | X | Thermocouple | X | X |
| reservoir | | | | |
| temperature | | | | |
| Fluid | X | Calculated | X | X |
| reservoir | | | | |
| level | | | | |
| variance | | | | |
| Fluid | X | Calculated | X | X |
| reservoir | | | | |
| temperature | | | | |
| temporal | | | | |
| variance | | | | |
| Fluid | X | Calculated | X | X |
| reservoir | | | | |
| temperature | | | | |
| spatial | | | | |
| variance | | | | |
| Pump power | X | Voltage sensor | X | X |
| input | | | | |
| Pump head | X | Calculated | X | X |
| Pump | X | Volumetric flow | X | X |
| discharge | | meter | | |
| rate | | | | |
| Pump | X | Calculated | X | X |
| discharge | | | | |
| rate variance | | | | |
| Valve | X | Position sensor | X | X |
| position | | | | |
| Flow cell | X | Volumetric flow | X | X |
| upstream | | meter | | |
| flow rate | | | | |
| Flow cell | X | Calculated | X | X |
| upstream | | | | |
| flow rate | | | | |
| variance | | | | |
| Flow cell | X | Volumetric flow | X | X |
| downstream | | meter | | |
| flow rate | | | | |
| Flow cell | X | Calculated | X | X |
| downstream | | | | |
| flow rate | | | | |
| variance | | | | |
| Flow cell | X | Thermocouple | X | X |
| upstream | | | | |
| temperature | | | | |
| Flow cell | X | Calculated | X | X |
| upstream | | | | |
| temperature | | | | |
| variance | | | | |
| Flow cell | X | Thermocouple | X | X |
| downstream | | | | |
| temperature | | | | |
| Flow cell | X | Calculated | X | X |
| downstream | | | | |
| temperature | | | | |
| variance | | | | |
| Total liquid | X | Calculated | X | X |
| fluid transfer | | | | |
| volume | | | | |
| Total gas | X | Calculated | X | X |
| fluid transfer | | | | |
| volume | | | | |
| Flow cell | X | Pressure sensor | X | X |
| pressure drop | | | | |
| Flow cell | X | Calculated | X | X |
| pressure drop | | | | |
| variance | | | | |
| Fluid pH | X | pH sensor | X | X |
| Fluid | X | Oxygen probe | X | X |
| entrained gas | | | | |
| Fluid bubble | X | Bubble sensor | X | X |
| volume | | | | |
| Fluid | X | Chromatography | X | X |
| composition | | | | |

Detection System

| | | | | | | |
|---|---|---|---|---|---|---|
| Solid support | X | | Surface | X | | |
| pitch | | | metrology | | | |
| Solid support | X | | Calculated | X | | |
| pitch | | | | | | |
| variance | | | | | | |
| Solid support | X | | X-ray | X | | |
| composition | | | diffraction | | | |
| Single | X | | Surface | X | X | |
| analyte | | | metrology | | | |
| feature | | | | | | |
| average | | | | | | |
| spacing | | | | | | |
| Single | X | | Calculated | X | X | |
| analyte | | | | | | |
| feature | | | | | | |
| average | | | | | | |
| spacing | | | | | | |
| standard | | | | | | |
| deviation | | | | | | |
| Surface | | X | Spectroscopy | X | X | |
| chemistry | | | | | | |
| density | | | | | | |
| Surface | | X | Calculated | X | X | |
| chemistry | | | | | | |
| density | | | | | | |
| variance | | | | | | |
| Solid support | | X | Optical | | X | X |
| background | | | microscopy | | | |
| fluorescence | | | | | | |
| Surface | | X | Optical | | X | X |
| chemistry | | | microscopy | | | |
| background | | | | | | |
| fluorescence | | | | | | |
| Solid support | X | | Refractometer | X | | |
| index of | | | | | | |
| refraction | | | | | | |
| Flow cell | X | | Refractometer | X | | |
| body index | | | | | | |
| of refraction | | | | | | |
| Optics | | X | Position sensor | | X | X |
| orientation | | | | | | |
| Optics lens | X | | Interferometer | X | | |
| curvature | | | | | | |
| Optics lens | X | | Calculated | X | | |
| curvature | | | | | | |
| variance | | | | | | |
| Optics index | X | | Refractometer | X | | |
| of refraction | | | | | | |
| Laser power | | X | Voltage sensor | | | X |
| input | | | | | | |
| Laser power | | X | Optical power | | | X |
| output | | | meter | | | |
| Laser | X | | Interferometer | X | X | |
| coherence | | | | | | |
| Laser power | | X | Optical power | | X | X |
| density | | | meter | | | |
| Laser power | | X | Calculated | | X | X |
| density | | | | | | |
| variance | | | | | | |
| Laser | X | | Light sensor | X | | |
| frequency | | | | | | |
| band | | | | | | |
| Translation | | X | Position sensor | | X | X |
| stage x-y | | | | | | |
| position | | | | | | |
| Translation | | X | Calculated | | X | X |
| stage x-y | | | | | | |
| position | | | | | | |
| variance | | | | | | |
| Translation | | X | Position sensor | | X | X |
| stage motor | | | | | | |
| speed | | | | | | |
| Translation | | X | Thermocouple | | | X |
| stage motor | | | | | | |
| temperature | | | | | | |
| Translation | | X | Calculated | | | X |
| stage motor | | | | | | |
| temperature | | | | | | |
| variance | | | | | | |
| Vibration | | X | Vibration | | X | X |
| magnitude | | | Sensor | | | |
| Vibration | | X | Vibration | | X | X |
| frequency | | | Sensor | | | |
| Sensor pixel | | X | Voltage Sensor | | X | X |
| voltage | | | | | | |

### Proteomic Assays

In some embodiments, methods and systems set forth herein are applied to single-analyte assays, including single-molecule proteomic assays. In some embodiments, the methods and systems set forth herein are applied to single-molecule proteomic assays for diverse purposes, including polypeptide identification, quantification, or characterization; proteoform identification, quantification, or characterization; polypeptide sequencing, and polypeptide functional assays (e.g., polypeptide binding events, enzymatic activity assays, etc.). Exemplary embodiments of the methods and system set forth herein are described below and in Example 1-3 and 6-10, and the skilled person will readily recognize innumerable variations in accordance with the methods and system set forth herein. In some embodiments, a proteomic assay is advantageously performed at the scale of detecting, identifying, characterizing, or quantifying a number of proteins that is equivalent to the number of proteins in a given proteome sample found in nature. In some embodiments, a proteome assay set forth herein is modified for use with fewer proteins than found in any given proteome. For example, in some embodiments, a proteome assay set forth herein is readily modified for use in detecting, identifying, characterizing, or quantifying a single protein or a plurality of proteins that includes fewer proteins than found in any given proteome.

In some aspects, described herein is a method of performing a single-molecule proteomic assay comprising performing an iterative process until a determinant criterium has been achieved, in which the iterative process comprises at least two cycles, each cycle comprising the steps of: determining a process metric for a single polypeptide based upon a single-polypeptide data set; implementing an action on a single-polypeptide system based upon the process metric, in which the single-polypeptide system comprises a detection system that is configured to obtain a physical measurement of the single polypeptide at single-molecule resolution; and updating the single-polypeptide data set after implementing the action on the single-polypeptide system.

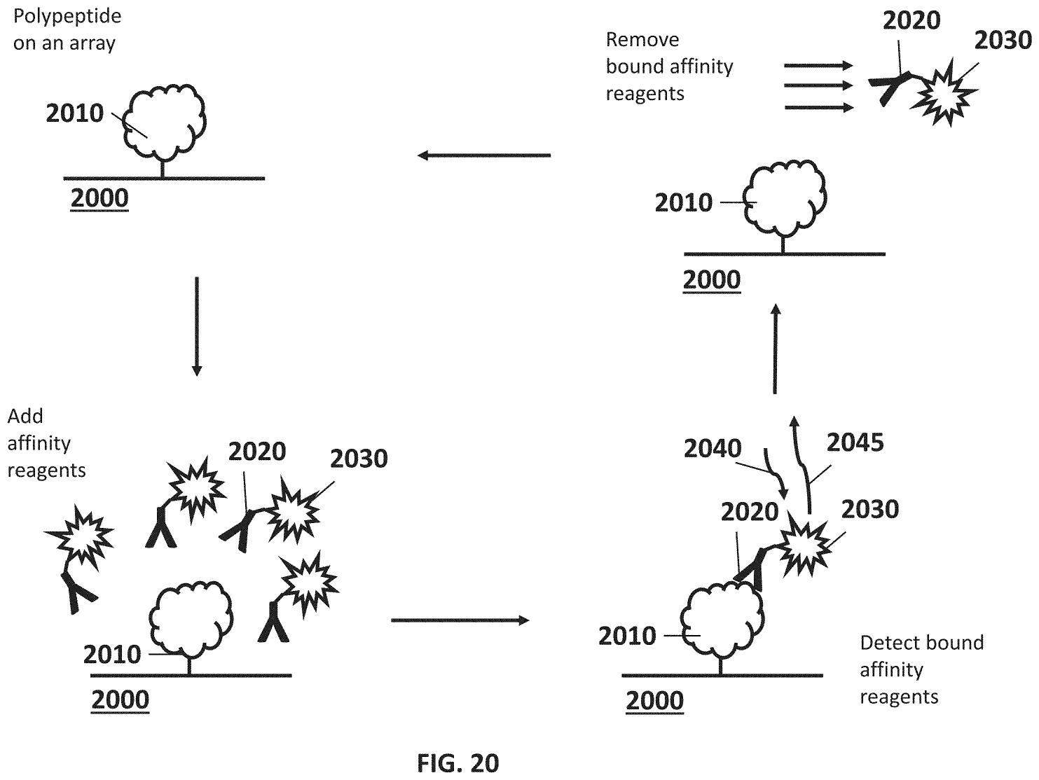

In some embodiments, the methods and systems set forth herein are applied to any of a variety of single-molecule proteomic assays. In some embodiments, single-molecule proteomic assays include fluorescence-based binding assays, barcode-based binding assays, fluorescence-based sequencing assays, and fluorescence/luminescence-based lifetime sequencing assays. FIGS. 20-23 describe features of some such assays, in accordance with certain embodiments of the assays. The use of fluorescent labels and fluorescent detection in the methods exemplified below and elsewhere herein is exemplary. In some embodiments, other detection techniques are used along with appropriate labels. In some embodiments, the assays need not use exogenous labels, for example, when probes, polypeptides or binding complexes are detected based on intrinsic properties.

FIG. 20 details a fluorescence-based binding proteomic assay, in accordance with some embodiments. In some embodiments, the fluorescence-based binding assay includes a series of affinity-based binding measurements that collectively characterize a single polypeptide or a plurality of polypeptides. In some embodiments, a polypeptide array 2000 comprising a single polypeptide 2010 bound at a resolvable address is provided to a single-analyte system. In some embodiments, the polypeptide 2010 on the array 2000 is subsequently contacted with a pool of affinity reagents 2020 with a known or characterized binding profile, thereby permitting an affinity reagent 2020 to bind to a polypeptide 2010. Each affinity reagent 2020 comprises a detectable label 2030 that is configured to transmit a signal to a detection system of the single-analyte system. After contacting the pool of affinity reagents 2020 with the array 2000, unbound affinity reagents 2020 are washed away, and a presence or absence of a signal is measured at the resolvable address (e.g., a fluorescence signal 2045 caused by an interaction between an excitation signal 2040 and the detectable label 2030). After measuring a presence or absence of signal at the resolvable address or a plurality of resolvable addresses, any bound affinity reagents 2020 are removed from the polypeptide 2010. In some embodiments, the process continues with additional cycles of the above-described affinity reagent binding measurements to produce a record of presence or absence of binding of each measured affinity reagent for each single polypeptide 2010 on the array 2000. In some embodiments, an iterative process as set forth herein is utilized during a fluorescence-based binding assay, for example to improve the quality of fluorescence imaging data and to alter a sequence of affinity reagents to obtain an improved characterization of a polypeptide.

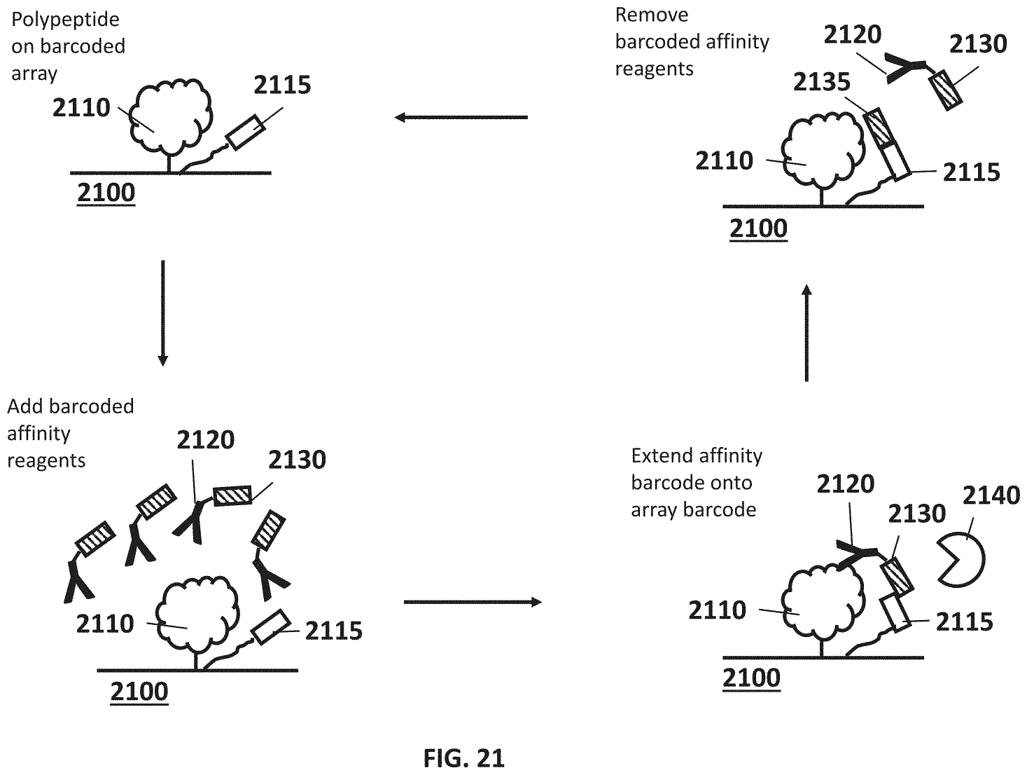

The use of fluorescence as a detection modality for the proteomic binding assay of FIG. 20 is exemplary. In some embodiments, other detection modalities are used. FIG. 21 details a barcode-based binding proteomic assay, in accordance with some embodiments. In some embodiments, the barcode-based binding assay includes a series of affinity-based binding events that are recorded by extension of an affinity reagent-based barcode onto a barcode associated with a single polypeptide. A polypeptide array 2100 comprising a single polypeptide 2110 at an address on the array 2100 with an associated address barcode 2115. In some embodiments, the array 2100 is subsequently contacted with a pool of affinity reagents 2120, thereby permitting an affinity reagent 2120 to bind to a polypeptide 2110. Each affinity reagent 2120 comprises an affinity barcode 2130 that comprises a sequence corresponding to the affinity reagent to which it is coupled (e.g., all affinity reagents with the same known or characterized binding profile will further comprise barcodes with identical sequences). After contacting the pool of affinity reagents 2120 with the array 2100, unbound affinity reagents 2120 are washed away, and the array 2100 is contacted with an enzyme that is configured to copy the affinity barcode 2130 onto the address barcode 2115 via an extension reaction. Optional extension reactions include, for example, polymerase-catalyzed addition of nucleotides to the address barcode 2115 using the affinity barcode 2130 as a template or ligase-catalyzed addition of oligonucleotides to the address barcode 2115 using the affinity barcode 2130 as a template. In some embodiments, after the extension reaction, any extension reactants are washed away, leaving an extended address-based barcode comprising the original address barcode sequence 2115 and a copy of the affinity barcode 2135. In some embodiments, the process continues with additional cycles of the above-described affinity reagent interaction barcode recording to produce a barcode record of each detected affinity reagent interaction for each polypeptide 2110 on the array 2100. In some embodiments, an iterative process as set forth herein is utilized during a barcode-based binding assay, for example to alter a sequence of affinity reagents to obtain an improved characterization of a polypeptide and to periodically check a reference single analyte to confirm the success of barcode extension cycles.

In some embodiments of a single-molecule polypeptide assay, a polypeptide is detected using one or more affinity reagents having known or measurable binding affinity for the polypeptide. In some embodiments, a polypeptide that is detected by binding to a known affinity reagent is identified based on the known or predicted binding characteristics of the affinity reagent. For example, in some embodiments, an affinity reagent that is known to selectively bind a candidate polypeptide suspected of being in a sample, without substantially binding to other polypeptides in the sample, is used to identify the candidate polypeptide in the sample merely by observing the binding event. In some embodiments, this one-to-one correlation of affinity reagent to candidate polypeptide is used for identification of one or more polypeptides. However, as the polypeptide complexity (e.g., the number and variety of different polypeptides) in a sample increases, or as the number of different candidate polypeptides to be identified increases, the time and resources to produce a commensurate variety of affinity reagents having one-to-one specificity for the polypeptides approaches limits of practicality.

In some embodiments, methods set forth herein are advantageously employed to overcome these constraints. In some embodiments, the methods are used to identify a number of different candidate polypeptides that exceeds the number of affinity reagents used. In some embodiments, this is achieved, for example, by using promiscuous affinity reagents that bind to multiple different candidate polypeptides suspected of being present in a given sample, and subjecting the polypeptide sample to a set of promiscuous affinity reagents that, taken as a whole, are expected to bind each candidate polypeptide in a different combination, such that each candidate polypeptide is expected to be encoded by a unique profile of binding and non-binding events. In some embodiments, promiscuity of an affinity reagent is a characteristic that is understood relative to a given population of polypeptides. In some embodiments, promiscuity arises due to the affinity reagent recognizing an epitope that is known to be present in a plurality of different candidate polypeptides suspected of being present in the given population of unknown polypeptides. For example, in some embodiments, epitopes having relatively short amino acid lengths such as dimers, trimers, or tetramers are expected to occur in a substantial number of different polypeptides in the human proteome. In some embodiments, a promiscuous affinity reagent recognizes different epitopes (e.g., epitopes differing from each other with regard to amino acid composition or sequence), the different epitopes being present in a plurality of different candidate polypeptides. For example, in some embodiments, a promiscuous affinity reagent that is designed or selected for its affinity toward a first trimer epitope binds to a second epitope that has a different sequence of amino acids when compared to the first epitope.

In some embodiments, although performing a single binding reaction between a promiscuous affinity reagent and a complex polypeptide sample yields ambiguous results regarding the identity of the different polypeptides to which it binds, the ambiguity is resolved in combination with the results of binding the constituents of the sample with other promiscuous affinity reagents. For example, in some embodiments, a plurality of different promiscuous affinity reagents is contacted with a complex population of polypeptides, in which the plurality is configured to produce a different binding profile for each candidate polypeptide suspected of being present in the population. In some such embodiments, each of the affinity reagents are distinguishable from the other affinity reagents, for example, due to unique labeling (e.g., different affinity reagents having different luminophore labels), unique spatial location (e.g., different affinity reagents being located at different addresses in an array), and/or unique time of use (e.g., different affinity reagents being delivered in series to a population of polypeptides). Accordingly, in some embodiments, the plurality of promiscuous affinity reagents produces a binding profile for each individual polypeptide that is decoded to identify a unique combination of epitopes present in the individual polypeptide. In some embodiments, this is in turn used to identify the individual polypeptide as a particular candidate polypeptide having the same or similar unique combination of epitopes. In some embodiments, the binding profile includes observed binding events as well as observed non-binding events. In some embodiments, this information is evaluated in view of the expectation that particular candidate polypeptides produce a similar binding profile, for example, based on presence and absence of particular epitopes in the candidate polypeptides.

In some embodiments, distinct and reproducible binding profiles is observed for one or more unknown polypeptides in a sample. However, in many embodiments one or more binding events produces inconclusive or even aberrant results and this, in turn, yields ambiguous binding profiles. For example, in some embodiments, observation of binding outcome for a single-molecule binding event are particularly prone to ambiguities due to stochasticity in the behavior of single molecules when observed using certain detection hardware. The present disclosure provides methods that provide accurate polypeptide identification despite ambiguities and imperfections that arises in many contexts. In some embodiments, methods for identifying, quantitating or otherwise characterizing one or more polypeptides in a sample utilize a binding model that evaluates the likelihood or probability that one or more candidate polypeptides that are suspected of being present in the sample will have produced an empirically observed binding profile. In some embodiments, the binding model includes information regarding expected binding outcomes (e.g., binding or non-binding) for binding of one or more affinity reagent with one or more candidate polypeptides. In some embodiments, the information includes an a priori characteristic of a candidate polypeptide, such as presence or absence of a particular epitope in the candidate polypeptide or length of the candidate polypeptide. In some embodiments, the information includes empirically determined characteristics such as propensity for the candidate polypeptide to bind individual affinity reagents. Moreover, in some embodiments, a binding model includes information regarding the propensity of a given candidate polypeptide generating a false positive or false negative binding result in the presence of a particular affinity reagent, and such information optionally is included for a plurality of affinity reagents.

In some embodiments, methods set forth herein are used to evaluate the degree of compatibility of one or more empirical binding profiles with results computed for various candidate polypeptides using a binding model. For example, in some embodiments, to identify an unknown polypeptide in a sample of many polypeptides, an empirical binding profile for the polypeptide is compared to results computed by the binding model for many or all candidate polypeptides suspected of being in the sample. In some embodiments of the methods set forth herein, identity for the unknown polypeptide is determined based on a likelihood of the unknown polypeptide being a particular candidate polypeptide given the empirical binding pattern or based on the probability of a particular candidate polypeptide generating the empirical binding pattern. In some embodiments, a score is determined from the measurements that are acquired for the unknown polypeptide with respect to many or all candidate polypeptides suspected of being in the sample. In some embodiments, a digital or binary score that indicates one of two discrete states is determined. In some embodiments, the score is non-digital or non-binary. For example, in some embodiments, the score is a value selected from a continuum of values such that an identity is made based on the score being above or below a threshold value. Moreover, in some embodiments, a score is a single value or a collection of values. Particularly useful methods for identifying polypeptides using promiscuous reagents, serial binding measurements and/or decoding with binding models are set forth, for example, in U.S. Pat. No. 10,473,654 US Pat. App. Pub. No. 2020/0318101 A1 or Egertson et al., BioRxiv (2021), DOI: 10.1101/2021.10.11.463967, each of which is incorporated herein by reference in its entirety for all purposes.

In some embodiments, such as detection assays, a polypeptide is cyclically modified and the modified products from individual cycles are detected. In some embodiments, a polypeptide is sequenced by a sequential process in which each cycle includes steps of detecting the polypeptide and removing one or more terminal amino acids from the polypeptide. In some embodiments, one or more of the steps includes adding a label to the polypeptide, for example, at the amino terminal amino acid or at the carboxy terminal amino acid. In some embodiments, a method of detecting a polypeptide includes steps of: exposing a terminal amino acid on the polypeptide; detecting a change in signal from the polypeptide; and identifying the type of amino acid that was removed based on the change detected in step. In some embodiments, the terminal amino acid is exposed, for example, by removal of one or more amino acids from the amino terminus or carboxyl terminus of the polypeptide. In some embodiments, steps of exposing the terminal amino acid through identifying the type of amino acid are repeated to produce a series of signal changes that is indicative of the sequence for the polypeptide.

In some embodiments, in a first configuration of a cyclical polypeptide detection method, one or more types of amino acids in the polypeptide is attached to a label that uniquely identifies the type of amino acid. In some such embodiments, the change in signal that identifies the amino acid is loss of signal from the respective label. For example, in some embodiments, lysines are attached to a distinguishable label such that loss of the label indicates removal of a lysine. In some embodiments, other amino acid types are attached to other labels that are mutually distinguishable from lysine and from each other. For example, in some embodiments, lysines are attached to a first label and cysteines are attached to a second label, the first and second labels being distinguishable from each other. Exemplary compositions and techniques that used to remove amino acids from a polypeptide and detect signal changes are those set forth in Swaminathan et al., Nature Biotech. 36:1076-1082 (2018); or U.S. Pat. No. 9,625,469 or 10,545,153, each of which is incorporated herein by reference in its entirety for all purposes. Methods and apparatus under development by Erisyon, Inc. (Austin, Tex.) are also be useful for detecting proteins.

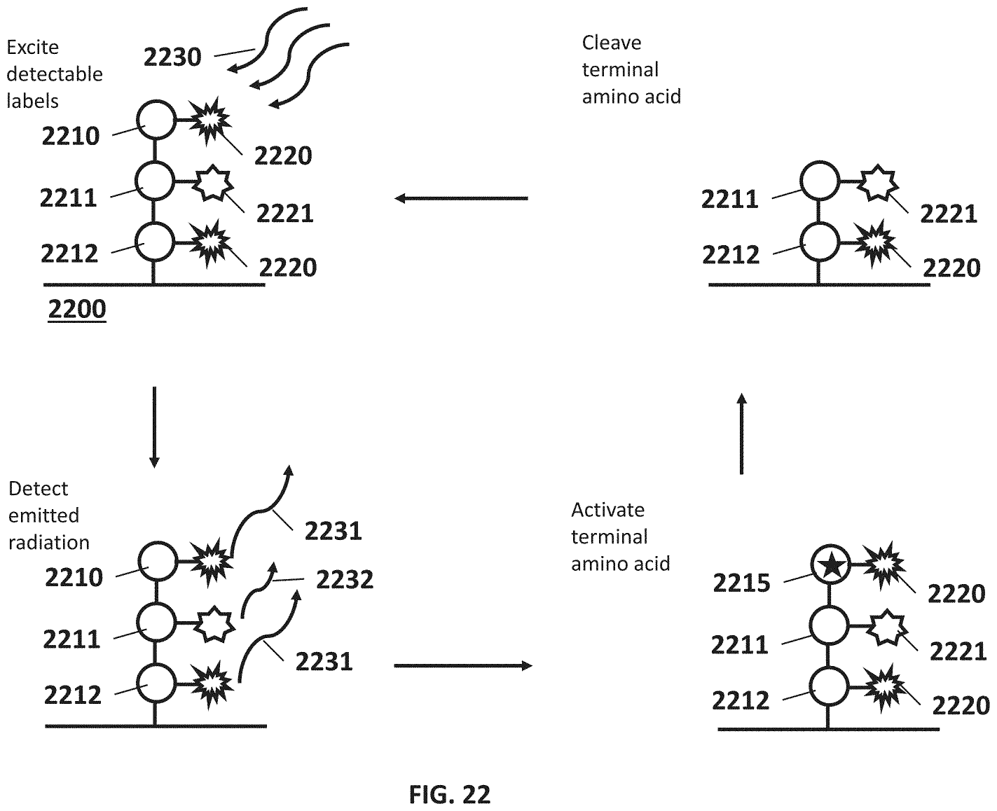

FIG. 22 details a fluorosequencing proteomic assay, in accordance with some embodiments. In some embodiments, a fluorosequencing assay employs Edman-type chemistry. In some embodiments, the assay includes a step-wise degradation of a fluorescently-labeled peptide to detect discrete changes in fluorescence corresponding with the removal of fluorescently-labeled amino acids. In some embodiments, a peptide includes two or more differing amino acids with differing fluorescent labels, such that a discrete fluorescence intensity change at a characteristic emission wavelength of one amino acid is correlated to the degradation of that amino acid from the peptide. FIG. 22 depicts an array 2200 comprising a peptide coupled at a resolvable address. In some embodiments, the peptide includes unknown amino acids 2210, 2211, and 2212, with associated fluorescent labels 2220 and 2221. In this example, the labels were added to the polypeptide using chemistry that is selective for a particular amino acid type, such that different labels are indicative of different types of amino acids (e.g., amino acids 2210 and 2212 bear the same type of label indicating that they are the same type of amino acid, whereas amino acids 2210 and 2211 bear different labels indicating that they are different types of amino acids). In some embodiments, the array 2200 comprising the peptide is excited to fluoresce by an excitation field 2230 to stimulate fluorescence from the fluorescent labels 2220 and 2221. In some embodiments, after excitation, fluorescent labels 2220 and 2221 emit characteristic light 2231 and 2232, respectively, whose intensities is detected by a detection device of a single-analyte system to measure the amount of labeled amino acids at the resolvable address. In some embodiments, after measuring the amounts of fluorescently-labeled amino acids, the terminal amino acid 2210 is activated by one or more activation reagents that are contacted with the array 2200 to form an activated terminal amino acid 2215. In some embodiments, after activation, the activated terminal amino acid 2215 is cleaved by one or more cleavage reagents that are contacted with the array. The resulting loss of signal, compared to fluorescence detected prior to cleavage, indicates that an amino acid of type 2210 was removed. In some embodiments, the process continues with additional cycles of fluorescence measurement and terminal amino acid removal to determine a series of labels removed. In some embodiments, the series of labels removed is used as a signature to identify the polypeptide for example by comparison to a polypeptide sequence database. In some embodiments, an iterative process as set forth herein is utilized during a fluorosequencing assay, for example to improve the quality of fluorescence imaging data and to periodically check a reference single analyte to confirm the success of degradation reactions.

In some embodiments, such as in a second configuration of a cyclical polypeptide detection method, a terminal amino acid of a polypeptide is recognized by an affinity agent that is specific for the terminal amino acid and/or specific for a label moiety that is present on the terminal amino acid. In some embodiments, the affinity agent is detected on an array, for example, due to a label on the affinity agent. In some embodiments, the label is a nucleic acid barcode sequence that is added to a primer nucleic acid upon formation of a complex. For example, in some embodiments, a barcode is added to the primer via ligation of an oligonucleotide having the barcode sequence or polymerase extension directed by a template that encodes the barcode sequence. In some embodiments, the formation of the complex and identity of the terminal amino acid is determined by decoding the barcode sequence. In some embodiments, multiple cycles produce a series of barcodes that is detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference in its entirety for all purposes. In some embodiments, methods and apparatus under development by Encodia, Inc. (San Diego, Calif.) are also useful for detecting proteins.

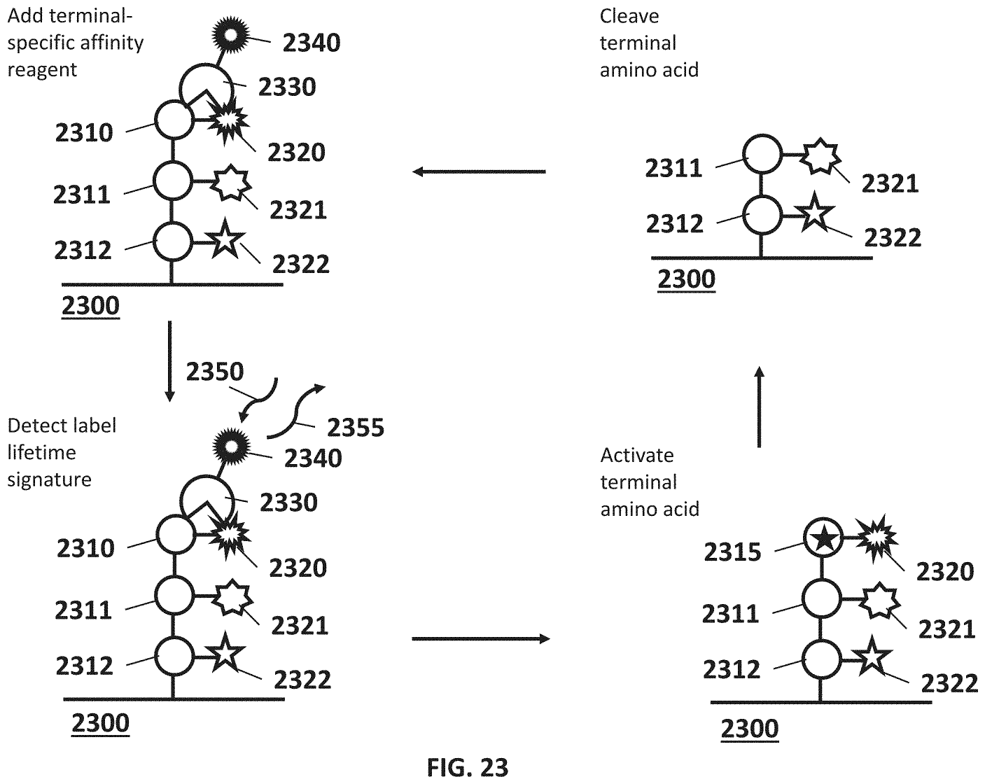

FIG. 23 details a fluorescence- or luminescence-based sequencing proteomic assay, in accordance with some embodiments. In some embodiments, a fluorescence- or luminescence-based sequencing assay includes step-wise affinity reagent-based determination of a terminal amino acid on a peptide, followed by removal of the terminal amino acid from the peptide. An array 2300 comprises a peptide at a resolvable address, where the peptide includes amino acids 2310, 2311, and 2312. In some embodiments, amino acids 2310, 2311, and 2312 have sidegroups (e.g., sidechains, modified sidechains, etc.) 2320, 2321, and 2322, respectively. The array 2300 is contacted with a pool of affinity reagents 2330 comprising detectable labels 2340. In some embodiments, an affinity reagent 2330 that recognizes terminal amino acid 2310 and/or sidegroup 2320 binds to the peptide. The array is then contacted with an excitation field 2350 that stimulates light emission 2355 from the detectable label 2340 of the affinity reagent 2330 captured at the address on the array 2300. In some embodiments, the light emission 2355 is measured by a detection device as an intensity or as a time-sequence to measure a fluorescence or luminescence lifetime for the detectable label. In some embodiments, the terminal amino acid 2310 is identified by matching the measured intensity or lifetime of the fluorescence or luminescence with the known lifetime for an affinity reagent with a known specificity for a terminal amino acid or sidegroup. In some embodiments, after measuring the fluorescence or luminescence at the address on the array 2300, the terminal amino acid 2310 is activated by one or more activation reagents that are contacted with the array 2300 to form an activated terminal amino acid 2315. In some embodiments, after activation, the activated terminal amino acid 2315 is cleaved by one or more cleavage reagents that are contacted with the array. In some embodiments, the process continues with additional cycles of affinity reagent binding lifetime measurements and degradation of terminal amino acids to determine a series of signals. In some embodiments, the series of signals is used as a signature to identify the polypeptide for example by comparison to a polypeptide sequence database. In some embodiments, an iterative process as set forth herein is utilized during a lifetime-based sequencing assay, for example to improve the quality of fluorescence imaging data and to periodically check a reference single analyte to confirm the success of degradation reactions.

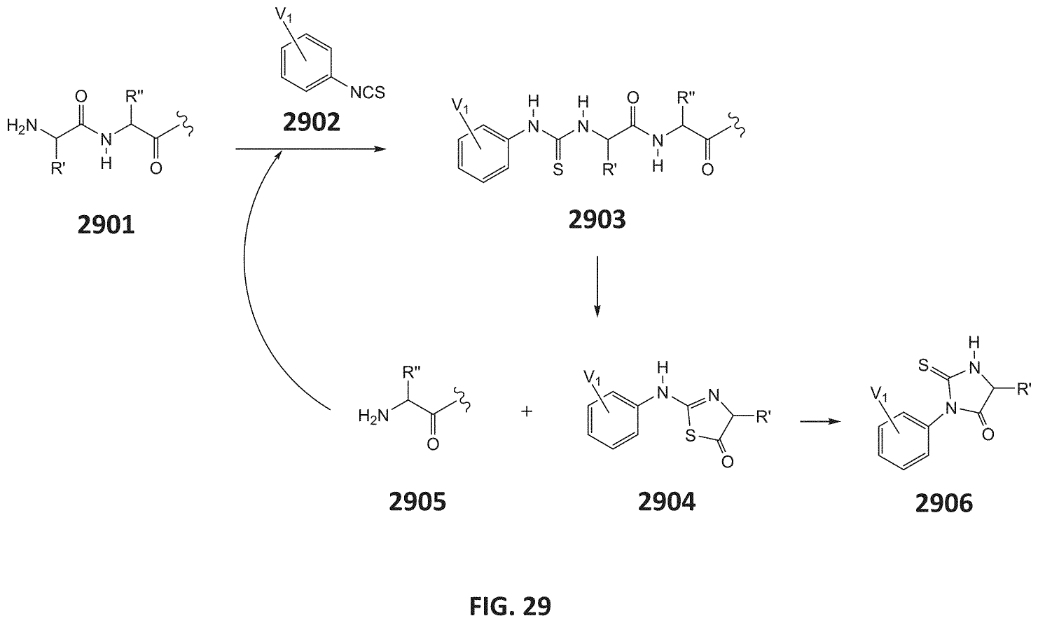

In some embodiments, a proteomic assay includes an Edman-type degradation assay. In some embodiments, an Edman-type degradation assay is utilized to determine a partial or complete sequence of a peptide or polypeptide. FIG. 29 shows a polypeptide 2901 being sequenced by a sequential process in which each cycle includes steps of labeling and removing N-terminal amino acids of a polypeptide isoform in a step-wise manner, and detecting released N-terminal labels. An example of this configuration is an Edman-type sequencing reaction in which a phenyl isothiocyanate 2902 reacts with a N-terminal amino group under mildly alkaline conditions, for example, about pH 8, to form an isolable, relatively stable cyclical phenylthiocarbamoyl Edman complex derivative 2903. In some embodiments, the phenyl isothiocyanate 2902 is substituted or unsubstituted with one or more functional groups, linker groups, or linker groups including functional groups (shown as a V1 substituent on the phenyl group of 2902). In some embodiments, an Edman-type sequencing reaction includes variations to reagents and conditions that yield a detectable removal of amino acids from a protein terminus, thereby facilitating determination of the amino acid sequence for a protein or portion thereof. For example, in some embodiments, the phenyl group is replaced with at least one aromatic, heteroaromatic or aliphatic group which participates in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). In some embodiments, under certain conditions, for example, acidic conditions of about pH 2, derivatized terminal amino acids are cleaved, for example, as a thiazolinone derivative 2904. In some embodiments, the thiazolinone amino acid derivative under acidic conditions forms a more stable phenylthiohydantoin (PTH) or similar amino acid derivative 2906 which is detected (for example, by chromatography, capillary electrophoresis, binding to an affinity reagent such as an antibody or aptamer, or mass spectrometry). In some embodiments, this procedure is repeated iteratively for residual polypeptide 2905 to identify the subsequent N-terminal amino acids and so forth as depicted in the cyclic nature of FIG. 29. In some embodiments, many variations of the Edman degradation have been described and are used including, for example, a one-step removal of an N-terminal amino acid using alkaline conditions. Additional details and information is found at Chang, J. Y., FEBS LETTS., 1978, 91(1), 63-68, which is hereby incorporated by reference in its entirety.

Non-limiting examples of V1 in 2902 include biotin and biotin analogs, fluorescent groups, click functionalities, for example, an azide or an acetylene. In some embodiments, V1 is part of these groups, for example, fluorescein isothiocyanate reacts with the N-terminus of a polypeptide in place of phenyl isothiocyanate. In some embodiments, V1 is a DNA, RNA, peptide or small molecule barcode or other tag which is further processed and/or detected. In some embodiments, barcodes include stable isotopes of hydrogen, carbon, nitrogen, oxygen, sulfur, phosphorus, boron or silicon. In some embodiments, barcodes including stable isotopes are detected by mass spectrometry. In some embodiments, V1 includes a metal complexing agent such as NTA (nitrolotriacetic acid) which binds strongly to certain metal ions, such as nickel (II) ions (Ni2+), where the Ni2+ ions links V1 to another molecular entity or surface comprising histidines or equivalents.

In some embodiments, affinity reagents described herein are used in combination with Edman-type sequencing reactions. For example, in some embodiments, an array including a plurality of polypeptides includes a first proteoform of a polypeptide comprising an N-terminal phosphotyrosine residue. In some embodiments, the polypeptide includes a second proteoform with a phosphotyrosine amino acid residue remote from its N-terminus. In some embodiments, a first affinity reagent having a first detectable label binds to the first proteoform of the polypeptide but not to the second proteoform of the polypeptide. In some embodiments, second affinity reagent having a second detectable label binds to the second proteoform of the polypeptide and not to the first proteoform of the polypeptide. In some embodiments, the two proteoforms of the polypeptide are characterized by analyzing signals from the first and second affinity reagents binding to their respective first and second proteoforms of the polypeptide. In some embodiments, the first and second labels re distinguishable from each other, but need not be, for example when used in separate cycles of a detection method set forth herein. In some embodiments, further characterization is performed by employing one or more Edman-type sequencing steps. In some embodiments, after contacting the array with first and second affinity reagents and detecting corresponding binding signals as described above, one or more Edman-type sequencing step is performed. Edman-type sequencing comprises at least two main steps, the first step comprises reacting an isothiocyanate or equivalent with polypeptide N-terminal residues at about pH 8. This forms a relatively stable Edman complex, for example, a phenylthiocarbamoyl complex. In some embodiments, the phenylthiocarbamoyl complex includes further chemical functionalities, for example, in some Edman-type methods it includes a fluorescent group, or a click chemistry functionality. The second Edman-type sequencing step comprises warming or heating the Edman complex until the N-terminal amino acid residue is removed. In some embodiments, a similar step is used in other Edman-type methods. In some embodiments, this removes all N-terminal residues of the polypeptides on the array including the N-terminal phosphotyrosine residue from the first proteoform of the polypeptide. In some embodiments, the array is contacted again with the first affinity reagent which now lacks a binding signal for the first proteoform of the polypeptide. In some embodiments, contacting the array with the second affinity reagent shows a positive binding result for the second proteoform of the polypeptide. In this way, in some embodiments, further characterization of at least the first proteoform of the polypeptide is achieved. In some embodiments, N-terminal residues cleaved by an Edman-type process, for example as phenylthiohydantoins are further analyzed. In some embodiments, the method is used for a polypeptide having an N-terminal PTM within about five or fewer amino acid residues of its N-terminus. In these embodiments, before an N-terminal amino acid residue comprising a PTM is cleaved, changes in binding signals is seen from the affinity reagents as PTM neighboring N-terminal amino acids are sequentially removed.

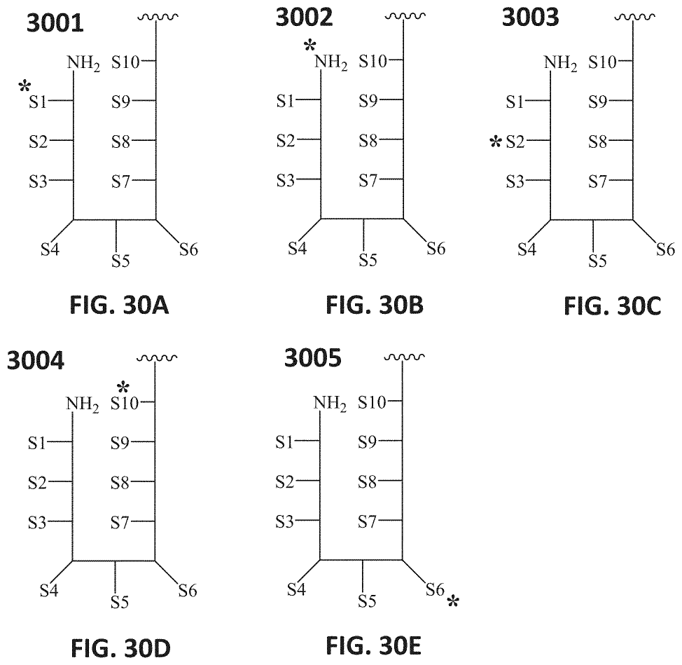

FIGS. 30A-E show five different truncated proteoforms of the same polypeptide where at least one PTM (*) resides in different locations in spatial proximity to the N-terminal portion of the polypeptide. FIG. 30A comprises a PTM on the side chain of N-terminal residue (S1). In some embodiments, a first affinity reagent to this polypeptide binds to an epitope, for example, the first three amino acid residues comprising at least the N-terminal primary amino group (NH2) and at least one of the amino acid side chains of the first three amino acid residues (S1*, S2 and S3) where a substantial amount of binding affinity occurs between the first affinity reagent and the PTM moiety. In some embodiments, removal of the N-terminal amino acid residue together with the PTM (*) by a first Edman-type degradation results in the first affinity reagent showing substantially less affinity to the shortened polypeptide to the extent that it would be considered to be non-binding to this epitope. In some embodiments, at the same time, a second affinity reagent shows substantial binding to one of the first Edman-type degradation intermediate products but show negligible binding to the polypeptide prior to performing the first Edman-type reaction. FIGS. 30B and 30C show similar losses of binding affinity to the same or different affinity reagents after the first Edman degradation reaction where the PTM resides within the binding epitope region of a first affinity reagent (contiguous epitope). In some embodiments, FIG. 30D shows the same trend even though the PTM is on amino acid residue number 10 (side chain=S10) as the polypeptide folds in such a manner where the S10 side-chain in the tertiary or quaternary structure of the polypeptide is in close proximity to the first three amino acid residues as part of a non-contiguous epitope for the first affinity reagent.

Referring to FIG. 30E where there is no PTM near the first three residues of the polypeptide, either contiguous or non-contiguous, in some embodiments, this polypeptide will not show a substantial change in binding (or non-binding) for the first affinity reagent either before or after a first Edman-type sequencing reaction. In some embodiments, such as in the case of FIG. 30E, a second affinity reagent which binds to the S6 region of the polypeptide (remote from the first amino acid residue) shows little or no change in binding when compared to both before and after the first Edman-type sequencing reaction for the first amino acid residue.

In some embodiments, affinity reagents described herein are used in combination with other chemical reagents which is used to modify proteoforms of polypeptides, for example, dansyl chloride is a chemical reagent used to modify protein amino groups including N-termini. Additional details and information is found at Walker, J. M., Methods Mol Biol. 1984; (1) 203-12. doi: 10.1385/0-89603-062-8:203, which is hereby incorporated by reference in its entirety for all purposes. In some embodiments, affinity reagents are used before, after, or both before and after such chemical modifications to further characterize proteoforms of polypeptides. For example, in some embodiments, an array including a plurality of polypeptides includes a first proteoform of a polypeptide comprising an N-terminal phosphotyrosine residue. In some embodiments, the polypeptide includes a second proteoform with a phosphotyrosine amino acid residue remote from its N-terminus. In some embodiments, a first affinity reagent having a first detectable label binds to the first proteoform of the polypeptide but not to the second proteoform of the polypeptide. In some embodiments, a second affinity reagent having a second detectable label binds to the second proteoform of the polypeptide and not to the first proteoform of the polypeptide. In some embodiments, the two proteoforms of the polypeptide are characterized by analyzing signals from the first and second affinity reagents binding to their respective first and second proteoforms of the polypeptide. In some embodiments, further characterization is performed by employing one or more steps using dansyl chloride. In some embodiments, after contacting the array with first and second affinity reagents and detecting corresponding binding signals, dansyl chloride is introduced to the array. In some embodiments, this labels all polypeptide N-termini with a dansyl group. Acid hydrolysis of the array yields a mixture of free amino acids plus dansyl amino acid derivatives of N-terminal amino acids. In some embodiments, these are detected using immobilized or free affinity reagents, for example, comprising FRET fluorescent groups which interact with the fluorescent dansyl group. In some embodiments, the affinity reagents to N-terminal dansyl groups are immobilized on solid supports, surfaces or beads and detected by, for example, fluorescence activated cell sorting. In some embodiments, the beads are tagged or barcoded, for example, with DNA barcodes that are cleaved and amplified by PCR and used to quantification of the captured affinity reagent.

In some embodiments, Edman-type reactions is thwarted by N-terminal modifications which is selectively removed, for example, N-terminal acetylation or formylation. Additional details and information is found at Gheorghe M. T., Bergman T. (1995) in Methods in Protein Structure Analysis, Chapter 8: Deacetylation and internal cleavage of Polypeptides for N-terminal Sequence Analysis. Springer, Boston, Mass. doi.org/10.1007/978-1-4899-1031-8_8, which is hereby incorporated by reference in its entirety for all purposes.

In some embodiments, a proteomic assay, such as the assay described in FIGS. 20-23, generates one or more single-polypeptide data sets that are utilized during a single-molecule process or an iterative process thereof. In some embodiments, a single-polypeptide data set includes data collected from any portion of a single-polypeptide proteomic assay, including pre-assay procedures, assay procedures, and post-assay procedures. Table III lists various exemplary pre-assay procedures, assay procedures, and post-assay procedures for certain proteomic assays, such as those described in FIGS. 20-23. A procedure is marked as “X” if it is likely to occur during the assay, and “O” if it optionally occurs during the assay. To the right, Table III lists a non-exhaustive, selected list of types of single-analyte data that, in some embodiments, are collected during each procedure. For example, in some embodiments, an array preparation process generates data such as array data (e.g., array composition, array pattern, array address spacing, array serial number, etc.), array metadata (e.g., manufacturer, manufacturing date, manufacturing instrument number, etc.), and array preparation history (e.g., array cleaning procedure parameters, array preparation procedure parameters, time-temperature histories, etc.). In some embodiments, an in-situ fluorescence detection procedure generates data such as fluorophore reagent data (e.g., fluorophore quantity, fluorophore concentration, buffer concentration, etc.), fluorophore reagent metadata (e.g., manufacture date, reagent preparer, etc.), fluorescence detection data (e.g., fluorescence intensity at each array address), and fluorescence data variability (e.g., fluorescence intensity measurement variance at each array address).

TABLE III

| | Method | | | | |
|---|---|---|---|---|---|
| | Fluorescence- | Barcode- | | Lifetime- | |
| | Based | Based | Fluoro- | Based | Selected Single- |
| | Binding | Binding | Sequencing | Sequencing | Molecule Data |
| Pre-Assay | | | | | |
| Procedures | | | | | |
| Sample | X | X | X | X | Sample data, |
| Collection | | | | | Sample metadata |
| Sample | X | X | X | X | Sample handling |
| Handling | | | | | history |
| Sample | X | X | X | X | Sample purification |
| Purification | | | | | history |
| Sample | ◯ | ◯ | X | X | Sample digestion |
| Digestion | | | | | history, Reagent |
| | | | | | data |
| Sample | ◯ | ◯ | X | ◯ | Sample labeling |
| Labeling | | | | | history, Reagent |
| | | | | | data |
| Sample | ◯ | X | — | — | Sample barcoding |
| Barcoding | | | | | history, Reagent |
| | | | | | data |
| Array | X | X | X | X | Array data, Array |
| Preparation | | | | | Metadata, Array |
| | | | | | Preparation history |
| Assay | | | | | |
| Procedures | | | | | |
| Multiple | X | X | X | X | Cycle number, |
| Cycles | | | | | Cycle history |
| Affinity- | X | X | — | X | Affinity binding |
| Reagent | | | | | profile, Affinity |
| Binding | | | | | reagent data, |
| | | | | | Affinity reagent |
| | | | | | metadata |
| Intra-Cycle | X | X | X | X | Rinse fluid |
| Rinsing | | | | | composition, Rinse |
| | | | | | fluid properties, |
| | | | | | Rinse fluid property |
| | | | | | variabilities* |
| Inter-Cycle | X | X | X | X | Rinse fluid |
| Rinsing | | | | | composition, Rinse |
| | | | | | fluid properties, |
| | | | | | Rinse fluid property |
| | | | | | variabilities* |
| Barcode | ◯ | X | ◯ | ◯ | Barcode reagent |
| Extension | | | | | data, Barcode |
| | | | | | reagent metadata, |
| | | | | | Barcode extension |
| | | | | | reaction history |
| In-Situ | X | — | X | — | Fluorophore reagent |
| Fluorescence | | | | | data, Fluorophore |
| Detection | | | | | reagent metadata, |
| | | | | | Fluorescence |
| | | | | | detection data, |
| | | | | | Fluorescence data |
| | | | | | variability* |
| Fluorescence/ | — | — | — | X | Fluoro-/Lumiphore |
| Luminescence | | | | | reagent data, Fluoro-/ |
| Lifetime | | | | | Lumiphore reagent |
| Measurement | | | | | metadata, Fluor-/ |
| | | | | | Luminescence |
| | | | | | detection data, |
| | | | | | Fluor-/ |
| | | | | | Luminescence data |
| | | | | | variability* |
| Barcode | ◯ | X | — | — | Barcode sequence |
| Sequencing | | | | | data, Barcode |
| | | | | | sequence |
| | | | | | variability* |
| Terminal | ◯ | ◯ | X | X | Cleavage reagent |
| Amino-Acid | | | | | data, Cleavage |
| Cleavage | | | | | reagent metadata |
| Post-Assay | | | | | |
| Procedures | | | | | |
| Polypeptide | ◯ | ◯ | — | — | Alteration reagent |
| Alteration | | | | | data, Alteration |
| | | | | | reagent metadata, |
| | | | | | Alteration assay |
| | | | | | data, Alteration |
| | | | | | assay variability |
| | | | | | data* |
| Polypeptide | ◯ | ◯ | — | — | Re-assay data, Re- |
| Re-Assay | | | | | assay variability |
| | | | | | data* |
| Polypeptide | ◯ | ◯ | — | — | Release reagent |
| Release | | | | | data, Release |
| | | | | | reagent metadata, |
| | | | | | Release assay data, |
| | | | | | Release assay |
| | | | | | variability* |
| Polypeptide | ◯ | ◯ | — | — | Collection reagent |
| Collection | | | | | data, Collection |
| | | | | | reagent metadata, |
| | | | | | Collection assay |
| | | | | | data, Collection |
| | | | | | assay variability* |

In some embodiments, a single-polypeptide data set generated during a single-molecule proteomic assay includes or issued to generate one or more process metrics, including uncertainty metrics for the proteomic assay. In some embodiments, one or more process metrics from a single-polypeptide data set is used to select, configure, and/or implement an action during an iterative process of the single-molecule proteomic assay. Table IV lists a non-exhaustive list of selected process metrics that, in some embodiments, is generated during or after the various procedures of a single-molecule proteomic assay listed in Table III. Table IV also includes some actions that, in some embodiments, are implemented during an iterative process of a single-polypeptide assay based upon the process metric annotated with an asterisk in each row. For example, in some embodiments, a barcoding efficiency for a plurality of polypeptides is determined. In some embodiments, based upon the determined barcoding efficiency, the proteomic assay is paused to determine a second barcoding efficiency on a reference second polypeptide array. In some embodiments, if results are found to disagree between the plurality of polypeptides and the reference array, a related process (e.g., re-performing a barcoding process) is performed before continuing the assay. In some embodiments, if the determined barcoding efficiency is above a threshold level, the proteomic assay is continued. In some embodiments, fluid flow variability during an intra-cycle rinse process is utilized to indicate improper function in a fluidics system of a single-polypeptide proteomic assay system. In some embodiments, if a measure of fluid flow variability (e.g., a variance of a flow rate, etc.) is found to exceed a threshold level, a single-polypeptide assay is paused to address a source of the fluid flow problem. In some embodiments, if the fluid flow variability is also determined to have affected physical measurements on a polypeptide, additional actions, such as altering an assay procedure sequence or deciding a next step (e.g., to repeat a possibly invalid measurement), is implemented. The skilled person will recognize that the precise embodiments of proteomic assays and single-analyte systems for performing the assays may affect the configuring of actions based upon available process metrics.

TABLE IV

| | | Iterative Process Actions* | | | | | |
|---|---|---|---|---|---|---|---|
| | Selected | | Alter | Identify | Perform | Call | Continue |
| | Process | Pause | Assay | Next Step | Related | to 2nd | Assay |
| | Metrics | Assay | Sequence | of Assay | Process | Analyte | Sequence |
| Pre-Assay | | | | | | | |
| Procedures | | | | | | | |
| Sample | Sample | — | X | — | X | X | X |
| Collection | Source, | | | | | | |
| | Sample | | | | | | |
| | Organism* | | | | | | |
| Sample | Mean | — | — | — | X | X | X |
| Handling | Storage | | | | | | |
| | Temperature*, | | | | | | |
| | Storage | | | | | | |
| | time length | | | | | | |
| Sample | Purification | | — | — | X | X | X |
| Purification | efficiency*, | | | | | | |
| | Total | | | | | | |
| | recovered | | | | | | |
| | quantity | | | | | | |
| Sample | Digestion | — | X | — | X | X | X |
| Digestion | time length, | | | | | | |
| | Digestion | | | | | | |
| | temperature* | | | | | | |
| Sample | Labeling | X | — | — | X | X | X |
| Labeling | efficiency*, | | | | | | |
| | Labels-per- | | | | | | |
| | molecule | | | | | | |
| Sample | Barcoding | X | — | — | X | X | X |
| Barcoding | efficiency*, | | | | | | |
| | Barcodes- | | | | | | |
| | per-molecule | | | | | | |
| Array | Array | X | — | — | X | — | X |
| Preparation | occupancy*, | | | | | | |
| | Arracy Co- | | | | | | |
| | localization | | | | | | |
| Assay | | | | | | | |
| Procedures | | | | | | | |
| Multiple | Cycle | — | X | X | — | — | X |
| Cycles | number, | | | | | | |
| | Total elapsed | | | | | | |
| | cycles* | | | | | | |
| Affinity- | Affinity | X | — | — | — | X | X |
| Reagent | reagent | | | | | | |
| Binding | concentration*, | | | | | | |
| | Affinity | | | | | | |
| | reagent | | | | | | |
| | identity | | | | | | |
| Intra-Cycle | Total fluid | X | X | X | — | — | X |
| Rinsing | volume, | | | | | | |
| | Fluid flow | | | | | | |
| | rate, Fluid | | | | | | |
| | flow | | | | | | |
| | variability* | | | | | | |
| Inter-Cycle | Total fluid | — | — | — | X | — | X |
| Rinsing | volume*, | | | | | | |
| | Fluid flow | | | | | | |
| | rate, Fluid | | | | | | |
| | flow | | | | | | |
| | variability | | | | | | |
| Barcode | Extension | X | X | X | X | — | X |
| Extension | temperature, | | | | | | |
| | Extension | | | | | | |
| | temperature | | | | | | |
| | variability* | | | | | | |
| In-Situ | Fluorescence | X | — | — | — | X | X |
| Fluorescence | intensity | | | | | | |
| Detection | count*, | | | | | | |
| | Fluorescence | | | | | | |
| | background | | | | | | |
| | count | | | | | | |
| Fluorescence/ | Signal | — | X | X | — | X | X |
| Luminescence | lifetime*, | | | | | | |
| Lifetime | Signal | | | | | | |
| Measurement | variability | | | | | | |
| Barcode | Sequence | X | X | X | — | X | X |
| Sequencing | counts, | | | | | | |
| | Sequence | | | | | | |
| | variability | | | | | | |
| | by sequence | | | | | | |
| | position* | | | | | | |
| Terminal | Reaction | X | — | — | X | X | X |
| Amino-Acid | time length, | | | | | | |
| Cleavage | Reactant | | | | | | |
| | concentration* | | | | | | |

In some embodiments, an iterative process performed during a single-molecule proteomic assay is discontinued when a determinant criterium has been achieved. In some embodiments, a determinant criterium is achieved when a process metric meets a defined criterium, or when a single-polypeptide characterization has been achieved. In some embodiments, a determinant criterium depends upon the nature of the proteomic assay. For example, in some embodiments, a barcode-based binding assay is configurable to achieve a characterization of a polypeptide proteoform but not a polypeptide amino acid sequence, whereas a fluorsequencing assay is configurable to achieve a characterization of a polypeptide amino acid sequence but not a polypeptide proteoform. In some embodiments, consequently, a differing determinant criterium is configured for a barcode-based binding assay compared to a fluorosequencing assay. In some embodiments, a determinant criterium for a single-polypeptide proteomic assay includes a total number of assay cycles (e.g., affinity-binding cycles, degradation cycles, etc.), a maximum number of assay cycles, a minimum number of assay cycles, a confidence level for a polypeptide identification traversing a threshold value, a confidence level for a polypeptide sequence traversing a threshold value, a confidence level for a polypeptide characteristic traversing a threshold value, attaining a polypeptide identity, attaining a polypeptide sequence, attaining a polypeptide characteristic, or a combination thereof.

### Single-Analyte Systems

Provided herein are systems for implementing single-analyte processes, including the synthesis, fabrication, manipulation, and assaying of single analytes of pluralities of single analytes according to any of the methods set forth herein. In some embodiments, the systems are configured to control a single-analyte process through an iterative process. In some embodiments, a single-analyte system is configured to acquire physical characterization measurements and other information that is utilized during an iterative process. For example, in some embodiments, a single-analyte system includes a detection system that is configured to acquire physical characterization measurements of a single analyte. In some embodiments, a single-analyte system includes a processor-implemented algorithm that controls one or more processes within a single-analyte system, including an iterative process. In some such embodiments, the detection system is in communication with the processor, such that signal information obtained by detecting one or more single analyte is transmitted to the processor as an input to the algorithm. In some embodiments, the processor is configured to transmit output information or commands from the algorithm to components of the system that effect one or more of the responsive actions set forth herein. For example, in some embodiments, a single-analyte system performs an iterative single-analyte process, and the algorithm is configured to identify or determine a process metric (e.g., uncertainty metric) based on data or information from the iterative single-analyte process. In some embodiments, the algorithm further evaluates the process metric (e.g., uncertainty metric) with respect to a determinant criterium, for example, to determine if a threshold has been crossed. In some embodiments, information from this determination, or an instruction derived by the algorithm from the information, is transmitted to a detection component, fluidics component or other component of the single-analyte system that is appropriate for taking a responsive action to modify a step (e.g., cycle, process or subprocess) of the iterative single-analyte process.

In some embodiments, a single-analyte system is configured to perform a single-analyte process such as a single-analyte assay process, a single-analyte synthesis process, a single-analyte fabrication process, a single-analyte manipulation process, or a combination thereof. In some embodiments, a single-analyte system is configured to perform a process comprising a first single-analyte process (e.g., a synthesis, a manipulation, etc.) and a second single-analyte assay process. In some embodiments, a single-analyte system is configured to perform a second single-analyte assay process before, during, or after a first single-analyte process. In some embodiments, a single-analyte system is configured to obtain a characterization of a single-analyte before, during or after a single-analyte process. For example, in some embodiments, a single-analyte process is performed on a single-analyte system to determine an intermediate product or a final product of a single-analyte synthesis or fabrication process. In some embodiments, the single-analyte system is configured to perform an identification assay, a quantification assay, a characterization assay, an interaction assay, or a combination thereof. Exemplary assays are set forth above and in the Examples section below.

In some embodiments, a single-analyte system includes a detection system. In some embodiments, a detection system includes any system or device that is configured to obtain a physical measurement of a single analyte. In some embodiments, a detection system is useful for any of a variety of methods or processes, such as the synthesis, fabrication, storage, stabilization, manipulation, utilization or assaying of a single analyte or a plurality of single analytes. For example, in some embodiments, a detection system is used to monitor the behavior or characteristics of a single analyte when undergoing such methods or processes. In some embodiments, a single-analyte system is configured to perform multiple utilities, such as synthesis and assaying of a single analyte, or manipulating and assaying of a single analyte.

In some embodiments, a detection system includes one or more components. In some embodiments, a detection system includes a single analyte or a plurality of single analytes, and a measurement device that is configured to obtain a physical measurement from the single analyte or the plurality of single analytes. In some embodiments, a detection system further comprises a retaining device that is configured to retain or include a single analyte or a plurality of single analytes. In some embodiments, a retaining device is coupled with a measurement device to facilitate the obtaining of a physical measurement of the single analyte. In some embodiments, a retaining device is configured such that a location and/or movement of a single analyte within the retaining device is constrained, limited, or free. In some embodiments, a retaining device is configured to retain a single analyte at a spatial location that is resolvable by a physical measurement, such as an optical, electrical, magnetic, radiological, chemical, or analytical measurement, or a combination thereof. For example, in some embodiments, a single analyte of a plurality of single analytes is located (e.g., by attachment) at a spatial location within a retaining device, and the location of the single analyte is resolvable from the locations of the other single analytes of the plurality of single analytes by a physical measurement. In some embodiments, a retaining device includes a plurality of single analytes in which each single analyte is located at a spatially-resolvable location within the retaining device. For example, in some embodiments, the single analytes is attached to respective sites in an array of single analytes. In some embodiments, each of the spatially-resolvable locations within the retaining device is unique. For example, in some embodiments, a different single analyte is located at each site and/or the sites is uniquely distinguishable based on unique characteristics of each site, whether the characteristic be location on a solid support or another type of characteristic such as shape, optical properties, or the like. In some embodiments, a retaining device includes a plurality of single analytes in which two or more single analytes is located at the same resolvable spatial location. In some embodiments, a retaining device includes a plurality of single analytes in which two or more single analytes is located at the same resolvable spatial location and at least one single analyte is located at a differing resolvable spatial location.

In some embodiments, a retaining device includes a flow cell, chip, or cartridge. In some embodiments, a flow cell includes a reaction chamber that includes one or more channels that direct fluid to a detection zone. In some embodiments, the detection zone is functionally coupled to a detector such that one or more single analyte present in the reaction chamber is observed. For example, in some embodiments, a flow cell includes single analytes attached to a surface in the form of an array of individually resolvable analytes. In some embodiments, ancillary reagents is iteratively delivered to the flow cell and washed away. In some embodiments, the flow cell includes an optically transparent material that permits the sample to be imaged, for example, after a desired reaction occurs. In some embodiments, an external imaging system is positioned to detect single analytes at a detection zone in the detection channel or on a surface in the detection channel. Exemplary flow cells, methods for their manufacture and methods for their use are described in US Pat. App. Publ. Nos. 2010/0111768 A1 or 2012/0270305 A1; or WO 05/065814, each of which is incorporated by reference herein in its entirety for all purposes.

In some embodiments, a retaining device is fluidically coupled to a fluidic system that is configured to transfer a fluid to or from the retaining device. In some embodiments, the fluidic system is configured to provide a liquid fluid or a gaseous fluid to the retaining device. In some embodiments, the retaining device us configured with an open channel architecture (e.g., one or more open fluidic channels). For example, in some embodiments, the retaining device is a well (e.g., a well in a multi-well plate) or reservoir that is accessible to a pipette or other aspiration device. In some embodiments, a retaining device is configured with a closed channel architecture (e.g., a flow cell or other device having one or more closed fluidic channels). In some embodiments, a fluidic system is configured to provide a fluid to a retaining device, including reagents, buffers, acids, bases, fluids comprising single-analytes, emulsions, suspensions, colloids, or a combination thereof. In some embodiments, a fluidics system is configured to provide a multiphase flow of two or more fluids. In some embodiments, a multiphase flow of two or more fluids is configured in a packet structure (e.g., a liquid packet with upstream and downstream gas packets, etc.). In some embodiments, a fluid that is provided to a retaining device includes one or more reagents used in a proteomics assay set forth herein, or known in the art. In some embodiments, a retaining device is configured to receive non-fluidic or semi-fluidic materials, including slurries, emulsions, foams, pastes, powders, gels, adhesives, or a combination thereof. In some embodiments, a fluidics system includes additional components that facilitate the transfer of fluids to or from a retaining device. In some embodiments, a fluidics system includes rigid or flexible tubing or piping. In some embodiments, tubing or piping is to provide fluidic connectivity between any portions of a fluidic system, including retaining devices, pumps, reservoirs, manifolds, etc. In some embodiments, tubing or piping is fixed to one or more system components, or is configured to be transferred between system components. For example, in some embodiments, a fluidics system includes a transferrable tubing line that is disconnected from a first port and subsequently re-connected to a second port. In some embodiments, a fluidics system includes fluid transfer components, such as pumps (e.g., positive-displacement pumps, negative-displacement pumps, vacuum pumps, peristaltic pumps, etc.), compressors, fans, blowers, and impellers. In some embodiments, a fluidics system includes fluid flow controlling elements that are configured to control the flow of fluid in the fluidics system, for example by stopping flow, starting flow, restricting flow, increasing flow, metering flow, or a combination thereof. In some embodiments, fluid controlling elements include valves (e.g., check valves, ball valves, solenoid valves, expansion valves, throttling valves, manifold valves, rotary valves, etc.), bubble traps, flow expanders, flow contractors, mass flow controllers, etc. In some embodiments, a fluidics system includes one or more sensors that are configured to provide data concerning the state of the fluidics system, for example for use by a fluid control algorithm, or for incorporation into a single-analyte data set as set forth herein. In some embodiments, a sensor is a digital or analog device. In some embodiments, value from a sensor is acquired automatically (e.g., via wireless transmitter) or manually (e.g., via a user recording the sensor value). In some embodiments, a sensor includes a fluidic sensor, including mass flow sensors, volumetric flow sensors, velocity gauges, pressure gauges, temperature gauges, fluid composition analyzers, pH sensors, bubble detectors, leak detectors, etc.

In some embodiments, a fluidic system is in communication with a processor that is configured to implement one or more algorithms as set forth herein. In some embodiments, a fluidics system is in communication with a processor that is configured to implement a fluidics control algorithm. In some embodiments, a fluidics system is in communication with a processor that is configured to implement an iterative process as set forth herein. In some embodiments, a fluidics system includes one or more sensors that communicate data to a processor that is configured to obtain or update a single-analyte data set as set forth herein. In some embodiments, a fluidics system includes one or more sensors that communicate data to a processor that is configured to determine one or more process metrics as set forth herein based upon the data transmitted by the sensor.

In some embodiments, a single-analyte system includes a retaining device comprising a surface. In some embodiments, the surface is configured to retain, bind, couple, or constrain a single analyte or a plurality of single analytes. In some embodiments, the surface comprises a solid support. In some embodiments, the solid support comprises a metal, a metal oxide, a glass, a ceramic, a semiconductor, a mineral, a polymer, a gel, or a combination thereof. In some embodiments, solid supports include, but are not limited to, gold, silver, copper, titanium oxide, zirconium oxide, alumina, silica, glass, fused silica, silicon, germanium, mica, and acrylics. In some embodiments, a surface comprises a phase boundary. In some embodiments, the phase boundary comprises a liquid/liquid boundary (e.g., water/oil), a liquid/gas boundary (e.g., water/air; oil/air), or a combination thereof.

In some embodiments, a single-analyte system comprises a retaining device including an array. In some embodiments, an array comprises a single analyte or a plurality of single analytes bound at regular, ordered, unordered, or random spatial locations on a surface. In some embodiments, the array comprises a patterned array or a non-patterned array. In some embodiments, the patterned array comprises a plurality of single analyte binding sites that are separated by interstitial regions that are configured to not bind the analytes. In some embodiments, a patterned array or a non-patterned array is formed on any suitable material, such as a solid support or a bead. In some embodiments, a patterned array or a non-patterned array includes one or more nano-wells or micro-wells. In some embodiments, a patterned array is formed by a suitable fabrication technique, such as photolithography, Dip-Pen nanolithography, nanoimprint lithography, nanosphere lithography, nanoball lithography, nanopillar arrays, nanowire lithography, scanning probe lithography, thermochemical lithography, thermal scanning probe lithography, local oxidation nanolithography, molecular self-assembly, stencil lithography, or electron-beam lithography.

In some embodiments, a non-patterned array comprises a surface that is configured to bind a plurality of single analytes. In some embodiments, a non-patterned array is formed by a natural segregation or separation of single analytes at discrete, resolvable spatial locations on an array surface. In some embodiments, a single-analyte system includes an array including a plurality of observable addresses, in which an address of the plurality of addresses comprises a single analyte or more than one single analyte.

In some embodiments, a system of the present disclosure employs any of a variety of stages to generate translational or rotational motion within the single-analyte system. In some embodiments, a translational or rotational stage is configured to produce a translational or rotational motion with any component of a single-analyte system set forth herein, including single analytes and arrays thereof, single-analyte retaining devices, fluidic systems, and measurement devices. In some embodiments, a stage is configured to translate a single analyte along a particular path, such as along a focus axis for an optical detection device. In some embodiments, a movement of a stage is described according to a coordinate system, such as an XYZ system (e.g., a Cartesian coordinate system), a spherical coordinate system, a cylindrical coordinate system, or a polar coordinate system. In some embodiments, point of reference for a coordinate system of a stage motion is configured with respect to the stage or a system component. In some embodiments, stage is configured to accommodate various component types. For example, in some embodiments, a stage is coupled with a retention system that is configured to securely hold or fasten a retaining device comprising a single analyte or an array of single analytes.

Particularly useful stages for translating a vessel or other specimen in x, y or z dimensions are set forth in US Pat. App. Pub. No. US 2019/0055598, US 2020/0393353, and US 2020/0290047, each of which is incorporated herein by reference in its entirety for all purposes. Those disclosures provide apparatus and methods that, in some embodiments, are used to observe a vessel by translational movement of the vessel relative to a detector. The scanning mechanism that is used to translate the vessel with respect to the detector is decoupled from the mechanism that is used to rotationally register the vessel with respect to the detector. In some embodiments, rotational registration of the vessel with respect to a detector is achieved by physically contacting the vessel with a reference surface, the reference surface being rotationally fixed with respect to the detector. For example, in some embodiments, the vessel is compressed to the reference surface by a preload. Separately, translation is achieved by a scan actuator (e.g., a pinion) that interacts directly with another surface of the vessel (e.g., a rack on a flow cell or cartridge that complements the pinion). The skilled person will readily recognize how such systems may be readily adapted to other system components to permit translational and/or rotational movements.

In some embodiments, a stage is coupled with one or more sensors that are configured to communicate position and/or orientation data to one or more algorithms as set forth herein. In some embodiments, a stage sensor is in communication with a processor that is configured to implement a positional or orientational control algorithm. In some embodiments, a stage sensor is in communication with a processor that is configured to implement an iterative process as set forth herein. In some embodiments, a stage is coupled to one or more sensors that communicate data to a processor that is configured to obtain or update a single-analyte data set as set forth herein. In some embodiments, a stage sensor includes one or more sensors that communicate data to a processor that is configured to determine one or more process metrics as set forth herein based upon the data transmitted by the sensor.

In some embodiments, a stage is in communication with a processor that is configured to implement one or more algorithms as set forth herein. In some embodiments, a stage is in communication with a processor that is configured to control position or motion of the stage. For example, in some embodiments, the processor is configured to implement an iterative process including, for example, steps of the process that include moving the stage.

In some embodiments, a single-analyte system comprises a detection system including a measurement device that is configured to perform the physical measurement of the single analyte. In some embodiments, the measurement device includes any instrument that observes a property, effect, characteristic, or interaction of a single analyte. In some embodiments, a measurement device is configured to provide a signal or input to a single analyte (e.g., exciting radiation, an electron beam, etc.). In some embodiments, a measurement device is configured to receive and/or detect a signal or output from a single analyte (e.g., a photon, an electron, a radioactive decay, etc.). In some embodiments, a measurement device includes one or more sensors that are configured to receive and/or detect a signal or output from a single-analyte system. In some embodiments, a measurement device is configured to obtain a physical measurement of a single analyte by any of a variety of mechanisms, including surface plasmon resonance, atomic force microscopy, fluorescent microscopy, fluorescence lifetime measurement, luminescent microscopy, luminescence lifetime measurement, optical microscopy, electron microscopy, Raman spectroscopy, mass spectrometry, or a combination thereof.

In some embodiments, a detection device is configured to communicate physical measurement data to one or more algorithms as set forth herein. In some embodiments, a detection device is in communication with a processor that is configured to implement a detection device control algorithm. For example, in some embodiments, a set of instructions configured by an iterative process is communicated to a processor that implements a detection device control algorithm, and the processor subsequently communicates the instructions to the detection device. In some embodiments, a detection device is in communication with a processor that is configured to implement an iterative process as set forth herein. In some embodiments, a detection device is coupled to one or more sensors that communicate data to a processor that is configured to obtain or update a single-analyte data set as set forth herein. In some embodiments, a detection device includes one or more sensors that communicate data to a processor that is configured to determine one or more process metrics as set forth herein based upon the data transmitted by the sensor.

In some embodiments, a detection device is in communication with a processor that is configured to implement one or more algorithms as set forth herein. In some embodiments, a detection device is in communication with a processor that is configured to control functions of the detection device such as detector sensitivity, gain, focus, acquisition duration, signal resolution (e.g., wavelength of detection) or the like. For example, in some embodiments, the processor is configured to implement an iterative process including, for example, steps of the process that include adjusting position or function of the detection device.

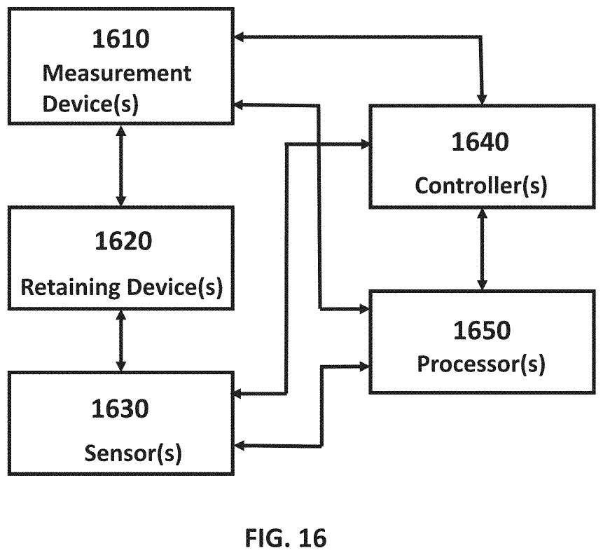

In some embodiments, a detection system within a single-analyte system includes one or more additional components selected from the group consisting of: a processor, a sensor, and a controller. FIG. 16 depicts a single-analyte system as described by its information connectivity, in accordance with some embodiments detailed herein. In some embodiments, one or more retaining devices 1620 is configured to send or receive signals (e.g., photons, electrons, electrical fields, magnetic fields, etc.) with one or more measurement devices 1610. In some embodiments, the measurement devices 1610 is configured to send or receive information (e.g., data, operation instructions) with one or more controllers 1640 and/or one or more processors 1650. In some embodiments, the one or more processors 1650 is located together (e.g., within a cloud server) or is distributed (e.g., a processor 1650 integrated within a controller 1640, a processor 1650 integrated with a measurement device 1610, etc.). In some embodiments, the one or more retaining devices 1620 is be configured to send or receive signals (e.g., photons, electrons, electrical fields, magnetic fields, etc.) with one or more sensors 1630. In some embodiments, the one or more sensors 1630 is configured to send or receive information (e.g., data, operation instructions) with one or more controllers 1640 and/or one or more processors 1650.

In some embodiments, the processor comprises a central processing unit, a graphics processing unit, a vision processing unit, a tensor processing unit, a neural processing unit, a physics processing unit, a digital signal processor, an image signal processor, a synergistic processing element, a field-programmable gate array, or a combination thereof. In some embodiments, a processor is configured to implement one or more algorithms. In some embodiments, a processor is configured to implement an algorithm that controls a single-analyte process, such as any single-analyte process set forth herein. In some embodiments, a processor is configured to implement an algorithm that implements an iterative process, such as any iterative process set forth herein. In some embodiments, a single-analyte system includes more than one processor. In some embodiments, a detection system includes a processor that is configured to perform one or more algorithms, such as one or more algorithms that perform a single-analyte process as set forth herein. In some embodiments, a single-analyte system includes a hard-wired or wireless connection to one or more processors that are configured to perform a single-analyte process. In some embodiments, a processor that is configured to perform one or more algorithms that perform a single-analyte process as set forth herein is located on a computer, a terminal station, a handheld device (e.g., a cell phone, a tablet, a remote control), a server (e.g., a cloud-based server), or a combination thereof.

In some embodiments, a detection system includes one or more sensors. In some embodiments, a sensor includes a sensor that is configured to obtain a physical measurement of a single-analyte, or a sensor that is configured to obtain a physical measurement of a single-analyte system parameter (e.g., temperature, pressure, flow rate, composition, pH, etc.). In some embodiments, the sensor comprises a thermal sensor, a pressure sensor, a force sensor, a flow sensor, a mechanical sensor, a chemical sensor, an optical sensor, a focus sensor, a camera, an electrical sensor, a speed sensor, a positional sensor, an ionizing radiation sensor, or a combination thereof.

In some embodiments, a detection system includes a controller. In some embodiments, a controller includes any device that is configured to control the physical or data transfer actions of the single-analyte system. In some embodiments, a controller is configured to received instructions for a single-analyte process as set forth herein from an algorithm, and optionally is further configured to implement the instructions on one or more hardware components of the single-analyte system. In some embodiments, a controller includes devices such as mass flow controllers, volumetric flow controllers, pressure controllers, level controllers, proportional/integral/derivative controllers, programmable logic controllers (PLC), distributed control systems (DCS), supervisory control, integrated circuit, field-programmable gate array (FPGA) and data acquisition controllers (SCADA), or a combination thereof. In some embodiments, a controller is configured to implement an action determined by an iterative loop as set forth herein on the single-analyte system.

In some embodiments, a single-analyte system is configured to collect a single-analyte data set. In some embodiments, a detection system includes one or more components that are configured to provide data for a single-analyte data set. In some embodiments, a single-analyte data set includes data obtained from a measurement device, a sensor, a processor, or a combination thereof. For example, in some embodiments, during a single-analyte synthesis process or a single-analyte assay process, a single-analyte data set includes physical characterization data of a single analyte, and optionally instrument metadata from one or more sensors, and further optionally one or more calculated or extracted process metrics as determined by a processor. In some embodiments, the single-analyte data set includes data collected from the measurement device or the one or more additional components. For example, in some embodiments, a single-analyte data set includes only physical measurement data of a single analyte. In some embodiments, a single-analyte data set includes one process metrics that are provided by a processor based upon data provided to the processor by a sensor. In some embodiments, the single-analyte data set includes data collected from the measurement device and the one or more additional component. For example, in some embodiments, during a single-analyte synthesis process or a single-analyte assay process, a single-analyte data set includes physical characterization data of a single analyte, and instrument metadata from one or more sensors, as well as one or more calculated or extracted process metrics as determined by a processor.

In some embodiments, a single-analyte system includes a single analyte or a plurality of single-analytes derived from any of a variety of sources including, for example, a biological source, a non-biological source, an industrial source, or a combination thereof. In some embodiments, a single-analyte system is configured to synthesize or fabricate a single analyte in situ. In some embodiments, a single-analyte system is configured to receive and/or retain a single analyte, for example from a sample comprising the single analyte.

In some embodiments, a single analyte is derived from a biological sample. In some embodiments, a biological sample includes a sample derived from a primarily biological sample, such as an animal, plant, fungus, bacterium, virus, archaea, or a fragment thereof. In some embodiments, a biological sample includes intact or disrupted biological organisms or biologically-derived particles, such as single cells, viral particles, vesicles, and multicellular tissues or organisms, and any components thereof. In some embodiments, a biological sample includes engineering organisms or fragments thereof, forensic samples, paleontological samples, bio-archeological samples, industrial samples (e.g., fermentation products) or a combination thereof. In some embodiments, a single analyte comprises a biomolecule or biomolecular complex such as a nucleic acid, a lipid, a polypeptide, a polysaccharide, a metabolite, a cofactor, or a combination thereof. In some embodiments, the biomolecule includes one or more isoforms or variants (e.g., polypeptide proteoforms, hemicelluloses, lignins, etc.). In some embodiments, a biomolecule includes a known, unknown, characterized, or uncharacterized structure, sequence, function, property, effect, behavior, or interaction. In some embodiments, a single-analyte process includes an assay to characterize a single analyte from a biological sample, such as an assay selected from a group consisting of a sequencing assay, a fluoro-sequencing assay, an affinity binding assay, a fluorescence lifetime assay, a luminescence lifetime assay, an electronic assay, an optical assay, and a combination thereof.

In some embodiments, a single analyte is derived from a non-biological sample. In some embodiments, a non-biological sample includes a sample that is derived from a primarily non-biological source, such as an industrial sample, a geological sample, an archeological sample, an extraterrestrial sample, or a combination thereof. In some embodiments, a non-biological sample includes biological analytes (e.g., a wastewater effluent). In some embodiments, a non-biological single analyte is a synthesized particle such as a nanoparticle, a crystalline particle, an amorphous particle, a catalytic particle, or a combination thereof. In some embodiments, the non-biological sample includes a polymer, a ceramic, a metal, a metal alloy, a semiconductor, a mineral, or a combination thereof.

In some embodiments, a single-analyte system includes one or more algorithms that are configured to implement various aspects of a single-analyte process as set forth herein. In some embodiments, a single-analyte system includes a plurality of algorithms configured to collectively implement all aspects of a single-analyte process. For example, in some embodiments, a single-analyte system includes a software package that implements a single-analyte process. In some embodiments, a single-analyte system includes one or more algorithms that are configured to communicate with one or more algorithms that are external to the single-analyte system. In some embodiments, an external algorithm includes an algorithm that is not located within a component of the single-analyte system, such as an external computer, an external server, a separate single-analyte system, etc. For example, in some embodiments, a single-analyte system includes an algorithm that is configured to query a database of an external vendor to obtain supplier-provided information on a reagent utilized during a single-analyte process. In some embodiments, a single-analyte system includes one or more algorithms (e.g., algorithms configured to collect a single-analyte data set and/or implement an iterative process as set forth herein) that communicate data to an external server that is configured to determine one or more process metrics based upon the communicated data.

In some embodiments, a single-analyte system includes a plurality of algorithms in which each algorithm of the plurality of algorithms performs a different function for the single-analyte system. In some embodiments, an algorithm of a plurality of algorithms performs a function such as data collection algorithm, data analysis, process configuration, system maintenance, system repair, process control, communications, and sending/receiving user inputs and or outputs. In some embodiments, each algorithm of a plurality of algorithms is performed on a single processor or set of processors (e.g., a computer, a server, a cloud server, etc.). In other embodiments, a first algorithm of a plurality of algorithms is performed on a first processor and a second algorithm of the plurality of algorithms is performed on a second process. For example, in some embodiments, a single-analyte system includes a detection device comprising an imaging sensor whose image data is collected and processed by a first processor (e.g., a graphics processing unit) before transferring the image data to a second processor (e.g., a central processing unit) for determination of a process metric.

In some embodiments, a single-analyte system includes two or more algorithms that are configured to perform a similar or identical function. For example, in some embodiments, a first algorithm processes a set of data to determine a first process metric and a second algorithm processes the same set of data to determine a differing process metric. In some embodiments, an algorithm processes a set of data on a first processor, and the same algorithm processes a different set of data on a different processor. In some embodiments, a single-analyte system is configured to implement two or more algorithms simultaneously. In some embodiments, a single-analyte system is configured to implement two or more algorithms sequentially. In some embodiments, a single-analyte system comprises two or more algorithms that are configured to implement an iterative process as set forth herein. In some embodiments, a single-analyte system is configured to simultaneously implement two or more algorithms that perform iterative processes. For example, in some embodiments, a single-analyte system is configured to intermittently implement a first iterative process that pauses a single-analyte process to correct a source of measurement uncertainty, and/or is configured to continuously implement a second iterative process that alters a sequence of steps of the single-analyte process. In some embodiments, a single-analyte system is configured to sequentially implement two or more algorithms that perform iterative processes. For example, in some embodiments, a single-analyte system implements a first iterative process that iterates through a sequence of measurements for a single analyte to determine one or more properties of the single analyte, then subsequently implements a second iterative process that utilizes the one or more properties of the single analyte to perform a manipulation of the single analyte.

In some embodiments, a single-analyte system is configured to implement two or more algorithms during a single-analyte process. In some embodiments, a single-analyte system is configured to implement two or more algorithms that perform iterative processes during a single-analyte process. In some embodiments, a single-analyte system implements a first algorithm that operates on a first time-scale and a second algorithm that operates on a second time-scale. In some embodiments, a time-scale for an algorithm refers to the relative or absolute time length upon which an algorithm completes a task, provides an output, accepts an input, or a combination thereof. For example, in some embodiments, an algorithm collects data from a single analyte on the time-scale of milli-seconds to seconds. In some embodiments, an algorithm performs a calculation based upon a single-analyte data set on the time-scale of minutes to hours. In some embodiments, a single-analyte system implements a first algorithm that operates on a first time-scale and a second algorithm that operates on a second time-scale, in which the first time-scale and the second time-scale are aligned, matched and/or overlapping. For example, in some embodiments, a first algorithm is configured to receive data from a second algorithm and analyze the data before the second algorithm has a new set of data. In some embodiments, a single-analyte system implements a first algorithm that operates on a first time-scale and a second algorithm that operates on a second time-scale, in which the first time-scale and the second time-scale are differing. For example, in some embodiments, a hardware driver algorithm completes numerous cycles of operation while an analysis algorithm is performing a single cycle of operation. In some embodiments, a single-analyte system is configured to implement a first iterative process algorithm that operates on a first time-scale and a second iterative process algorithm that operates on a second time-scale.

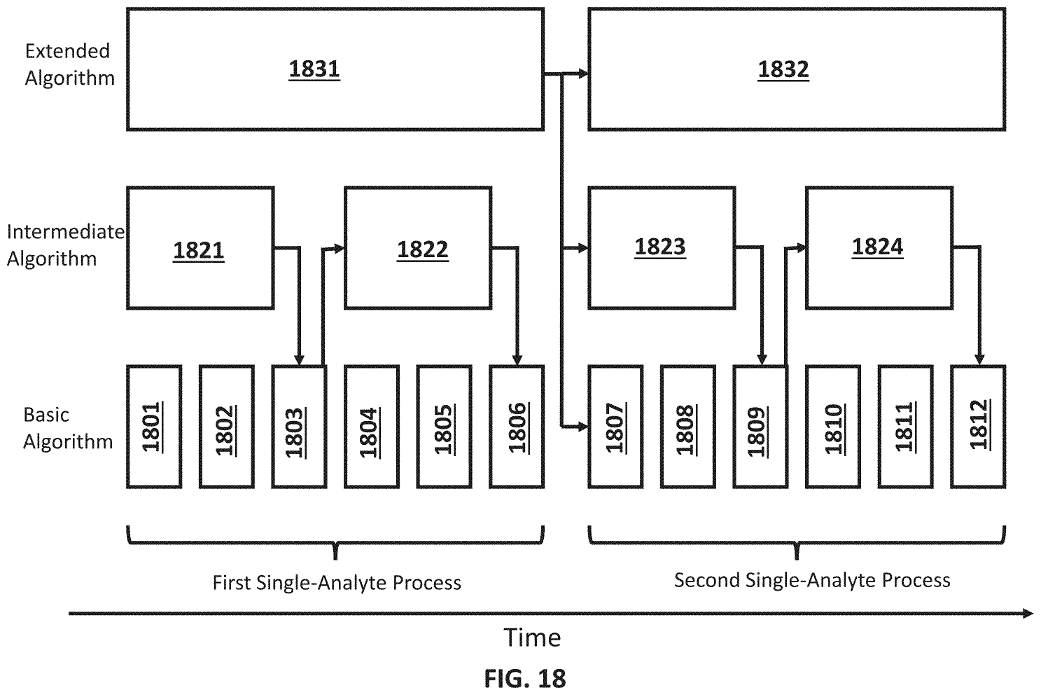

FIG. 18 illustrates an algorithm time-scale scheme for a single-analyte system. In some embodiments, the single-analyte system is configured to implement a plurality of sequential basic algorithms 1801-1806 with short time-scales during a first single-analyte process. In some embodiments, the single-analyte system is further configured to run an intermediate time-scale algorithm 1821 that runs simultaneously with algorithms 1801 and 1802, but completes in time to provide an input into algorithm 1803. In some embodiments, the single-analyte is further configured to run a second medium time-scale 1822 that is configured to receive an input from algorithm 1803 and complete in time to provide an input to short time-scale algorithm 1806. In some embodiments, the intermediate time-scale algorithms 1821 and 1822 is configured to receive inputs from basic algorithms 1801, 1802, 1804, and 1805. In some embodiments, the single-analyte system is configured to run an extended time-scale algorithm 1831 that does not complete its task until the completion of the single-analyte process. In some embodiments, the extended time-scale algorithm 1831 receives one or more inputs from intermediate algorithms 1821 and 1822. In some embodiments, the single-analyte system is further configured to implement a second plurality of algorithms, including basic algorithms 1807-1812, intermediate algorithms 1823 and 1824, and extended algorithm 1832 during a second single-analyte process. In some embodiments, the operation and/or interplay of the algorithms of the second single-analyte process proceeds similarly to the first single-analyte process. In some embodiments, the extended algorithm 1831 provides inputs to algorithms 1807, 1823, and/or 1832. The skilled person will readily recognize numerous variations of sequencing and interaction between a plurality of algorithms while implementing a single-analyte process as set forth herein.

In some embodiments, a single-analyte system is configured to utilize a plurality of algorithms during the implementation of a single-analyte process. In some embodiments, a single-analyte system includes decentralized, distributed, or centralized algorithms that are configured to implement a single-analyte process. In some embodiments, a single-analyte system includes one or more centralized algorithms (e.g., process control algorithms, image processing images, data processing algorithms, etc.) that are configured to communicate with a decentralized set of algorithms. For example, in some embodiments, a centralized algorithm that implements an iterative process as set forth herein exports a single-analyte data set to a set of decentralized algorithms that perform calculations with the single-analyte data set. In some embodiments, a decentralized algorithm is configured to push information (e.g., data, calculated values, updated models, updated algorithms) to a single-analyte system. In some embodiments, a decentralized or distributed network of algorithms includes a plurality of algorithms in which each algorithm of the plurality of algorithms is configured to determine the same information. For example, in some embodiments, each algorithm of a plurality of algorithms in a decentralized or distributed network of algorithms is configured to each determine a same uncertainty metric from a single-analyte data set. In some embodiments, a decentralized or distributed network of algorithms is configured to include a range of computational models, computational schemes, and/or processing times scales. For example, in some embodiments, each algorithm of a decentralized network of algorithms is configured to independently calculate the same process metric via differing computational models. In some embodiments, a distributed network of algorithms is configured to independently apply a stochastic algorithm (e.g., same initial conditions producing differing results) to generate a range of predictions or outcomes for the same calculation. In some embodiments, a decentralized or distributed network of algorithms is configured to implement an ensemble machine-learning method such as stacking or blending.

In some embodiments, two or more algorithms are invoked during a single-analyte process when processing data, analyzing data, or deciding an action during an iterative process. In some embodiments, two or more algorithms are configured to be invoked in a series or hierarchical fashion. For example, in some embodiments, a first algorithm is configured to perform a calculation based upon data from a single-analyte data set. In some embodiments, if the calculation is deemed insufficient or low confidence based upon an uncertainty metric for the calculation (e.g., a confidence interval), then a second algorithm of differing computational complexity is called to perform the calculation. In some embodiments, two or more algorithms are configured to be invoked in a parallel fashion. For example, in some embodiments, a single-analyte data set is simultaneously transferred to two or more algorithms of differing computational complexity. In some embodiments, an iterative process possesses a time deadline by which at least one of the algorithms must deliver a result. In some embodiments, if each algorithm produces a result, the most accurate or confident result is applied for making a decision regarding an implemented action on the single-analyte system; otherwise, the first completed algorithm is utilized for decision purposes after the deadline has expired. In some embodiments, one or more algorithms is selected for performing computations for any method set forth herein based upon an a priori or a posteriori selection method.

In some embodiments, a single-analyte system of the present disclosure is configured to implement a machine-learning or training algorithm. In some embodiments, a machine-learning or training algorithm is configured to perform an iterative process, as set forth herein. In some embodiments, a machine-learning or training algorithm is configured to calculate one or more process metrics from a single-analyte data set. In some embodiments, a machine-learning or training algorithm is configured to update a single-analyte data set based upon performed calculations. In some embodiments, a single-analyte system includes an algorithm that is configured to implement a method such as machine learning, deep learning, statistical learning, supervised learning, unsupervised learning, clustering, expectation maximization, maximum likelihood estimation, Bayesian inference, non-Bayesian inference, linear regression, logistic regression, binary classification, multinomial classification, or other pattern recognition algorithm. In some embodiments, machine learning algorithms include support vector machines (SVMs), neural networks, convolutional neural networks (CNNs), deep neural networks, cascading neural networks, k-Nearest Neighbor (k-NN) classification, random forests (RFs), and other types of classification and regression trees (CARTs).

The present disclosure provides a non-transitory information-recording medium that has, encoded thereon, instructions for the execution of one or more steps of the methods set forth herein, for example, when these instructions are executed by an electronic computer in a non-abstract manner. This disclosure further provides a computer processor (e.g., not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Any claim that is explicitly limited to physical, tangible and non-abstract subject matter, will be understood to be directed to non-abstract subject matter, when taken as a whole. As used herein, the term “non-abstract” is the converse of “abstract” as that term has been interpreted by controlling precedent of the U.S. Supreme Court and the Federal Circuit as of the priority date of this application

In some embodiments, an algorithm or plurality of algorithms set forth herein effects an improvement in a technology or field. For example, in some embodiments, a single-analyte process comprising one or more algorithms configure to implement iterative processes improves the function of a single-analyte system as set forth herein. In some embodiments, a single-analyte process comprising one or more algorithms configure to implement iterative processes improves the reliability and/or predictability of single-analyte processes for biotechnology, chemical, and physical applications. In some embodiments, an algorithm of a single-analyte process is implemented on a non-generic computer. For example, in some embodiments, a single-analyte process is implemented on a single-analyte system comprising a plurality of processors, in which each processor of the plurality of processors is associated with a different system component, and in which each processor of the plurality of processors implements a differing algorithm that contributes to the performance of the single-analyte process. In some embodiments, an algorithm of a single-analyte process includes a non-generic implementation of a computer. For example, in some embodiments, the efficiency of a repeated single-analyte process inherently increased over time due to the ability of an algorithm to apply a machine-learning model to prior performances of the single-analyte process. In some embodiments, a single-analyte system as set forth herein is configured to integrate one or more building blocks of human ingenuity into something more.

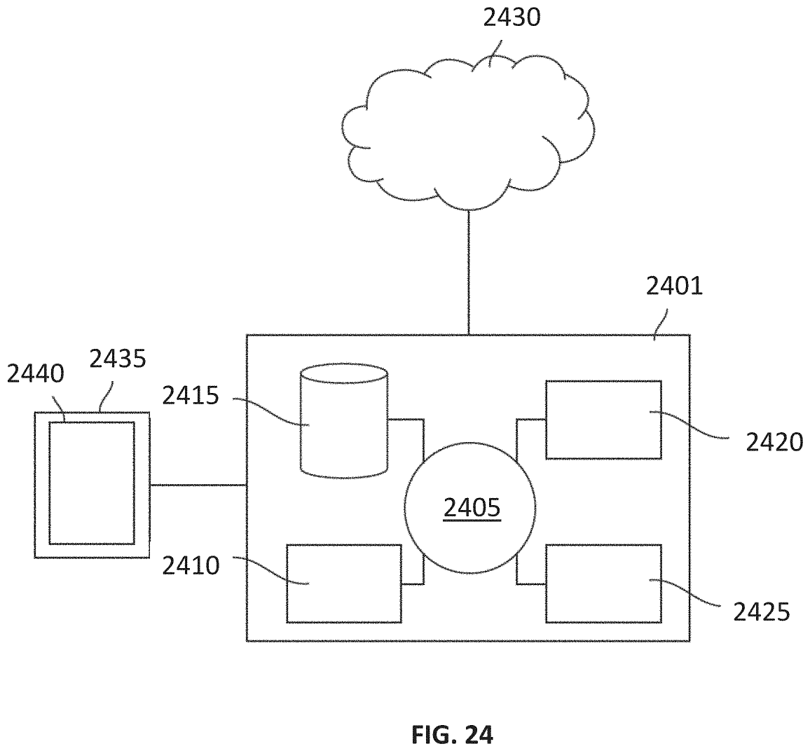

The present disclosure provides computer control systems that are programmed to implement methods of the disclosure. FIG. 24 shows a computer system 2401 that is programmed or otherwise configured to: determine a process metric based upon a single-analyte data set, implement an action on a single-analyte system based upon the process metric, and update the single-analyte data set after implementing the action on the single-analyte system.

In some embodiments, the computer system 2401 regulates various aspects of methods and systems of the present disclosure, such as, for example, determining a process metric based upon a single-analyte data set, implementing an action on a single-analyte system based upon the process metric, and updating the single-analyte data set after implementing the action on the single-analyte system.

In some embodiments, the computer system 2401 is an electronic device of a user or a computer system that is remotely located with respect to the electronic device. In some embodiments, the electronic device is a mobile electronic device. The computer system 2401 includes a central processing unit (CPU, also “processor” and “computer processor” herein) 2405, which is a single core or multi core processor, or a plurality of processors for parallel processing. The computer system 2401 also includes memory or memory location 2410 (e.g., random-access memory, read-only memory, flash memory), electronic storage unit 2415 (e.g., hard disk), communication interface 2420 (e.g., network adapter) for communicating with one or more other systems, and peripheral devices 2425, such as cache, other memory, data storage and/or electronic display adapters. The memory 2410, storage unit 2415, interface 2420 and peripheral devices 2425 are in communication with the CPU 2405 through a communication bus (solid lines), such as a motherboard. In some embodiments, the storage unit 2415 is a data storage unit (or data repository) for storing data. In some embodiments, the computer system 2401 is operatively coupled to a computer network (“network”) 2430 with the aid of the communication interface 2420. In some embodiments, the network 2430 is the Internet, an internet and/or extranet, or an intranet and/or extranet that is in communication with the Internet. In some embodiments, the network 2430 is a telecommunication and/or data network. In some embodiments, the network 2430 includes one or more computer servers, which enables distributed computing, such as cloud computing. For example, in some embodiments, one or more computer servers enabled cloud computing over the network 2430 (“the cloud”) to perform various aspects of analysis, calculation, and generation of the present disclosure, such as, for example, determining a process metric based upon a single-analyte data set, implementing an action on a single-analyte system based upon the process metric, and updating the single-analyte data set after implementing the action on the single-analyte system. In some embodiments, such cloud computing is provided by cloud computing platforms such as, for example, Amazon Web Services (AWS), Microsoft Azure, Google Cloud Platform, and IBM cloud. In some embodiments, the network 2430, with the aid of the computer system 2401, implements a peer-to-peer network, which enables devices coupled to the computer system 2401 to behave as a client or a server.

In some embodiments, the CPU 2405 executes a sequence of machine-readable instructions, which is embodied in a program or software. In some embodiments, the instructions are stored in a memory location, such as the memory 2410. In some embodiments, the instructions are directed to the CPU 2405, which subsequently program or otherwise configure the CPU 2405 to implement methods of the present disclosure. In some embodiments, the CPU 2405 performs fetch, decode, execute, and writeback.

In some embodiments, the CPU 2405 is part of a circuit, such as an integrated circuit. In some embodiments, one or more other components of the system 2401 is included in the circuit. In some embodiments, the circuit is an application specific integrated circuit (ASIC).

In some embodiments, the storage unit 2415 stores files, such as drivers, libraries and saved programs. In some embodiments, the storage unit 2415 stores user data, e.g., user preferences and user programs. In some embodiments, the computer system 2401 includes one or more additional data storage units that are external to the computer system 2401, such as located on a remote server that is in communication with the computer system 2401 through an intranet or the Internet.

In some embodiments, the computer system 2401 communicates with one or more remote computer systems through the network 2430. For instance, in some embodiments, the computer system 2401 communicates with a remote computer system of a user. Examples of remote computer systems include personal computers (e.g., portable PC), slate or tablet PC's (e.g., Apple iPad, Samsung Galaxy Tab), telephones, Smart phones (e.g., Apple iPhone, Android-enabled device, Blackberry), or personal digital assistants. In some embodiments, the user accesses the computer system 2401 via the network 2430.

In some embodiments, methods as described herein are implemented by way of machine (e.g., computer processor) executable code stored on an electronic storage location of the computer system 2401, such as, for example, on the memory 2410 or electronic storage unit 2415. In some embodiments, the machine executable or machine-readable code is provided in the form of software. In some embodiments, during use, the code is executed by the processor 2405. In some embodiments, the code is retrieved from the storage unit 2415 and stored on the memory 2424 for ready access by the processor 2405. In some embodiments, the electronic storage unit 2415 is precluded, and machine-executable instructions are stored on memory 2410.

In some embodiments, the code is pre-compiled and configured for use with a machine having a processor adapted to execute the code, or is compiled during runtime. In some embodiments, the code is supplied in a programming language that is selected to enable the code to execute in a pre-compiled or as-compiled fashion.

Aspects of the systems and methods provided herein, such as the computer system 2401, can be embodied in programming. In some embodiments, various aspects of the technology is thought of as “products” or “articles of manufacture” typically in the form of machine (or processor) executable code and/or associated data that is carried on or embodied in a type of machine readable medium. In some embodiments, machine-executable code is stored on an electronic storage unit, such as memory (e.g., read-only memory, random-access memory, flash memory) or a hard disk. In some embodiments, “storage” type media includes any or all of the tangible memory of the computers, processors or the like, or associated modules thereof, such as various semiconductor memories, tape drives, disk drives and the like, which provide non-transitory storage at any time for the software programming. In some embodiments, all or portions of the software at times is communicated through the Internet or various other telecommunication networks. In some embodiments, such communications, for example, enable loading of the software from one computer or processor into another, for example, from a management server or host computer into the computer platform of an application server. Thus, in some embodiments, another type of media that bears the software elements includes optical, electrical and electromagnetic waves, such as used across physical interfaces between local devices, through wired and optical landline networks and over various air-links. In some embodiments, the physical elements that carry such waves, such as wired or wireless links, optical links or the like, also is considered as media bearing the software. As used herein, unless restricted to non-transitory, tangible “storage” media, terms such as computer or machine “readable medium” refer to any medium that participates in providing instructions to a processor for execution.

Hence, in some embodiments, a machine readable medium, such as computer-executable code, takes many forms, including but not limited to, a tangible storage medium, a carrier wave medium or physical transmission medium. In some embodiments, non-volatile storage media include, for example, optical or magnetic disks, such as any of the storage devices in any computer(s) or the like, such as is used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. In some embodiments, carrier-wave transmission media takes the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer reads programming code and/or data. In some embodiments, many of these forms of computer readable media are involved in carrying one or more sequences of one or more instructions to a processor for execution.

In some embodiments, the computer system 2401 includes or is in communication with an electronic display 2435 that comprises a user interface (UI) 2440 for providing, for example, user input of single-analyte data, rules for configuring actions based upon process metrics, and/or decisions on implementing an action on a single-analyte system. Examples of UIs include, without limitation, a graphical user interface (GUI) and web-based user interface.

In some embodiments, methods and systems of the present disclosure are implemented by way of one or more algorithms. In some embodiments, an algorithm is implemented by way of software upon execution by the central processing unit 2405. In some embodiments, the algorithm, for example, determines a process metric based upon a single-analyte data set, implement an action on a single-analyte system based upon the process metric, and update the single-analyte data set after implementing the action on the single-analyte system.

### EXAMPLES

### Example 1: Single-Molecule Proteomic Assay

A proteomic assay is performed by a barcode-based affinity binding assay. An embodiment of the assay is depicted in FIG. 21. The assay utilizes affinity reagent binding patterns acquired through multiple cycles of affinity reagent binding to identify and/or characterize a plurality of polypeptides on a polypeptide array. In some embodiments, each polypeptide on the polypeptide array is configured to be co-located with a barcode that is extended to include an affinity reagent barcode during each cycle in which an affinity reagent interacts with the polypeptide.

Single-Analyte System: The barcode-based affinity binding assay is implemented on a single-analyte system including a polypeptide array disposed within a removable flow cell. The flow cell included a plurality of fluidic ports and channels that permit fluidic communication between the polypeptide array within the flow cell and a fluidics system. The fluidics system comprises an upstream section and a downstream section, with both sections including connecting tubing, valves, pumping devices, and a network of sensors (e.g., flow sensors, pressure sensors, temperature sensors). The fluidics system provides fluidic communication between a plurality of reagent reservoirs including fluids for various processes, including rinsing, affinity reagent binding, affinity reagent removal, and barcode extension reactions. The fluidics system also provides fluidic communication to a downstream next-generation sequencing (NGS) cartridge. The removable flow cell is disposed within a stationary flow cell holder that forms secure fluidic connections between the fluidic system and the flow cell, and includes a Pelletier thermocycling device that allows the temperature within the flow cell to be altered. Opposed to one surface of the flow cell is a laser and optical lens system that is configured to release nucleic acid barcodes from selected addresses via the cleaving of photolabile linkers. The flow cell includes the polypeptide array disposed within a main fluidic chamber, as well as a secondary polypeptide array disposed within a second fluidic chamber that is fluidically isolated from the main fluidic chamber. The secondary polypeptide array is configured to include a second patterned array with a plurality of polypeptide binding sites, for example to include control or standard polypeptides, or a replicate polypeptide sample compared to the main polypeptide array. The single-analyte system is integrated by a process control system including a processor and a process control algorithm that is in communication with the network of sensors and provides actuation to a plurality of system components, including fluidic pumps, fluidic valves, the laser and optical components, and the NGS cartridge. The single-analyte further comprises a communication network that is configured to send and receive data from a user-controlled device including a processor (e.g., a tablet or a desktop computer) and a server including a plurality of processors (e.g., a cloud server). The user-controlled device and/or the server include one or more algorithms that are configured to implement the barcode-based affinity binding assay.

Process Outcomes and Process Metrics: The barcode-based binding single-analyte system is configured to perform various analyses, including polypeptide identification, polypeptide proteoform identification, polypeptide quantification, and polypeptide proteoform quantification. Identification includes determining an identity of a determinable polypeptide and/or proteoform present on the polypeptide array. Quantification includes determining a tabulated count of one or more identified polypeptides and/or proteoforms present on the polypeptide array. Each polypeptide identity is automatically configured to be obtained when the confidence level of the identification exceeds 99.99999%. In some embodiments, a human user specifies a barcode-binding assay to achieve a specific analysis, such as identifying or quantifying the presence of a certain polypeptide, or identifying and/or quantifying as many polypeptides from a sample including a polypeptide as possible. The chosen analysis automatically defines an outcome for the barcode-based binding assay. In the case where an assay is configured to quantify the presence of a single type of polypeptide from a possibly heterogeneous mixture of polypeptides, the assay has a primary defined outcome of achieving an identification of at least 60% of the polypeptides on the polypeptide array, and a secondary defined outcome of achieving proper barcode extension on 90% of possible extension reactions, with a targeted outcome of achieving proper barcode extension on 99% of possible extension reactions. Based upon the established outcomes, the most relevant process metrics for the assay are affinity reagent concentration, affinity reagent quantity, affinity reagent binding time, affinity reagent binding temperature, polymerase concentration, polymerase extension time, polymerase extension temperature, NGS sequence read error rate, and polypeptide identity count. Additionally, relevant uncertainty metrics include flow cell temperature spatial variance, flow cell temperature temporal variance, and Q score.

Overall Process Structure: A removable flow cell is added to the flow cell holder. The flow cell undergoes a sequence of processes to deposit a polypeptide array within the flow cell, including pre-deposition rinsing, passivation of non-specific binding sites, deposition of sample polypeptides on a patterned array, and co-localization of a nucleic acid barcode including a photolabile linker at each site where a polypeptide is bound to the array. Simultaneous to the deposition of the polypeptide array, a control array is formed in a secondary fluidic chamber of the flow cell via the same process as the main polypeptide array. The control array includes a homogeneous array of a known and characterized polypeptide to serve as an internal standard for cycle-by-cycle process success. After both arrays are formed, the single-analyte system is configured to automatically perform two test rounds of affinity reagent binding of a standard affinity reagent on the control array, with each round including a polymerase extension reaction to capture the binding of the standard affinity reagent to the control polypeptides by a barcode extension reaction. After the two rounds of affinity reagent binding on the control array, a small portion of the control array is irradiated by the laser optical system to release barcodes from this portion of the array. The released barcodes are fluidically transferred to the NGS cartridge for sequencing to confirm the success of the two test rounds. After confirming the proper function on the fluidics and the NGS system, a preliminary single-analyte data set is read to obtain user-supplied information on the sample source. Based upon the sample source, the assay control algorithm calls up a second single-analyte data set including cumulative data on prior assay structure for the same sample type. The cumulative data is utilized to provide a sequence of affinity reagent binding cycles for identifying the polypeptides on the polypeptide array. After determining a sequence of affinity reagent binding cycles for the polypeptide array, an iterative process is initiated.

Configuring Actions: Based upon the specified outcomes and the available process metrics, actions are configured for the barcode-based affinity binding assay. In some embodiments, such as for the case of quantification of a single polypeptide within a possibly heterogeneous polypeptide mixture, the above-described outcomes are utilized to automatically configure a set of computer-implemented actions that is implemented during an iterative process. The actions utilize process metrics determined from single-molecule polypeptide data sets to establish rules for when to select and implement an action. Table V lists outcomes, relevant process metrics, and process metric rules for achieving the polypeptide quantification. Table VI lists process metric rules, actions, and action procedures for achieving the polypeptide quantification. For example, in some embodiments, based upon the rule that the NGS sequence read error rate must be no more than a threshold value of 0.1%, the single-analyte system is configured to implement an action to pause an assay if the NGS sequence read error rate exceeds the threshold value. The pausing action further includes procedures to divert flow of nucleic acids from a first NGS cartridge to a second NGS cartridge, and to release a set of control nucleic acid barcodes to the second NGS cartridge. In some embodiments, if the NGS sequence read error rate for the second cartridge falls beneath the threshold value of 0.1%, the assay is resumed.

Performing the Assay: A barcode-based binding assay is initiated on a single-molecule assay system. A human user places a sample vessel comprising a prepared polypeptide sample for analysis in the system. The system scans a QR code on the sample vessel and retrieves sample information from a database including sample data. The sample data, including sample source, sample collection information, sample storage history, and sample preparation information, is added to an assay data set for the barcode-based binding assay. The user specifies the desired analysis of the polypeptide sample through a software user interface and then instructs the system to initiate the assay. The algorithm extracts the sample type and assay specification from the assay data set and calls to a second data set of cumulative data that includes stored assay sequences from prior assay runs. The algorithm defines a preliminary sequence of steps for the assay utilizing the cumulative data set, including two cycles of performance testing on a control array, and a preliminary sequence of affinity binding measurements that are estimated to achieve the user-specified analysis based upon the cumulative data. The sample polypeptides are drawn from the sample vessel into the fluidics system and deposited on a patterned array within a flow cell. After forming the polypeptide array, the initial performance testing is performed on the control array. Once proper function of the system has been confirmed, an iterative process is initiated, and the pre-defined sequence of affinity binding measurements is started.

During the fifth cycle of affinity reagent binding, the system control algorithm extracts the polymerase extension temperature data and determines that the temperature has exceeded the normal range during the cycle. The control algorithm implements an action to pause the assay and call to the control array. A subset of polypeptides on the control array are released to the NGS cartridge for sequencing to determine the success rate of the extension reaction for the cycle. Based upon the sequencing data from the NGS cartridge, the control algorithm determines that only 98% of extension reactions were completed during the cycle. The assay control algorithm reconfigures the assay sequence to include an additional cycle of affinity reagent binding utilizing the same affinity reagent as used during the fifth cycle. After the completion of the assay, the binding measurement data is analyzed with and without the re-measured binding data. It is determined from the re-analysis that without repeating the fifth cycle, 20% of determined polypeptide identities would not have attained the minimum identification confidence level at the completion of the assay.

TABLE V

| | | Process Metric | |
|---|---|---|---|
| Outcome | Process Metric | Determination | Process Metric Rule |
| Identify 60% of | Affinity reagent | Fluidic sensors | Affinity reagent |
| polypeptides on the | concentration | | dependent; ±1% of |
| polypeptide array | | | defined value for |
| | | | particular affinity |
| | | | reagent |
| | Affinity reagent | Fluidic sensors | Affinity reagent |
| | quantity | | dependent; ±1% of |
| | | | defined value for |
| | | | particular affinity |
| | | | reagent |
| | Affinity reagent | Algorithm timer | Affinity reagent |
| | binding time | | dependent; 1 sec < t < |
| | | | 5 min |
| | Affinity reagent | IR camera | 24° C. ≤ Average |
| | binding temperature | | temperature ≤ 26° C. |
| | Control polypeptide | Algorithm-based | Total count ≥ 80% |
| | identity count | computation | occupied control |
| | | | array sites |
| | Flow cell temperature | Algorithm-based | ≤0.3 (° C.)^{∧}2 |
| | spatial variance | computation | |
| | Flow cell temperature | Algorithm-based | ≤0.5 (° C.)^{∧}2 |
| | temporal variance | computation | |
| | Q score | Algorithm-based | Q score ≥ 30 |
| | | computation | |
| Achieve barcode | Affinity reagent | Algorithm timer | Affinity reagent |
| extension on 90% of | binding time | | dependent; 1 sec < t < |
| possible extension | | | 5 min |
| reactions | | | |
| | Affinity reagent | IR camera | 24° C. ≤ Average |
| | binding temperature | | temperature ≤ 26° C. |
| | Polymerase | Fluidic sensors | 1.5-2.5 units per |
| | concentration | | cycle |
| | Polymerase extension | Algorithm timer | 15 sec < t < 30 sec |
| | time | | |
| | Polymerase extension | IR camera | 68° C. ± 1° C. |
| | temperature | | |
| | Flow cell temperature | Algorithm-based | ≤0.3 (° C.)^{∧}2 |
| | spatial variance | computation | |
| | Flow cell temperature | Algorithm-based | ≤0.5 (° C.)^{∧}2 |
| | temporal variance | computation | |

TABLE VI

| Process Metric | Observed Condition | Action | Action Procedures |
|---|---|---|---|
| Affinity reagent | Concentration outside | Pause the assay | Pause the assay; |
| concentration | of normal range for | | remove bound |
| | particular affinity | | affinity reagents; |
| | reagent | | discharge flow cell; |
| | | | rinse flow cell; |
| | | | recharge flow at |
| | | | proper concentration |
| | Concentration in | Continue assay | — |
| | normal range | | |
| Affinity reagent | Quantity outside of | Pause the assay | Pause the assay; |
| quantity | defined value for | | remove bound |
| | particular affinity | | affinity reagents; |
| | reagent | | discharge flow cell; |
| | | | rinse flow cell; |
| | | | recharge flow at |
| | | | proper concentration |
| | Quantity in normal | Continue assay | — |
| | range | | |
| Affinity reagent | Binding time outside | Alter assay sequence | Complete cycle; |
| binding time | normal range | | record cycle data in |
| | | | assay data set; |
| | | | reperform cycle; |
| | | | record cycle data in |
| | | | assay data set |
| | | Call to second | Perform cycle on |
| | | analyte | control array; |
| | | | reperform cycle on |
| | | | control array; record |
| | | | cycle data in assay |
| | | | data set |
| | Binding time in | Continue assay | — |
| | normal range | | |
| Affinity reagent | Binding temperature | Alter assay sequence | Complete cycle; |
| binding | outside of normal | | record cycle data in |
| temperature | range | | assay data set; |
| | | | reperform cycle; |
| | | | record cycle data in |
| | | | assay data set |
| | | Call to second | Perform cycle on |
| | | analyte | control array; |
| | | | reperform cycle on |
| | | | control array; record |
| | | | cycle data in assay |
| | | | data set |
| | Binding temperature | Continue assay | — |
| | in normal range | | |
| Control polypeptide | <80% of occupied | If performing a pre- | Release portion of |
| identity count | control array sites | defined sequence: | control array to NGS |
| | identified | continue assay | cartridge; determine |
| | | | % of control sites |
| | | | with identifiable |
| | | | polypeptides; |
| | | | continue binding |
| | | | cycles if <80% |
| | | If a pre-defined | Release portion of |
| | | sequence is complete: | control array to NGS |
| | | alter the assay | cartridge; determine |
| | | sequence | % of control sites |
| | | | with identifiable |
| | | | polypeptides; |
| | | | configure additional |
| | | | sequence of binding |
| | | | cycles if <80% |
| | ≥80% of occupied | Continue assay | Continue assay; |
| | control array sites | | proceed to |
| | identified | | sequencing of |
| | | | polypeptide array |
| | | | barcodes |
| Flow cell | Flow cell temperature | Pause the assay | Pause the assay; |
| temperature spatial | variance outside of | | check function of |
| variance | normal range | | Pelletier device; if |
| | | | improper, discontinue |
| | | | assay; if device is |
| | | | functional, stabilize |
| | | | temperature and |
| | | | resume assay |
| | Flow cell temperature | Continue assay | — |
| | variance in normal | | |
| | range | | |
| Flow cell | Flow cell temperature | Pause the assay | Pause the assay; |
| temperature | variance outside of | | check function of |
| temporal variance | normal range | | Pelletier device; if |
| | | | improper, discontinue |
| | | | assay; if device is |
| | | | functional, stabilize |
| | | | temperature and |
| | | | resume assay |
| | Flow cell temperature | Continue assay | — |
| | variance in normal | | |
| | range | | |
| Polymerase | Concentration outside | Call to second | Release portion of |
| concentration | of normal range for | analyte | control array to NGS |
| | particular affinity | | cartridge; determine |
| | reagent | | % of control sites |
| | | | with completed |
| | | | extension reactions; if |
| | | | >99.9%, then |
| | | | continue assay; if not, |
| | | | alter assay sequence |
| | | Alter assay sequence | Complete cycle; |
| | | | record cycle data in |
| | | | assay data set; |
| | | | reperform cycle; |
| | | | record cycle data in |
| | | | assay data set |
| | Concentration in | Continue assay | — |
| | normal range | | |
| Polymerase | Extension time | Call to second | Release portion of |
| extension time | outside of normal | analyte | control array to NGS |
| | range | | cartridge; determine |
| | | | % of control sites |
| | | | with completed |
| | | | extension reactions; if |
| | | | >99.9%, then |
| | | | continue assay; if not, |
| | | | alter assay sequence |
| | | Alter assay sequence | Complete cycle; |
| | | | record cycle data in |
| | | | assay data set; |
| | | | reperform cycle; |
| | | | record cycle data in |
| | | | assay data set |
| | Extension time in | Continue assay | — |
| | normal range | | |
| Polymerase | Extension | Call to second | Release portion of |
| extension | temperature outside | analyte | control array to NGS |
| temperature | of normal range | | cartridge; determine |
| | | | % of control sites |
| | | | with completed |
| | | | extension reactions; if |
| | | | >99.9%, then |
| | | | continue assay; if not, |
| | | | alter assay sequence |
| | | Alter assay sequence | Complete cycle; |
| | | | record cycle data in |
| | | | assay data set; |
| | | | reperform cycle; |
| | | | record cycle data in |
| | | | assay data set |
| | Extension | Continue assay | — |
| | temperature in | | |
| | normal range | | |
| NGS sequence Q | Q score < 30 | Pause the assay | Pause the assay; |
| score | | | divert released |
| | | | barcodes to second |
| | | | NGS cartridge; alter |
| | | | assay sequence |
| | | Alter assay sequence | Determine cycles |
| | | | affected by NGS |
| | | | error rate; reconfigure |
| | | | assay sequence to |
| | | | repeat affected |
| | | | cycles; reperform |
| | | | affected cycles; |
| | | | record all |
| | | | reperformed cycles in |
| | | | assay data set |
| | Q score ≥ 30 | Continue assay | — |

### Example 2: Single-Molecule Proteomic Assay

A proteomic assay is performed by a fluorosequencing assay. An embodiment of the assay is depicted in FIG. 22. The assay utilizes cycles of fluorescence measurement and terminal amino acid degradation to iteratively determine the amino acid sequence of each polypeptide on a polypeptide array. Each polypeptide on the polypeptide array is configured to located at an optically-resolvable address that permits a unique single-molecule fluorescence measurement to be obtained for each polypeptide.

Single-Analyte System: The fluorosequencing assay is implemented on a single-analyte system including a polypeptide array disposed within a removable flow cell. The flow cell includes a plurality of fluidic ports and channels that permit fluidic communication between the polypeptide array within the flow cell and a fluidics system. The fluidics system comprises an upstream section and a downstream section, with both sections including connecting tubing, valves, pumping devices, and a network of sensors (e.g., flow sensors, pressure sensors, temperature sensors). The fluidics system provides fluidic communication between a plurality of reagent reservoirs including fluids for various processes, including rinsing, imaging, Edman-type terminal amino acid activation, and terminal amino acid removal. The removable flow cell is disposed within a stationary flow cell holder that forms secure fluidic connections between the fluidic system and the flow cell, and includes a Pelletier thermocycling device that allows the temperature within the flow cell to be altered. Opposed to one surface of the flow cell is a detection device including a laser, optical lens system, and sensor that is configured to provide an exciting radiation to the polypeptide array and detect emitted fluorescent radiation. The flow cell includes the polypeptide array disposed within a main fluidic chamber, as well as a secondary polypeptide array disposed within a second fluidic chamber that is fluidically isolated from the main fluidic chamber. The secondary polypeptide array is configured to include a second patterned array with a plurality of polypeptide binding sites, for example to include control or standard polypeptides, or a replicate polypeptide sample compared to the main polypeptide array. The single-analyte system is integrated by a process control system including a processor and a process control algorithm that is in communication with the network of sensors and provides actuation to a plurality of system components, including fluidic pumps, fluidic valves, and the laser and optical components. The single-analyte further comprises a communication network that is configured to send and receive data from a user-controlled device including a processor (e.g., a tablet or a desktop computer) and a server including a plurality of processors (e.g., a cloud server). The user-controlled device and/or the server includes one or more algorithms that are configured to implement the polypeptide fluorosequencing assay.

Process Outcomes and Process Metrics: The fluorosequencing single-analyte system is configured to perform various analyses, including polypeptide identification and polypeptide quantification. Identification includes determining an identity of a determinable polypeptide and/or proteoform present on the polypeptide array. Quantification includes determining a tabulated count of one or more identified polypeptides and/or proteoforms present on the polypeptide array. Each polypeptide identity is automatically configured to be obtained when the confidence level of the identification exceeds 99.99999%. In some embodiments, a human user specifies a fluorosequencing assay to achieve a specific analysis, such as quantifying all identifiable polypeptides from a polypeptide sample. The chosen analysis automatically defines an outcome for the fluorosequencing assay. In the case where an assay is configured to identify unknown polypeptides from a sample, the assay has a primary defined outcome of achieving an identification of at least 90% of the polypeptides in the polypeptide sample, and a secondary defined outcome of obtaining sequence reads on 90% of fluorescently-labeled amino acids at a sequence read confidence level of 99.9%. Based upon the established outcomes, the most relevant process metrics for the assay are activation reagent concentration, activation temperature, cleavage reagent concentration, cleavage temperature, observed flow cell autofluorescence, and polypeptide complete sequence count. Additionally, relevant uncertainty metrics include flow cell autofluorescence spatial variance, flow cell autofluorescence temporal variance, amino acid calling error probability, and sequence alignment score.

Overall Process Structure: Prior to performing a fluorosequencing assay, a polypeptide sample is treated with a set of sidechain reactive fluorescent dyes that differentially label cysteine, lysine, tyrosine, and tryptophan amino acid residues. A removable flow cell is added to the flow cell holder. A background fluorescence measurement of the flow cell and patterned array is collected before deposition of polypeptides to determine the baseline fluorescence at each address on the array. Background fluorescence measurements in the four wavelength channels corresponding to the four labeled amino acids are used to populate a single-analyte data set. The flow cell undergoes a sequence of processes to deposit a polypeptide array within the flow cell, including pre-deposition rinsing, passivation of non-specific binding sites, deposition of labeled polypeptides on a patterned array, and post-deposition determination of each occupied site on the array. Simultaneous to the deposition of the polypeptide array, a control array is formed in a secondary fluidic chamber of the flow cell via the same process as the main polypeptide array. The control array includes a heterogeneous array of a known and characterized polypeptides to serve as an internal standard for cycle-by-cycle process success. After confirming the proper function of the fluidics system, a preliminary single-analyte data set is obtained by providing an exciting radiation field to the polypeptide array and the control array, then observing emitted fluorescent radiation at each address on the array. The preliminary fluorescence of each address on the array is read in four wavelength channels corresponding to the four labeled amino acids present in each polypeptide and the data is added to a single-molecule fluorosequencing data set. After collecting the initial fluorescence data for each address on the polypeptide array and control array, an iterative process is initiated to control the cyclical degradation fluorosequencing process.

Configuring Actions: Based upon the specified outcomes and the available process metrics, actions are configured for the polypeptide fluorosequencing assay. In some embodiments, such as for the case of identifying polypeptides within a possibly heterogeneous polypeptide mixture, the above-described outcomes are utilized to automatically configure a set of computer-implemented actions that is implemented during an iterative process. The actions utilize process metrics determined from single-molecule polypeptide data sets to establish rules for when to select and implement an action. Table VII lists outcomes, relevant process metrics, and process metric rules for achieving the polypeptide quantification. Table VIII lists process metric rules, actions, and action procedures for achieving the polypeptide identification. For example, in some embodiments, an uncertainty metric of flow cell background fluorescence spatial variance is calculated to provide a measure of spatial changes in the background fluorescence. In some embodiments, if the background fluorescence spatial variance is observed to increase, the assay is paused to determine a source of the increasing spatial variability of background fluorescence. In some embodiments, if possible, the variability is addressed (e.g., photobleaching regions of increased fluorophore non-specific binding), before the assay is resumed. In some embodiments, if the source of background fluorescence spatial variability is addressed, addresses of increased background fluorescence are identified and excluded from further analysis.

Performing the Assay: A fluorosequencing assay is initiated on a single-molecule fluorosequencing system. A human user obtains a sample comprising polypeptides and places the sample in an automated sample preparation instrument. A user inputs sample information into a fluorosequencing assay control algorithm interface that is transferred to a single-polypeptide data set, and the sample preparation instrument also transfers sample preparation data to the single-polypeptide data set for the fluorosequencing assay. After sample preparation is complete, the labeled polypeptide sample is transferred by a robotic pumping system from the sample preparation instrument to the single-polypeptide fluorosequencing assay system. The polypeptide sample is deposited on the patterned array within the flow cell and an initial set of fluorescence measurements is recorded in the single-polypeptide data set for all four wavelength channels at each address on the polypeptide array and the control array. The algorithm configures a sequence of degradation cycles and an iterative process is initiated.

The sequence of degradation cycles is continued without any determined need to deviate from the sequence until a pre-programmed pause after the tenth cycle. The tenth set of fluorescence measurements is compared to the background fluorescence measurements collected before polypeptide deposition at each array address to determine if any detectable amount of fluorescence remains at each array address. Each array address is assigned an assay completion process metric value of “COMPLETE” or “INCOMPLETE” based on the absence or presence of detected fluorescence, respectively. The assay completion process metric values are compiled in a total assay completion curated process metric that is calculated as the percentage of all array addresses with a value of “COMPLETE.” The total assay completion curated process metric is calculated as 13% after the tenth degradation cycle, and the curated process metric value is added to the single-polypeptide data set. The assay is continued one cycle at a time and the total assay completion process metric is recalculated after each cycle. After eighteen cycles, the total assay completion process metric indicates that greater than 99.9999% of array addresses have returned to the background level of fluorescence. The assay is automatically discontinued and assay sequence results are compiled in the single-polypeptide data set. The single-polypeptide data set is provided to a polypeptide identification algorithm that infers the identities of polypeptides present in the sample based upon the observed polypeptide sequence at each array address. In some embodiments, after polypeptide identification, 95% of array addresses produce an amino acid sequence that was identified as deriving from a known polypeptide, thereby achieving the primary defined outcome for the fluorosequencing assay.

TABLE VII

| | | Process Metric | |
|---|---|---|---|
| Outcome | Process Metric | Determination | Process Metric Rule |
| Identify 90% of | Observed flow cell | Optical microscopy | Average <5% of |
| polypeptides from a | autofluorescence | | maximum pixel |
| polypeptide sample | | | intensity for each |
| | | | imaging region |
| | Flow cell | Algorithm-based | <(50 intensity |
| | autofluorescence | computation | counts/μm^{∧}2)^{∧}2 |
| | spatial variance | | |
| | Flow cell | Algorithm-based | <0.5% change per |
| | autofluorescence | computation | degradation cycle |
| | temporal standard | | |
| | deviation | | |
| | Polypeptide complete | Algorithm-based | >99% of sites |
| | sequence count | computation | completely |
| | | | sequenced |
| | Amino acid calling | Algorithm-based | ≤1 in 1000 |
| | error probability | computation | |
| | Sequence alignment | Algorithm-based | >0.9 |
| | score | computation | |
| Obtain sequence | Activation reagent | Fluidic sensors | Reagent dependent; |
| reads on 90% of | concentration | | ±1% of defined value |
| amino acids at a | | | for particular |
| 99.9% confidence | | | activation reagent |
| level | Activation | IR camera | 30° C. ± 1° C. |
| | temperature | | |
| | Cleavage reagent | Fluidic sensors | Reagent dependent; |
| | concentration | | ±1% of defined value |
| | | | for particular |
| | | | cleavage reagent |
| | Cleavage temperature | IR camera | 30° C. ± 1° C. |
| | Amino acid calling | Algorithm-based | ≤1 in 1000 |
| | error probability | computation | |
| | Sequence alignment | Algorithm-based | >0.9 |
| | score | computation | |

TABLE VIII

| Process Metric | Observed Condition | Action | Action Procedures |
|---|---|---|---|
| Observed flow cell | Observed flow cell | Pause the assay | Pause the assay; |
| autofluorescence | autofluorescence | | determine source of |
| | above the maximum | | autofluorescence; if |
| | threshold value | | resolvable, address |
| | | | then unpause the |
| | | | assay |
| | Observed flow cell | Continue the assay | — |
| | autofluorescence | | |
| | below the maximum | | |
| | threshold value | | |
| Flow cell | Flow cell | Pause the assay | Pause the assay; |
| autofluorescence | autofluorescence | | determine source of |
| spatial variance | spatial variance | | autofluorescence; if |
| | outside normal range | | localized, mark |
| | | | affected addresses for |
| | | | exclusion from data |
| | | | analysis |
| | Flow cell | Continue the assay | — |
| | autofluorescence | | |
| | spatial variance | | |
| | within normal range | | |
| Flow cell | Flow cell | Pause the assay | Pause the assay; |
| autofluorescence | autofluorescence | | determine source of |
| temporal standard | temporal variance | | autofluorescence; if |
| deviation | outside normal range | | localized, mark |
| | | | affected addresses for |
| | | | exclusion from data |
| | | | analysis |
| | Flow cell | Continue the assay | — |
| | autofluorescence | | |
| | temporal variance | | |
| | within normal range | | |
| Polypeptide complete | Sequence count | Pause the assay | Pause the assay; pass |
| sequence count | above minimum | | sequence data to data |
| | threshold | | analysis algorithm; |
| | | | discontinue if >90% |
| | | | of polypeptides are |
| | | | identifiable |
| | Sequence count | Continue the assay | — |
| | below minimum | | |
| | threshold | | |
| Activation reagent | Concentration outside | Pause the assay | Pause the assay; |
| concentration | of normal range for | | discharge flow cell; |
| | particular activation | | rinse flow cell; |
| | reagent | | recharge flow at |
| | | | proper concentration |
| | Concentration in | Continue the assay | — |
| | normal range | | |
| Activation | Activation | Call to second | Proceed with |
| temperature | temperature outside | analyte | cleavage on control |
| | of normal range | | array; if cleavage |
| | | | reaction proceeds |
| | | | normally, proceed |
| | | | with cleavage on |
| | | | sample array |
| | Binding temperature | Continue the assay | — |
| | in normal range | | |
| Cleavage temperature | Binding temperature | Alter assay sequence | Complete cycle; |
| | outside of normal | | record cycle data in |
| | range | | assay data set; check |
| | | | control array |
| | | | cleavage efficiency, |
| | | | if below normal then |
| | | | repeat cleavage |
| | | | reaction |
| | Binding temperature | Continue the assay | — |
| | in normal range | | |
| Cleavage reagent | Concentration outside | Pause the assay | Pause the assay; |
| concentration | of normal range for | | discharge flow cell; |
| | particular cleavage | | rinse flow cell; |
| | reagent | | recharge flow at |
| | | | proper concentration |
| | Concentration in | Continue the assay | — |
| | normal range | | |
| Amino acid calling | Calling error | Alter assay sequence | Reperform |
| error probability | probability above | | fluorescence |
| | maximum threshold | | measurement; |
| | | | recalculate amino |
| | | | acid calling error |
| | | | probability; continue |
| | | | repeating |
| | | | fluorescence |
| | | | measurement until |
| | | | calling error |
| | | | probability meets or |
| | | | exceeds the rule |
| | Calling error | Continue the assay | — |
| | probability below | | |
| | maximum threshold | | |
| Sequence alignment | Score above the | Alter assay sequence | Discontinue |
| score | threshold score | | sequencing |
| | | | measurements; exit |
| | | | iterative process and |
| | | | proceed to data |
| | | | analysis |
| | Score below the | Continue the assay | — |
| | threshold score | | |

### Example 3: Single-Molecule Proteomic Assay

A proteomic assay is performed by a fluorescence-lifetime binding assay. An embodiment of the assay is depicted in FIG. 23. The assay utilizes cycles of luminescently-labeled affinity reagent binding and terminal amino acid degradation to iteratively determine the amino acid sequence of each polypeptide on a polypeptide array. Each polypeptide on the polypeptide array is configured to located at an optically-resolvable address that permits a unique single-molecule fluorescence measurement to be obtained for each polypeptide.

Single-Analyte System: The fluorescence-lifetime binding assay is implemented on a single-analyte system including a polypeptide array disposed within a removable flow cell. The flow cell includes a plurality of fluidic ports and channels that permit fluidic communication between the polypeptide array within the flow cell and a fluidics system. The fluidics system comprises an upstream section and a downstream section, with both sections including connecting tubing, valves, pumping devices, and a network of sensors (e.g., flow sensors, pressure sensors, temperature sensors). The fluidics system provides fluidic communication between a plurality of reagent reservoirs including fluids for various processes, including rinsing, affinity reagent binding, imaging, Edman-type terminal amino acid activation, and terminal amino acid removal. The removable flow cell is disposed within a stationary flow cell holder that forms secure fluidic connections between the fluidic system and the flow cell, and includes a Pelletier thermocycling device that allows the temperature within the flow cell to be altered. Opposed to one surface of the flow cell is a detection device including a laser, optical lens system, and sensor that is configured to provide an exciting radiation to the polypeptide array and detect emitted fluorescent radiation. The flow cell includes the polypeptide array disposed within a first fluidic chamber, as well as a secondary polypeptide array disposed within a second fluidic chamber that is fluidically isolated from the first fluidic chamber. The secondary polypeptide array is configured to include a second patterned array with a plurality of polypeptide binding sites, for example to include control, standard polypeptides, replicate, or duplicate polypeptides compared to the first polypeptide array. The single-analyte system is integrated by a process control system including a processor and a process control algorithm that is in communication with the network of sensors and provides actuation to a plurality of system components, including fluidic pumps, fluidic valves, and the laser and optical components. The single-analyte system further comprises a communication network that is configured to send and receive data from a user-controlled device including a processor (e.g., a tablet or a desktop computer) and a server including a plurality of processors (e.g., a cloud server). The user-controlled device and/or the server includes one or more algorithms that are configured to implement the fluorescence lifetime binding assay.

Process Outcomes and Process Metrics: The fluorescence lifetime single-analyte system is configured to perform various analyses, including polypeptide identification and polypeptide quantification. Identification includes determining an identity of a determinable polypeptide and/or proteoform present on the polypeptide array. Quantification includes determining a tabulated count of one or more identified polypeptides and/or proteoforms present on the polypeptide array. Each polypeptide identity is configured to be obtained when the confidence level of the identification exceeds a value that is input by a user of the fluorescence lifetime assay system. In some embodiments, a human user specifies a fluorescence lifetime assay to achieve a specific analysis, such as quantifying all identifiable polypeptides from a polypeptide sample. The user-chosen analysis automatically defines an outcome for the fluorosequencing assay. In the case where an assay is configured to identify unknown polypeptides from a sample, the assay has a primary defined outcome of achieving an identification of at least 90% of the polypeptides in the polypeptide sample, and a secondary defined outcome of obtaining sequence reads on 90% of amino acids at a sequence read confidence level of 99.9%. Based upon the established outcomes, the most relevant process metrics for the assay are affinity reagent concentration, affinity reagent binding time, affinity reagent binding temperature, observed flow cell autofluorescence, fluorescence average signal-to-noise ratio, and polypeptide complete sequence count. Additionally, relevant uncertainty metrics include flow cell autofluorescence spatial variance, flow cell autofluorescence standard deviation, amino acid calling error probability, and sequence alignment score.

Overall Process Structure: In a separate instrument, a mixture of polypeptides is degraded into peptides of 10-20 amino acids in length by enzymatic digestion. A homogeneous peptide standard is injected into the digested peptides. The homogeneous peptide standard includes an engineered peptide including a sequence of fluorescently-labeled, non-natural amino acids that are configured to not be bound by affinity reagents of the binding assay. The peptide mixture, including the standard peptides, is purified and captured to provide a peptide sample. A removable flow cell is added to the flow cell holder. The flow cell undergoes a sequence of processes to deposit a polypeptide array within the flow cell, including pre-deposition rinsing, passivation of non-specific binding sites, and deposition of peptide sample on the first patterned array. A duplicate sample split off from the peptide sample is deposited on the second patterned array to form two isolated arrays including polypeptides from the same sample. An iterative process is initiated once the polypeptide arrays are prepared. Each cycle of the iterative process is utilized to select and configure the next step of the assay for each array. In some embodiments, an Edman-type degradation process for terminal amino acids is only initiated when 99.9999% of the array addresses have had at least two agreeing observed affinity reagent binding events. The observed affinity reagent bindings events are determined by measuring a fluorescence lifetime signal at each array address. The system is configured to utilized 20 different affinity reagents, each having a uniquely resolvable fluorescence lifetime signal. The iterative process repeats affinity reagent binding steps until the condition for a degradation step is met, then performs the degradation before resuming affinity reagent binding measurements.

Configuring Actions: Based upon the specified outcomes and the available process metrics, actions are configured for the lifetime fluorescence measurement binding assay. In some embodiments, such as for the case of identifying polypeptides within a possibly heterogeneous polypeptide mixture, the above-described outcomes are utilized to automatically configure a set of computer-implemented actions that is implemented during an iterative process. The actions utilize process metrics determined from single-molecule polypeptide data sets to establish rules for when to select and implement an action. Table IX lists outcomes, relevant process metrics, and process metric rules for achieving the polypeptide quantification. Table X lists process metric rules, actions, and action procedures for achieving the polypeptide identification. For example, in some embodiments, if the affinity reagent binding temperature is outside of the normal range, an iterative process reconfigured the assay sequence to include an additional binding measurement for the same affinity reagent at the specified temperature. In some embodiments, the iterative process obtains data from a control second analyte to assess the likelihood that the anomalous binding temperature affected the results.

Performing the Assay: A lifetime fluorescence binding assay is initiated on a single-molecule detection system. A human user obtains a sample comprising polypeptides and places the sample in an automated sample preparation instrument. A user inputs sample information into a fluorosequencing assay control algorithm interface that is transferred to a single-polypeptide data set, and the sample preparation instrument also transfers sample preparation data to the single-polypeptide data set for the lifetime fluorescence binding assay. After sample preparation is complete, the peptides derived from the polypeptide sample are transferred by a robotic pumping system from the sample preparation instrument to the single-polypeptide fluorescence lifetime binding assay system. The peptides are divided into two fractions and simultaneously deposited on the first and second patterned arrays within the flow cell. An initial fluorescence lifetime measurement is performed and the fluorescence lifetime signals from each array address on both arrays is transferred to a data analysis algorithm on a remote server. The data analysis algorithm analyzes the fluorescence lifetime signature at each array address to determine if the signal indicates the presence or absence of the standard peptide. The data analysis results including initial identities (sample or standard) for each array address are added to a single-polypeptide data set for the assay. An iterative process is initiated, and step-wise binding measurements are begun. Each cycle of the iterative process includes two or more affinity reagent binding fluorescence lifetime measurements and a terminal amino acid degradation. Each affinity reagent binding measurement is stored within a first single-polypeptide data set including the raw measurement data. After two affinity reagent binding measurements have been collected, the fluorescence lifetime measurement data is exported to the data analysis algorithm. The data analysis algorithm determines a measurement confidence score for each address on the array and then tabulates the percentage of addresses with a sufficient confidence score to identify the terminal amino acid. In some embodiments, if the percentage of addresses with an identified terminal amino acid is not greater than 90%, the data analysis algorithm instructs the single-molecule fluorescence lifetime binding assay system to perform an additional round of affinity reagent measurements. After each round, the additional fluorescence lifetime measurement data is added to the first single-polypeptide data set and the data is returned to the data analysis algorithm. Once measurement confidence scores have been achieved for a sufficient number of array addresses, the data analysis algorithm records the preliminary identification and measurement confidence score for each array address in a second single-polypeptide data set, and instructs the system to perform an Edman-type terminal amino acid degradation, then resume affinity reagent binding measurements on the new terminal amino acids. The iterative process is continued independently on each array until three consecutive fluorescence binding measurements indicate less than 0.001% of array addresses with available amino acids to bind affinity reagents.

During the single-polypeptide fluorescence lifetime binding assay, the first and second arrays are maintained at differing temperatures during the affinity reagent binding measurements. The first polypeptide array is maintained at a temperature of 24° C.±0.1° C. during the affinity reagent binding and fluorescence lifetime measurements, and the second polypeptide array is maintained at a temperature of 26° C.±0.1° C. Due to the difference in binding conditions between the polypeptide arrays, the single-polypeptide fluorescence lifetime assays achieve completion after a differing number of processes. The lower temperature array is found to require fewer binding measurements over the course of the assay, resulting in a shorter elapsed assay process time. However, the data analysis of the inferred peptide amino acid sequences from the lower temperature array are found to produce lower confidence level polypeptide identifications, and the lower temperature array is determined to not meet the targeted outcome of identifying 90% of polypeptides from the polypeptide sample. After completion of the single-polypeptide fluorescence lifetime binding assay, a cumulative data set is updated to include the raw measurement data from the single-polypeptide data set and the temperature effect data. A subsequent single-polypeptide fluorescence lifetime binding assay is performed on a polypeptide sample from the same source as the original assay. At the initiation of the subsequent assay, the cumulative data is recalled from the cumulative data set, and the subsequent assay is configured to perform affinity reagent binding measurements at 26° C.

TABLE IX

| | | Process Metric | |
|---|---|---|---|
| Outcome | Process Metric | Determination | Process Metric Rule |
| Identify 90% of | Observed flow cell | Optical microscopy | Average <5% of |
| polypeptides from a | autofluorescence | | maximum pixel |
| polypeptide sample | | | intensity for each |
| | | | imaging region |
| | Flow cell | Algorithm-based | <(50 intensity |
| | autofluorescence | computation | counts/μm^{∧}2)^{∧}2 |
| | spatial variance | | |
| | Flow cell | Algorithm-based | <0.5% change per |
| | autofluorescence | computation | degradation cycle |
| | temporal standard | | |
| | deviation | | |
| | Polypeptide complete | Algorithm-based | >99% of sites |
| | sequence count | computation | completely |
| | | | sequenced |
| | Amino acid calling | Algorithm-based | ≤1 in 1000 |
| | error probability | computation | |
| | Sequence alignment | Algorithm-based | >0.9 |
| | score | computation | |
| Obtain sequence | Affinity reagent | Fluidic sensors | Affinity reagent |
| reads on 90% of | binding concentration | | dependent; ±1% of |
| amino acids at a | | | defined value for |
| 99.9% confidence | | | particular affinity |
| level | | | reagent |
| | Affinity reagent | Algorithm timer | Affinity reagent |
| | binding time | | dependent; 1 sec < t < |
| | | | 5 min |
| | Affinity reagent | IR camera | 24° C. ≤ Average |
| | binding temperature | | temperature ≤ 26° C. |
| | Amino acid calling | Algorithm-based | ≤1 in 1000 |
| | error probability | computation | |
| | Sequence alignment | Algorithm-based | >0.9 |
| | score | computation | |
| | Fluorescence average | Optical sensors | Average ≥ 2x |
| | signal-to-noise ratio | | background |
| | | | fluorescence |

TABLE X

| Process Metric | Observed Condition | Action | Action Procedures |
|---|---|---|---|
| Affinity reagent | Concentration outside | Pause the assay | Pause the assay; |
| concentration | of normal range for | | remove bound |
| | particular affinity | | affinity reagents; |
| | reagent | | discharge flow cell; |
| | | | rinse flow cell; |
| | | | recharge flow at |
| | | | proper concentration |
| | Concentration in | Continue assay | — |
| | normal range | | |
| Affinity reagent | Binding time outside | Alter assay sequence | Complete cycle; |
| binding time | normal range | | record cycle data in |
| | | | assay data set; |
| | | | reperform cycle; |
| | | | record cycle data in |
| | | | assay data set |
| | | Call to second | Perform cycle on |
| | | analyte | standard peptide; |
| | | | reperform cycle on |
| | | | control array; record |
| | | | cycle data in assay |
| | | | data set |
| | Binding time in | Continue assay | — |
| | normal range | | |
| Affinity reagent | Binding temperature | Alter assay sequence | Complete cycle; |
| binding | outside of normal | | record cycle data in |
| temperature | range | | assay data set; |
| | | | reperform cycle; |
| | | | record cycle data in |
| | | | assay data set |
| | | Call to second | Perform cycle on |
| | | analyte | standard peptide; |
| | | | reperform cycle on |
| | | | control array; record |
| | | | cycle data in assay |
| | | | data set |
| | Binding temperature | Continue assay | — |
| | in normal range | | |
| Observed flow cell | Observed flow cell | Pause the assay | Pause the assay; |
| autofluorescence | autofluorescence | | determine source of |
| | above the maximum | | autofluorescence; if |
| | threshold value | | resolvable, address |
| | | | then unpause the |
| | | | assay |
| | Observed flow cell | Continue the assay | — |
| | autofluorescence | | |
| | below the maximum | | |
| | threshold value | | |
| Fluorescence | Signal-to-noise ratio | Pause the assay | Pause the assay; |
| average signal-to- | below minimum | | remove bound |
| noise ratio | threshold | | affinity reagents; |
| | | | discharge flow cell; |
| | | | rinse flow cell; |
| | | | recharge flow with |
| | | | affinity reagent |
| | | Alter assay sequence | Repeat affinity |
| | | | reagent binding and |
| | | | re-calculate signal-to- |
| | | | noise ratio; repeat |
| | | | until ratio exceeds |
| | | | threshold |
| | Signal-to-noise ratio | Continue the assay | — |
| | above minimum | | |
| | threshold | | |
| Flow cell | Flow cell | Pause the assay | Pause the assay; |
| autofluorescence | autofluorescence | | determine source of |
| spatial variance | spatial variance | | autofluorescence; if |
| | outside normal range | | localized, mark |
| | | | affected addresses for |
| | | | exclusion from data |
| | | | analysis |
| | Flow cell | Continue the assay | — |
| | autofluorescence | | |
| | spatial variance | | |
| | within normal range | | |
| Flow cell | Flow cell | Pause the assay | Pause the assay; |
| autofluorescence | autofluorescence | | determine source of |
| temporal standard | temporal variance | | autofluorescence; if |
| deviation | outside normal range | | localized, mark |
| | | | affected addresses for |
| | | | exclusion from data |
| | | | analysis |
| | Flow cell | Continue the assay | — |
| | autofluorescence | | |
| | temporal variance | | |
| | within normal range | | |
| Amino acid calling | Calling error | Alter assay sequence | Reperform |
| error probability | probability above | | fluorescence |
| | maximum threshold | | measurement; |
| | | | recalculate amino |
| | | | acid calling error |
| | | | probability; continue |
| | | | repeating |
| | | | fluorescence |
| | | | measurement until |
| | | | calling error |
| | | | probability meets or |
| | | | exceeds the rule |
| | Calling error | Continue the assay | — |
| | probability below | | |
| | maximum threshold | | |
| Sequence alignment | Score above the | Alter assay sequence | Discontinue |
| score | threshold score | | sequencing |
| | | | measurements; exit |
| | | | iterative process and |
| | | | proceed to data |
| | | | analysis |
| | Score below the | Continue the assay | — |
| | threshold score | | |

### Example 4. Single-Molecule Synthesis Process

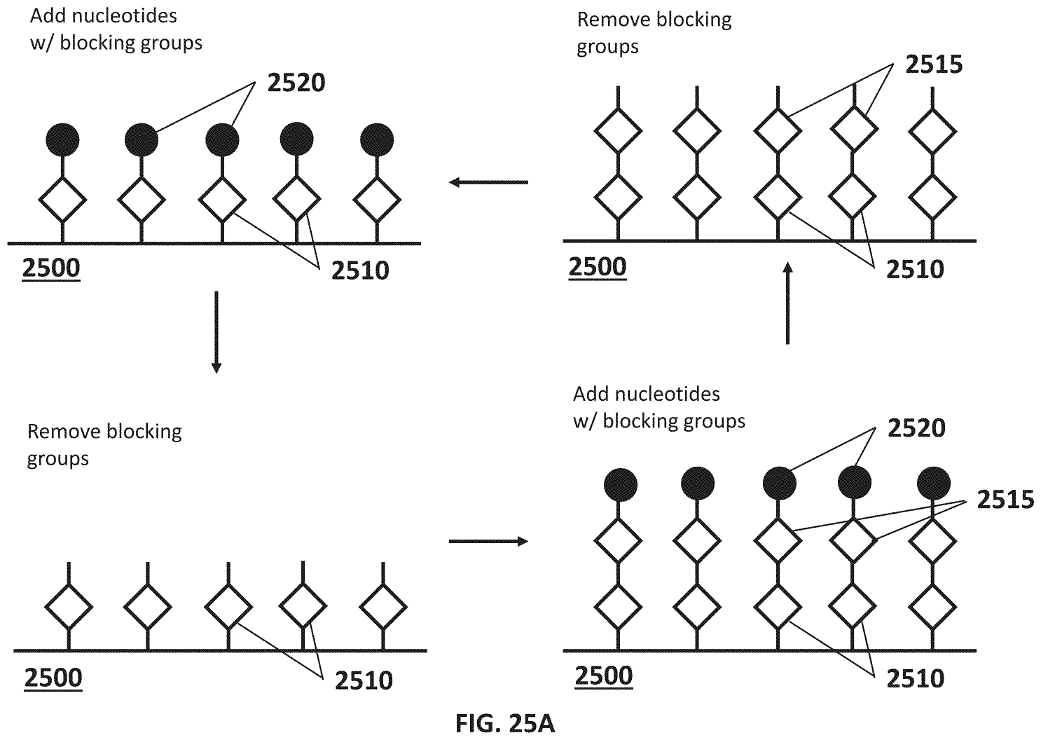

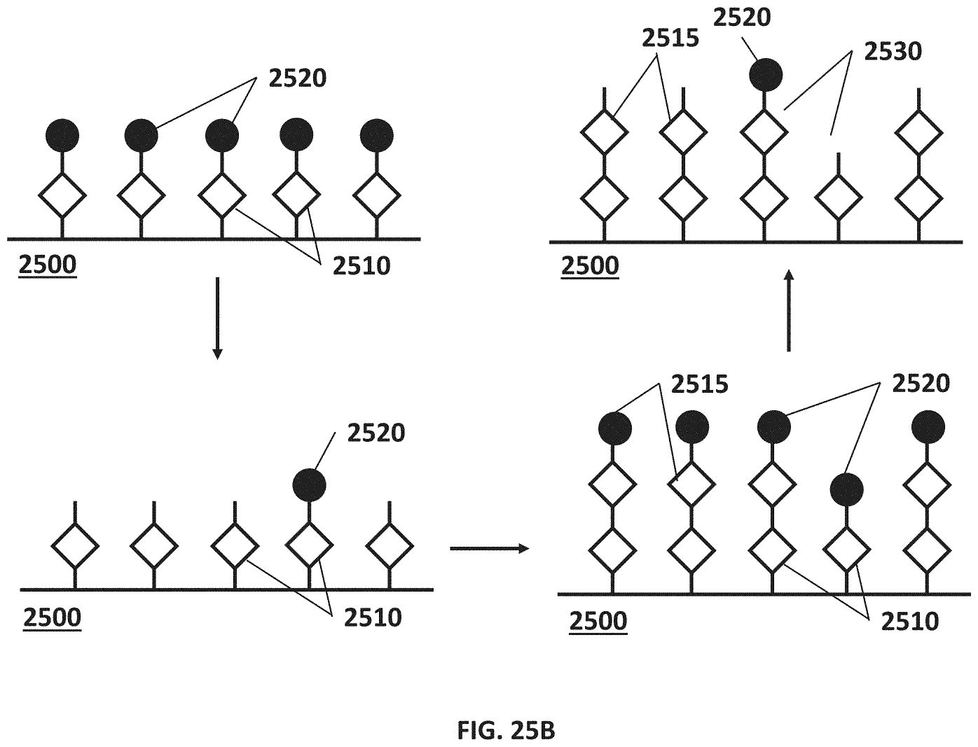

A single-molecule synthesis process is utilized to produce single-stranded oligonucleotides with a controlled nucleotide sequence. A schematic illustrating the basic process is provided in FIG. 25A. An array of oligonucleotides is formed by depositing the first nucleotide 2510 of the nucleotide sequence on a solid support 2500 at a unique, observable position on the solid support 2500 surface. Each nucleotide 2510 is provided with a fluorescent blocking group 2520. In some embodiments, an optical fluorescence measurement of the array was made to identify the presence at each site on the solid support 2500 surface of the deposited nucleotides 2510. After depositing the first nucleotide 2510 and making a fluorescence measurement, the blocking groups 2520 are removed by a cleavage reaction. The exposed first oligonucleotides 2510 are then reacted with a second oligonucleotide 2515 that is also provided with a blocking group 2520. In some embodiments, the successful conjugation of the second oligonucleotide 2515 to the first oligonucleotide 2510 was confirmed via a fluorescence measurement at each site on the array. The synthesis proceeds via cyclical nucleotide conjugation, fluorescence measurement, and blocking group removal until the oligonucleotide synthesis is complete.

The oligonucleotide synthesis process is observed to be prone to spatial variation in synthesis efficiency due to fluid stagnation and incomplete mixing, especially near edges of the array. FIG. 25B illustrates the effect of variation on process efficiency. Incomplete removal of all blocking groups 2520 from the first oligonucleotide 2510 renders some first oligonucleotides 2510 incapable of conjugating to second nucleotides 2515. In some embodiments, subsequent failure to remove blocking groups in further cycles lead to an increase in the number of oligonucleotides with synthesis errors, leading to oligonucleotides with erroneous nucleotide sequences 2530. In some embodiments, the synthesis errors increase through each cycle, leading to a significant yield of erroneous oligonucleotides by the end of the synthesis process.

An iterative process is utilized to increase the yield of oligonucleotides with accurate nucleotide sequences. A user seeking to obtain oligonucleotides inputs the desired nucleotide sequence into an internet-based interface and the request is routed to a single-molecule synthesis system that performs the synthesis. The requested nucleotide sequence is utilized to configure a pre-determined sequence of steps for the synthesis process, including cycles of nucleotide conjugation, unused nucleotide removal, fluorescent measurement of conjugated nucleotides, removal of blocking groups, fluorescent measurement of removed blocking groups, and post-cycle rinsing. The iterative process is configured to collect fluorescent measurements for each unique oligonucleotide and store them in a single-analyte data set. The fluorescent measurements are provided to a data analysis algorithm that converts the measured fluorescence intensities at each spatial address including an oligonucleotide into inferred likelihood of successful nucleotide conjugation (during the conjugation step) or inferred likelihood of blocking group removal (during the removal step). The data analysis algorithm calculates a process metric of percentage of oligonucleotides with proper observed fluorescence (e.g., presence of fluorescence after conjugation, absence of fluorescence after blocking group removal). The data analysis algorithm also calculates an uncertainty metric of a spatial variance of improper observed fluorescence. An iterative process is initiated to alter the pre-determined sequence of steps if the observed process metric and uncertainty metric do not meet established criteria. The criteria are determined based upon a user-input sequence uniformity level for the final oligonucleotides. For example, in some embodiments, for a high-uniformity product of 99.9% sequence accuracy, the rule for the percentage of oligonucleotides with proper observed fluorescence is greater than 99.99999%, and the rule for spatial variance of observed fluorescence is less than 0.00001 (errors/μm²)². For this case, an iterative process is utilized to repeat a sequence of nucleotide conjugation, post-conjugation rinse, and fluorescence measurement until the percentage of oligonucleotides with proper observed fluorescence is greater than 99.99999%, and the spatial variance of proper observed fluorescence is less than 0.00001 (errors/μm²)². The iterative process is then exited, and a new iterative process is initiated to control the accuracy of the blocking group removal process. That iterative process is utilized to repeat a sequence of blocking group removal, post-removal rinse, and fluorescence measurement until the percentage of oligonucleotides with proper observed fluorescence is greater than 99.99999%, and the spatial variance of proper observed fluorescence is less than 0.00001 (errors/μm².

### Example 5. Single-Molecule Device Fabrication

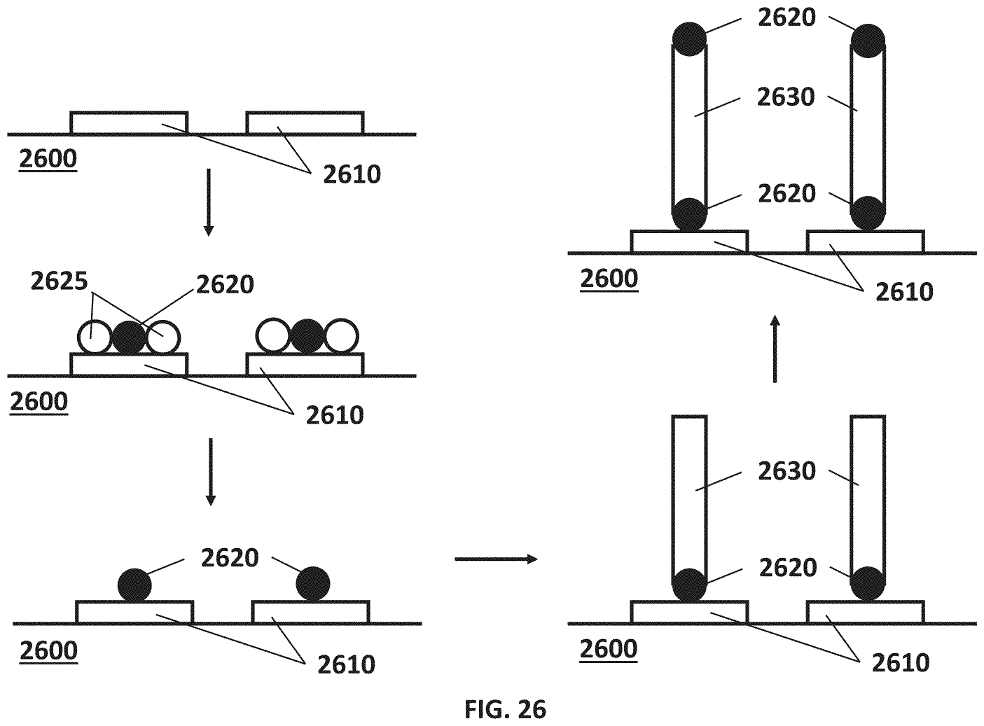

A single-molecule sensing device is fabricated by a controlled single-molecule fabrication process. The is detailed in FIG. 26. A solid support 2600 with binding sites 2610 is provided to a single-molecule fabrication instrument. Nanoparticle complexes comprising metal nanoparticles 2620 joined with fluorescent organic spacer particles 2625 are contacted with the solid support 2600, thereby allowing the nanoparticle complexes to deposit at each binding site 2610. After complex deposition, each binding site 2610 is optically observed to determine the presence of fluorescence, thereby suggesting the deposition of a nanoparticle complex at the binding site 2610. The fluorescent organic spacer particles 2625 are thermally released, leaving binding sites 2610 with a single metal nanoparticle 2620. The metal nanoparticles 2620 are then heated to a high temperature in the presence of a hydrocarbon gas, causing the catalytic formation of a single-walled carbon nanotube (SWNT) 2630 from the metal nanoparticle 2620. The fabrication is completed by depositing another metal nanoparticle 2620 at the terminus of each SWNT 2630. The final fabrication at each binding site is confirmed by atomic force microscopy.

Iterative processes are implemented during the fabrication to maximize the number of binding sites with proper fabrications at each step of the fabrication process. Separate iterative processes are implemented for complex deposition, spacer removal, nanotube formation, and final nanoparticle deposition. It is known that achieving proper and uniform SWNT formation requires careful control of the process temperature during the catalytic reaction. An iterative process for SWNT formation is configured to pause the fabrication process if the standard deviation of the process temperature during the catalytic reaction exceeds 5° C. or if the absolute value of the difference between the actual temperature and the set point temperature for the reaction is more than 20° C.

It so happens that the sensing device fabrication process is occurring in a laboratory in suburban Saskatchewan one frigid Saturday afternoon in January. An errant hockey puck impacts a laboratory window, as will happen from time to time, thereby admitting the bitterly cold air into the climate-controlled laboratory. A process control algorithm that implements the iterative process for the SWNT fabrication step retrieves the in-situ time-temperature history data from a single-molecule data set and determines based upon a trend of increasing standard deviation in the process temperature with time that the fabrication system is struggling to maintain a proper reaction temperature.

Upon making this determination, the process control algorithm sends a message to the cellular telephone of an on-call technician. The message reads, “Sorry to bother you but we have a bit of a temperature problem on the fabrication system. Should fabrication proceed or pause?” Upon receiving the message, the technician transmits an instruction back to the control algorithm to pause the fabrication process indefinitely. Upon reaching the laboratory later that afternoon, the technician performs a manual inspection of an in-process sensing device and determines that the temperature instability has caused irreparable damage to the devices in fabrication. The in-process devices are discarded, thereby excluding a defective batch from inventory. The technician then proceeds to tape cardboard over the hole in the window and gets the system prepared to start a new batch of sensing devices.

### Example 6. Single-Molecule Proteomic System Description

A single-molecule proteomic system is configured to perform a fluorescence-based affinity reagent binding assay such as the assay described in FIG. 20. The system includes a flow cell and a fluidics system, a detection device adjacent to the flow cell, a network of sensors, a process control system, and a network of processors. The flow cell is configured to display a polypeptide array such that each polypeptide on the polypeptide array is individually observable by the detection device at an individual address. The fluidics system is configured to store, transfer, and dispose of fluids throughout the single-molecule proteomic system, including transferring fluids to the flow cell and out of the flow cell. The detection device is configured to provide exciting radiation to the polypeptide array and detect emitted fluorescent radiation from individual addresses on the polypeptide array. The sensors are configured to collect physical measurement data from a plurality of individual components of the single-molecule proteomic system, such as temperature sensors, flow rate sensors, pressure sensors, and chemical sensors. The process control system is configured to actuate a plurality of components of the single-molecule proteomic system, such as actuating fluidic valves, actuating fluidic pumps, and actuating translational stage that control flow cell position and orientation. The network of processors is configured to obtain a single-polypeptide data set from the detection device and/or the network of sensors and utilize the single-polypeptide data set to implement one or more actions during a single-polypeptide fluorescence-based affinity binding assay.

The single-molecule proteomic system is configured to include a flow cell. The flow cell includes a solid support that is configured to display a polypeptide array. The solid support is a rigid, substantially planar body including at least one surface that is configured as a polypeptide display area. The polypeptide display area is patterned to control the deposition of polypeptides at individual, separated sites on the surface of the solid support. The solid support is joined to a second rigid, substantially planar body that is optically opaque adjacent to the polypeptide display area. The second body includes multiple fluidic lanes that are fabricated on the surface of the second body that contacts the solid support. Each fluidic lane includes a fluidic channel that is configured to transfer fluids through the flow cell, and a chamber that is configured to allow the contact of a fluid with the surface of the solid support including the polypeptide display area. Each fluidic lane has two fluidic port, one at each terminus of the fluidic lane. The fluidic lanes connect to a manifold that is configured to provide fluidic communication between the fluidics system and the flow cell through the fluidic ports of each lane. The single-molecule proteomic system is configured to provide polypeptides that are deposited on the solid support to form the polypeptide array, or receive a flow cell with a pre-formed polypeptide array. The multiple fluidic lanes of the flow cell are configured to permit flexible use, such as lanes dedicated to arrays of sample polypeptides and lanes dedicated to display of arrays of control polypeptides.

The flow cell of the single-molecule proteomics system is connected to a fluidics system. The fluidics system is configured to provide a plurality of fluids to the flow cell when the fluidics system is actuated by the process control system. The fluidics system includes a network of fluidic lines that are configured to inject and/or extract fluids from the flow cell. In some embodiments, the upstream region of the fluidics system includes a plurality of reservoirs including necessary process reagents, including buffers and affinity reagents. The upstream region also includes mixing manifolds that are configured to contact two or more fluids and completely mix them before the mixed fluid is transferred to the flow cell. The movement of fluids to and from the flow cell is accomplished by two pumps. The two pumps are configured to provide bidirectional fluid flow to the flow cell, such as driving a fluid through a fluidic lane from either fluidic port, or oscillating a packet of fluid back and forth through a fluidic lane. The fluidics system also includes a series of valves that are configured to control the direction and routing of fluids. Each fluidic lane is connected to at least one valve that controls fluid flow through the lane by process control system actuation. Additionally, valves are configured in upstream and downstream regions of the fluidics system to prevent unwanted flow of process reagents, such as the flow of used affinity reagents back to the storage reservoirs. The fluidics system further comprises a receiver that is configured to collect a prepared polypeptide sample and store it until the initiation of depositing a polypeptide array.

The flow cell of the single-molecule proteomic system is positioned adjacent to an objective of a detection device. The detection device is configured to transmit light radiation at an excitation wavelength from a laser through an optical system and through the objective to the flow cell. The excitation radiation is transmitted to the polypeptide array through the optically-opaque portion of the second body of the flow cell. The optical system is further configured to direct the excitation radiation to only a portion of the polypeptide array. The portion of the polypeptide array illuminated by the impinging laser radiation is controlled by a series of translational and/or rotational stages that are configured to incrementally adjust the position of the flow cell relative to the detection device. The optical system of the detection device is further configured to receive emitted fluorescent radiation from the polypeptide array, through the objective and optical system to a light sensor. The light sensor includes a pixel-based array that is configured to convert photons captured at a pixel into a voltage signal. In some embodiments the light sensor is configured to receive light from the same portion of the polypeptide array illuminated by the excitation laser. In some embodiments, each pixel on the array is corresponded to a physical address on the array where a fluorescent photon was emitted.

A network of sensors is integrated throughout the single-molecule proteomic assay system. The network of sensors is configured to provide physical measurement data from throughout the system. The sensors are configured to be located at locations that permit accurate measurement without impeding system functions. Sensors are integrated into particular components of the single-molecule proteomic assay system, including the fluidic system, flow cell, and detection device. The fluidic system includes a network of sensors, individually or collectively configured to collect data concerning fluid conditions and fluid transfer operations. The fluid system sensors are configured to transmit sensor data to a processor associated with the process control system. The flow cell includes a network of sensors, individually or collectively configured to collect data concerning flow cell fluid conditions, flow cell position and flow cell orientation. The flow cell sensors are configured to transmit sensor data to a processor associated with the process control system. The detection device includes a network of sensors, individually or collectively configured to collect data concerning detection device function, including aperture position sensors, dust sensors, and ambient light sensors. The detection device sensors are configured to transmit sensor data to a processor associated with a process control system.

The process control system integrates the hardware components of the single-molecule proteomic assay system with the processors. The process control system includes a network of electrical and data connections (e.g., wired or wireless data transmission lines), individually or collectively configured to provide control signals to the hardware components of the proteomic system. The network of electrical connections includes additional electronic components that are configured to generate electrical signals, including a voltage source. The process control system is configured to receive physical measurement data from the network of sensors and/or the detection device and transfer the data to a processor. The process control system is further configured to receive instructions from a processor and convert the instructions into electrical signals that actuate hardware components of the proteomic system. The network of electrical connections is configured to transmit the electrical signals from the process control system to a hardware component, thereby effecting the actuation of the hardware component. For example, the process control system has a data connection to an x-y position sensor for a translation stage that is configured to control flow cell position. The process control system is configured to relay the position data from the position sensor to a data processor. In turn, the data processor returns instructions to the process control system to alter the position of the translation stage. The process control system converts the instructions into a series of electrical impulses that actuate the translation stage to alter the position of the translation stage according to the instructions.

The single-molecule proteomic assay system also includes a network of processors. Two processors are physically located within the proteomic system. The first on-board processor is configured to receive data from the network of sensors and process the data on a process control algorithm that is implemented on the first on-board processor. The second on-board processor is configured to receive light sensor data from the optical system of the detection device and process the data on an image analysis algorithm that is implemented on the second on-board processor. The two on-board processors are further configured to collect and compose sensed data or data derived from the sensed data into single-polypeptide data sets and transmit the single-polypeptide data sets to a network of external processors. The network of external processors includes a processor associated with a terminal computer that is configured to implement a user interface algorithm for initiation, control, and termination of system processes. The network of external processors also includes a plurality of processors associated with mobile devices (e.g., tablets, cellular phones, etc.) that are configured to implement a user interface algorithm for remote control of system processes. The network of processors further includes a series of processors that are configured to implement an assay algorithm that implements a single-analyte process, such as the single-analyte processes set forth herein.

### Example 7. Single-Molecule Proteomic Assay Description

A single-molecule fluorescence-based affinity binding assay is implemented on the system described in Example 6. The assay provides a characterizing analysis of each observed polypeptide of an array of polypeptides at single-polypeptide resolution. In some embodiments, the assay is configured to provide identification of individual polypeptides, quantification of polypeptides at single-polypeptide resolution, and polypeptide property identification at single-polypeptide resolution.

A fluorescence-based binding assay is initiated with the formation of a polypeptide array. A series of fluids are transferred reagent reservoirs through each of four fluidic lanes of the flow cell to prepare the solid support surface for polypeptide deposition. The first fluid rinses particulate or adsorbed matter from the solid support surface and carries any removed matter out of the flow cell to a waste reservoir. A second fluid provides a passivation agent to the solid support surface to passivate any potential non-specific binding sites. An optional third fluid performs a final rinse of each fluidic lane before polypeptide deposition. After the flow cell preparation steps are complete, a polypeptide sample is split into three equal volumes and injected by the fluidics system into three of the four available fluidic lanes. In parallel, a control polypeptide mixture is injected by the fluidics system into the fourth fluidic lane. The injected fluids each comprise single polypeptides covalently conjugated to structured nucleic acid particles (SNAPs). The SNAPS are configured to deposit the single polypeptides at unique sites on the solid support surface to form an array of single polypeptides. The injected fluids are quiescently incubated in each fluidic lane for 1 minute to facilitate deposition of polypeptide-SNAP conjugates onto the solid support surface, then the incubated fluid volumes are gently oscillated back and forth in the fluidic lanes for 1 minute by patterned switching between the two bidirectional pumps. The injected fluids are again quiescently incubated for 1 minute to permit additional polypeptide-SNAP conjugate deposition. Any unbound polypeptide-SNAP conjugates are carried out of the flow cell by the injection of a rinsing fluid through each fluidic lane.

After formation of the four polypeptide arrays (3 sample, 1 control) in the four fluidic lanes, each polypeptide array is imaged to determine the addresses on the array that are occupied by a polypeptide-conjugates. Each array is subdivided into 1000 overlapping imaging regions. The imaging regions are sufficiently overlapped to ensure adequate cross-registration of images so that features are consistently identified during image analysis. Each imaging region is illuminated by a 488 nm laser to produce fluorescence from Alexa-Fluor 488 dye molecules that are coupled to the SNAP portion of each SNAP-polypeptide conjugate. Emitted fluorescence is detected in each imaging region by the light sensor of the optical system. Each image of each imaging region is transmitted to an on-board graphics processor unit (GPU) along with x-y position data provided by the process control algorithm from data obtained from the flow cell position sensor. The GPU corrects, processes, and registers each image to populate an initial single-polypeptide data set for each fluidic lane with data regarding the occupancy and physical location of each resolvable address on the solid support surface along with image processing quality metrics for each imaging region or array address.

The fluorescence-based affinity reagent binding assay is initiated after polypeptide array formation and initial imaging registration. The assay is cyclical, with each cycle including the steps of rinsing the flow cell, injecting a volume of Alexa-Flour 647-labeled affinity reagents into a fluidic channel including sample polypeptides, incubating the affinity reagents with the sample polypeptides, rinsing the fluidic channel to remove unbound affinity reagents, illuminating each imaging region of the sample polypeptide array with 647 nm light to excite fluorescence from any bound labeled affinity reagents, imaging each imaging region of the sample polypeptide array to determine the location of emitted fluorescent light, injecting an affinity reagent removal fluid into the fluidic channel, incubating the affinity reagent removal fluid with the sample polypeptides, rinsing the fluidic channel to remove released affinity reagents, and providing a final rinse of the flow cells to ensure removal of all process reagents. In some embodiments, each cycle include staggered operations for the remaining two sample fluidic lanes (if utilized), and optionally the control fluidic lane. For example, in some embodiments, affinity reagents are injected into the second sample fluidic lane as the first fluidic lanes is being imaged, and so forth. Each sensed fluorescence image for each imaging region of a polypeptide array is passed serially or in parallel from the optical system to the GPU image correction, processing, and registration. During each cycle, image data is added to the single-polypeptide data set for the fluidic lane, including data concerning the presence or absence of a detected affinity reagent at each array address along with image processing quality metrics for each imaging region or array address. During assay operations, the network of sensors transmits sensor data from each sensor at intervals requested by the process control algorithm. The process control algorithm records all sensor data in a second single-polypeptide data set for each lane, including time stamps and process codes for ongoing system processes at each time stamp. After a cycle has been completed for each applicable fluidic lane, a new cycle is initiated until the assay control algorithm determines that all affinity reagent binding measurements have been completed. The single-polypeptide data sets for a utilized fluidic lane, including the first single-polypeptide data set including the imaging data and the second single-polypeptide data set including the time-series of sensor data, are passed to the assay control algorithm for further analysis upon completion of data preparation by the GPU.

An iterative process is implemented during the fluorescence-based affinity reagent binding assay in one of two fashions. In a first fashion, a pre-determined sequence of affinity reagent measurements is selected by the assay control algorithm. An iterative process is implemented after an initial sequence of affinity reagent measurements has been performed to establish iterative control of the process outcome. The iterative process is terminated when a determinant criterium has been achieved. In a second fashion, a first affinity reagent measurement is selected by the assay control algorithm and each subsequent affinity reagent binding measurement or sequence of affinity binding reagent measurements is thereafter determined by an iterative process until a determinant criterium is achieved to exit the iterative process. In some embodiments, after the completion of an iterative process, additional affinity reagent binding measurements are performed before the assay is completed.

### Example 8. Defining Outcomes in a Single-Molecule Proteomic Assay

A fluorescence-based affinity reagent binding assay as described in Example 7 is performed on a single-analyte system as described in Example 6. The assay is initiated by a user who provides a polypeptide sample to the system and specifies the type of fluorescence-based affinity reagent binding assay to be performed. The user is prompted by the assay interface algorithm to select the type of assay to be performed and the stringency of the final result (i.e., least stringent, medium, or high stringency). The user inputs are provided to the assay control algorithm and the assay algorithm utilizes the inputs to configure outcomes for the assay.

Outcomes are automatically configured by the assay control algorithm for the fluorescence-based affinity reagent binding assay based upon the user inputs provided to the assay control algorithm. Each type of assay has three primary outcomes: a defined outcome for deliverable polypeptide information based upon the selected type of assay; a defined outcome for information confidence level based upon the selected stringency; and a targeted outcome for the assay length. Table XI provides listings of assay type, assay description, and outcome specifications for each of the three configured outcomes.

Most assays that are performed on the single-analyte system are configured to identify at least 90% of the available polypeptides on a polypeptide array. The defined outcome of 90% identification of individual polypeptides is based upon a pre-determined rate of attrition for polypeptides from the polypeptide array, as well as the small probability that some polypeptides will not be identifiable based upon the observed affinity binding measurements. The targeted outcome of total cycle number is based upon the type of selected assay. In some embodiments, assays that produce more limited information (e.g., single-species quantification) are accomplished using a smaller set of affinity reagents due to the predictability of high-probability affinity reagent binding patterns for a specific polypeptide.

TABLE XI

| | | | |
|---|---|---|---|
| Assay Description | Outcome 1 | Outcome 2 | Outcome 3 |

Whole Sample Identification

| | | | |
|---|---|---|---|
| Known Sample | Determine the | Confidence scores for | ≤300 measurement |
| Source: Determine | individual identity of | each polypeptide | cycles |
| the individual identity | >90% of | identification: | |
| of each polypeptide | polypeptides on the | Least: >0.9 | |
| on the polypeptide | array | Medium: >0.99 | |
| array | | High: >0.999 | |
| Unknown Sample | Determine the | Confidence scores for | ≤300 measurement |
| Source: Determine | individual identity of | each polypeptide | cycles |
| the individual identity | >50% of | identification: | |
| of each polypeptide | polypeptides on the | Least: >0.9 | |
| on the polypeptide | array | Medium: >0.99 | |
| array | | High: >0.999 | |

Whole Sample Quantification

| | | | |
|---|---|---|---|
| Known Sample | Determine the | Confidence scores for | ≤300 measurement |
| Source: Determine | individual identity of | each polypeptide | cycles |
| the individual identity | >90% of | identification: | |
| of each polypeptide | polypeptides on the | Least: >0.9 | |
| on the polypeptide | array; tally 90% of | Medium: >0.99 | |
| array, then tally the | identified | High: >0.999 | |
| quantity of each | polypeptides | | |
| identified species of | | | |
| polypeptide | | | |
| Unknown Sample | Determine the | Confidence scores for | ≤300 measurement |
| Source: Tally the | individual identity of | each polypeptide | cycles |
| number of | >50% of | identification: | |
| polypeptides with | polypeptides on the | Least: >0.9 | |
| probabilistically | array; tally 90% of | Medium: >0.99 | |
| aligned binding | characterized | High: >0.999 | |
| profiles; provide | polypeptides | | |
| identities for | | | |
| polypeptides if | | | |
| possible | | | |

Single Species Identification

| | | | |
|---|---|---|---|
| Identify the presence | Determine the | Confidence scores for | ≤50 measurement |
| on a polypeptide | individual identity of | each polypeptide | cycles |
| array of at least one | >90% of | identification: | |
| copy of one known | polypeptides on the | Least: >0.99 | |
| species of | array; identify | Medium: >0.999 | |
| polypeptide | presence or absence | High: >0.9999 | |
| | of target polypeptide | | |
| | among identified | | |
| | polypeptides | | |

Single Species Quantification

| | | | |
|---|---|---|---|
| Quantify the number | Determine the | Confidence scores for | ≤50 measurement |
| on a polypeptide | individual identity of | each polypeptide | cycles |
| array of copies of one | >90% of | identification: | |
| known species of | polypeptides on the | Least: >0.99 | |
| polypeptide | array; identify the | Medium: >0.999 | |
| | number of copies of | High: >0.9999 | |
| | the target polypeptide | | |
| | among the identified | | |
| | polypeptides | | |

Polypeptide Panel Identification

| | | | |
|---|---|---|---|
| Identify the presence | Determine the | Confidence scores for | ≤200 measurement |
| on a polypeptide | individual identity of | each polypeptide | cycles |
| array of at least one | >90% of | identification: | |
| copy of each species | polypeptides on the | Least: >0.99 | |
| of polypeptide for a | array; identify | Medium: >0.999 | |
| group of known | presence or absence | High: >0.9999 | |
| species of | of target polypeptide | | |
| polypeptides | among identified | | |
| | polypeptides | | |

Polypeptide Panel Quantification

| | | | |
|---|---|---|---|
| Quantify the number | Determine the | Confidence scores for | ≤200 measurement |
| of copies on a | individual identity of | each polypeptide | cycles |
| polypeptide array of | >90% of | identification: | |
| each observed species | polypeptides on the | Least: >0.99 | |
| of group of known | array; quantify the | Medium: >0.999 | |
| species of | number of copies of | High: >0.9999 | |
| polypeptides | the target | | |
| | polypeptides among | | |
| | the identified | | |
| | polypeptides | | |

Non-Native Polypeptide Identification

| | | | |
|---|---|---|---|
| Identify the presence | Determine the | Confidence scores for | ≤350 measurement |
| on a polypeptide | individual identity of | each polypeptide | cycles |
| array of at least one | >90% of | identification: | |
| non-native | polypeptides on the | Least: >0.9 | |
| polypeptide in a | array; determine the | Medium: >0.99 | |
| sample from a known | identity of at least | High: >0.999 | |
| source (e.g., bacterial | one non-native | | |
| polypeptide in a | polypeptide | | |
| human sample) | | | |

Non-Native Polypeptide Quantification

| | | | |
|---|---|---|---|
| Quantify the number | Determine the | Confidence scores for | ≤350 measurement |
| of a non-native | individual identity of | each polypeptide | cycles |
| polypeptides on a | >90% of | identification: | |
| polypeptide array | polypeptides on the | Least: >0.9 | |
| from a sample with a | array; tally all | Medium: >0.99 | |
| known source (e.g., | identified non-native | High: >0.999 | |
| bacterial polypeptide | polypeptides | | |
| in a human sample) | according to identity | | |

Proteoform Identification

| | | | |
|---|---|---|---|
| Identify the presence | Determine the | Confidence scores for | ≤50 measurement |
| on a polypeptide | individual identity of | each polypeptide | cycles |
| array of one or more | >90% of | identification: | |
| polypeptide | polypeptides on the | Least: >0.9 | |
| proteoforms for a | array; identify the | Medium: >0.99 | |
| species of | presence or absence | High: >0.999 | |
| polypeptide | of at least one copy | | |
| | of each of the | | |
| | targeted proteoforms | | |

Proteoform Quantification

| | | | |
|---|---|---|---|
| Quantify the copy | Determine the | Confidence scores for | ≤50 measurement |
| numbers of each | individual identity of | each polypeptide | cycles |
| proteoform of a set of | >90% of | identification: | |
| proteoforms for a | polypeptides on the | Least: >0.9 | |
| species of | array; tally at least | Medium: >0.99 | |
| polypeptide | 90% of identified | High: >0.999 | |
| | polypeptides to | | |
| | quantify copy | | |
| | numbers of each | | |
| | proteoform | | |

### Example 9. Analyzing Process Metrics and Rules in a Single-Analyte System

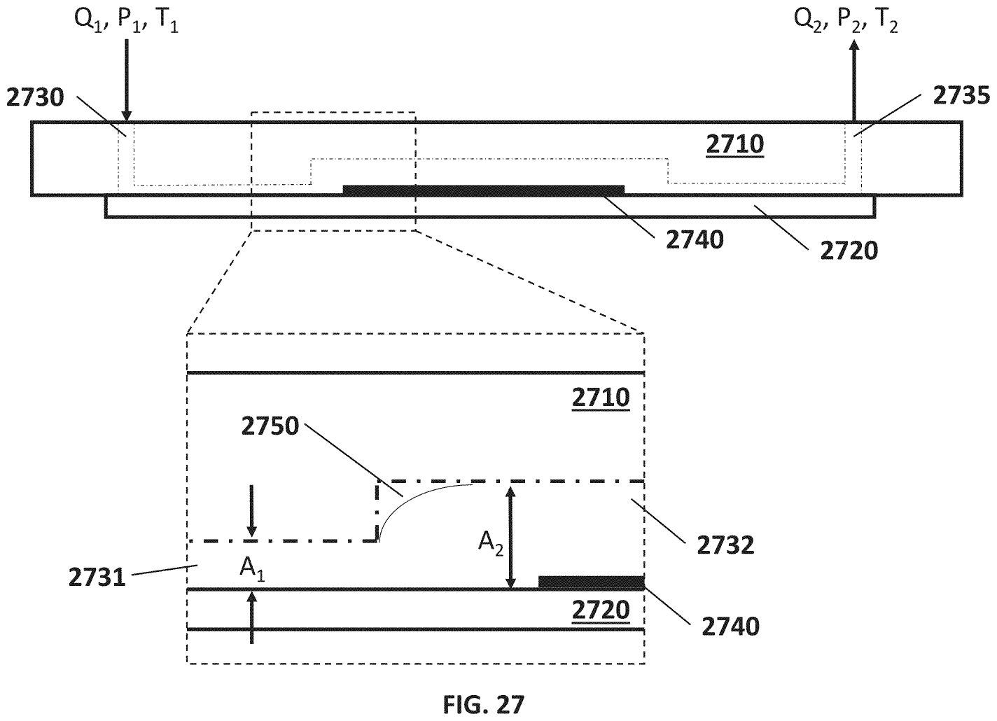

A flow cell for a single-analyte system, such as the system described in Example 6, is analyzed to determine the impact of various process metrics on the success of rinse steps during fluidic operations. FIG. 27 depicts a cross-sectional schematic of a fluidic lane of a flow cell comprising a rigid, substantially planar solid support 2720 that is joined to a rigid, substantially planar second body 2710. The second body 2710 includes a fluidic lane including two ports 2730 and 2735, as well as flow channels 2731 and a chamber 2732 including a polypeptide display region 2740. The flow channels 2731 are characterized as having an average first cross-sectional area A₁ that is orthogonal to the fluid flow direction, and the chamber 2732 has a larger cross-sectional area A₂ that is orthogonal to the fluid flow direction. Consequently, for a given fluid flow rate, the average fluid velocity in the chamber 2732 is expected to be less than the average fluid velocity in the flow channels 2731.

Sensors are located in the fluidics system external to ports 2730 and 2735. The sensors are able to provide measurements of process metrics such as fluid volumetric flow rate Q, fluid pressure P, and fluid temperature T upstream and downstream of the flow cell, depending upon which direction fluid is being driven. In turn, in some embodiments, the measured process metrics is used to estimate additional flow process metrics such as average fluid channel velocity, average fluid chamber velocity, fluid entrance viscosity, fluid exit viscosity, and flow cell pressure drop. In some embodiments, variability metrics are calculated for fluid flow measurements provided by the sensors. For example, a difference in measured volumetric flow rate between an inlet port and an outlet port of the flow cell provides an approximate uncertainty metric for the volumetric flow measurement. In some embodiments, variances or standard deviations of sensed parameters are calculated from time-series data (e.g., flow rate vs. time during steady-state flow) to provide uncertainty metrics for fluid flow.

An important consideration in flow cell operations is the potential for reagent accumulation in stagnant flow regions 2750 of the flow cell. In some embodiments, residual reagents from a prior fluidic operation affect subsequent assay steps. For example, in some embodiments, residual affinity reagents from a first binding measurement mixes with different affinity reagents from a subsequent binding measurement, potentially creating false positive binding events. Likewise, in some embodiments, residual affinity reagent removal reagents diffuse from stagnant regions 2750 to the polypeptide display region, potentially causing unwanted dissociation of affinity reagents from polypeptide binding targets. Prior to the deployment of a fluorescence-based affinity reagent binding assay system, flow cells are thoroughly tested to determine rinsing protocols that most effectively remove process reagents from stagnant regions. In some embodiments, pre-deployment testing also includes the development of algorithm-based models for estimating the amount of residual reagent after each wash cycle so that the binding measurement data is adjusted to account for this source of measurement uncertainty.

A set of dry flow cells are used to measure the effectiveness of rinse procedures. The entirety of each fluidic lane is measured by fluorescent microscopy to establish the background fluorescence of the flow cell materials in the optical path to the fluidic lane. Each 100 microliter (μl) fluidic lane is divided into 100 imaging regions so that background fluorescence is measured in high resolution. After background fluorescence has been spatially measured, a fluid including a measured concentration of fluorescent dye is injected into the flow cell. After each fluidic lane has been completely filled with the fluorescent fluid, each fluidic lane is again measured by fluorescence microscopy to establish the maximum spatial distribution of fluorescence at time zero. Next, a rinse buffer including no fluorescent dye is injected into each fluidic lane. The rinse buffer is injected into each fluidic lane in 5 μl increments, thereby displacing 5 μl of fluid from the fluidic lane. Each injection of rinse buffer takes 5 seconds (s). After each rinse buffer injection, fluid flow is paused by closing valves on both sides of the flow cell, and each fluidic lane is imaged by fluorescence microscopy. The fluid is displaced in 5 μl increments for 100 iterations until each fluidic lane has received 5 volumes worth of rinse buffer. The fluid displacement measurements are repeated with faster 5 μl fluid injection times of 1 s, 0.5 s, and 0.1 s.

After each set of 100 images per fluidic lane are collected, the images are provided to an image analysis algorithm implemented on a graphics processor unit (GPU). The image algorithm integrates the sensor-derived photon counts over the entire fluidic channel to calculate the total fluorescence of the polypeptide display region of the fluidic lane at the imaging time. The image analysis algorithm also generates a spatial map of sensor-derived photon counts for the entire fluidic lane at the imaging time. After all measurements are completed, the image analysis algorithm utilizes the time-sequenced data to determine the time (tₘᵢₙ) until fluorescence has been returned to background total photon count in the polypeptide display region as a function of fluid injection rate. The data collected after tₘᵢₙ is further analyzed to determine the total remaining photon counts in stagnant regions. The total remaining photon counts in stagnant regions are regressed as a function of time and flow rate to determine a rinsing model for the flow cell. The model provides average removal of fluids from stagnant regions of the flow cell as a function of time and rinsing fluid flow rate. The model output is stored in a single-polypeptide reference data set including t₉₀, t₉₉, and t_{99.9} values (rinse times for 90%, 99% or 99.9% rinsing of stagnant regions) as a function of flow rate.

A rule concerning maximum flow rate is implemented for a fluorescence-based affinity reagent binding assay to prevent damage to the polypeptide array by flow turbulence. The maximum flow rate for the flow cell is limited to a volumetric flow rate of 10 microliters/second (μl/s). Based upon the rule, the assay control algorithm automatically configures rinse processes to occur at a flow rate of no more than 10 μl/s. The assay control algorithm defaults to configure rinse processes for affinity reagent removal to have a low stringency. Rinse processes are configured to occur at 10 μl/s for a length of time corresponding to the t₉₀ for that flow rate.

### Example 10. Image Processing Process Metrics

A fluorescence-based affinity reagent binding assay is performed utilizing systems and methods described in Example 6-9. An iterative process is implemented during the fluorescence-based affinity reagent binding assay to, in part, ensure that affinity reagent binding measurements produce data quality that is sufficient for polypeptide characterization. The iterative process is utilized to obtain a plurality of image quality metrics from fluorescent microscopy images and determine if further actions need to be implemented due to data quality issues.

Each affinity reagent binding measurement comprises a set of 1000 fluorescence microscopy images of a polypeptide array that has been incubated with a fluorescently-labeled affinity reagent. Each image captures a region of the array that at least partially overlaps with a region captured by an adjacent image. Due to the ordered patterning of polypeptide binding sites, fluorescent microscopy images are expected to demonstrate ordered patterns with fluorescence detected at array addresses where affinity reagents transmit a fluorescent signal when irradiated by an exciting radiation field provided by a visible laser. Fluorescence is detected by the capture of emitted fluorescent photons on a CMOS sensor. The resolution of the fluorescent detection system is sufficient that each array address is detected by a plurality of pixels.

Fluorescence-based affinity reagent binding measurements are selected and performed by a single-analyte process algorithm that includes an iterative process algorithm. In some embodiments, the iterative process algorithm that controls the image analysis process is a nested iterative process within a larger iterative process controlling measurement sequences. Each round of affinity reagent binding measurements includes capturing the set of 1000 fluorescence microscopy images. As each fluorescence microscopy image of the set of 1000 fluorescence microscopy images is collected, the image is provided to an image processing algorithm that is implemented on a graphics processor unit (GPU) included within the single-analyte system. The image processing algorithm on the GPU implements a trained image classification algorithm that identifies clusters of pixels that have detected emitted photons. The image classification algorithm is trained to determine a peak intensity metric, an intensity paraboloid metric, and a peak signal-to-noise metric for each identified cluster of pixels on each collected microscopy image. Any array address with peak intensity metric, intensity paraboloid, and peak signal-to-noise-ratio metrics that exceed defined threshold values is assigned a binding metric of “BIND.” All other array addresses that fail to meet one or more threshold values are assigned a binding metric of “NO BIND.” The calculated image classification metrics for each image are stored in a single-analyte data set for that image, with the single-analyte data set comprising the image classification metrics for each identified array address. Each image single-analyte data set is provided to the image processing algorithm after image processing is complete. The image processing algorithm aligns overlapping image regions and aligns them based upon fluorescence signal patterns. Calculated image classification metrics for each imaged array address are transferred by the image processing algorithm into a compiled full array single-analyte data set, with overlapping addresses from each image averaged before being stored in the full array single-analyte data set.

The full array single-analyte data set is passed from the image processing algorithm to a decision algorithm of the iterative process algorithm. The full array single-analyte data set is also simultaneously passed to a cloud-based, decentralized network that implements multiple complex decision algorithms. The decision algorithm of the iterative process algorithm calculates a total observed binding count for the affinity reagent (i.e., the total number of sites with a “BIND” metric). The decision algorithm provides sample information (e.g., sample type) and the affinity reagent information (e.g., affinity reagent identity) to a cumulative databased comprising single-analyte data sets from prior single-analyte processes and requests a predicted total observed binding count for the current measurement. In some embodiments, based upon the predicted total observed binding count calculated from the cumulative data source, the decision algorithm configures a rule that the observed total binding count must be no more than 20% higher than the predicted total binding count and no less than 80% lower than the predicted total binding count (e.g., more sensitive to false positives than false negatives). In some embodiments, if the observed total binding count falls within the range defined by the rule, the binding measurement is accepted and the decision algorithm instructs the iterative process algorithm to perform the next step of the single-analyte process. In some embodiments, if the observed total binding count falls outside the range defined by the rule, the binding measurement is rejected and the decision algorithm instructs the iterative process to re-perform the binding measurement after all other binding measurements in a pre-determined measurement sequence have been completed.

In parallel, the full array single-analyte data set is passed to the cloud-based, decentralized network of decision algorithms. The decentralized network of decision algorithms apply differing models that calculate the likelihood that the observed fluorescence binding data is due to an outlying condition (e.g., a rarely-observed phenotype) rather than measurement error or bias. In some embodiments, some algorithms of the decentralized network of decision algorithms continually update based upon the receipt of new single-analyte data sets for differing affinity reagent binding measurements. In some embodiments, if one or more algorithms of the decentralized network of algorithms determines a likelihood that the observed fluorescence binding data is due to an outlying condition, the algorithm will push an instruction back to the iterative process algorithm to retain the binding data for the measured affinity reagent and forego re-performing the binding measurement at the end of the single-analyte process.

### Example 11. Inferential Determination of Process Error

A fluorescence-based affinity reagent binding assay is performed utilizing systems and methods described in Example 6-10. A human user provides to a single-analyte system a sample including purified polypeptides that are each individually conjugated to a single-nucleic acid deposition group. The nucleic acid deposition groups are labeled with 10 Alexa Fluor-488 fluorophores that are utilized by the single-analyte system to identify the presence of nucleic acid deposition group and polypeptide when deposited on a solid support.

The single-analyte system performs a sequence of pre-iterative steps to prepare the system for data collection. The sample including the purified polypeptides is pumped into a fluidic cell in the single-analyte system by a fluidics system. The sample is directed to a solid support within the fluidic cell that includes a patterned deposition array that is configured to electrostatically bind the nucleic acid deposition groups at individual sites on the patterned array. The sample is contacted with the solid support for 5 minutes, then a rinsing buffer is passed through the fluidic cell by the fluidics system for 30 seconds to remove any unbound sample. In some embodiments, after the rinsing is completed, the entire polypeptide-deposited array is imaged by fluorescence microscopy at 488 nm and the initial imaging data is stored in a preliminary single-analyte data set that is used to determine which array addresses are occupied by a polypeptide. Concurrently, a set of instrument metadata, including sensor measurements from an array of sensors throughout the single-analyte system, is stored in a second single-analyte data set.

An iterative process is initiated and the preliminary single-analyte data set is provided to an image analysis algorithm. The image analysis algorithm utilizes the fluorescence microscopy data to determine the initial observed total site occupancy of the patterned polypeptide array according to the method described in Example 10. The initial observed total site occupancy metric is calculated by the image analysis algorithm. According to the rule configured for the initial observed total site occupancy metric (>95% array site initial occupancy), the metric falls far below the threshold value for proceeding with the fluorescence-based affinity binding assay. In some embodiments, the process control algorithm implements an action to pause the assay until the cause of the poor array occupancy is determined.

Based upon the low initial observed total site occupancy metric, the system sets five hypotheses for sources of the failure: defective fluidic cartridge; imaging sensor malfunction; exciting laser malfunction; or improper sample deposition; or improper sample preparation. A decision algorithm of the iterative process algorithm applies an inferential approach to determine the most probable cause of the poor array occupancy.

Laser diode sensor measurements are pulled from the second single-analyte data set and provided to the decision algorithm. The laser diode sensor measurements are determined to show normal laser function at expected intervals corresponding to the laser actuation. Hypothesis 3 is determined to be low likelihood and is de-prioritized. Next, the single-analyte system re-initiates the imaging sensor and collects a new image at a control region of the array. The new image is processed by the image analysis algorithm and the data is compared to an image of the same control region from the prior data set. Minimal differences in array patterning are observed. Hypothesis 2 is de-prioritized.

The decision algorithm requests information regarding outcomes of single-analyte processes utilizing fluidic cells with the same batch number as the fluidic cell utilized in the current run. The decision algorithm queries two data sources: a cumulative database of completed assay data; and any instruments currently running a single-analyte process. The decision algorithm forwards the batch number of the current fluidic cell and requests outcome data from the two data sources. Data returned to the decision algorithm from operating instruments indicates that 10% of instruments utilizing fluidic cells from the same batch are experiencing similar low initial observed array occupancy rates. Data returned to the decision algorithm from the cumulative dataset indicates that about 50% of arrays were properly prepared by a second round of sample incubation, although less than 1% of the recovered arrays had an initial observed array occupancy rate as low as the current array.

Based upon the data provided from the two data sources, the decision algorithm infers that the most likely cause of the failure is hypothesis 1, a defective fluidic cartridge. The decision algorithm provides a prompt to an operator requesting feedback on whether to proceed with a second sample incubation to further test the favored hypothesis. The operator receives a prompt on a portable device requesting input regarding the array occupancy problem and transmits an instruction back to the instrument to not proceed with further testing. The single-analyte system discontinues the process and discards the fluidic cartridge. The operator provides an instruction to re-initiate the assay with remaining sample. The instrument carries out the user-provided instruction with a fluidic cell chosen from a different batch number than the previous cell.

### Example 12. Iterative Decoding During a Single-Molecule Assay

A fluorescence-based affinity reagent binding assay is performed utilizing systems and methods described in Example 6-11. A human user provides to the single-analyte system a sample including polypeptides derived from human blood serum. The blood serum sample has been provided by a patient in remission from colon cancer to determine if any deleterious isoforms of cancer biomarker p53 are detected within the blood serum sample following a round of chemotherapy. The user instructs the system to implement a fluorescence-based affinity reagent binding assay and specifies that the system is to identify the presence or absence of a panel of twelve p53 isoforms. The user specifies high stringency for the analysis. High stringency indicates a 99.9% likelihood that the observed set of affinity reagent binding measurements corresponds to the called polypeptide identity.

Based upon the specified isoform panel analysis, the assay control algorithm recalls a single-polypeptide data set including cumulative data from prior analyses of p53 isoforms on the system. The assay control algorithm utilizes the cumulative data to configure a series of 30 affinity reagents that are calculated to have a greater than 99% chance of producing a high stringency identification of any of the twelve p53 isoforms. The assay control algorithm configures a sequence of affinity reagent measurements of the 30 affinity reagents, with the measurement sequence structured to begin with affinity reagents that most distinguish p53 isoforms from non-p53 polypeptides, followed by affinity reagents that distinguish various p53 isoforms from each other.

A polypeptide array is prepared from the blood serum sample. The polypeptide array includes approximately 9.5×10⁹ polypeptides from the serum sample, and an additional 0.5×10⁹ internal standard polypeptides as an internal control. The polypeptide array is prepared to ensure that at least 99% of unique polypeptide binding sites are occupied by a polypeptide, and at least 99% of occupied polypeptide binding sites include no more than one polypeptide. Each polypeptide binding site is separated from adjacent polypeptide binding sites by 300 nm such that each binding site is individually resolvable by fluorescence optical microscopy. Presence or absence of binding of each affinity reagent is measured at each array binding site by detecting the presence or absence of a fluorescent signal from fluorescently-labeled affinity reagents at the binding sites for each affinity reagent.

The assay, as configured based upon the cumulative data, requires the system to perform the steps of: performing binding measurements of the first 10 affinity-reagents (p53-identifying), pausing to determine which array sites are most likely to include p53, and performing binding measurements for the remaining 20 affinity reagents (isoform specific reagents. During the performing the binding measurements, an iterative process is invoked to monitor fluorescence microscopy imaging data quality metrics and alter the assay sequence to repeat measurements if images are of insufficient quality. During the pausing, array sites that are unlikely to include p53 isoforms are excluded removed from a single-polypeptide data set to decrease the time for data analysis. A site is excluded from further analysis if the site has a calculated likelihood score for each p53 isoform of less than 0.01. During the performing binding, a second iterative process is invoked to pause the assay when at least ten sites have been identified as including a deleterious p53 isoform.

The identity of the polypeptide at each array site is determined using a likelihood score. Based upon the high stringency criterium for the assay, a polypeptide at an array site is considered to be identified when the likelihood score exceeds 0.999. The assay is configured to discontinue when at least ten sites attain a likelihood score of 0.999 for a deleterious p53 isoform. In some embodiments, the likelihood score is calculated as:

$$LS ( {I}_{n} ) = \frac{L ( {I}_{n} )}{{\sum }_{1}^{N} L ( {I}_{n} )} \qquad ( 1 )$$

where LS(Iₙ) is the likelihood score of a polypeptide at an array site being a polypeptide with identity Iₙ, L(Iₙ) is the likelihood function of a polypeptide at an array site being Iₙ given the observations made at the array site, and Pₙ represents an n^{th} protein from a set of N proteins from which Iₙ is identified. The likelihood function is calculated as:

L(Iₙ)=Π₁^{M}P(θₘ=Iₙ) (2)

where P(θₘ=Iₙ) is the probability of observation θₘ being made for polypeptide identity Iₙ and the likelihood function is the product of the probabilities of observation for In over M observations. For example, if three observations of a polypeptide array site are made, and the likelihoods of the observed measurements being made are 10%, 25%, and 99% respectively if the polypeptide is assumed to be p53, then the likelihood function at the array site is calculated from equation 2 as:

L(p53)=(0.10)*(0.25)*(0.99)=0.02475.

In some embodiments, this calculation is repeated for every possible polypeptide amongst a set of known polypeptides. In some embodiments, the likelihood functions for each possible polypeptide are used in equation 1 to calculate the likelihood score for each polypeptide.

The polypeptide array comprising the polypeptides from the blood serum sample is analyzed on the single-analyte system. After the completion of the first iterative process, binding measurements for the first 10 unique affinity reagents at each site on the polypeptide array are analyzed to compute a likelihood score for each p53 isoform. Approximately 70,000 sites are determined to have a likelihood score above the minimum threshold of 0.01. An iterative process is initiated and the binding of the next affinity reagent is measured. After each binding measurement, the likelihood score for each of the 70,000+p53 candidates is calculated. An additional termination process metric for confirmed identities of deleterious p53 candidates is populated in a single-polypeptide data set. The termination process metric is incremented up by a unit each time a candidate polypeptide has an identity likelihood score of above 0.999 for five consecutive measurement cycles.

After the 17^{th} unique affinity reagent has been measured, a first deleterious p53 isoform achieves the criterium of a likelihood score of 0.999 for five consecutive measurement cycles, and the termination process metric is incremented to 1 in the single-polypeptide data set. After the 24^{th} unique affinity is measured on the polypeptide array, 11 deleterious p53 isoforms are identified by the likelihood score criterium. The iterative process is terminated, having achieved the determinant criterium of greater than 10 identified deleterious p53 isoforms. The single-analyte process is discontinued, and the remaining 6 unique affinity reagents are not utilized. Based upon the presence of the deleterious p53 isoforms, a medical professional determines that a trace amount of cancer cells remain and prescribes an additional round of chemotherapy.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces disclosed herein and described with respect to the Figures. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims,

1. A method for controlling an iterative detection process for detecting a polypeptide at single-molecule resolution, the method comprising performing an iterative detection process in a detection system until a determinant criterion has been achieved, wherein the detection system is configured to obtain a physical measurement of the polypeptide at single-polypeptide resolution, and wherein the iterative detection process comprises at least two cycles, each cycle comprising the steps of:
a) determining an uncertainty metric for the polypeptide based upon a data set acquired from the detection system;
b) implementing an action on the detection system based upon the uncertainty metric; and
c) updating the data set after implementing the action on the detection system.

2. The method of claim 1, wherein the determinant criterion comprises an unforced determinant criterium.

3. The method of claim 2, wherein the unforced determinant criterion is selected from the group consisting of:
ii. a fixed number of the cycles;
iii. a maximum number of the cycles;
iv. a minimum number of the cycles;
v. the uncertainty metric traversing a threshold value;
vi. a categorized value of the uncertainty metric changing from a first categorized value to a second categorized value;
vii. a trend in the uncertainty metric; and
viii. a pattern in the uncertainty metric.

4. (canceled)

5. (canceled)

6. The method of claim 3, wherein the fixed number of cycles, the maximum number of cycles, or the minimum number of cycles is determined after completing a first cycle of the at least two cycles.

7. The method of claim 3, wherein the fixed number of cycles, the maximum number of cycles, or the minimum number of cycles is determined based upon a default value or a user-defined value.

8. (canceled)

9. The method of claim 7, wherein the fixed number of cycles, the maximum number of cycles, or the minimum number of cycles is determined after completing a first cycle of the at least two cycles.

10. (canceled)

11. (canceled)

12. The method of claim 3, wherein the threshold value is determined based upon a preliminary data set.

13. The method of claim 3, wherein the threshold value is a default value or a user-defined value.

14. The method of claim 3, wherein the first categorized value or the second categorized value is a member of a binary pair group.

15. The method of claim 3, wherein the determinant criterion comprises the categorized value of a first uncertainty metric changing and the categorized value of a second uncertainty metric changing.

16. The method of claim 3, wherein the determinant criterion comprises the categorized value of a first uncertainty metric changing and the categorized value of a second uncertainty metric not changing.

17. The method of claim 3, wherein the trend comprises an increasing, decreasing, or neutral trend for the uncertainty metric over at least two of the cycles.

18. The method of claim 3, wherein the pattern comprises a converging, diverging, oscillatory, or static pattern for the uncertainty metric.

19. The method of claim 3, wherein the obtaining a final characterization of the single analyte comprises identifying the single analyte, determining a physical property of the single analyte, determining an interaction of the single analyte, determining a structure of the single analyte, or a combination thereof.

20. The method of claim 3, wherein the method comprises performing the iterative process until two or more determinant criteria have been achieved.

21. The method of claim 1, wherein the determinant criterion comprises a forced determinant criterion.

22. The method of claim 21, wherein the forced determinant criterion comprises a user input or a system feedback.

23. The method of claim 22, wherein the user input comprises an input selected from the group consisting of:
i. an instruction to discontinue the iterative detection process;
ii. an instruction to discontinue the iterative detection process;
iii. an instruction to alter a sequence of steps of the iterative detection process;
iv. an instruction to alter a sequence of steps of the iterative detection process;
v. information identifying a trend in the uncertainty metric;
vi. information identifying a pattern in the uncertainty metric;
vii. information identifying a categorized value of the uncertainty metric; and
viii. information identifying of a characterization of the polypeptide.

24. The method of claim 22, wherein the determinant criterion comprises feedback selected from the groups consisting of:
i. a reagent level or rate of consumption;
ii. an addressable hardware failure mode;
iii. a non-addressable hardware failure mode;
iv. a software failure mode;
v. an environmental condition; and
vi. an unexpected external condition.

25. The method of claim 1, wherein the action is selected from the groups consisting of:
i. pausing the iterative detection process;
ii. altering a sequence of steps for the iterative detection process;
iii. identifying a next step of a sequence of steps for the iterative detection process;
iv. performing a related process on the polypeptide; and
v. performing a related process on a second polypeptide.

26. The method of claim 25, wherein the pausing the iterative detection process further comprises an action selected from the group consisting of reconfiguring the detection system, recalibrating the detection system, repairing the detection system, transmitting an instruction or information to a second detection system, adding a second polypeptide to the detection system, stabilizing the polypeptide in the detection system, refreshing a computer-implemented algorithm, updating a computer-implemented algorithm, receiving a user input, and a combination thereof.

27. The method of claim 25, further comprising, after step b) and before step c) resuming the single-analyte process.

28-133. (canceled)

134. The method of claim 1, wherein the single analyte is derived from a biological sample.

135. The method of claim 134, wherein the single analyte comprises a nucleic acid, a lipid, a polypeptide, a polysaccharide, a metabolite, a cofactor, or a combination thereof.

136-170. (canceled)

171. A method for controlling a single-analyte process, the method comprising performing an iterative process until a determinant criterion has been achieved, wherein the iterative process comprises at least two cycles, each cycle comprising the steps of:
a) combining data from a single-analyte data set comprising data from more than one data source to determine a process metric for a single analyte;
b) implementing an action on a single-analyte system based upon the process metric, wherein the single-analyte system comprises a detection system that is configured to obtain a physical measurement of the single analyte at single-analyte resolution; and
c) updating the single-analyte data set after implementing the action on the single-analyte system.

172. A method for controlling the processes of a single-analyte process, the method comprising performing an iterative process until a determinant criterion has been achieved, wherein the iterative process comprises at least two cycles, each cycle comprising the steps of:
a) determining a process metric for a single analyte based upon a single-analyte data set; and
b) implementing an action on a single-analyte system that alters a source of uncertainty based upon the process metric, wherein the single-analyte system comprises a detection system that is configured to obtain a physical measurement of the single analyte at single-analyte resolution; and c) updating the single-analyte data set after implementing the action on the single-analyte system.

173. A method for controlling the processes of a single-analyte process, the method comprising performing an iterative process until a completion criterion has been achieved, wherein the iterative process comprises at least two cycles, each cycle comprising the steps of:
a) determining a curated uncertainty metric for a plurality of single analytes based upon a single-analyte data set;
b) implementing an action on a single-analyte system based upon the curated uncertainty metric, wherein the single-analyte system comprises a detection system that is configured to obtain a physical measurement at single-analyte resolution of each single analyte of the plurality of single analytes; and
c) updating the single-analyte data set after implementing the action on the single-analyte system.
